(19) United States
(12) Patent Application Publication
(10) Pub. No.: US 2024/0400554 A1
(43) Pub. Date: Dec. 5, 2024

(54) MONOACYLGLYCEROL LIPASE (MAGL) INHIBITORS FOR THE TREATMENT OF PAIN AND RELATED MEDICAL DISORDERS

(71) Applicant: Humanwell Pharmaceutical US, Inc., Ballwin, MO (US)

(72) Inventors: Suresh Kuarm Bowroju, Ballwin, MO (US); Shuo Zhao, Ballwin, MO (US); Subo Liao, Ballwin, MO (US); Shivansh Kaushik, Ballwin, MO (US); Ning Zhang, Ballwin, MO (US); Jun Yang, Ballwin, MO (US); Hao Zhou, Yichang (CN); Shilin Li, Yichang (CN); Xiongfeng Deng, Yichang (CN); Zhenglei Guo, Yichang (CN); Rong Liu, Yichang (CN); Yao He, Yichang (CN); Guangrui Shi, Yichang (CN)

(21) Appl. No.: 18/633,016

(22) Filed: Apr. 11, 2024

Related U.S. Application Data
(60) Provisional application No. 63/459,493, filed on Apr. 14, 2023.

(51) Int. Cl. C07D 471/04; A61K 31/4545; A61K 31/496; A61K 31/5377; A61P 29/00; C07D 471/14

(52) U.S. Cl. CPC C07D 471/04; A61K 31/4545; A61K 31/496; A61K 31/5377; A61P 29/00; C07D 471/14

(57) ABSTRACT

The present invention discloses a novel class of monoacylglycerol lipase (MAGL) small molecule inhibitors, as well as pharmaceutical compositions, preparation methods, and uses thereof. The MAGL small molecule inhibitors are represented by formula (I), with specific substituents and definitions as described in the specification. The disclosed MAGL small molecule inhibitors exhibit excellent MAGL enzyme inhibitory activity. The invention includes these compounds or their pharmaceutical compositions for the treatment and/or prevention of MAGL-related conditions such as multiple sclerosis, Alzheimer's disease, Parkinson's disease, amyotrophic lateral sclerosis, traumatic brain injury, neurotoxicity, stroke, epilepsy, anxiety, migraine, depression, hepatocellular carcinoma, colorectal cancer lesions, ovarian cancer, neuropathic pain, chemotherapy-induced neuropathy, acute pain, chronic pain, and/or pain-associated spasticity.

### CROSS REFERENCE TO RELATED APPLICATIONS

This application claims the benefit of U.S. Provisional Application No. 63/459,493, filed Apr. 14, 2023 the disclosure of which is herein incorporated by reference in its entirety.

### FIELD OF THE INVENTION

The present disclosure generally relates to novel small heterocyclic molecules as monoacylglycerol lipase (MAGL) inhibitors for the treatment of pain and related disorders.

### BACKGROUND OF THE INVENTION

Monoacylglycerol lipase (MAGL) is a primary enzyme that regulates the endocannabinoid system in the central nervous system (CNS). MAGL is expressed throughout the brain and in most brain cell types, including neurons, astrocytes, oligodendrocytes and 20 microglia cells (Chanda, P. K., et al., Mol. Pharmacol. 2010, 78, 996; Viader, A., et al, Cell rep 2015, 12, 798). Among endocannabinoids, 2-AG (2-arachidonoylglycerol), a major contributor in the brain, is hydrolyzed to arachidonic acid (AA) by MAGL, a serine hydrolase abundant. Therefore, MAGL inhibition causes an elevation in 2-AG levels as well as a depletion in AA levels in the brain and thus regulates various neuroinflammatory effects, for example, pain sensation and memory. Further, disorders such as chronic pain, anxiety and depression have been linked to regulation of endocannabinoid system signaling activities. Impeding MAGL reduces neuroinflammation and is protective in models of neurodegeneration (Nomura et al, Science 2011, 334, 809; Chen et al, Cell Rep 2012, 2, 1329; Piro et al, Cell Rep 2012 1, 617).

Crystal Structure of the Human MAGL was reported (Labar et al, Chembochem 2010, 11, 218). The catalytic triad of MAGL is characterized by Ser122, Asp239, and His269 residues. Based on the crystal structures of MAGL, several irreversible MAGL inhibitors were identified and investigated for various CNS activities (Maximilian et al. ChemMedChem 2022, e202100757; Bertrand et al. J. Mol. Biol. 2010, 396, 663-673; Griebel et al. Sci. Rep. 2015, 5, 7642; Christopher et al. J. Med. Chem. 2017, 60, 9860-9873; Laura et al. J. Med. Chem. 2018, 61, 3008-3026; Justin et al. J. Med. Chem. 2018, 61, 9062-9084; Shivani et al. Arch Pharm. 2022; e2200081; Michael et al. ChemMedChem 2021, 16, 1-16; Jayendra et al. Bioorg. Med. Chem. Lett. 2015, 25, 1436-1442; Jae et al. Chemistry & Biology 2012, 19, 579-588; Margherita et al. RSC Adv., 2016, 6, 64651-64664).

Patents have also been filed by the pharmaceutical companies to protect their inventions on MAGL irreversible inhibitors including U.S. Pat. No. 9,567,302 B2, U.S. Pat. No. 10,093,630 B2, U.S. Pat. No. 9,771,341 B2, U.S. Pat. No. 9,828,379 B2, U.S. Pat. No. 9,487,495 B2, US20180134674A1, US20180134675A1, US20180256566A1, WO2018053447A1, WO2018093949A1, WO2018217805A1, WO2018217809A1, WO2019046318A1, WO2019046330A1, WO2019222266A1, WO 2010/056309 A3, EP 3 328 849 B1, EP 3 571 202 B1, U.S. Pat. No. 10,858,373 B2, US 2009/0082435 A1, and US20160139162A1 etc.

Herein described are a series of novel MAGL inhibitors for the treatment of pain and related diseases.

### SUMMARY OF THE INVENTION

The present disclosure is directed to a compound of formula (I), or its stereoisomer, pharmaceutically acceptable salt, solvate, deuterated derivative, metabolite, or prodrug;

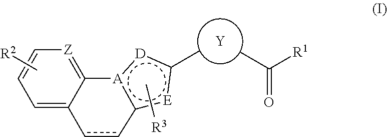

wherein,
R¹ is selected from the following:

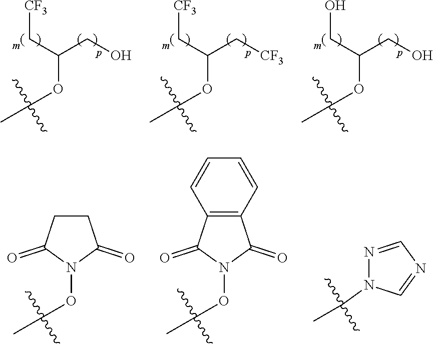

wherein, m=0, 1, 2; p=0, 1, 2;
Y ring is selected from the following:

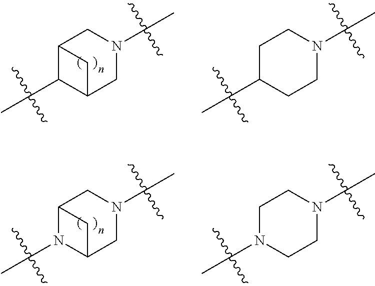

wherein, n=0, 1, 2;
A, D, and E are independently selected from C or N;
when both A and D are N, the two N atoms are connected by a single bond;
Z is C or N;
R² is H, deuterium, or halogen;
R³ is selected from the group consisting of H, deuterium, halogen, C1-C6 alkyl, substituted C1-C6 alkyl, C3-C6 cycloalkyl, substituted C3-C6 cycloalkyl, amino, nitrogen-containing alkyl, halogenated alkyl, hydroxyl, aryl, and substituted aryl; wherein the substituted C1-C6 alkyl, substituted C3-C6 cycloalkyl, substituted aryl are substituted with 1-3 substituents independently selected from the following groups: halogen, C1-C3 alkyl, C1-C3 alkoxy, halogenated C1-C3 alkyl, heteroaryl, or heterocycle.

The present disclosure is further related to a pharmaceutical composition comprising a compound or its stereoisomers, pharmaceutically acceptable salts, solvates, deuterated derivatives, metabolites, or prodrugs as described above, and a pharmaceutically acceptable carrier or excipient.

Another aspect of the present disclosure relates to a method of inhibiting enzymatic activity against monoacylglycerol lipase (MAGL). The method includes administering the compound or composition described above.

Yet another aspect of the present disclosure relates to a method of treating and/or preventing diseases or disorders related to monoacylglycerol lipase (MAGL). The method includes administering the compound or composition described above. The diseases or disorders related to MAGL neuroinflammation, neurodegenerative diseases, pain, cancer, and/or psychiatric disorders. The diseases or disorders related to MAGL further include multiple sclerosis, Alzheimer's disease, Parkinson's disease, amyotrophic lateral sclerosis, traumatic brain injury, neurotoxicity, stroke, epilepsy, anxiety, migraines, depression, hepatocellular carcinoma, colorectal cancer lesions, ovarian cancer, neuropathic pain, chemotherapy-induced neuropathy, acute pain, chronic pain, and/or spasticity conditions related to pain.

### DETAILED DESCRIPTION

The following is an overview of the subject matter of a detailed description of the invention. This overview is not intended to limit the scope of the claims.

This invention provides a new class of MAGL inhibitors, their preparation methods, and applications. The compounds of this invention exhibit good MAGL inhibitory activity and can provide effective pain relief, meeting patient needs.

In a first aspect, this invention presents compounds as represented by formula (I), or their stereoisomers, pharmaceutically acceptable salts, solvates, deuterated derivatives, metabolites, or prodrugs, in its embodiments:

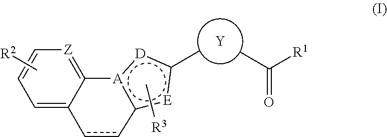

wherein,
R¹ is selected from the following:

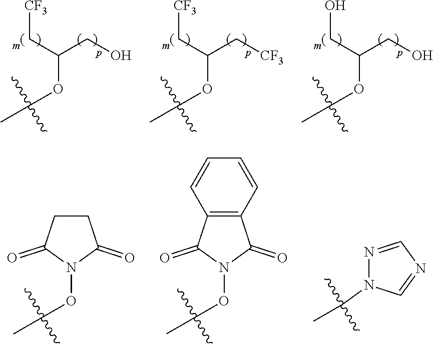

wherein, m=0, 1, 2; p=0, 1, 2;
Y ring is selected from the following:

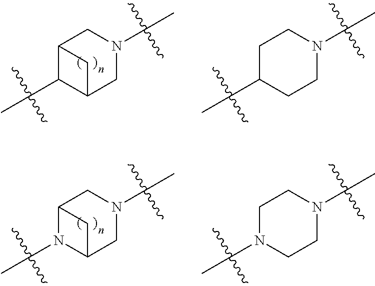

wherein, n=0, 1, 2;
A, D, and E are independently C or N, with the two N atoms being directly bonded by a single bond when both A and D are N;
Z is C or N;
R² is H, deuterium, or halogen;
R³ is selected from the group consisting of H, deuterium, halogen, C1-C6 alkyl, substituted C1-C6 alkyl, C3-C6 cycloalkyl, substituted C3-C6 cycloalkyl, amino, nitrogen-containing alkyl, halogen-containing alkyl, hydroxyl, and aryl, substituted aryl; the substituted C1-C6 alkyl, substituted C3-C6 cycloalkyl, substituted aryl are substituted by 1-3 substituents independently selected from the group consisting of halogen, C1-C3 alkyl, C1-C3 alkoxy, halogenated C1-C3 alkyl, heteroaryl, and heterocycle.

Specifically, a compound as represented by formula (II), or its stereoisomers, pharmaceutically acceptable salts, solvates, deuterated derivatives, metabolites, or prodrugs;

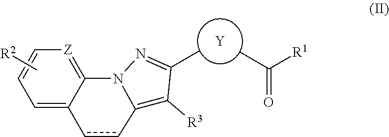

wherein,
R¹ is selected from the following:

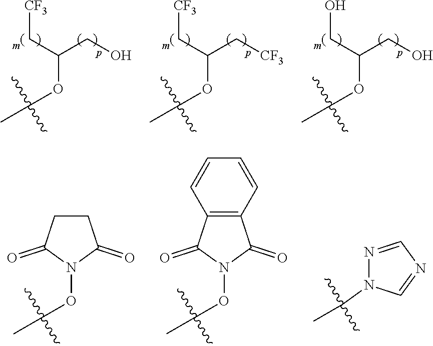

wherein, m=0, 1, 2; p=0, 1, 2;
Y ring is selected from the following:

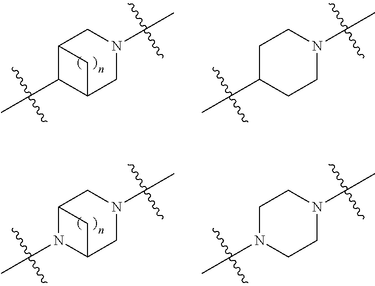

wherein, n=0, 1, 2;
Z is C or N;
R² is H, deuterium, or halogen;
R³ is selected from the group consisting of H, deuterium, halogen, C1-C6 alkyl, substituted C1-C6 alkyl, C3-C6 cycloalkyl, substituted C3-C6 cycloalkyl, amino, nitrogen-containing alkyl, halogen-containing alkyl, hydroxyl, aryl, and substituted aryl; the substituted C1-C6 alkyl, substituted C3-C6 cycloalkyl, substituted aryl are substituted by 1-3 substituents independently selected from the group consisting of halogen, C1-C3 alkyl, C1-C3 alkoxy, halogenated C1-C3 alkyl, heteroaryl, and heterocycle.

In some specific embodiments, when A and D are N atoms and E is a C atom, as represented by formulas (II-1), (II-2), and (II-3):

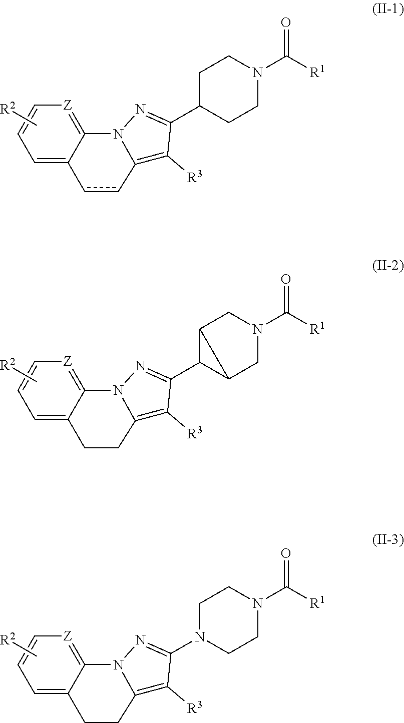

wherein,
R¹ is selected from the following:

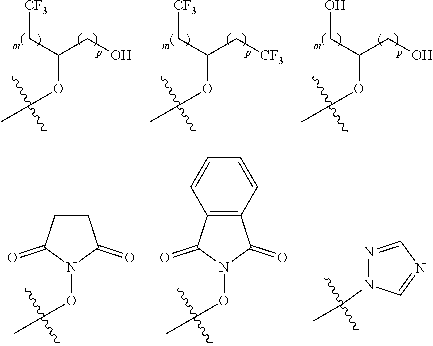

wherein, m=0, 1; p=0, 1;
Z is C or N;
R² is H, deuterium, fluorine, chlorine, bromine, or iodine;
R³ is selected from the group consisting of H, deuterium, fluorine, chlorine, bromine, iodine, methyl, ethyl, propyl, isopropyl, butyl, tert-butyl, cyclopropyl, cyclobutyl, cyclopentyl, cyclohexyl, amino, methylamino, ethylamino, dimethylamino, diethylamino, 1,4-oxazinyl, piperazinyl, piperidinyl, trifluoromethyl, hydroxyl, phenyl, and pyridinyl; the substituted C1-C6 alkyl, substituted C3-C6 cycloalkyl, substituted aryl are substituted by 1-3 substituents independently selected from the group consisting of fluorine, chlorine, bromine, methyl, ethyl, propyl, methoxy, ethoxy, trifluoromethyl, pyridine, piperidine, and piperazine.

Preferably, R¹ is selected from the following:

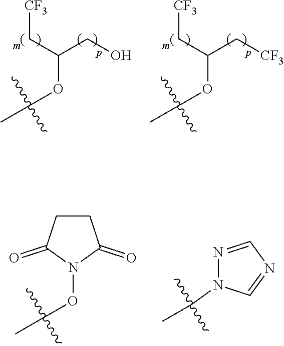

wherein, m=0; p=0, 1.

Preferably, Z is C or N.

Preferably, the position of R² is as follows, preferably fluorine.

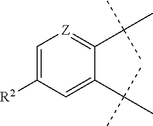

Preferably, R³ is selected from the group consisting of H, deuterium, fluorine, chlorine, bromine, amino, diethylamino, 1,4-oxazinylcyclohexyl, cyano, methyl, cyclopropyl, isopropyl, trifluoromethyl, hydroxy, and pyridyl; the mentioned substituted C1-C6 alkyl, substituted C3-C6 cycloalkyl, substituted aryl are substituted by 1-3 substituents independently selected from the following groups fluorine, chlorine, bromine, methyl, ethyl, propyl, methoxy, ethoxy, trifluoromethyl, pyridine, piperidine, and piperazine.

Specifically, compounds as shown in formula (III), or their stereoisomers, pharmaceutically acceptable salts, solvates, deuterated forms, metabolites, or prodrugs;

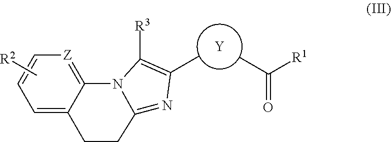

wherein,
R¹ is selected from the following:

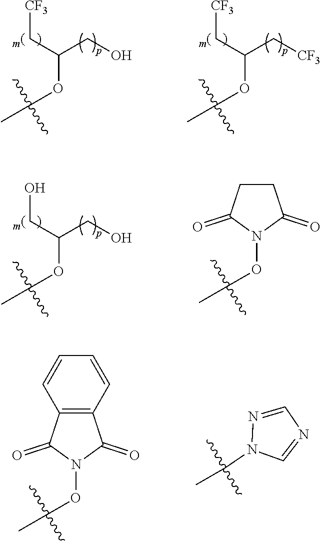

wherein, m=0, 1; p=0, 1;
Y ring is selected from the following:

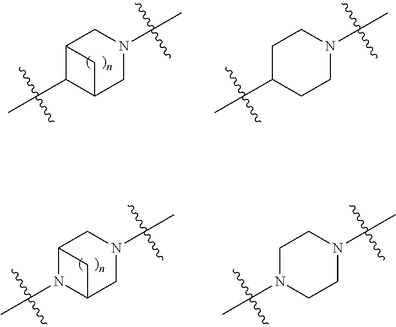

wherein, n=0;
Z is C or N;
R² is H, deuterium, or halogen;
R³ is selected from the group consisting of H, deuterium, halogen, C1-C6 alkyl, substituted C1-C6 alkyl, C3-C6 cycloalkyl, substituted C3-C6 cycloalkyl, amino, nitrogen-containing alkyl, halogen-containing alkyl, hydroxy, aryl, and substituted aryl; the substituted C1-C6 alkyl, substituted C3-C6 cycloalkyl, substituted aryl are substituted by 1-3 substituents independently selected from the following groups halogen, C1-C3 alkyl, C1-C3 alkoxy, halogenated C1-C3 alkyl, heteroaryl, and heterocycle.

In some specific embodiments, when A and E are N atoms, D is a C atom, Z is a C atom or N atom, as shown in formulas (III-1), (III-2):

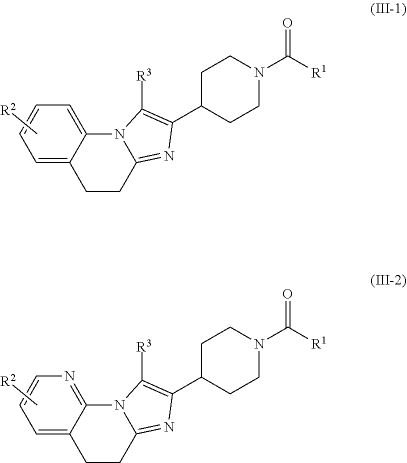

wherein,
R¹ is selected from the following:

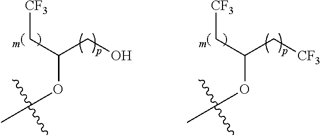

wherein, m=0, 1; p=0, 1;
R² is H, deuterium, fluorine, chlorine, bromine, or iodine;
R³ is selected from the group consisting of H, deuterium, fluorine, chlorine, bromine, iodine, methyl, ethyl, propyl, isopropyl, butyl, tert-butyl, cyclopropyl, cyclobutyl, cyclopentyl, cyclohexyl, amino, methylamino, ethylamino, dimethylamino, diethylamino, 1,4-oxazinylcyclohexyl, piperazinyl, piperidinyl, trifluoromethyl, hydroxy, phenyl, and pyridyl; the substituted C1-C6 alkyl, substituted C3-C6 cycloalkyl, substituted aryl are substituted by 1-3 substituents independently selected from the group consisting of fluorine, chlorine, bromine, methyl, ethyl, propyl, methoxy, ethoxy, trifluoromethyl, pyridine, piperidine, and piperazine;
preferably, R¹ is selected from the following:
wherein, m=0; p=1;

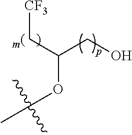

preferably, the position of R² is as follows, preferably fluorine;

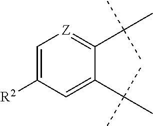

preferably, R³ is H, fluorine, chlorine, or bromine.

Specifically, compounds as shown in formula (IV), or their stereoisomers, pharmaceutically acceptable salts, solvates, deuterated forms, metabolites, or prodrugs;

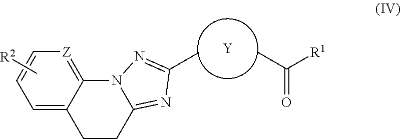

wherein,
R¹ is selected from the following:

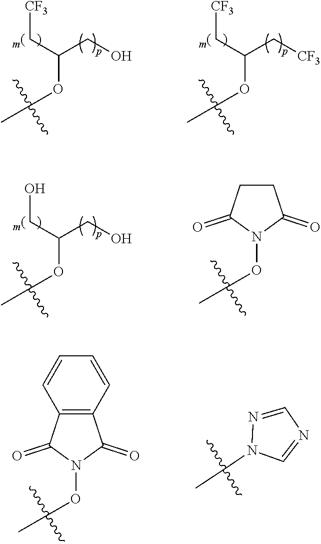

wherein, m=0, 1; p=0, 1;
Y ring is selected from the following:

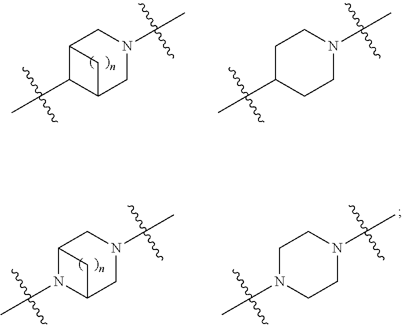

wherein, n=0, 1, 2;
R² is H, deuterium, or halogen.

In some specific embodiments, when A, D, and E are all N atoms, and Z is a C atom or N atom, as shown in formulas (IV-1) and (IV-2);

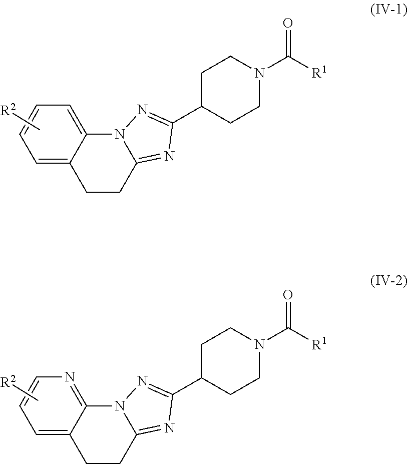

wherein,
R¹ is selected from the following:
wherein, m=0, 1; p=0, 1;

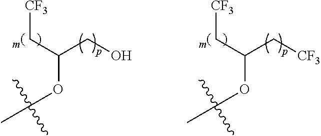

R² is H, deuterium, fluorine, chlorine, bromine, or iodine.

Preferably, R¹ is selected from the following:
wherein, m=0; p=0, 1.

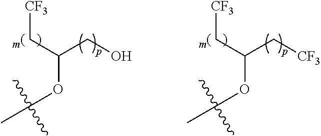

Preferably, the position of R² is as follows, preferably fluorine

Furthermore, the compounds of formula (I) are selected from the compounds shown below, as well as their stereoisomers, pharmaceutically acceptable salts, solvates, deuterated forms, metabolites, or prodrugs:

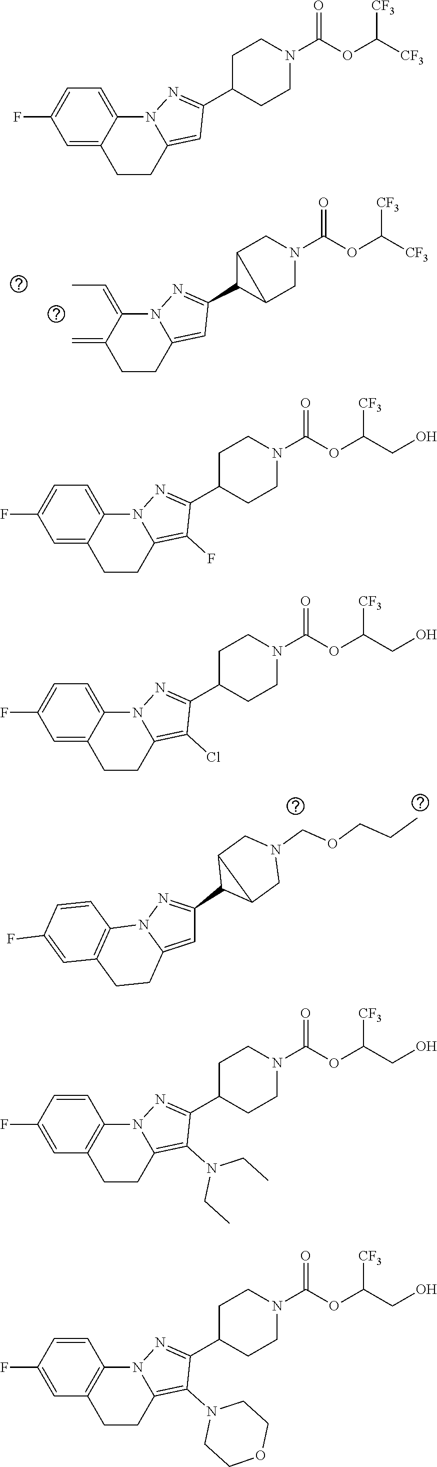

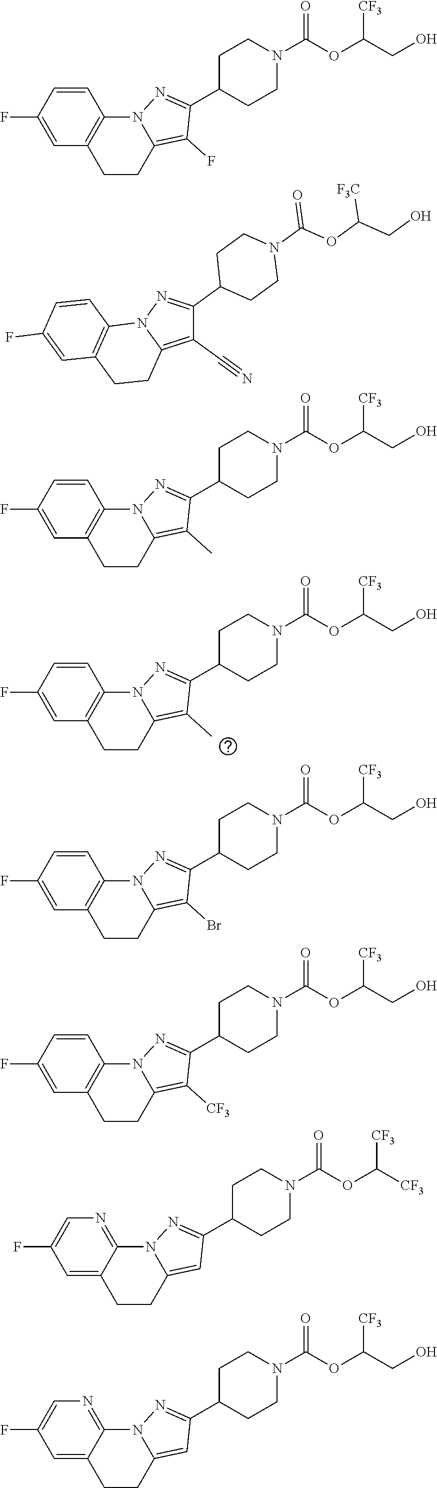

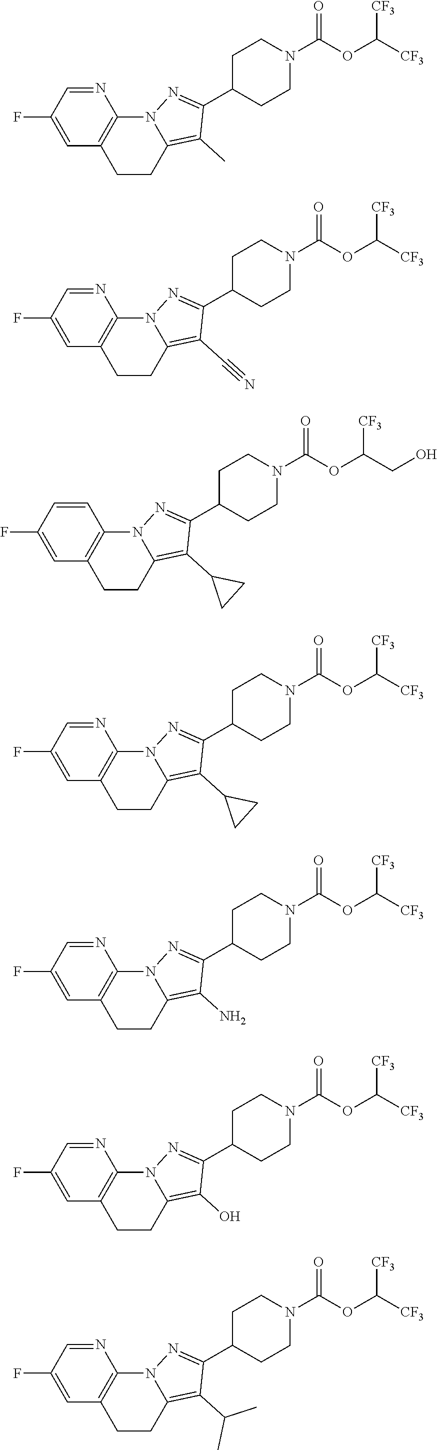

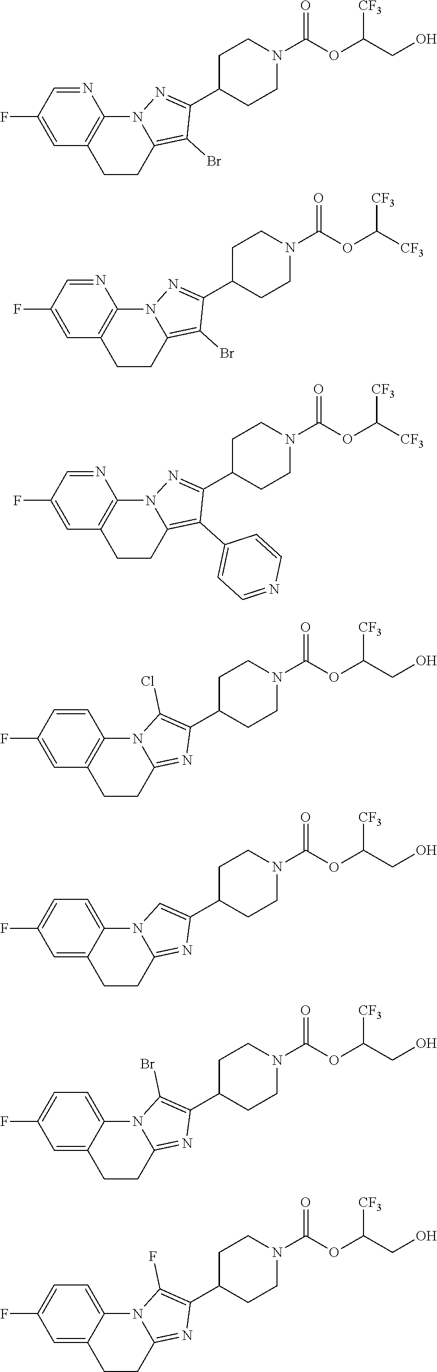

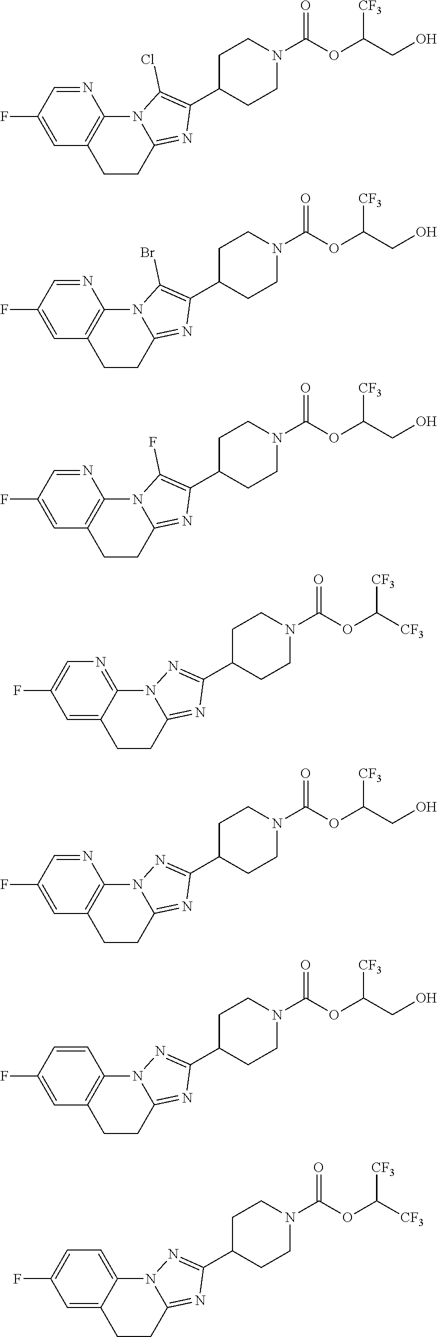

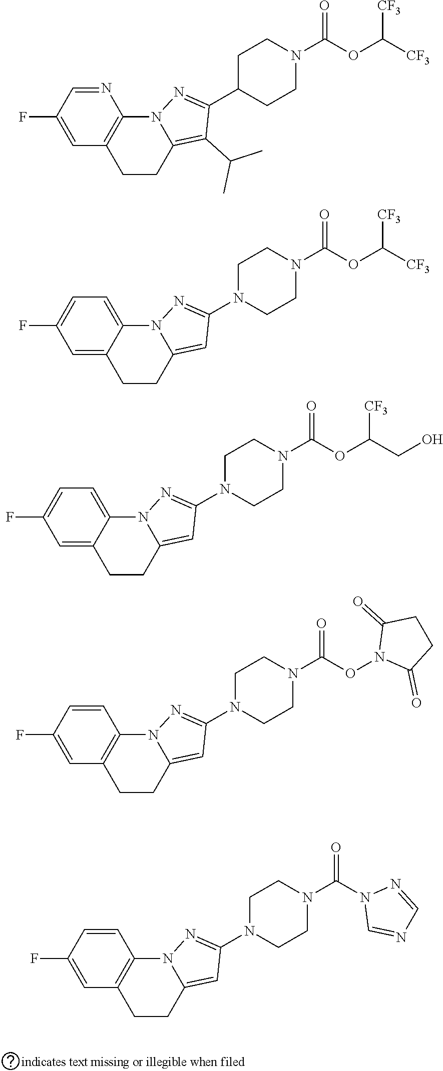 [SMILES: CC(C)c1c(C2CCN(C(=O)OC(C(F)(F)F)C(F)(F)F)CC2)nn2c1CCc1cc(F)cnc1-2 ; O=C(OC(C(F)(F)F)C(F)(F)F)N1CCN(c2cc3n(n2)-c2ccc(F)cc2CC3)CC1 ; O=C(OC(CO)C(F)(F)F)N1CCN(c2cc3n(n2)-c2ccc(F)cc2CC3)CC1 ; O=C(ON1C(=O)CCC1=O)N1CCN(c2cc3n(n2)-c2ccc(F)cc2CC3)CC1 ; O=C(N1CCN(c2cc3n(n2)-c2ccc(F)cc2CC3)CC1)n1cncn1]

On another hand, this invention provides a pharmaceutical composition, which contains a compound of formula (I) and its stereoisomers, pharmaceutically acceptable salts, solvates, deuterated forms, metabolites, or prodrugs, and at least one pharmaceutically acceptable excipient or carrier.

A third aspect of the invention provides a method for synthesizing compounds, the preparation method includes the following steps:

A compound of formula (I-1) reacts with a compound of formula (I-2) in acetonitrile to obtain a compound of formula (I-3), which is a compound of formula (I):

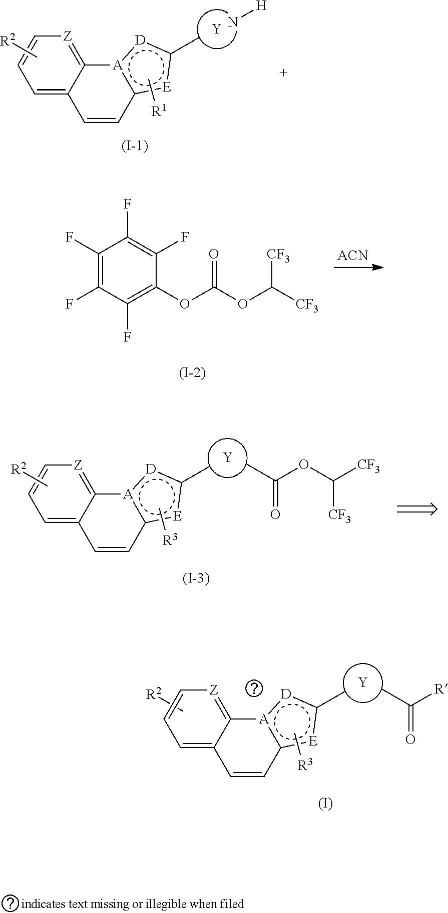

Alternatively,
1) A compound of formula (I-1) reacts with a compound of formula (I-4) in acetonitrile to obtain a compound of formula (I-5);

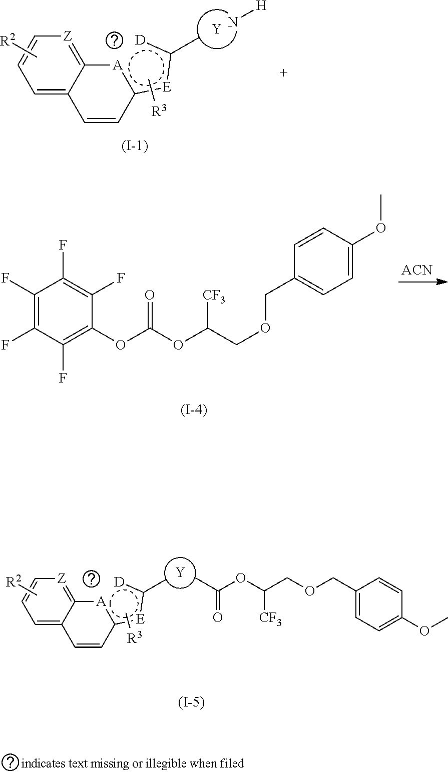

2) Under acidic conditions, the PMB group is removed from a compound of formula (I-5) to obtain a compound of formula (I-6), which is a compound of formula (I);

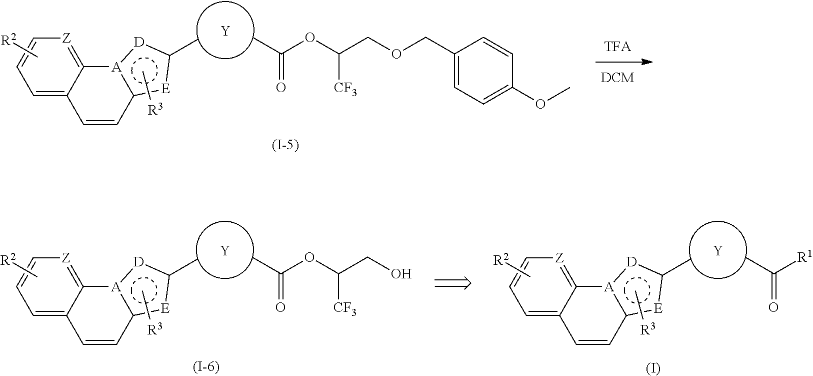

Alternatively, a compound of formula (I-1) reacts with a compound of formula (1-7) under alkaline conditions to obtain a compound of formula (I-8), which is a compound of formula (I);

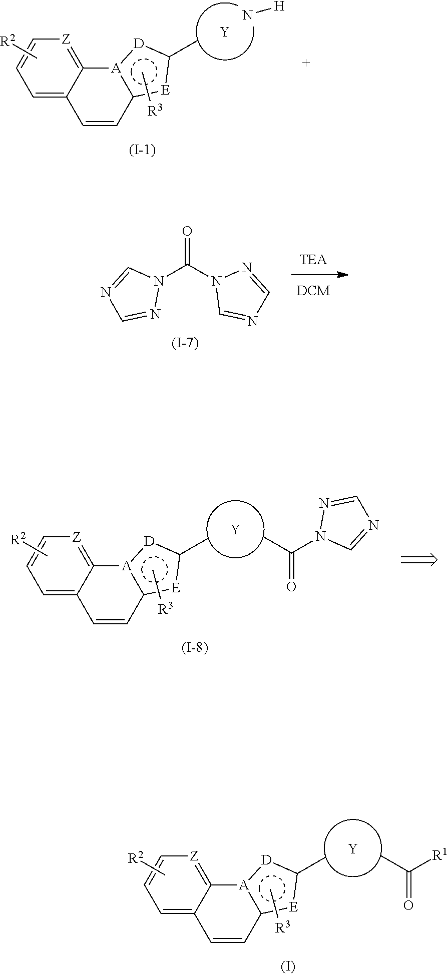

Alternatively, a compound of formula (I-1) reacts with a compound of formula (I-9) under alkaline conditions to obtain a compound of formula (I-10), which is a compound of formula (I);

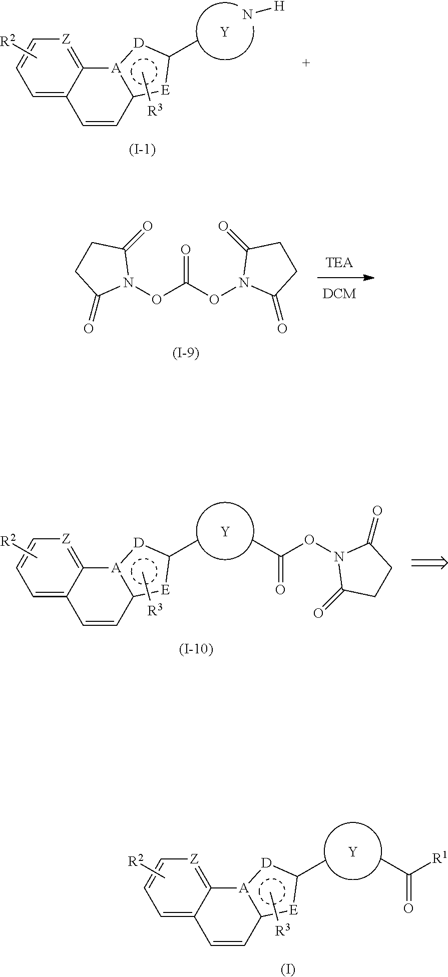

A fourth aspect of the invention provides a method for treating MAGL-related diseases or disorders. The method involves administering to an individual in need a composition containing a compound of formula (I).

Without being bound by any theory, compounds of formula (I) are considered to primarily act as inhibitors mediating the activity of MAGL. The binding at this site is believed to be capable of treating related conditions such as neuropathic pain.

### Beneficial Effects

The compounds of this invention feature a novel structure and have demonstrated good MAGL inhibitory activity in vitro tests. Through in vivo testing, it has been discovered that they possess effective therapeutic and/or preventative actions against MAGL-related neuroinflammation, neurodegenerative diseases, pain, cancer, and/or psychiatric disorders, particularly showing significant analgesic effects. Specifically, this includes conditions such as multiple sclerosis, Alzheimer's disease, Parkinson's disease, amyotrophic lateral sclerosis, traumatic brain injury, neurotoxicity, stroke, epilepsy, anxiety, migraines, depression, hepatocellular carcinoma, colorectal cancer lesions, ovarian cancer, neuropathic pain, chemotherapy-induced neuropathy, acute pain, chronic pain, and/or pain-related spastic states.

### Definition and Explanation of Terms

Unless otherwise specified, the definitions of groups and terms recorded in the specification and claims of this application, including their definitions as examples, exemplary, preferred, recorded in tables, and specifically in embodiments, can be combined and recombined with each other. Subsequent group definitions and compound structures should be considered within the scope recorded in the specification.

The compounds described herein can have asymmetric centers. The compounds of the invention containing asymmetrically substituted atoms can be separated into optically active or racemic forms. Unless a specific stereochemistry or isomeric form is particularly noted, all chiral, achiral, racemic forms, as well as all geometric isomers of structures are applicable.

The term “alkyl” used herein refers to lower alkyls having 1 to 6 carbon atoms in the main chain, up to 20 carbon atoms in total. They can be straight-chain, branched-chain, or cyclic, including methyl, ethyl, propyl, isopropyl, butyl, hexyl, etc.

The term “aryl” used herein alone or as part of another group represents any optionally substituted conjugated planar ring or ring system containing delocalized electrons. These aryl groups are preferably single-ring (e.g., furan or benzene), two-ring, or three-ring groups having 5-14 atoms in the ring portion. The term “aromatic” includes the defined “aryl.”

The term “aryl” or “Ar” used herein alone or as part of another group denotes any optionally substituted homocyclic aromatic group, preferably single-ring or two-ring groups having 6 to 10 carbon atoms in the ring portion, such as phenyl, biphenyl, naphthyl, substituted phenyl, substituted biphenyl, or substituted naphthyl.

The term “carbocycle” or “carbocyclic” used herein alone or as part of another group denotes any optionally substituted, aromatic or non-aromatic, homocycle or ring system where all atoms in the ring are carbon, preferably with 5 or 6 carbon atoms per ring. Exemplary substituents include one or more of the following groups: hydrocarbyl, substituted hydrocarbyl, alkyl, alkoxy, acyl, acyloxy, alkenyl, alkenyloxy, aryl, aryloxy, amino, amido, acetal, aminocarbonyl, carbocyclic, cyano, ester, ether, halogen, heterocycle, hydroxy, ketone, ketale, phosphate, nitro, and thio.

The term “heteroaryl” used herein alone or as part of another group denotes an aromatic group with at least one heteroatom in at least one ring, preferably with 5 or 6 atoms per ring, optionally substituted. Heteroaryl groups preferably have 1 or 2 oxygen atoms and/or 1 to 4 nitrogen atoms and are connected to the rest of the molecule through carbon. Example groups include furanyl, benzofuranyl, oxazolyl, isoxazolyl, oxadiazolyl, benzoxazolyl, benzoxadiazolyl, pyrrolyl, pyrazolyl, imidazolyl, triazolyl, tetrazolyl, pyridyl, pyrimidinyl, pyrazinyl, quinazolinyl, indolyl, isaindolyl, indolizinyl, benzimidazolyl, indazolyl, benzotriazolyl, tetrazolopyrazinyl, carbozolyl, purinyl, quinolyl, isoquinolyl, imidazopyridinyl, etc. Exemplary substituents include one or more of the following groups: hydrocarbyl, substituted hydrocarbyl, alkyl, alkoxy, acyl, acyloxy, alkenyl, alkenyloxy, aryl, aryloxy, amino, amido, acetal, aminocarbonyl, carbocyclic, cyano, ester, ether, halogen, heterocycle, hydroxy, ketone, ketale, phosphate, nitro, and thio.

The term “heterocycle” or “heterocyclic” used herein alone or as part of another group denotes any optionally substituted, fully saturated or unsaturated, mono- or bicyclic, aromatic or non-aromatic group with at least one heteroatom in at least one ring, preferably with 5 or 6 atoms per ring. Heterocyclic groups preferably have 1 or 2 oxygen atoms and/or 1 to 4 nitrogen atoms and are connected to the rest of the molecule through carbon or a heteroatom. Exemplary heterocyclic groups include the heteroaryl compounds as described above. Exemplary substituents include one or more of the following groups: hydrocarbyl, substituted hydrocarbyl, alkyl, alkoxy, acyl, acyloxy, alkenyl, alkenyloxy, aryl, aryloxy, amino, amido, acetal, aminocarbonyl, carbocyclic, cyano, ester, ether, halogen, heterocycle, hydroxy, ketone, ketale, phosphate, nitro, and thio.

The term “protecting group” used herein denotes a group that can protect a specific moiety, where the protecting group can be removed after the protected reaction without interfering with the rest of the molecule. When the moiety is an oxygen atom (forming a protected hydroxyl group), exemplary protecting groups include ethers (e.g., allyl, triphenylmethyl (trityl or Tr), benzyl, p-methoxybenzyl (PMB), p-methylphenyl (PMP)), acetals (e.g., methoxymethyl (MOM), β-methoxyethoxymethyl (MEM), tetrahydropyranyl (THP), ethoxyethyl (EE), methylthiomethyl (MTM), 2-methoxy-2-propyl (MOP), 2-trimethylsilyloxyethyl (SEM)), esters (e.g., benzoate (Bz), carbonates like allyl carbonate, 2,2,2-trichloroethyl carbonate (Troc), 2-trimethylsilyl ethyl carbonate), silyl ethers (e.g., trimethylsilyl (TMS), triethylsilyl (TES), triisopropylsilyl (TIPS), triphenylsilyl (TPS), tert-butyldimethylsilyl (TBDMS), tert-butyldiphenylsilyl (TBDPS)), etc. When the moiety is a nitrogen atom (thus forming a protected amine), exemplary protecting groups include benzyl (e.g., p-methoxybenzyl (PMP), 3,4-dimethoxybenzyloxy (PMB)), esters (e.g., benzoate (Bz)), carbonyls (e.g., p-methoxybenzyl carbonyl (Moz), tert-butoxycarbonyl (BOC), 9-fluorenylmethoxycarbonyl (Fmoc)), acetyl, carbamates, n-silyl, etc. Various protecting groups and their synthetic methods can be found in “Greene's Protective Groups in Organic Synthesis” (4th edition) by P. G. M. Wuts and T. W. Greene, John Wiley & Sons, Inc.

The term “substituted hydrocarbyl” used herein refers to a hydrocarbyl moiety substituted with at least one non-carbon atom, including moieties where carbon chain atoms are replaced by heteroatoms such as nitrogen, oxygen, silicon, phosphorus, boron, or halogen, and moieties where the carbon chain includes additional substituents. These substituents include alkyl, alkoxy, acyl, acyloxy, alkenyl, alkenyloxy, aryl, aryloxy, amino, amido, acetal, aminocarbonyl, carbocyclic, cyano, ester, ether, halogen, heterocycle, hydroxy, ketone, ketale, phosphate, nitro, and thio.

The terms “comprises,” “comprising,” and “having” are used herein to indicate inclusive possession, implying that besides the listed elements, other elements may also be present. After a detailed description of the invention, it is apparent that modifications and variations can be made without departing from the scope defined in the attached claims.

The term “neurodegenerative diseases” refers to a state of disease where neuronal cells in the brain and spinal cord lose their function. The brain and spinal cord consist of neurons with various functions such as controlling movement, processing sensory information, and making decisions. Generally, cells in the brain and spinal cord do not regenerate, and damage may be irreversible. This includes multiple sclerosis, Alzheimer's disease, Parkinson's disease, amyotrophic lateral sclerosis, traumatic brain injury, neurotoxicity, stroke, Huntington's disease, cerebellar ataxia, etc.

The term “psychiatric disorders” refers to a general term for disturbances in cognitive, emotional, behavioral, and volitional mental activities caused by dysfunction of brain function. Common types include mood disorders, organic brain disorders, etc. This includes anxiety disorders, obsessive-compulsive disorder, depression, bipolar disorder, schizophrenia, manic-depressive illness, menopausal psychiatric disorder, post-traumatic stress disorder, paranoid psychosis, and various organic lesion-associated psychiatric disorders, etc.

Examples of pain include acute pain, chronic pain, pain caused by soft tissue injury or peripheral injury, postherpetic neuralgia, occipital neuralgia, trigeminal neuralgia, glossopharyngeal neuralgia or intercostal neuralgia, central pain, neuropathic pain, migraine, pain associated with osteoarthritis or rheumatoid arthritis, pain associated with contusions, sprains, or trauma, spinal pain, pain caused by spinal cord or brainstem injury, lower back pain, sciatica, toothache, myofascial pain syndrome, vulvotomy pain, gout pain, pain caused by burns, cardiac pain, muscle pain, eye pain, inflammatory pain, orofacial pain, abdominal pain, pain associated with dysmenorrhea, labor pain or endometriosis, somatic pain, pain associated with nerve or nerve root damage, pain associated with amputation, trigeminal neuralgia, neuritis or vasculitis, pain caused by diabetic neuropathy (or diabetic peripheral neuropathy), pain caused by chemotherapy-induced neuropathy, atypical facial neuralgia, lower back neuralgia, trigeminal neuralgia, occipital neuralgia, glossopharyngeal neuralgia or intercostal neuralgia, neuralgia related to HIV, neuralgia related to AIDS, hyperalgesia, burn pain, sudden pain, pain caused by chemotherapy, occipital neuralgia, psychogenic pain, pain associated with gallstones, neuropathic or non-neuropathic pain associated with cancer, phantom limb pain, functional abdominal pain, headache, acute or chronic tension headache, sinus headache, cluster headache, temporomandibular joint pain, maxillary sinus pain, pain caused by ankylosing spondylitis, postoperative pain, scar pain, chronic non-neuropathic pain, pain attributed to hyperlipidemia, fibromyalgic pain, and fibromyalgia.

Other features and advantages of the invention will be set forth in the following description, and, in part, will become apparent from the description, or may be learned by practice of the invention. The objectives and other advantages of the invention may be realized and attained by the structures particularly pointed out in the written description, claims, and drawings.

### EXAMPLES

The following text will provide a more detailed explanation of the general formula compounds of this invention, their preparation methods, and applications, in conjunction with specific embodiments. The embodiments provided below are only for illustrative and interpretive purposes of this invention and should not be construed as limiting the scope of this invention. All technical solutions realized based on the content described above fall within the scope intended to be protected by this invention.

Unless otherwise specified, the raw materials and reagents used in the following examples are commercially available products or can be prepared by known methods.

This application employs the following abbreviations:
ACN: Acetonitrile
CDI: N,N′-Carbonyldiimidazole
CuI: Copper(I) iodide
DCM: Dichloromethane
DIEA: N,N-Diisopropylethylamine
DMF: N,N-Dimethylformamide
DPPA: Diphenyl phosphorazidate
DPPF: 1,1′-Bis(diphenylphosphino)ferrocene
EtOAc: Ethyl acetate
EtOH: Ethanol
K₂CO₃: Potassium carbonate
LDA: Lithium diisopropylamide
NaH: Sodium hydride
Na₂SO₄: Sodium sulfate
NaBH₄: Sodium borohydride
NBS: N-Bromosuccinimide
NCS: N-Chlorosuccinimide
NIS: N-Iodosuccinimide
NMP: N-Methyl-2-pyrrolidone
NSFI: N-Fluorobis(phenylsulfonyl)imide
PE: Petroleum ether
t-BuOK: Potassium tert-butoxide
TEA: Triethylamine
TFA: Trifluoroacetic acid
THE: Tetrahydrofuran

Compounds are named according to the conventional naming rules in the field, and commercially available reagents are named using the names from suppliers' catalogs.

¹H NMR data are collected and recorded on a Bruker Avance Neo 400 MHz liquid superconducting nuclear magnetic resonance spectrometer at 400 MHz, using CDCl₃, DMSO-d6, MeOD as solvents, and TMS (5=0) as an internal standard to report chemical shift δ values (ppm). Mass spectra are collected and recorded using a Waters ACQUITY UPLC, with detection on an ACQUITY UPLC BEH C8, 50 mm2.1 mm, 1.7 μm (20180306-C8-08) chromatographic column. Mobile phase A: 0.01% TFA/H₂O; Mobile phase B: CH₃CN; Flow rate: 0.2 mL/min; Column temperature: 30° C.; Detection wavelength: UV-210 nm. High-performance liquid chromatography (HPLC) is determined using a Thermo UltiMate 3000 liquid chromatograph, with detection on a Venusil ASB C18 (4.6250 mm, 5 μm) chromatographic column. Mobile phase A: phosphoric acid aqueous solution at pH=1.5; Mobile phase B: CH₃CN; Flow rate: 1.0 mL/min; Column temperature: 35° C.; Detection wavelength: UV-215 nm; Injection volume: 2 μL; Gradient elution conditions: Elute at a flow rate of 1.0 mL/min throughout, first with 95% A and 5% B for 10 min, then with 20% A and 80% B for 5 min, and finally with 95% A and 5% B for 5 min. The percentages refer to the volume percentage of the mobile phase in the elution solution.

### Example 1: Synthesis of 1,1,1,3,3,3-hexafluoropropan-2-yl 4-(7-fluoro-4,5-dihydropyrazolo[1,5-a]quinolin-2-yl)piperidine-1-carboxylate

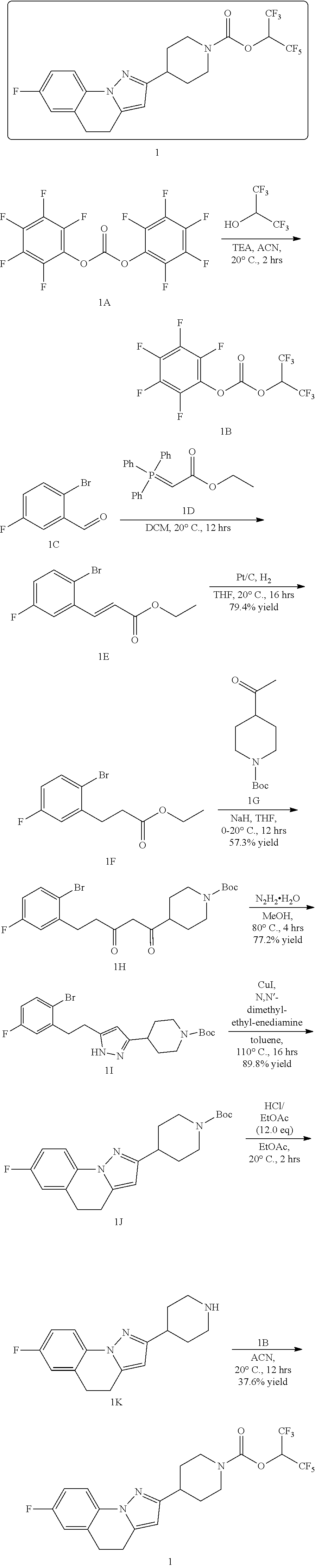

### Scheme-1: Synthesis of 1,1,1,3,3,3-hexafluoropropan-2-yl 4-(7-fluoro-4,5-dihydropyrazolo[1,5-a]quinolin-2-yl)piperidine-1-carboxylate

### Procedure for the preparation of 1,1,1,3,3,3-hexafluoropropan-2-yl (perfluorophenyl) carbonate (1B)

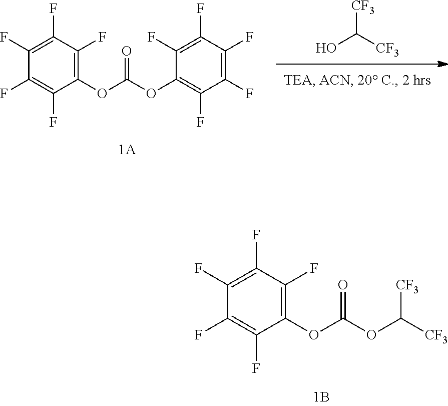 [SMILES: O=C(Oc1c(F)c(F)c(F)c(F)c1F)Oc1c(F)c(F)c(F)c(F)c1F ; OC(C(F)(F)F)C(F)(F)F ; O=C(Oc1c(F)c(F)c(F)c(F)c1F)OC(C(F)(F)F)C(F)(F)F]

To a solution of 1A (1.00 g, 2.54 mmol, 1.00 eq) in ACN (10.0 mL) was added TEA (1.28 g, 12.7 mmol, 1.77 mL, 5.00 eq) and 1,1,1,3,3,3-hexafluoropropan-2-ol (1.28 g, 7.61 mmol, 3.00 eq). The mixture was stirred at 20° C. for 2 hrs. TLC (Petroleum ether/Ethyl acetate=5:1) indicated 1A (R_{f}=0.71) was consumed completely and one new spot (R_{f}=0.03) formed. Compound 1B (0.96 g, crude) was obtained as a light yellow liquid to use the next step.

### Procedure for the preparation of ethyl (E)-3-(2-bromo-5-fluorophenyl)acrylate (1E)

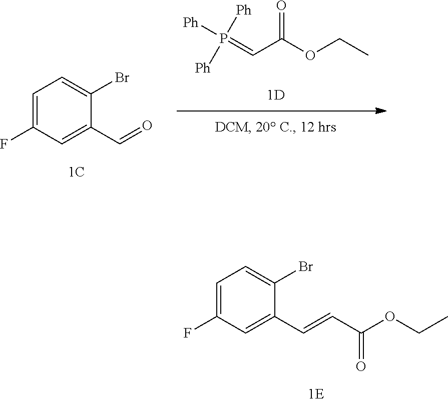 [SMILES: CCOC(=O)C=P(c1ccccc1)(c1ccccc1)c1ccccc1 ; O=Cc1cc(F)ccc1Br ; CCOC(=O)/C=C/c1cc(F)ccc1Br]

To a solution of 1C (25.0 g, 123 mmol, 1.00 eq) in DCM (250 mL) was added 1D (42.9 g, 123 mmol, 1.00 eq). The mixture was stirred at 20° C. for 12 hrs. The reaction mixture was filtered and concentrated under reduced pressure to give a residue. The residue was purified by column chromatography (SiO₂, Petroleum ether/Ethyl acetate=20/1 to 10/1, R_{f}=0.50) to give compound 1E (32.0 g) as a yellow oil.

¹H NMR: (400 MHz, CDCl₃): δ 7.95-7.99 (m, 1H), 7.56-7.59 (m, 1H), 7.29-7.32 (m, 1H), 6.97-6.99 (m, 1H), 3.91-4.00 (m, 1H), 6.37 (d, J=16 Hz, 1H), 4.29 (q, J=7.2 Hz, 2H), 1.35 (t, J=7.2 Hz, 3H).

### Procedure for the preparation of ethyl 3-(2-bromo-5-fluorophenyl)propanoate (1F)

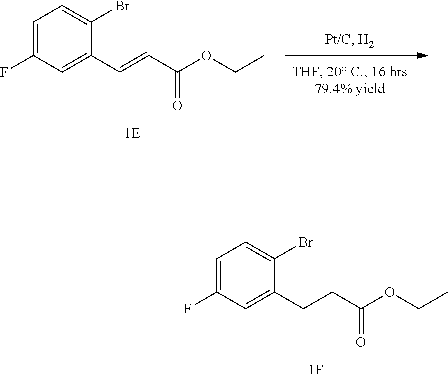

To a solution of 1E (200 mg, 732 μmol, 1.00 eq) in THF (20.0 mL) was added Pt/C (30.8 mg, 7.32 μmol, 5.00% purity, 0.01 eq). The mixture was stirred at 20° C. for 16 hrs under H₂ atmosphere. The reaction mixture was filtered, and filter cake was washed with THF (10.0 mL×3) and concentrated under reduced pressure to give compound 1F (160 mg, 582 μmol, 79.4% yield) was obtained as a colorless oil. LCMS: Rt=0.467 min, MS=277.1, M+H⁺

¹H NMR: (400 MHz, CDCl₃): δ 7.46-7.50 (m, 1H), 6.98-7.01 (m, 1H), 6.80-6.84 (m, 1H), 4.15 (q, J=14.0 Hz, 2H), 3.04 (d, J=7.6 Hz, 2H), 2.64 (d, J=7.6 Hz, 2H), 1.23 (d, J=6.0 Hz, 2H).

### Procedure for the preparation of tert-butyl 4-(5-(2-bromo-5-fluorophenyl)-3-oxopentanoyl)piperidine-1-carboxylate (1H)

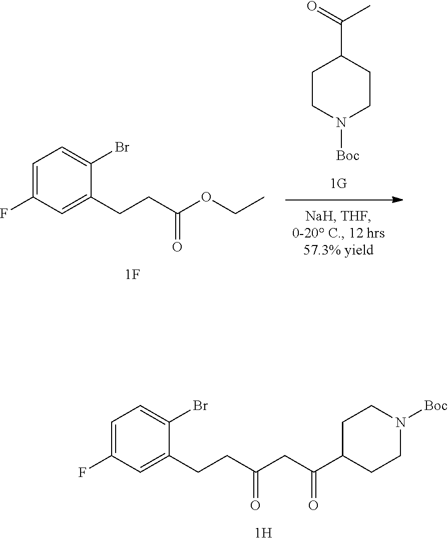 [SMILES: CCOC(=O)CCc1cc(F)ccc1Br ; CC(=O)C1CCN(C(=O)OC(C)(C)C)CC1 ; CC(C)(C)OC(=O)N1CCC(C(=O)CC(=O)CCc2cc(F)ccc2Br)CC1]

To a solution of 1F (2.00 g, 8.80 mmol, 1.00 eq) and 1G (3.15 g, 11.4 mmol, 1.30 eq) in THF (20.0 mL) was added NaH (528 mg, 13.2 mmol, 60% purity, 1.50 eq) at 0° C. The mixture was stirred at 20° C. for 16 hrs. The reaction mixture was quenched by addition NH₄Cl (20.0 mL) at 0° C., and then diluted with EtOAc (20.0 mL) and extracted with EtOAc (20.0 mL×3). The combined organic layers were washed with brine (20.0 mL), dried over Na₂SO₄, filtered and concentrated under reduced pressure to give a residue. The residue was purified by column chromatography (SiO₂, Petroleum ether/Ethyl acetate=10/1 to 4/1, Petroleum ether/Ethyl acetate=2/1, R_{f}=0.10) to obtained compound 1H (2.30 g, 5.04 mmol, 57.3% yield) as a light-yellow oil.

LCMS: Rt=0.558 min, MS=480.3, M+Na⁺

### Procedure for the preparation of tert-butyl 4-(5-(2-bromo-5-fluorophenethyl)-1H-pyrazol-3-yl)piperidine-1-carboxylate (1l)

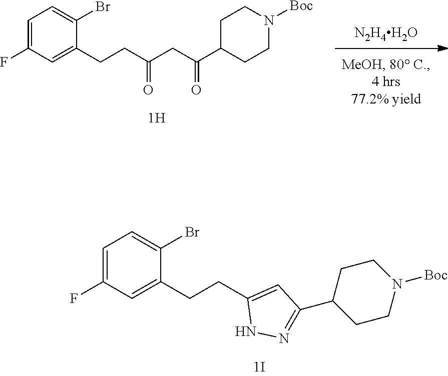 [SMILES: CC(C)(C)OC(=O)N1CCC(C(=O)CC(=O)CCc2cc(F)ccc2Br)CC1 ; "N2H4·H2O" ; CC(C)(C)OC(=O)N1CCC(c2cc(CCc3cc(F)ccc3Br)[nH]n2)CC1]

To a solution of 1H (1.95 g, 4.27 mmol, 1.00 eq) in MeOH (19.5 mL) was added NH₂NH₂·H₂O (428 mg, 8.55 mmol, 415 μL, 2.00 eq). The mixture was stirred at 80° C. for 4 hrs. The reaction mixture was quenched by addition of H₂O (5.00 mL) at 20° C. Then the mixture was filtered and concentrated under reduced pressure to give a residue. The residue was purified by column chromatography (SiO₂, Petroleum ether/Ethyl acetate=5/1 to 1/1, Petroleum ether/Ethyl acetate=1/1, R_{f}=0.15) to obtained compound 11 (1.50 g, 3.30 mmol, 77.2% yield, 99.5% purity) as a light-yellow gum.

LCMS: Rt=0.468 min, MS=398.3, M-56+H⁺

### Procedure for the preparation of tert-butyl 4-(7-fluoro-4,5-dihydropyrazolo [1,5-a]quinolin-2-yl)piperidine-1-carboxylate (1J)

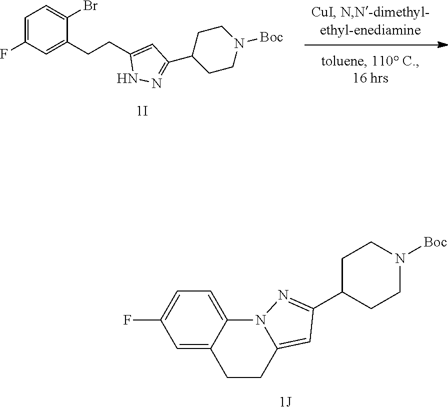

To a solution of 11 (1.50 g, 3.30 mmol, 1.00 eq) in toluene (50.0 mL) was added CuI (31.4 mg, 165 μmol, 0.05 eq), K₂CO₃ (911 mg, 6.59 mmol, 2.00 eq) and N,N′-dimethylethane-1,2-diamine (72.6 mg, 824 μmol, 88.7 μL, 0.25 eq). The mixture was stirred at 110° C. for 16 hrs. The reaction mixture was filtered and concentrated under reduced pressure to give a residue. The residue was purified by column chromatography (SiO₂, Petroleum ether/Ethyl acetate=10/1 to 5/1, Petroleum ether/Ethyl acetate=5/1, R_{f}=0.61) to obtained compound 1J (1.10 g, 2.96 mmol, 89.8% yield, 100% purity) as a white solid.

LCMS: Rt=0.524 min, MS=394.4, M+Na⁺

### Procedure for the preparation of 7-fluoro-2-(piperidin-4-yl)-4,5-dihydropyrazolo[1,5-a]quinoline (1K)

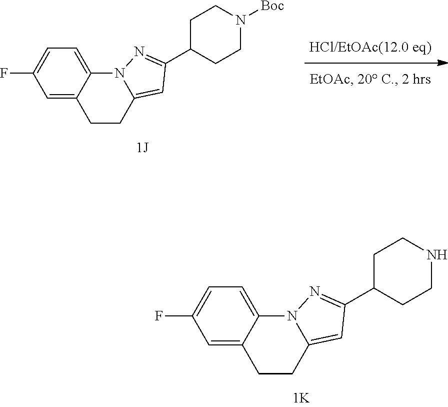 [SMILES: CC(C)(C)OC(=O)N1CCC(c2cc3n(n2)-c2ccc(F)cc2CC3)CC1 ; "HCl/EtOAc(12.0 eq)" ; Fc1ccc2c(c1)CCc1cc(C3CCNCC3)nn1-2]

To a solution of compound 1J (1.10 g, 2.96 mmol, 1.00 eq) in EtOAc (11.0 mL) was added HCl/EtOAc (4.00 M, 8.88 mL, 12.0 eq). The reaction mixture was concentrated under reduced pressure to give compound 1K (1.10 g, crude, HCl) as a light-yellow solid.

LCMS: Rt=0.300 min, MS=272.4, M+H⁺

### Procedure for the preparation of 1,1,1,3,3,3-hexafluoropropan-2-yl 4-(7-fluoro-4,5-dihydropyrazolo[1,5-a]quinolin-2-yl)piperidine-1-carboxylate (1)

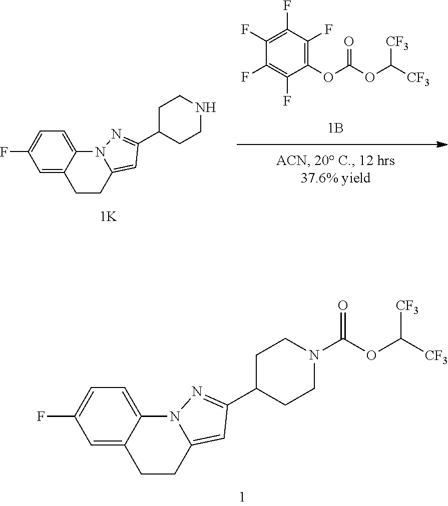 [SMILES: Fc1ccc2c(c1)CCc1cc(C3CCNCC3)nn1-2 ; O=C(Oc1c(F)c(F)c(F)c(F)c1F)OC(C(F)(F)F)C(F)(F)F ; O=C(OC(C(F)(F)F)C(F)(F)F)N1CCC(c2cc3n(n2)-c2ccc(F)cc2CC3)CC1]

To a solution of 1K (230 mg, 848 μmol, 1.00 eq) in ACN (2.30 mL) was added 1B (641 mg, 1.70 mmol, 2.00 eq). The mixture was stirred at 20° C. for 12 hrs. The reaction mixture was concentrated under reduced pressure to give a residue. The residue was purified by prep-HPLC (column: Phenomenex luna C18 150*25 mm*10 um; mobile phase: [water (FA)-ACN]; gradient: 80%-100% B over 10 min) to obtain compound 1 (150 mg, 319 μmol, 37.6% yield, 98.9% purity) as a yellow gum.

LCMS: Rt=0.544 min, MS=466.4, M+H⁺

¹H NMR: (400 MHz, CDCl₃): δ 7.78-7.82 (m, 1H), 6.94-7.03 (m, 2H), 5.98 (s, 1H), 5.76-5.82 (m, 1H), 4.19-4.26 (m, 2H), 3.09-3.14 (m, 2H), 2.94-3.00 (m, 5H), 2.07 (d, J=12.8 Hz, 2H), 1.61-1.78 (m, 2H).

### Example 2: Synthesis of 1,1,1-trifluoro-3-hydroxypropan-2-yl 4-(7-fluoro-4,5-dihydropyrazolo[1,5-a]quinolin-2-yl)piperidine-1-carboxylate

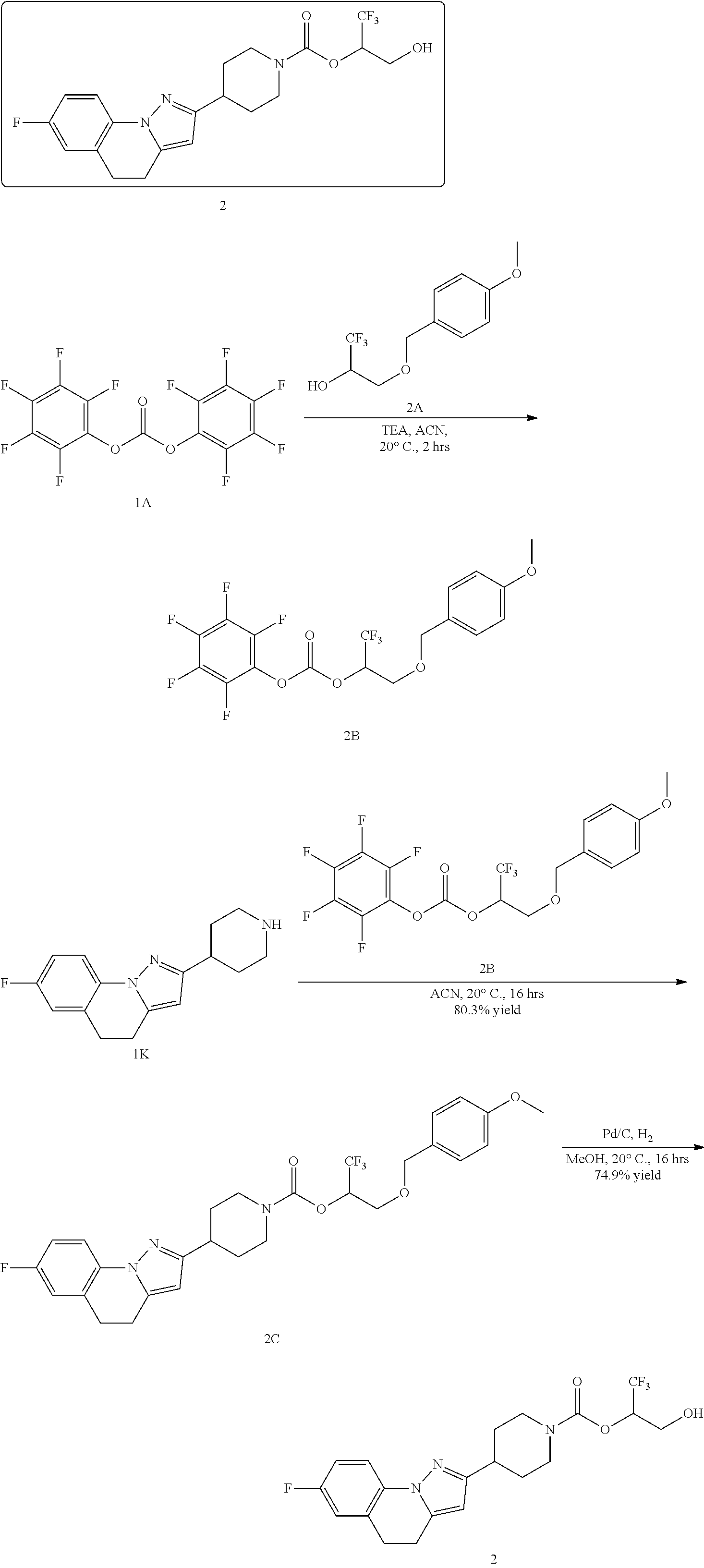

### Scheme-2: Synthesis of 1,1,1-trifluoro-3-hydroxypropan-2-yl 4-(7-fluoro-4,5-dihydropyrazolo[1,5-a]quinolin-2-yl)piperidine-1-carboxylate

### Procedure for the preparation of perfluorophenyl (1,1,1-trifluoro-3-((4-methoxybenzyl)oxy)propan-2-yl) carbonate (2B)

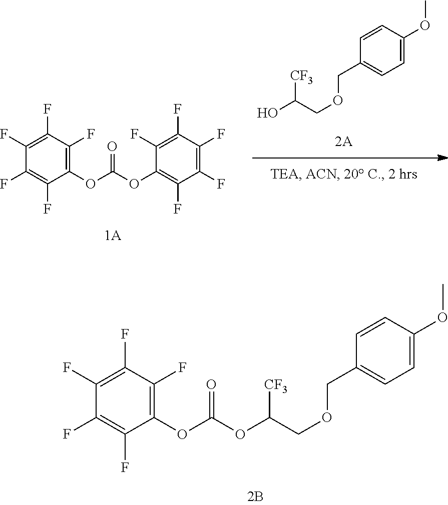 [SMILES: O=C(Oc1c(F)c(F)c(F)c(F)c1F)Oc1c(F)c(F)c(F)c(F)c1F ; COc1ccc(COCC(O)C(F)(F)F)cc1 ; COc1ccc(COCC(OC(=O)Oc2c(F)c(F)c(F)c(F)c2F)C(F)(F)F)cc1]

To a solution of 1A (300 mg, 761 μmol, 1.00 eq) in ACN (3.00 mL) was added TEA (385 mg, 3.81 mmol, 529 μL, 5.00 eq) and 2A (228 mg, 913 μmol, 1.20 eq). The mixture was stirred at 20° C. for 2 hrs. Compound 2B (350 mg, crude) was obtained as a light-yellow liquid to use the next step.

### Procedure for the preparation of 1,1,1-trifluoro-3-((4-methoxybenzyl)oxy)propan-2-yl 4-(7-fluoro-4,5-dihydropyrazolo[1,5-a]quinolin-2-yl)piperidine-1-carboxylate (2C)

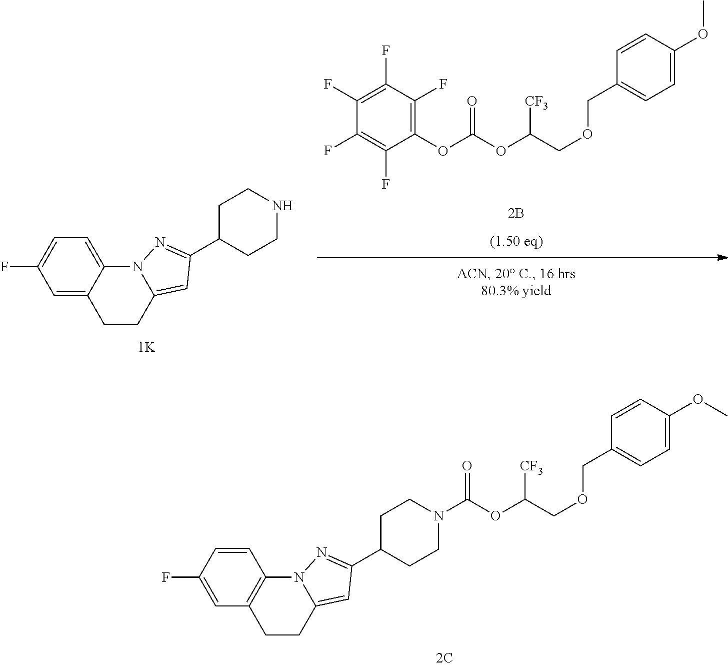 [SMILES: COc1ccc(COCC(OC(=O)Oc2c(F)c(F)c(F)c(F)c2F)C(F)(F)F)cc1 ; Fc1ccc2c(c1)CCc1cc(C3CCNCC3)nn1-2 ; COc1ccc(COCC(OC(=O)N2CCC(c3cc4n(n3)-c3ccc(F)cc3CC4)CC2)C(F)(F)F)cc1]

To a solution of compound 1K (140 mg, 455 μmol, 1.00 eq, HCl) in ACN (1.50 mL) was added 2B (314 mg, 682 μmol, 1.50 eq). The mixture was stirred at 20° C. for 16 hrs. The reaction mixture was concentrated under reduced pressure to give a residue. The residue was purified by prep-TLC (Petroleum ether/Ethyl acetate=5/1, R_{f}=0.16) to obtain compound 2C (200 mg, 365 μmol, 80.3% yield) as a light-yellow gum.

LCMS: Rt=0.558 min, MS=548.5, M+H⁺

### Procedure for the preparation of 1,1,1-trifluoro-3-hydroxypropan-2-yl 4-(7-fluoro-4,5-dihydropyrazolo[1,5-a]quinolin-2-yl)piperidine-1-carboxylate (2)

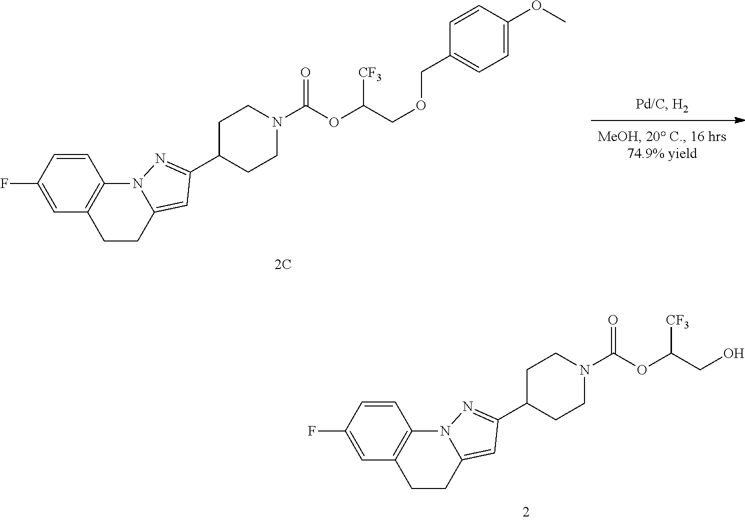 [SMILES: COc1ccc(COCC(OC(=O)N2CCC(c3cc4n(n3)-c3ccc(F)cc3CC4)CC2)C(F)(F)F)cc1 ; O=C(OC(CO)C(F)(F)F)N1CCC(c2cc3n(n2)-c2ccc(F)cc2CC3)CC1]

To a solution of 2C (200 mg, 365 μmol, 1.00 eq) in MeOH (2.00 mL) was added Pd/C (38.9 mg, 36.5 μmol, 10% purity, 0.10 eq) under N₂ atmosphere. The suspension was degassed and purged with H₂ for 3 times. The mixture was stirred under H₂ at 20° C. for 16 hrs. The reaction mixture was filtered and concentrated under reduced pressure to give a residue. The residue was purified by prep-HPLC (column: Phenomenex luna C18 150*25 mm*10 um; mobile phase: [water (FA)-ACN]; gradient: 49%-79% B over 10 min) to obtain compound 2 (117 mg, 273 μmol, 74.9% yield, 99.9% purity) as a colorless gum.

LCMS: Rt=0.461 min, MS=428.8, M+H⁺

¹H NMR: (400 MHz, CDCl₃)

δ 7.77-7.81 (m, 1H), 6.94-7.03 (m, 2H), 5.97 (s, 1H), 5.27 (s, 1H), 4.15-4.33 (m, 2H), 4.00-4.03 (m, 1H), 3.89-3.98 (m, 1H), 2.91-3.04 (m, 7H), 2.45 (s, 1H), 2.03 (d, J=16.0 Hz, 2H), 1.56-1.77 (m, 2H).

### Example 3: 1,1,1-trifluoro-3-hydroxypropan-2-yl 4-(3,7-difluoro-4,5-dihydropyrazolo[1,5-a]quinolin-2-yl)piperidine-1-carboxylate

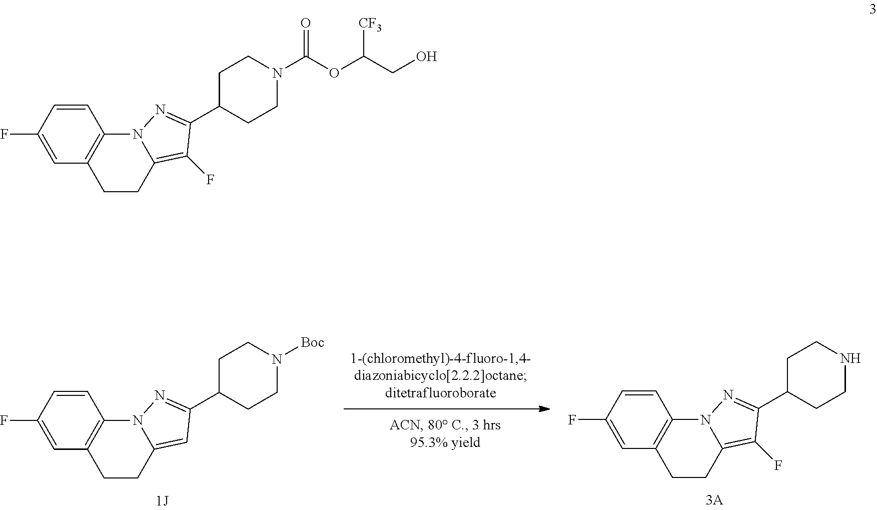 [SMILES: O=C(OC(CO)C(F)(F)F)N1CCC(c2nn3c(c2F)CCc2cc(F)ccc2-3)CC1 ; CC(C)(C)OC(=O)N1CCC(c2cc3n(n2)-c2ccc(F)cc2CC3)CC1 ; Fc1ccc2c(c1)CCc1c(F)c(C3CCNCC3)nn1-2]

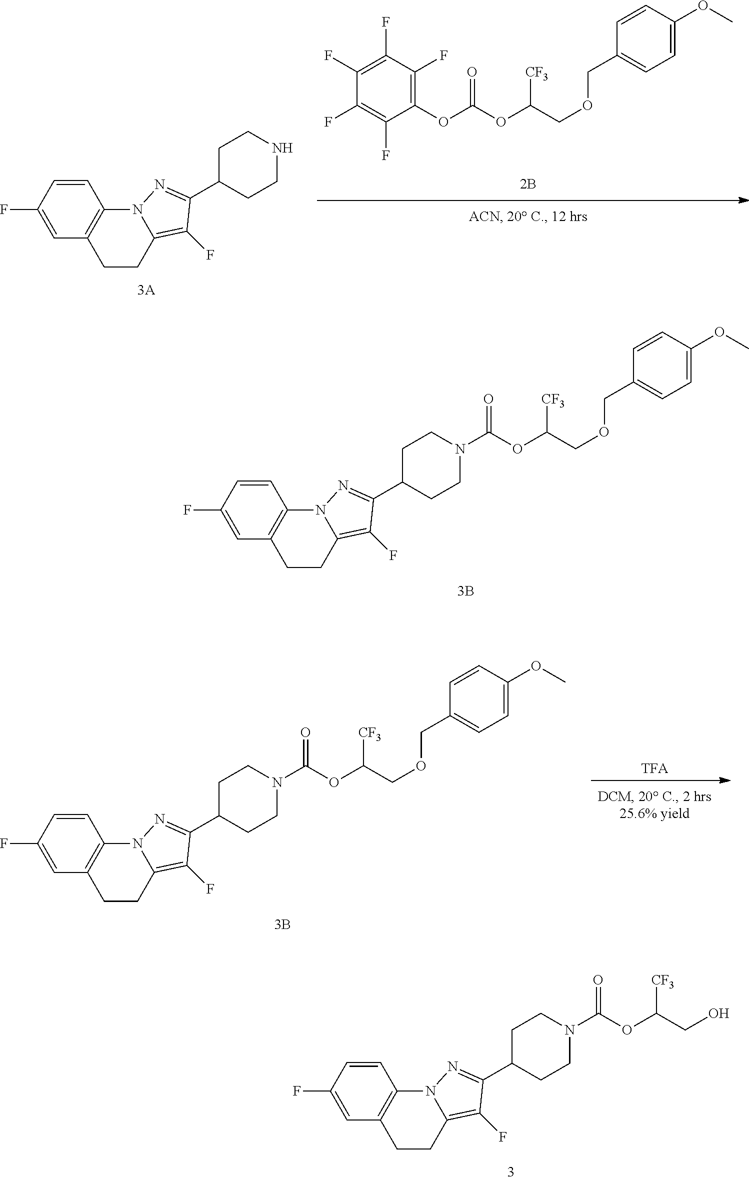 [SMILES: Fc1ccc2c(c1)CCc1c(F)c(C3CCNCC3)nn1-2 ; COc1ccc(COCC(OC(=O)Oc2c(F)c(F)c(F)c(F)c2F)C(F)(F)F)cc1 ; COc1ccc(COCC(OC(=O)N2CCC(c3nn4c(c3F)CCc3cc(F)ccc3-4)CC2)C(F)(F)F)cc1 ; "TFA" ; COc1ccc(COCC(OC(=O)N2CCC(c3nn4c(c3F)CCc3cc(F)ccc3-4)CC2)C(F)(F)F)cc1 ; O=C(OC(CO)C(F)(F)F)N1CCC(c2nn3c(c2F)CCc2cc(F)ccc2-3)CC1]

### Scheme-3: Synthesis of 1,1,1-trifluoro-3-hydroxypropan-2-yl 4-(3,7-difluoro-4,5-dihydropyrazolo[1,5-a]quinolin-2-yl)piperidine-1-carboxylate (3)

### Procedure for the preparation of 3,7-difluoro-2-(piperidin-4-yl)-4,5-dihydropyrazolo[1,5-a]quinoline (3A)

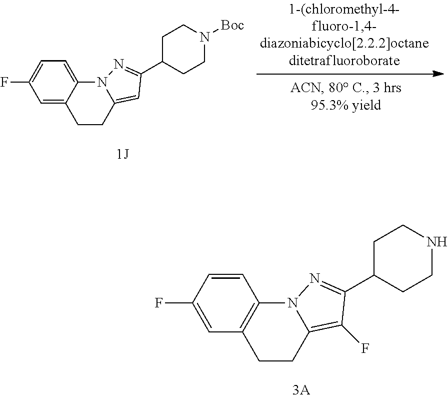 [SMILES: CC(C)(C)OC(=O)N1CCC(c2cc3n(n2)-c2ccc(F)cc2CC3)CC1 ; Fc1ccc2c(c1)CCc1c(F)c(C3CCNCC3)nn1-2]

A mixture of compound 1J (800 mg, 2.15 mmol, 1.00 eq), 1-(chloromethyl)-4-fluoro-1,4-diazoniabicyclo[2.2.2]octaneditetrafluoroborate (839 mg, 2.37 mmol, 1.10 eq) in ACN (8.00 mL) was degassed and purged with N₂ for 3 times, and then the mixture was stirred at 80° C. for 3 hrs under N₂ atmosphere. The crude product was used into the next step without further work-up. The residue was purified by prep-HPLC (column: Phenomenex luna C18 150×25 mm×10 um; mobile phase: [water (FA)-ACN]; gradient: 10%-40% B over 10 min) to give compound 3A (600 mg, 2.05 mmol, 95.3% yield, 98.9% purity).

LCMS: Rt=0.280 min, MS=290.3, M+H⁺

### Procedure for preparation of 1,1,1-trifluoro-3-hydroxypropan-2-yl 4-(3,7-difluoro-4,5-dihydropyrazolo[1,5-a]quinolin-2-yl)piperidine-1-carboxylate (3)

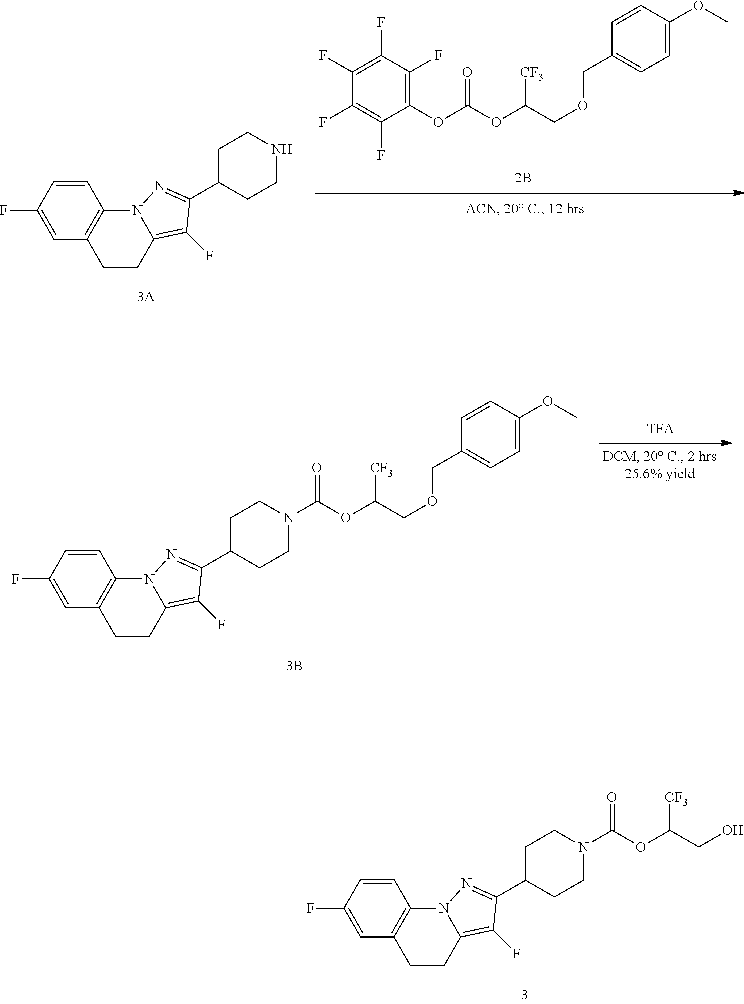 [SMILES: Fc1ccc2c(c1)CCc1c(F)c(C3CCNCC3)nn1-2 ; COc1ccc(COCC(OC(=O)Oc2c(F)c(F)c(F)c(F)c2F)C(F)(F)F)cc1 ; COc1ccc(COCC(OC(=O)N2CCC(c3nn4c(c3F)CCc3cc(F)ccc3-4)CC2)C(F)(F)F)cc1 ; O=C(OC(CO)C(F)(F)F)N1CCC(c2nn3c(c2F)CCc2cc(F)ccc2-3)CC1]

The compound was prepared according to the same synthesis step of example 2 starting from 3A to afford compound 3 (130 mg, 271 μmol, 25.6% yield, 93.0% purity) as a yellow solid.

LCMS: Rt=0.451 min, MS=446.4, M+H⁺

¹H NMR: (400 MHz, DMSO_d₆)

δ 7.75-7.79 (m, 1H), 6.99-7.03 (m, 1H), 6.94-6.97 (m, 1H), 5.26-5.30 (m, 1H), 4.01-4.03 (m, 2H), 3.85-3.92 (m, 1H), 3.02-3.14 (m, 1H), 2.02-2.99 (m, 2H), 2.92-2.94 (m, 6H), 1.91-2.01 (m, 2H), 1.66-1.88 (m, 2H).

### Example 4: 1,1,1-trifluoro-3-hydroxypropan-2-yl 4-(3-chloro-7-fluoro-4,5-dihydropyrazolo[1,5-a]quinolin-2-yl)piperidine-1-carboxylate

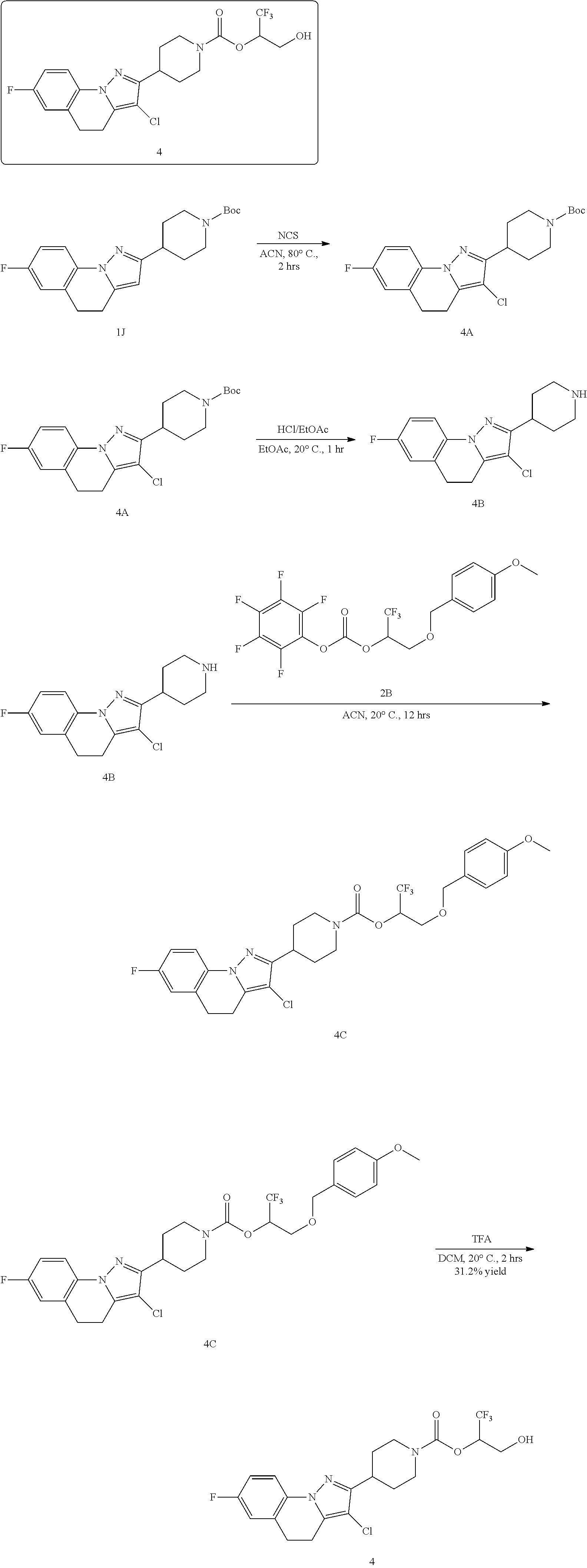

### Scheme-4: Synthesis of 1,1-trifluoro-3-hydroxypropan-2-yl 4-(3-chloro-7-fluoro-4,5-dihydropyrazolo[1,5-a]quinolin-2-yl)piperidine-1-carboxylate (4)

### Procedure for preparation of tert-butyl 4-(3-chloro-7-fluoro-4,5-dihydropyrazolo[1,5-a]quinolin-2-yl)piperidine-1-carboxylate (4A)

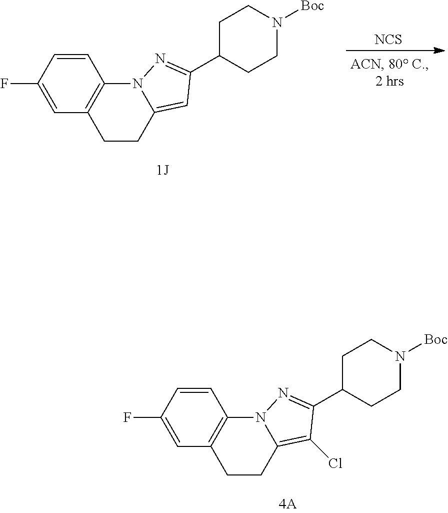 [SMILES: CC(C)(C)OC(=O)N1CCC(c2cc3n(n2)-c2ccc(F)cc2CC3)CC1 ; "NCS" ; CC(C)(C)OC(=O)N1CCC(c2nn3c(c2Cl)CCc2cc(F)ccc2-3)CC1]

A mixture of compound 1J (500 mg, 1.35 mmol, 1.00 eq), NCS (216 mg, 1.62 mmol, 1.20 eq) in ACN (8.00 mL) was degassed and purged with N₂ for 3 times, and then the mixture was stirred at 80° C. for 2 hrs under N₂ atmosphere. The reaction mixture was concentrated under reduced pressure to remove solvent. The crude product was used into the next step without further purification to give compound 4A (550 mg, crude) was obtained as a yellow solid.

LCMS: Rt=0.530 min, MS=428.3, M+H⁺

### Procedure for preparation of 1,1-trifluoro-3-hydroxypropan-2-yl 4-(3-chloro-7-fluoro-4,5-dihydropyrazolo[1,5-a]quinolin-2-yl)piperidine-1-carboxylate (4)

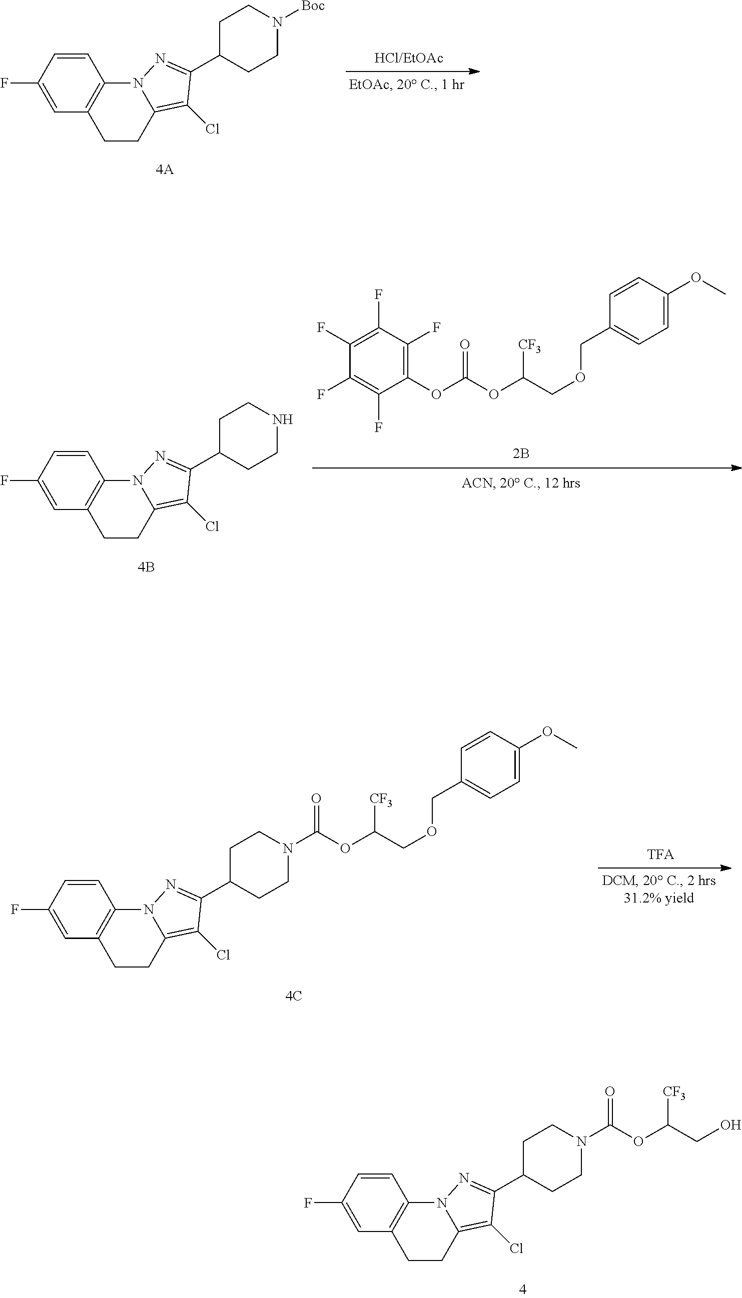 [SMILES: CC(C)(C)OC(=O)N1CCC(c2nn3c(c2Cl)CCc2cc(F)ccc2-3)CC1 ; Fc1ccc2c(c1)CCc1c(Cl)c(C3CCNCC3)nn1-2 ; COc1ccc(COCC(OC(=O)Oc2c(F)c(F)c(F)c(F)c2F)C(F)(F)F)cc1 ; COc1ccc(COCC(OC(=O)N2CCC(c3nn4c(c3Cl)CCc3cc(F)ccc3-4)CC2)C(F)(F)F)cc1 ; O=C(OC(CO)C(F)(F)F)N1CCC(c2nn3c(c2Cl)CCc2cc(F)ccc2-3)CC1]

The compound was prepared according to the same synthesis step of example 2 starting from 4A to afford compound 4 (271 mg, 509 μmol, 31.2% yield, 100% purity) was obtained as a white solid.

LCMS: Rt=0.475 min, MS=462.1, M+H⁺

¹H NMR: (400 MHz, DMSO_d₆)

δ 7.70-7.72 (m, 1H), 7.26-7.28 (m, 1H), 7.16-7.19 (m, 1H), 5.28-5.30 (m, 1H), 5.24-5.26 (m, 1H), 4.06-4.09 (m, 2H), 3.67-3.75 (m, 1H), 3.29-3.31 (m, 1H), 2.98-3.02 (m, 2H), 2.92-2.94 (m, 5H), 1.89-1.93 (m, 2H), 1.67-1.68 (m, 2H).

### Example 5: 1,1,1-trifluoro-3-hydroxypropan-2-yl 4-(3-bromo-7-fluoro-4,5-dihydropyrazolo[1,5-a]quinolin-2-yl)piperidine-1-carboxylate

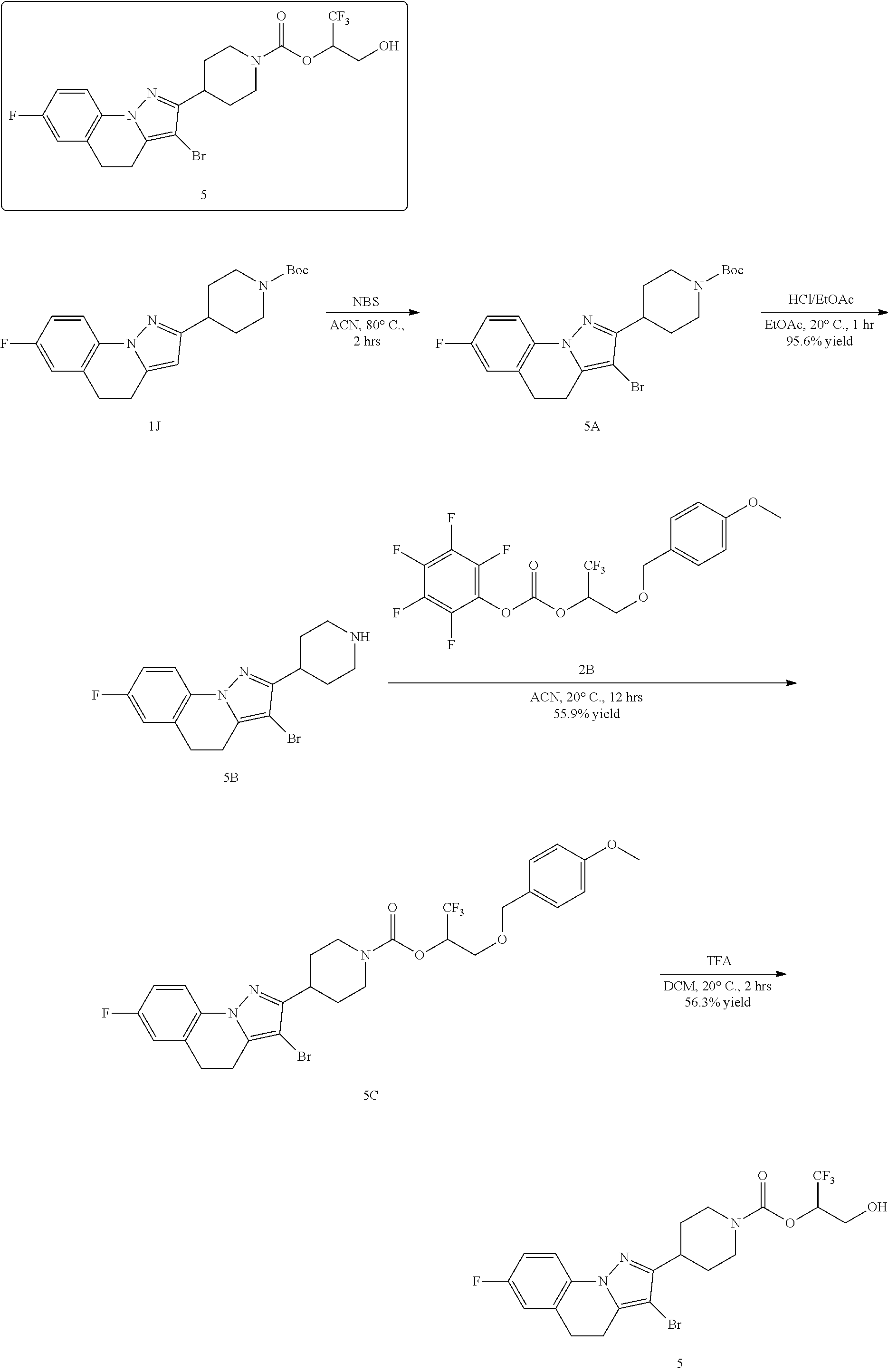

### Scheme-5: Synthesis of 1,1-trifluoro-3-hydroxypropan-2-yl 4-(3-bromo-7-fluoro-4,5-dihydropyrazolo[1,5-a]quinolin-2-yl)piperidine-1-carboxylate (5)

### Procedure for preparation of tert-butyl 4-(3-chloro-7-fluoro-4,5-dihydropyrazolo[1,5-a]quinolin-2-yl)piperidine-1-carboxylate (5A)

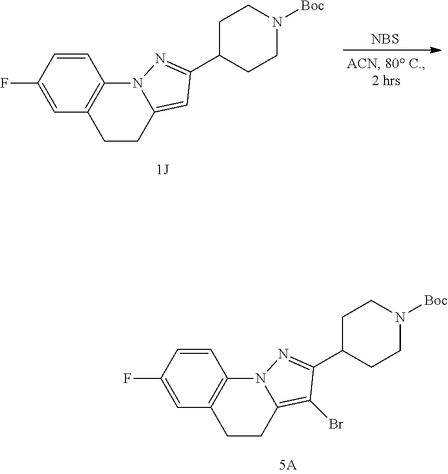 [SMILES: CC(C)(C)OC(=O)N1CCC(c2cc3n(n2)-c2ccc(F)cc2CC3)CC1 ; "NBS" ; CC(C)(C)OC(=O)N1CCC(c2nn3c(c2Br)CCc2cc(F)ccc2-3)CC1]

A mixture of compound 1J (500 mg, 1.35 mmol, 1.00 eq), NBS (359 mg, 2.02 mmol, 1.50 eq) in DCM (5.00 mL) was degassed and purged with N₂ for 3 times, and then the mixture was stirred at 20° C. for 2 hrs under N₂ atmosphere. The reaction was quenched by addition of 20.0 mL of saturated ammonium chloride, then acidify the clear solution with diluted hydrochloride acid, extracted with EtOAc. The combined organic layers were washed with brine, dried over Na₂SO₄, filtered and concentrated under reduced pressure to give a residue. The crude product was used into the next step without further purification to give compound 5A (780 mg, crude) was obtained as a yellow oil.

LCMS: Rt=0.542 min, MS=472.3, M+Na⁺

### Procedure for preparation of 1,1-trifluoro-3-hydroxypropan-2-yl 4-(3-bromo-7-fluoro-4,5-dihydropyrazolo[1,5-a]quinolin-2-yl)piperidine-1-carboxylate (5)

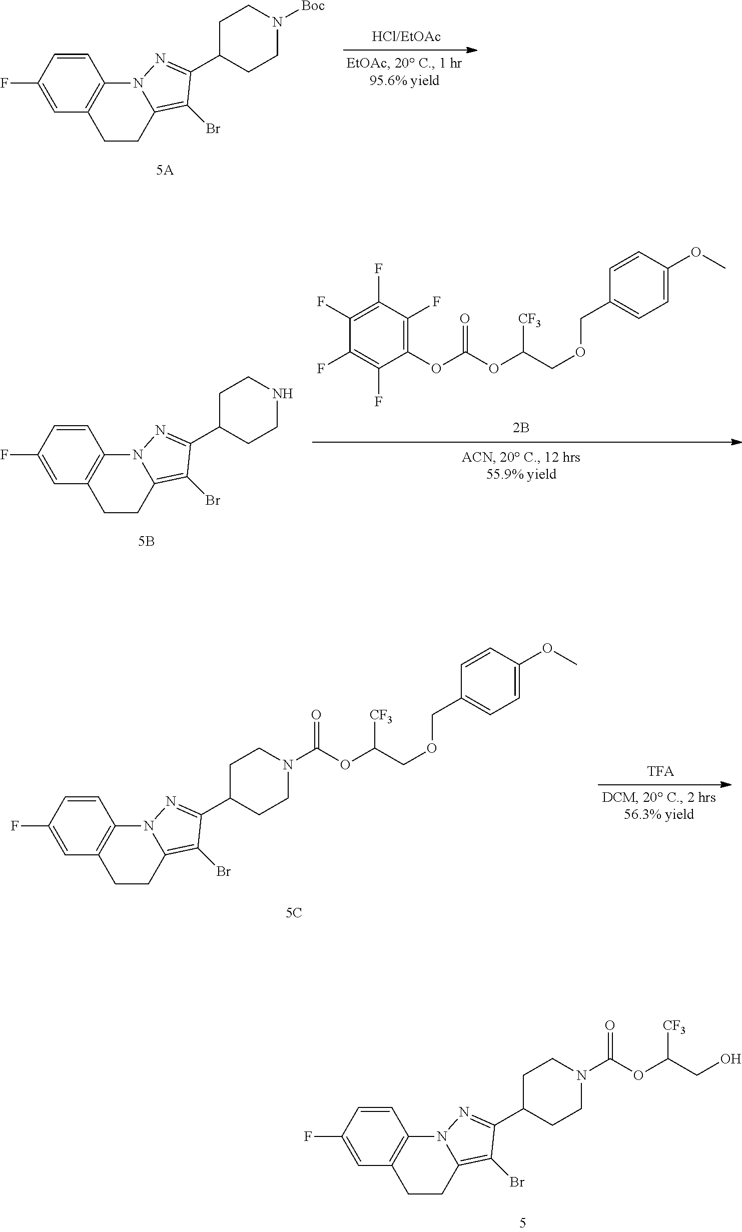 [SMILES: CC(C)(C)OC(=O)N1CCC(c2nn3c(c2Br)CCc2cc(F)ccc2-3)CC1 ; Fc1ccc2c(c1)CCc1c(Br)c(C3CCNCC3)nn1-2 ; COc1ccc(COCC(OC(=O)Oc2c(F)c(F)c(F)c(F)c2F)C(F)(F)F)cc1 ; COc1ccc(COCC(OC(=O)N2CCC(c3nn4c(c3Br)CCc3cc(F)ccc3-4)CC2)C(F)(F)F)cc1 ; O=C(OC(CO)C(F)(F)F)N1CCC(c2nn3c(c2Br)CCc2cc(F)ccc2-3)CC1]

The compound was prepared according to the same synthesis step of example 2 starting from 5A to afford compound 5 (150 mg, 389 μmol, 56.6% yield, 98.4% purity) was obtained as a white solid.

LCMS: Rt=0.475 min, MS=508.2, M+H⁺

¹H NMR: (400 MHz, DMSO_d₆)

δ 7.70-7.74 (m, 1H), 7.26-7.29 (m, 1H), 7.15-7.19 (m, 1H), 5.27-5.31 (m, 1H), 5.25-5.26 (m, 1H), 4.10 (s, 1H), 4.07 (s, 1H), 3.33-3.75 (m, 1H), 3.28-3.29 (m, 1H), 2.94-2.99 (m, 2H), 2.92-2.94 (m, 5H), 1.90-1.93 (m, 2H), 1.67-1.69 (m, 2H).

### Example 6: 1,1,1-trifluoro-3-hydroxypropan-2-yl 4-(7-fluoro-3-methyl-4,5-dihydropyrazolo[1,5-a]quinolin-2-yl)piperidine-1-carboxylate

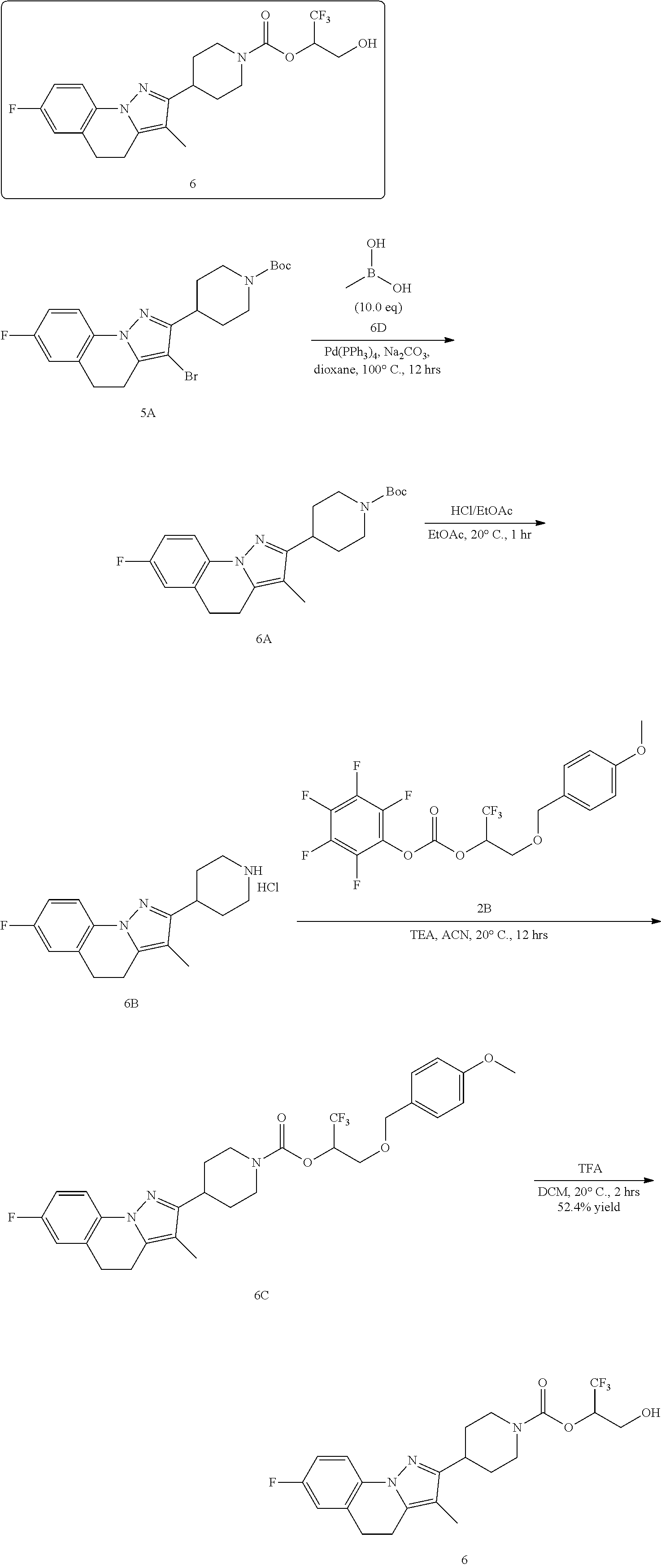

### Scheme-6: Synthesis of 1,1,1-trifluoro-3-hydroxypropan-2-yl 4-(7-fluoro-3-methyl-4,5-dihydropyrazolo[1,5-a]quinolin-2-yl)piperidine-1-carboxylate (6)

### Procedure for preparation of tert-butyl 4-(7-fluoro-3-methyl-4,5-dihydropyrazolo[1,5-a]quinolin-2-yl)piperidine-1-carboxylate (6A)

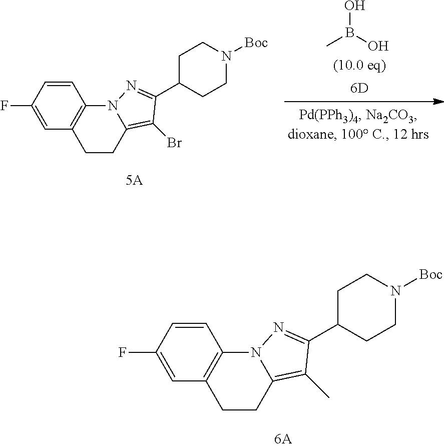

To a solution of compound 5A (300 mg, 666 μmol, 1.00 eq) in dioxane (5.00 mL) and H₂O (1.00 mL) and was added Pd(PPh₃)₄ (76.9 mg, 66.6 μmol, 0.10 eq) and Na₂CO₃ (211 mg, 2.00 mmol, 3.00 eq) and compound 6D (398 mg, 6.66 mmol, 10.0 eq). The mixture was stirred at 100° C. for 12 hrs under N₂. The reaction mixture was quenched by addition H₂O 3.00 mL and extracted with EtOAc. The combined organic layers were washed brine, and dried over Na₂SO₄, filtered, and concentrated under reduced pressure to give the residue. The residue was purified by prep-HPLC (column: CD05-Phenomenex luna C18 150×40×10 um; mobile phase: [water (FA)-ACN]; gradient: 60%-90% B over 11 min) to give compound 6A (200 mg, crude) as a yellow oil.

LCMS: Rt=0.489 min, MS=386.2, M+H⁺

### Procedure for preparation of 1,1,1-trifluoro-3-hydroxypropan-2-yl 4-(7-fluoro-3-methyl-4,5-dihydropyrazolo[1,5-a]quinolin-2-yl)piperidine-1-carboxylate (6)

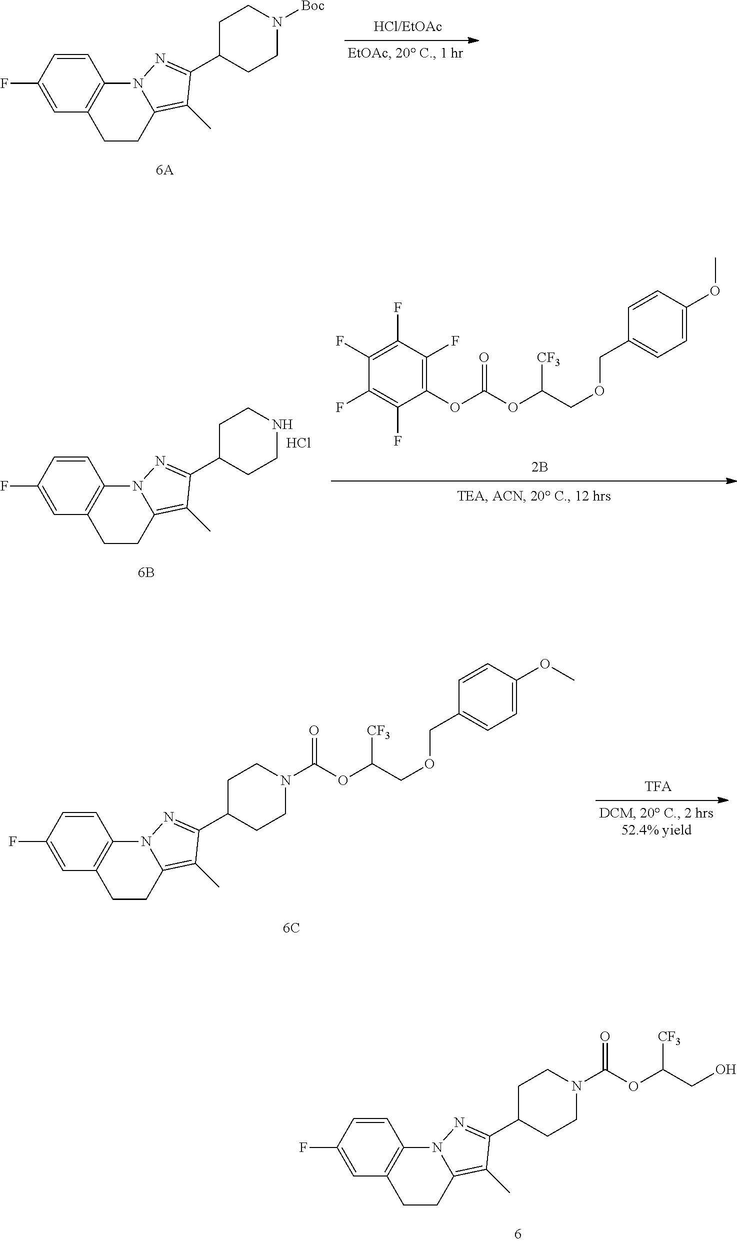 [SMILES: Cc1c(C2CCN(C(=O)OC(C)(C)C)CC2)nn2c1CCc1cc(F)ccc1-2 ; Cc1c(C2CCNCC2)nn2c1CCc1cc(F)ccc1-2 ; Cl ; COc1ccc(COCC(OC(=O)Oc2c(F)c(F)c(F)c(F)c2F)C(F)(F)F)cc1 ; COc1ccc(COCC(OC(=O)N2CCC(c3nn4c(c3C)CCc3cc(F)ccc3-4)CC2)C(F)(F)F)cc1 ; Cc1c(C2CCN(C(=O)OC(CO)C(F)(F)F)CC2)nn2c1CCc1cc(F)ccc1-2]

The compound was prepared according to the same synthesis step of example 2 starting from 6A to afford compound 6 (100 mg, 224 μmol, 52.4% yield, 98.9% purity) as a white solid.

LCMS: Rt=0.498 min, MS=442.1, M+H⁺

¹H NMR: (400 MHz, CDCl₃): δ 7.74-7.78 (m, 1H), 6.91-6.98 (m, 2H), 5.25-5.28 (m, 1H), 4.22-4.30 (m, 2H), 4.00-4.04 (m, 1H), 3.87-3.92 (m, 1H), 3.00-3.11 (m, 2H), 2.85-2.93 (m, 5H), 2.01 (s, 3H), 1.89-1.93 (m, 4H).

### Example 7: 1,1,1-trifluoro-3-hydroxypropan-2-yl 4-(7-fluoro-3-(trifluoromethyl)-4,5-dihydropyrazolo[1,5-a]quinolin-2-yl)piperidine-1-carboxylate

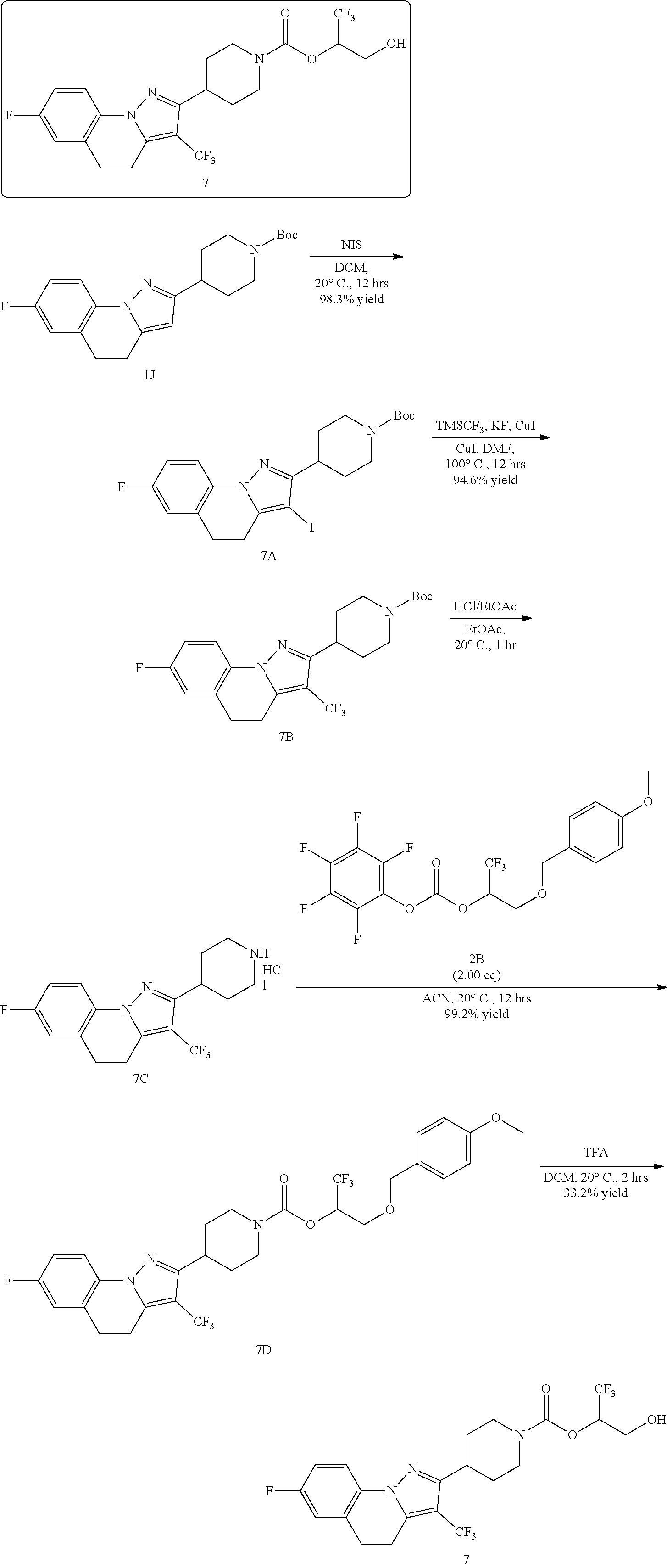

### Scheme-7: Synthesis of 1,1,1-trifluoro-3-hydroxypropan-2-yl 4-(7-fluoro-3-(trifluoromethyl)-4,5-dihydropyrazolo[1,5-a]quinolin-2-yl)piperidine-1-carboxylate (7)

### Procedure for preparation of tert-butyl 4-(7-fluoro-3-iodo-4,5-dihydropyrazolo[1,5-a]quinolin-2-yl)piperidine-1-carboxylate (7A)

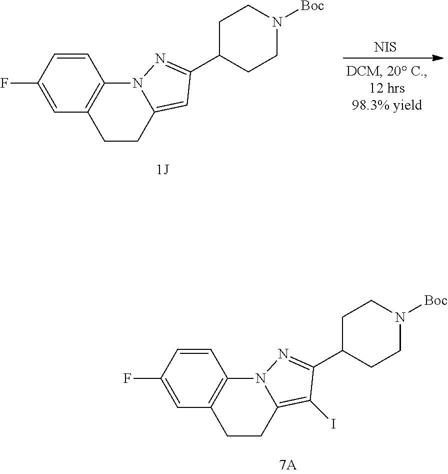 [SMILES: CC(C)(C)OC(=O)N1CCC(c2cc3n(n2)-c2ccc(F)cc2CC3)CC1 ; "NIS" ; CC(C)(C)OC(=O)N1CCC(c2nn3c(c2I)CCc2cc(F)ccc2-3)CC1]

A mixture of 1J (300 mg, 808 μmol, 1.00 eq), NIS (273 mg, 1.21 mmol, 1.50 eq), in DCM (5.00 m L) was degassed and purged with N₂ for 3 times, and then the mixture was stirred at 20° C. for 12 hrs under N₂ atmosphere. The reaction mixture was concentrated under reduced pressure to remove solvent. The residue was purified by column chromatography (SiO₂, Petroleum ether/Ethyl acetate=5/1, R_{f}=0.66) to give 7A (395 mg, 794 μmol, 98.3% yield) as a yellow solid.

LC-MS: Rt=0.580 min, MS=442.0, M-56+H⁺

### Procedure for preparation of tert-butyl 4-(7-fluoro-3-(trifluoromethyl)-4,5-dihydropyrazolo[1,5-a]quinolin-2-yl)piperidine-1-carboxylate (7B)

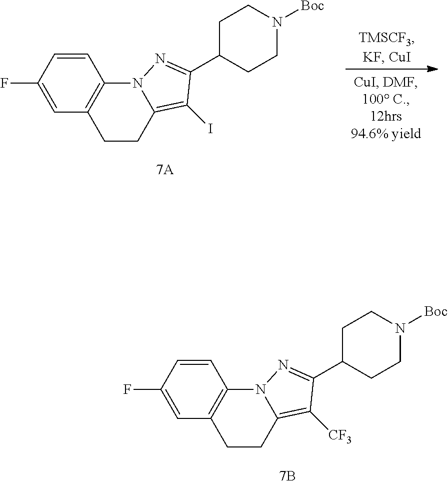

A mixture of 7A (395 mg, 794 μmol, 1.00 eq), TMSCF₃ (565 mg, 3.97 mmol, 5.00 eq), KF (55.4 mg, 953 μmol, 1.20 eq) and CuI (227 mg, 1.19 mmol, 1.50 eq) in DMF (4.00 mL) was degassed and purged with N₂ for 3 times, and then the mixture was stirred at 100° C. for 12 hrs under N₂ atmosphere. The reaction mixture was concentrated under reduced pressure to remove solvent. The residue was purified by flash silica gel chromatography (ISCO®; 4 g Sepa Flash® Silica Flash Column, Eluent of 0-20% Ethyl acetate/Petroleum ether gradient @ 100 mL/min) to give 7B (330 mg, 751 μmol, 94.6% yield) as a yellow solid.

LC-MS: Rt=0.578 min, MS=384.1, M+H⁺

### Procedure for preparation of 1,1,1-trifluoro-3-hydroxypropan-2-yl 4-(7-fluoro-3-methyl-4,5-dihydropyrazolo[1,5-a]quinolin-2-yl)piperidine-1-carboxylate (7)

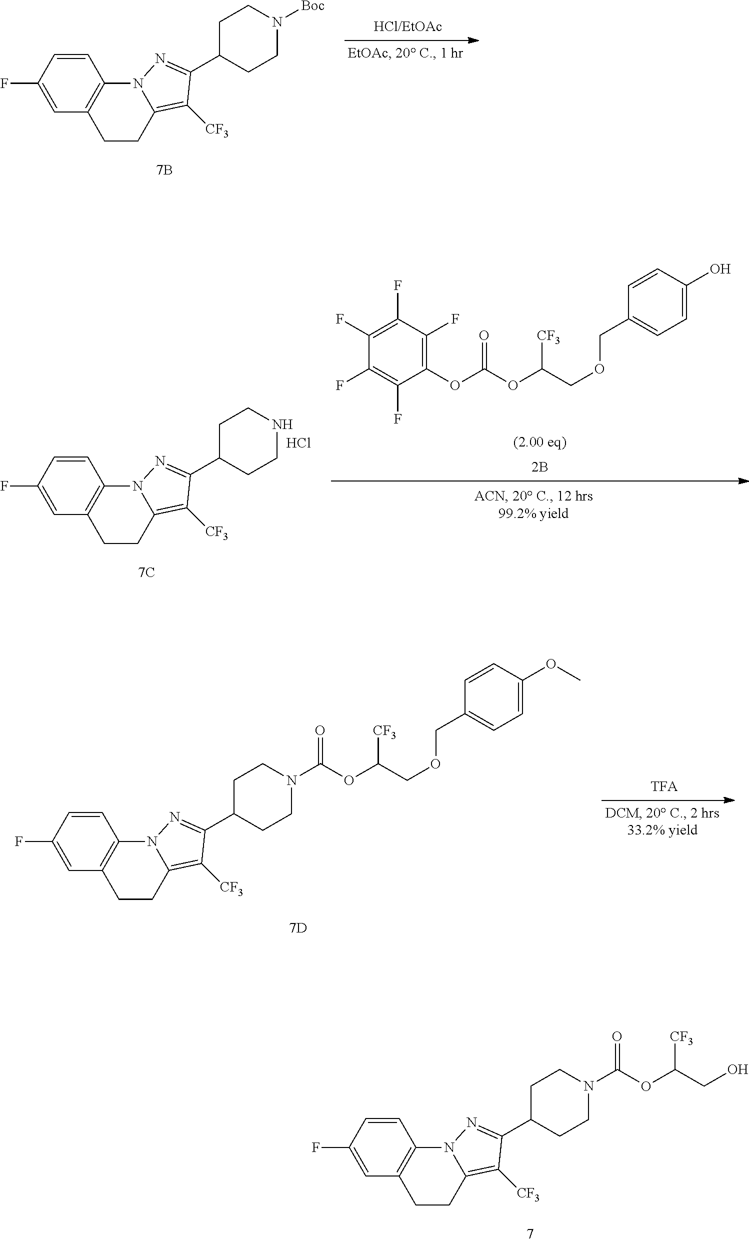 [SMILES: CC(C)(C)OC(=O)N1CCC(c2nn3c(c2C(F)(F)F)CCc2cc(F)ccc2-3)CC1 ; Fc1ccc2c(c1)CCc1c(C(F)(F)F)c(C3CCNCC3)nn1-2 ; Cl ; O=C(Oc1c(F)c(F)c(F)c(F)c1F)OC(COCc1ccc(O)cc1)C(F)(F)F ; COc1ccc(COCC(OC(=O)N2CCC(c3nn4c(c3C(F)(F)F)CCc3cc(F)ccc3-4)CC2)C(F)(F)F)cc1 ; O=C(OC(CO)C(F)(F)F)N1CCC(c2nn3c(c2C(F)(F)F)CCc2cc(F)ccc2-3)CC1]

The compound was prepared according to the same synthesis step of example 2 starting from 7B to afford compound 7 (50.0 mg, 97.0 μmol, 33.2% yield, 96.1% purity) as a white solid.

LCMS: Rt=0.522 min, MS=496.1, M+H⁺

¹H NMR: (400 MHz, CDCl₃): δ 7.82-7.86 (m, 1H), 6.97-7.01 (m, 2H), 5.28 (s, 1H), 4.05-4.26 (m, 2H), 4.01-4.02 (m, 1H), 3.91-4.01 (m, 1H), 2.98-3.07 (m, 7H), 2.48-2.96 (m, 1H), 1.91-1.93 (m, 4H)

### Example 8: 1,1,1-trifluoro-3-hydroxypropan-2-yl 4-(7-fluoro-4,5-dihydropyrazolo[1,5-a]quinolin-2-yl-3-d)piperidine-1-carboxylate

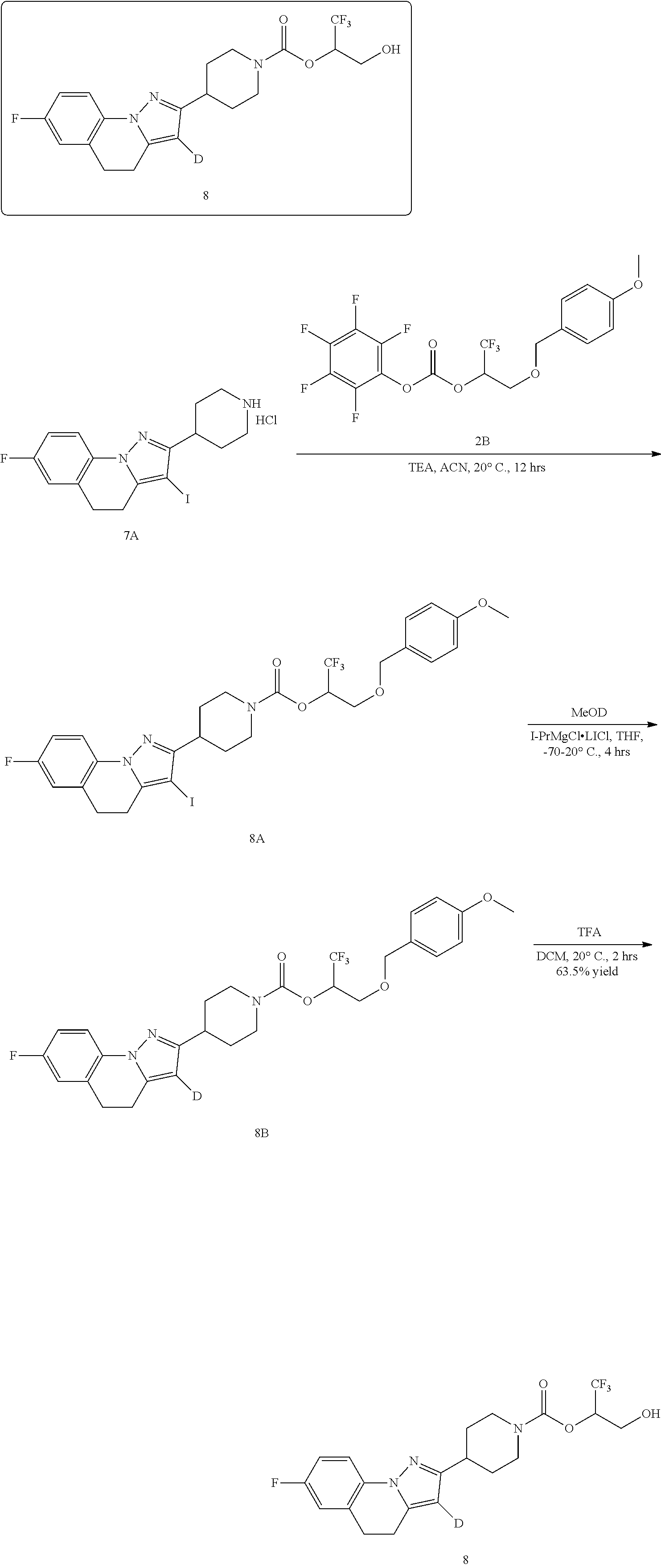

### Scheme-8: Synthesis of 1,1,1-trifluoro-3-hydroxypropan-2-yl 4-(7-fluoro-4,5-dihydropyrazolo[1,5-a]quinolin-2-yl-3-d)piperidine-1-carboxylate (8)

### Procedure for preparation of 1,1,1-trifluoro-3-((4-methoxybenzyl)oxy)propan-2-yl 4-(7-fluoro-3-iodo-4,5-dihydropyrazolo[1,5-a]quinolin-2-yl)piperidine-1-carboxylate (8A)

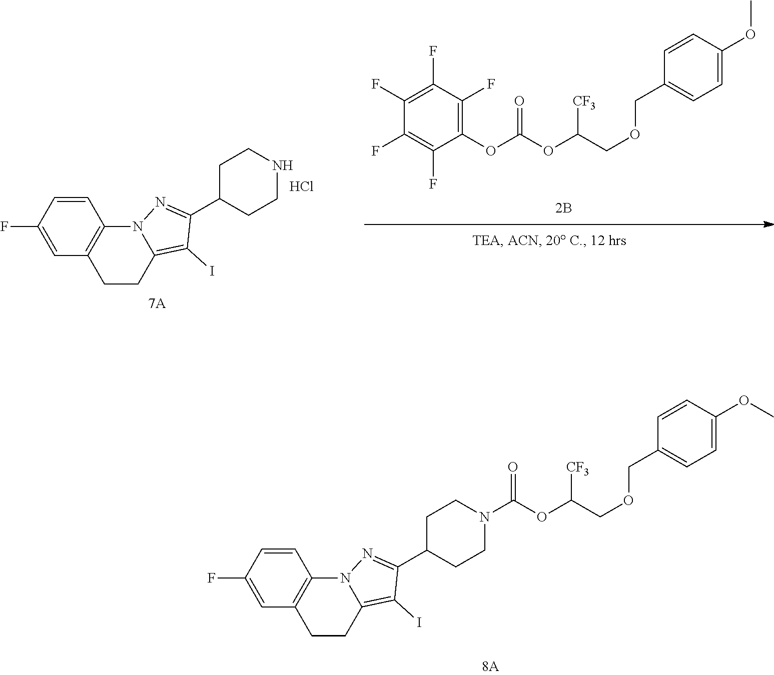 [SMILES: Fc1ccc2c(c1)CCc1c(I)c(C3CCNCC3)nn1-2 ; Cl ; COc1ccc(COCC(OC(=O)Oc2c(F)c(F)c(F)c(F)c2F)C(F)(F)F)cc1 ; COc1ccc(COCC(OC(=O)N2CCC(c3nn4c(c3I)CCc3cc(F)ccc3-4)CC2)C(F)(F)F)cc1]

To a solution of compound 7A (350 mg, 881 μmol, 1.00 eq) in ACN (5.00 mL) was added TEA (445 mg, 4.41 mmol, 613 μL, 5.00 eq) and compound 2B (486 mg, 1.06 mmol, 1.20 eq). The mixture was stirred at 20° C. for 12 hrs. The reaction mixture was filtered and concentrated under reduced pressure to give a residue. The residue was purified by prep-HPLC (column: CD05-Phenomenex luna C18 150×40×10 um; mobile phase: [water (FA)-ACN]; gradient: 70%-100% B over 11 min) to give compound 8A (300 mg, crude) as a yellow solid.

LCMS: Rt=0.479 min, MS=674.1, M+H⁺

### Procedure for preparation of 1,1,1-trifluoro-3-((4-methoxybenzyl)oxy)propan-2-yl 4-(7-fluoro-4,5-dihydropyrazolo[1,5-a]quinolin-2-yl-3-d)piperidine-1-carboxylate (8B)

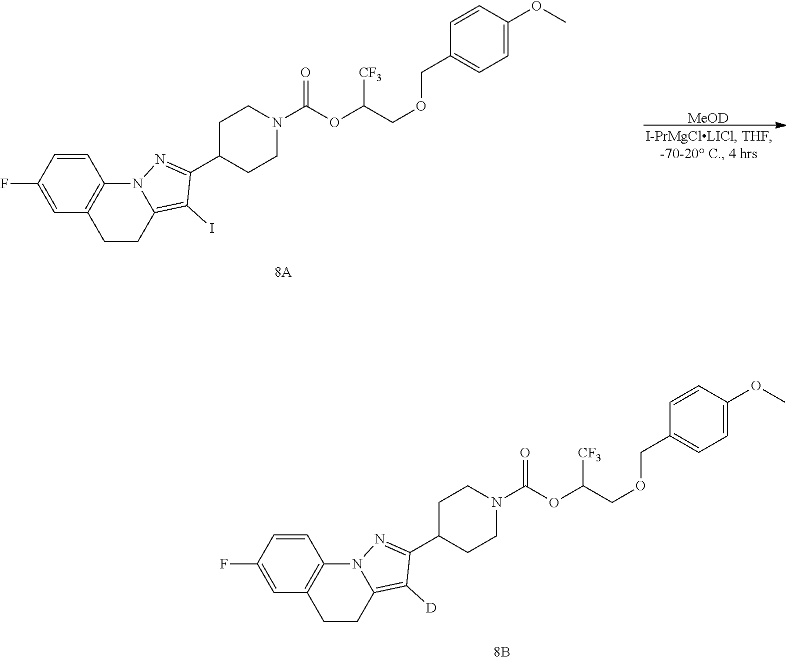 [SMILES: "MeOD" ; COc1ccc(COCC(OC(=O)N2CCC(c3nn4c(c3I)CCc3cc(F)ccc3-4)CC2)C(F)(F)F)cc1 ; [2H]c1c(C2CCN(C(=O)OC(COCc3ccc(OC)cc3)C(F)(F)F)CC2)nn2c1CCc1cc(F)ccc1-2]

To a solution of compound 8A (50.0 mg, 74.2 μmol, 1.00 eq) in THF (0.50 mL) was degassed and purged with N₂ for 3 times, followed by the mixture was cooled to −70° C. and treated slowly with i-PrMgCl·LiCl (1.3 M, 142 μL, 2.50 eq) under N₂ atmosphere. The mixture was stirred for 1 hr under N₂, and MeOD (742 μmol, 1.00 mL, 10.0 eq) was added and the reaction mixture was allowed to warm up to 20° C. Then the mixture was stirred at 20° C. for 3 hrs. The reaction mixture was quenched by addition NH₄Cl (1.00 mL), and the reaction mixture was diluted with water (3.00 mL) and extracted with EtOAc. The combined organic layers were washed with brine, dried over Na₂SO₄, filtered and concentrated under reduced pressure to give compound 8B (40.0 mg, crude) as a yellow solid.

LCMS: Rt=3.478 min, MS=549.1, M+H⁺

### Procedure for preparation of 1,1,1-trifluoro-3-hydroxypropan-2-yl 4-(7-fluoro-4,5-dihydropyrazolo[1,5-a]quinolin-2-yl-3-d)piperidine-1-carboxylate (8)

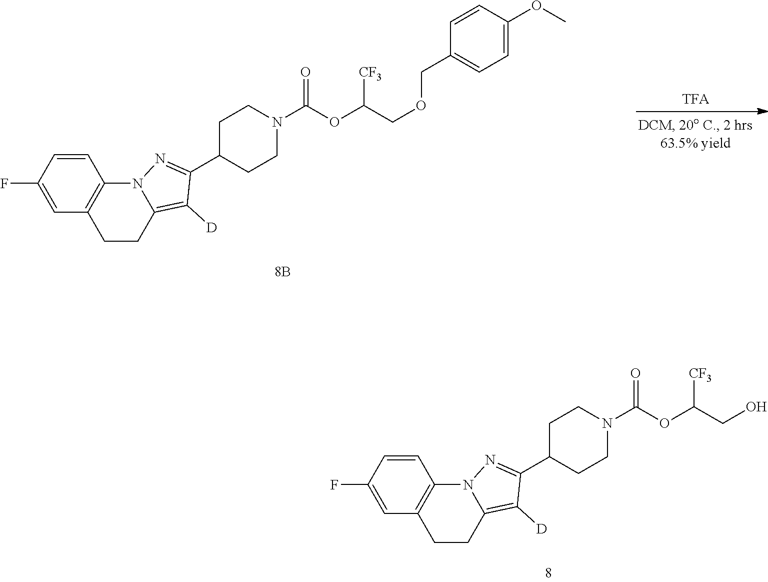 [SMILES: [2H]c1c(C2CCN(C(=O)OC(COCc3ccc(OC)cc3)C(F)(F)F)CC2)nn2c1CCc1cc(F)ccc1-2 ; [2H]c1c(C2CCN(C(=O)OC(CO)C(F)(F)F)CC2)nn2c1CCc1cc(F)ccc1-2]

To a solution of compound 8B (40.0 mg, 72.9 μmol, 1.00 eq) in DCM (0.5 mL) was added TFA (41.5 mg, 364 μmol, 27.0 μL, 5.00 eq). The mixture was stirred at 20° C. for 2 hrs. The reaction mixture was quenched by addition sodium bicarbonate saturated solution 5.00 mL at 0° C. and extracted with DCM. The combined organic layers were washed with brine, dried over Na₂SO₄, filtered and concentrated under reduced pressure to give a residue. The residue was purified by prep-HPLC (column: Phenomenex luna C18 150×25 mm×10 um; mobile phase: [water (FA)-ACN]; gradient: 44%-74% B over 10 min) to give compound 8 (20.0 mg, 46.3 μmol, 63.5% yield, 99.2% purity) as a yellow solid.

LCMS: Rt=0.476 min, MS=429.1, M+H⁺

¹H NMR: (400 MHz, CDCl₃): δ 7.76-7.80 (m, 1H), 6.93-7.02 (m, 2H), 5.26-5.29 (m, 1H), 4.19-4.27 (m, 2H), 3.99-4.03 (m, 1H), 3.86-3.91 (m, 1H), 2.90-3.11 (m, 7H), 1.94-2.05 (m, 2H), 1.69-1.79 (m, 2H).

### Example 9: 1,1,1-trifluoro-3-hydroxypropan-2-yl 4-(7-fluoro-3-morpholino-4,5-dihydropyrazolo[1,5-a]quinolin-2-yl)piperidine-1-carboxylate

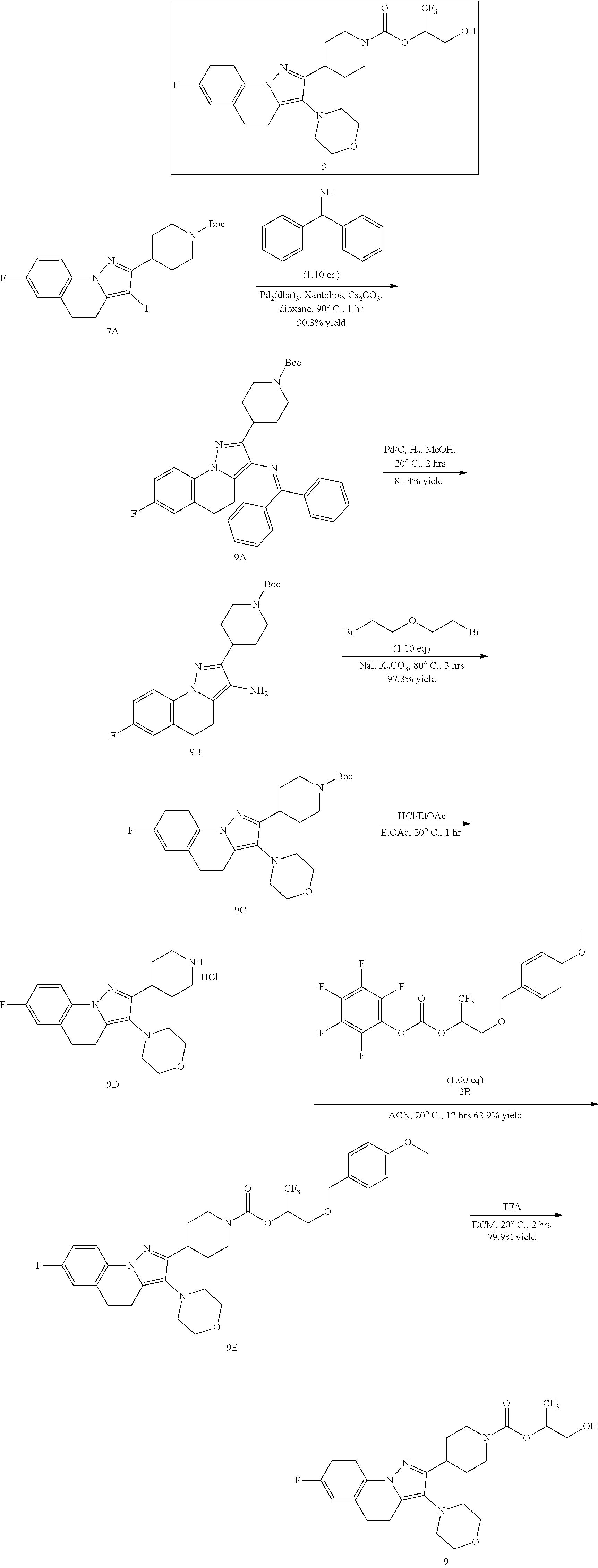

### Scheme-9: Synthesis of 1,1,1-trifluoro-3-hydroxypropan-2-yl 4-(7-fluoro-3-morpholino-4,5-dihydropyrazolo[1,5-a]quinolin-2-yl)piperidine-1-carboxylate (9)

### Procedure for preparation of tert-butyl 4-(3-((diphenylmethylene)amino)-7-fluoro-4,5-dihydropyrazolo[1,5-a]quinolin-2-yl)piperidine-1-carboxylate (9A)

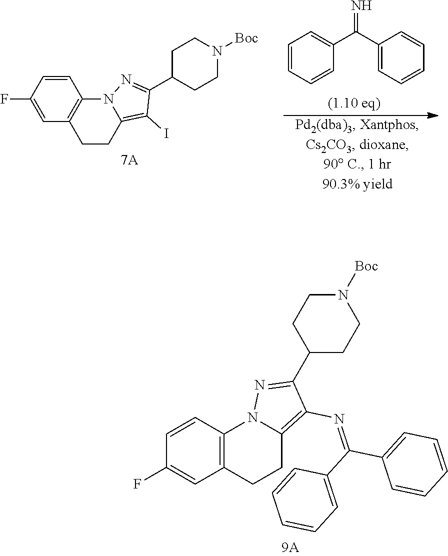 [SMILES: CC(C)(C)OC(=O)N1CCC(c2nn3c(c2I)CCc2cc(F)ccc2-3)CC1 ; N=C(c1ccccc1)c1ccccc1 ; "Pd2(dba)3, Xantphos,Cs2CO3, dioxane,90° C., 1 hr90.3% yield" ; CC(C)(C)OC(=O)N1CCC(c2nn3c(c2N=C(c2ccccc2)c2ccccc2)CCc2cc(F)ccc2-3)CC1]

A mixture of compound 7A (700 mg, 1.41 mmol, 1.00 eq), benzophenone imine (281 mg, 1.55 mmol, 260 μL, 1.10 eq), Cs₂CO₃ (1.38 g, 4.22 mmol, 3.00 eq), Xantphos (163 mg, 282 μmol, 0.20 eq) and Pd₂(dba)₃ (129 mg, 141 μmol, 0.10 eq) in dioxane (10.0 mL) was degassed and purged with N₂ for 3 times, and then the mixture was stirred at 90° C. for 1 hr under N₂ atmosphere. The reaction mixture was concentrated under reduced pressure to remove solvent. The residue was purified by flash silica gel chromatography (ISCO®; 12 g SepaFlash® Silica Flash Column, Eluent of 0-5% Ethyl acetate/Petroleum ether gradient @ 30 mL/min) to give compound 9A (700 mg, 1.27 mmol, 90.3% yield) as a yellow solid.

LCMS: Rt=0.547 min, MS=551.3, M+H⁺,

### Procedure for preparation of tert-butyl 4-(3-amino-7-fluoro-4,5-dihydropyrazolo[1,5-a]quinolin-2-yl)piperidine-1-carboxylate (9B)

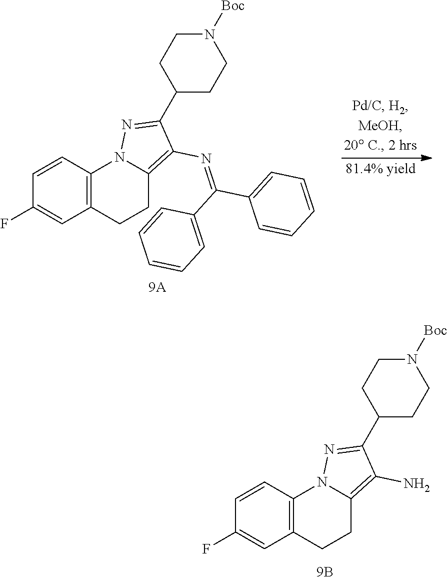 [SMILES: CC(C)(C)OC(=O)N1CCC(c2nn3c(c2N=C(c2ccccc2)c2ccccc2)CCc2cc(F)ccc2-3)CC1 ; CC(C)(C)OC(=O)N1CCC(c2nn3c(c2N)CCc2cc(F)ccc2-3)CC1]

To a solution compound 9A (700 mg, 1.27 mmol, 1.00 eq) in MeOH (1.00 mL) was added Pd/C (22.6 mg, 127 μmol, 60.0% purity, 0.10 eq) under N₂ atmosphere. The suspension was degassed and purged with H₂ for 3 times. The mixture was stirred under H₂ (50 psi) at 20° C. for 12 hrs. The reaction was quenched by addition of 30.0 mL of saturated ammonium chloride, then acidify the clear solution with diluted hydrochloride acid, extracted with EtOAc. The combined organic layers were washed with brine, dried over Na₂SO₄, filtered and concentrated under reduced pressure to give a residue. The residue was purified by flash silica gel chromatography (ISCO®; 12 g Sepa Flash® Silica Flash Column, Eluent of 0˜68% Ethyl acetate/Petroleum ether gradient @80 mL/min) to give compound 9B (400 mg, 1.04 mmol, 81.4% yield) as a yellow solid.

LCMS: Rt=0.338 min, MS=331.1, M-56+H⁺,

### Procedure for preparation of tert-butyl 4-(7-fluoro-3-morpholino-4,5-dihydropyrazolo[1,5-a]quinolin-2-yl)piperidine-1-carboxylate (9C)

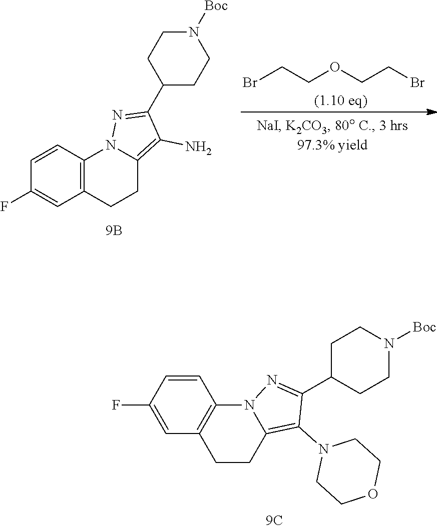 [SMILES: CC(C)(C)OC(=O)N1CCC(c2nn3c(c2N)CCc2cc(F)ccc2-3)CC1 ; BrCCOCCBr ; CC(C)(C)OC(=O)N1CCC(c2nn3c(c2N2CCOCC2)CCc2cc(F)ccc2-3)CC1]

A mixture of compound 9B (200 mg, 518 μmol, 1.00 eq), 2-Bromoethyl ethyl ether (144 mg, 621 μmol, 78.1 μL, 1.10 eq), KI (8.59 mg, 51.8 μmol, 0.10 eq), K₂CO₃ (215 mg, 1.55 mmol, 3.00 eq) in ACN (2.00 mL) was degassed and purged with N₂ for 3 times, and then the mixture was stirred at 80° C. for 3 hrs under N₂ atmosphere. The reaction mixture was concentrated under reduced pressure to remove solvent. The residue was purified by flash silica gel chromatography (ISCO®; 40 g Sepa Flash® Silica Flash Column, Eluent of 0˜ 28% Ethylacetate/Petroleum ether gradient @ 100 mL/min) to give compound 9C (230 mg, 504 μmol, 97.3% yield) as a white solid.

LCMS: Rt=0.478 min, MS=401.1, M+H⁺,

### Procedure for preparation of 1,1,1-trifluoro-3-hydroxypropan-2-yl 4-(7-fluoro-3-morpholino-4,5-dihydropyrazolo[1,5-a]quinolin-2-yl)piperidine-1-carboxylate (9)

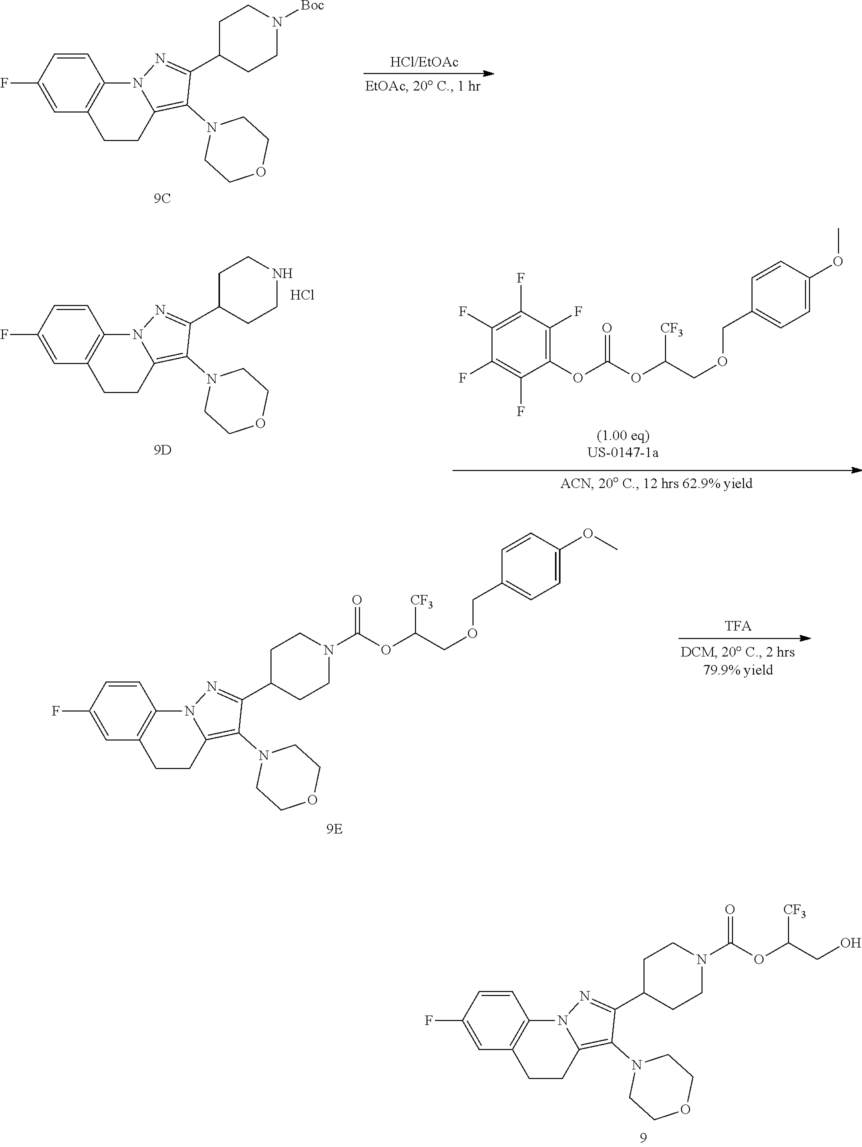 [SMILES: CC(C)(C)OC(=O)N1CCC(c2nn3c(c2N2CCOCC2)CCc2cc(F)ccc2-3)CC1 ; Fc1ccc2c(c1)CCc1c(N3CCOCC3)c(C3CCNCC3)nn1-2 ; Cl ; COc1ccc(COCC(OC(=O)Oc2c(F)c(F)c(F)c(F)c2F)C(F)(F)F)cc1 ; COc1ccc(COCC(OC(=O)N2CCC(c3nn4c(c3N3CCOCC3)CCc3cc(F)ccc3-4)CC2)C(F)(F)F)cc1 ; O=C(OC(CO)C(F)(F)F)N1CCC(c2nn3c(c2N2CCOCC2)CCc2cc(F)ccc2-3)CC1]

The compound was prepared according to the same synthesis step of example 2 starting from 9C to afford compound 9 (110 mg, 215 μmol, 79.9% yield, 100% purity) as a white solid.

LCMS: Rt=0.547 min, MS=551.3, M+H⁺.

¹H NMR: (400 MHz, CDCl₃): δ 7.77-7.81 (m, 1H), 6.93-6.96 (m, 1H), 5.29-5.33 (m, 1H), 4.02-4.23 (m, 1H), 4.02-4.03 (m, 2H), 4.02-4.03 (m, 1H), 3.80-3.92 (m, 1H), 3.80 (s, 4H), 2.94-3.01 (m, 11H), 2.42-2.48 (m, 1H), 1.58-1.96 (m, 4H).

### Example 10: 1,1,1-trifluoro-3-hydroxypropan-2-yl 4-(3-(diethylamino)-7-fluoro-4,5-dihydropyrazolo[1,5-a]quinolin-2-yl)piperidine-1-carboxylate

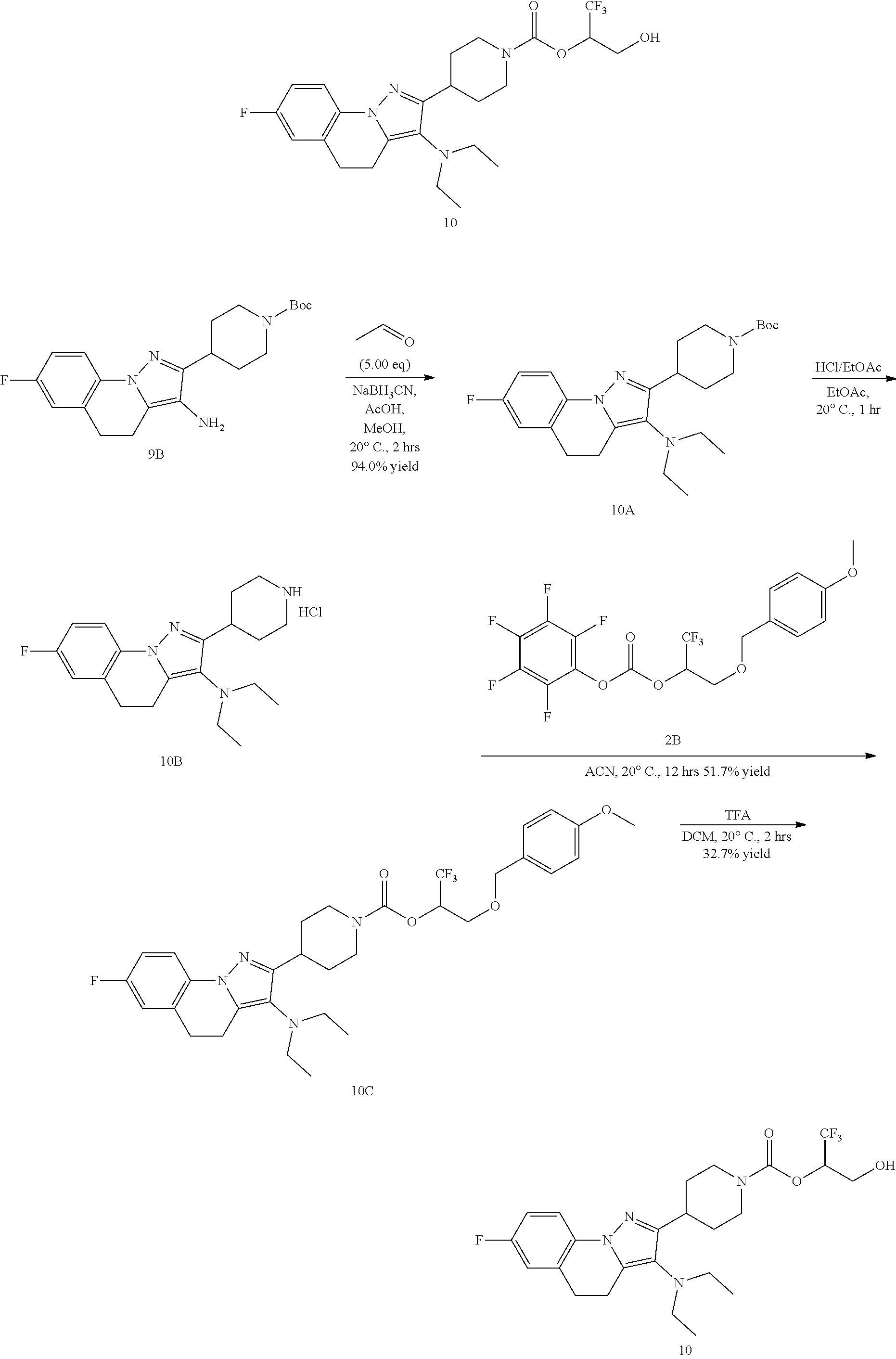

### Scheme-10: Synthesis of 1,1,1-trifluoro-3-hydroxypropan-2-yl 4-(3-(diethylamino)-7-fluoro-4,5-dihydropyrazolo[1,5-a]quinolin-2-yl)piperidine-1-carboxylate (10)

### Procedure for preparation of tert-butyl 4-(3-(diethylamino)-7-fluoro-4,5-dihydropyrazolo[1,5-a]quinolin-2-yl)piperidine-1-carboxylate (10A)

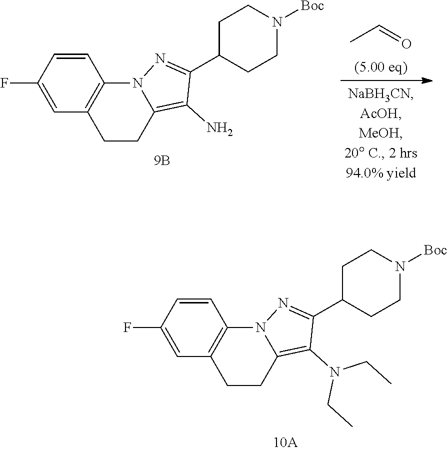

To a solution of compound 9B (65.0 mg, 168 μmol, 1.00 eq), acetaldehyde (185 mg, 1.68 mmol, 236 μL, 5.00 eq) in MeOH (1.00 mL) was added NaBH₃CN (21.1 mg, 336 μmol, 2.00 eq). The mixture was stirred at 20° C. for 2 hrs. The reaction mixture was concentrated under reduced pressure to remove solvent. The residue was purified by prep-TLC (SiO₂, Petroleum ether:Ethyl acetate=2:1, R_{f}=0.59) to give compound 10A (70.0 mg, 158 μmol, 94.0% yield) as a white solid.

LCMS: Rt=0.384 min, MS=443.2, M+H⁺

### Procedure for preparation of 1,1,1-trifluoro-3-hydroxypropan-2-yl 4-(3-(diethylamino)-7-fluoro-4,5-dihydropyrazolo[1,5-a]quinolin-2-yl)piperidine-1-carboxylate (10)

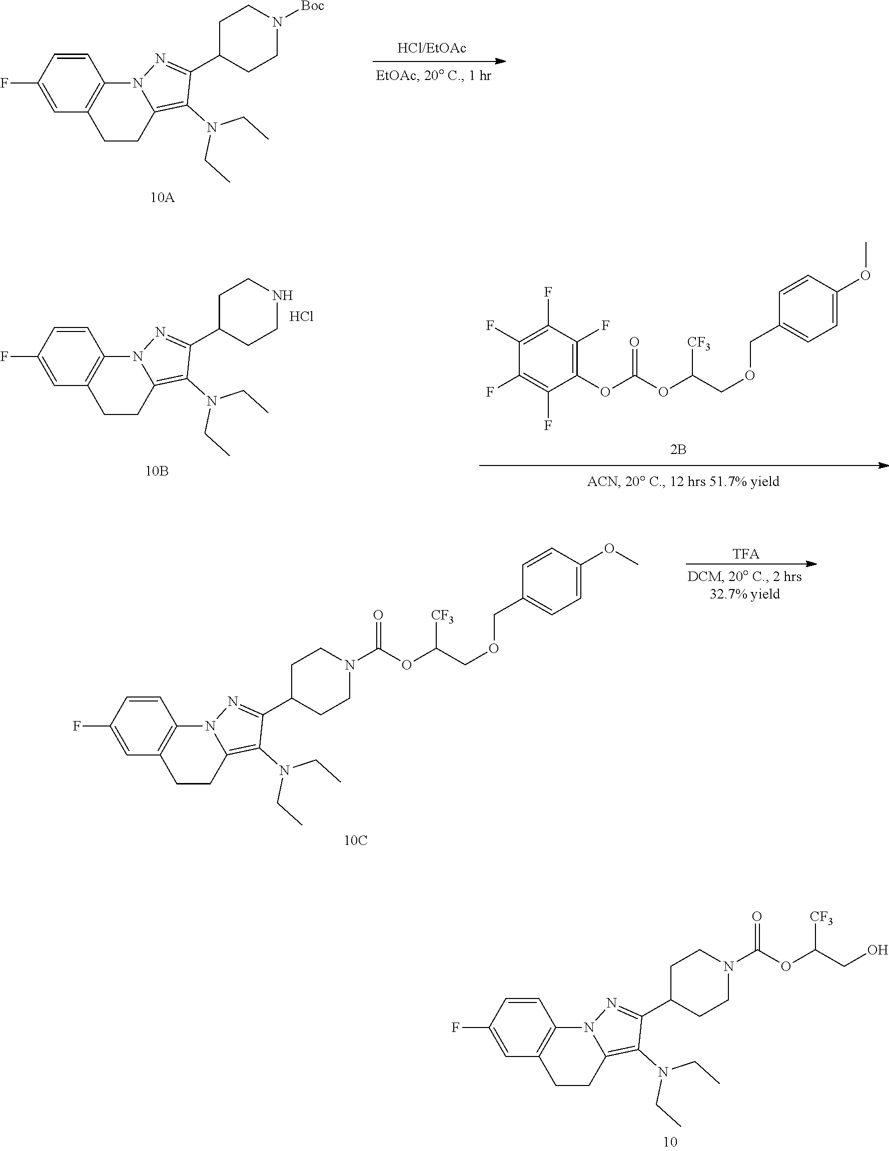 [SMILES: CCN(CC)c1c(C2CCN(C(=O)OC(C)(C)C)CC2)nn2c1CCc1cc(F)ccc1-2 ; CCN(CC)c1c(C2CCNCC2)nn2c1CCc1cc(F)ccc1-2 ; Cl ; COc1ccc(COCC(OC(=O)Oc2c(F)c(F)c(F)c(F)c2F)C(F)(F)F)cc1 ; CCN(CC)c1c(C2CCN(C(=O)OC(COCc3ccc(OC)cc3)C(F)(F)F)CC2)nn2c1CCc1cc(F)ccc1-2 ; CCN(CC)c1c(C2CCN(C(=O)OC(CO)C(F)(F)F)CC2)nn2c1CCc1cc(F)ccc1-2]

The compound was prepared according to the same synthesis step of example 2 starting from 10A to afford compound 10 (20.0 mg, 39.6 μmol, 32.7% yield, 98.7% purity) as a white solid.

LCMS: Rt=0.364 min, MS=499.2, M+H⁺

¹H NMR: (400 MHz, CDCl₃): δ 7.77-7.79 (m, 1H), 6.99-7.00 (m, 1H), 6.91-6.92 (m, 1H), 5.25-5.30 (m, 1H), 1.19-4.23 (m, 2H), 4.00-4.03 (m, 1H), 3.90-3.91 (m, 1H), 2.95-3.04 (m, 11H), 2.91 (s, 1H), 1.77 (m, 4H), 1.00-1.16 (m, 6H)

### Example 11: 1,1,1,3,3,3-hexafluoropropan-2-yl 6-(7-fluoro-4,5-dihydropyrazolo[1,5-a]quinolin-2-yl)-3-azabicyclo[3.1.0]hexane-3-carboxylate

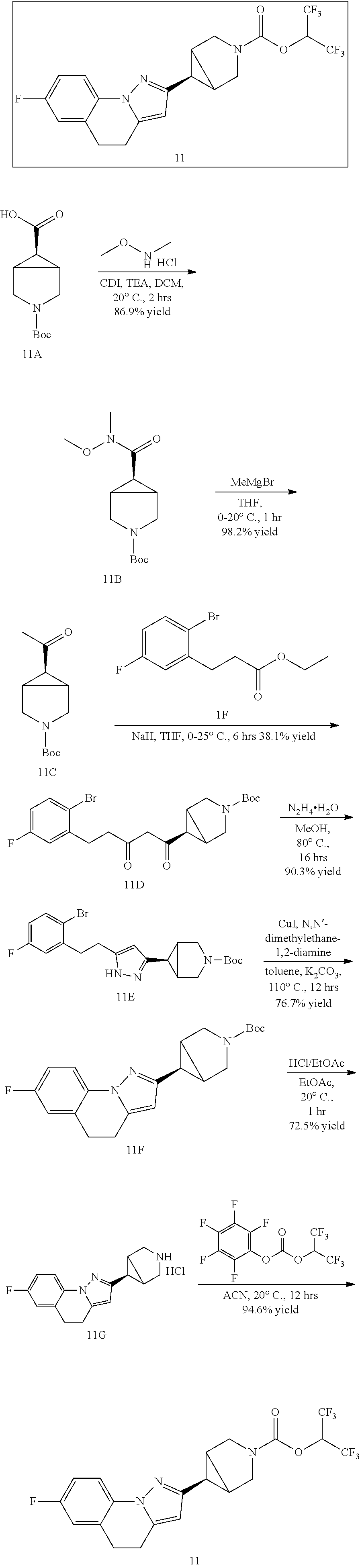

### Scheme-11: Synthesis of 1,1,1,3,3,3-hexafluoropropan-2-yl 6-(7-fluoro-4,5-dihydropyrazolo[1,5-a]quinolin-2-yl)-3-azabicyclo[3.1.0]hexane-3-carboxylate(11)

### Procedure for preparation of tert-butyl (1R,5S,6r)-6-(methoxy(methyl)carbamoyl)-3-azabicyclo[3.1.0]hexane-3-carboxylate (11B)

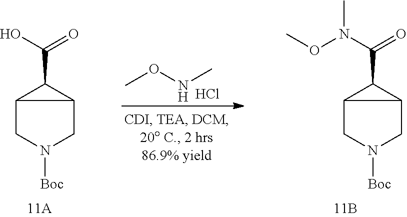

To a solution of compound 11A (1.00 g, 4.40 mmol, 1.00 eq) in DCM (10.0 mL) was added CDI (856 mg, 5.28 mmol, 1.20 eq), and then N-methoxymethanamine (472 mg, 4.84 mmol, 1.10 eq, HCl) and TEA (890 mg, 8.80 mmol, 1.22 mL, 2.00 eq) in DCM (3.00 mL) was added dropwise at 25° C. The mixture was stirred at 25° C. for 2 hrs. The reaction mixture was quenched by addition 1.00 M HCl 10.0 mL at 0° C., and then extracted with DCM. The combined organic layers were washed with brine, dried over Na₂SO₄, filtered and concentrated under reduced pressure to give compound 11B (1.10 g, 3.83 mmol, 86.9% yield, 94.0% purity) as a yellow liquid.

LCMS: Rt=0.333 min, MS=215.3, M-56+H⁺

¹H NMR: (400 MHz, CDCl₃): δ 3.734 (s, 3H), 3.65-3.70 (m, 1H), 3.55-3.58 (m, 1H), 3.44-3.50 (m, 2H), 3.20 (s, 3H), 2.05-2.08 (m, 2H), 1.97 (s, 1H), 1.45 (s, 9H).

### Procedure for preparation of tert-butyl (1R,5S,6r)-6-acetyl-3-azabicyclo[3.1.0]hexane-3-carboxylate (11C)

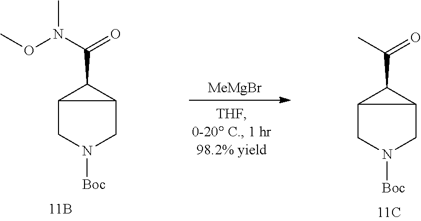

To a solution of compound 11B (1.10 g, 4.07 mmol, 1.00 eq) in THF (10.0 mL) was added dropwise MeMgBr (3.00 M, 3.05 mL, 2.25 eq) at −78° C. After addition, the resulting mixture was stirred at 0° C. for 1 hr. The mixture was quenched by NH₄Cl at 0° C. and extracted with EtOAc. Then the combined organic layers were washed with brine, dried over Na₂SO₄, filtered and concentrated under reduced pressure to give compound 11C (900 mg, 3.99 mmol, 98.2% yield) as a yellow oil.

LCMS: Rt=0.317 min, MS=170.3, M-56+H⁺

### Procedure for preparation of 1,1,1-trifluoro-3-hydroxypropan-2-yl 4-(7-fluoro-3-morpholino-4,5-dihydropyrazolo[1,5-a]quinolin-2-yl)piperidine-1-carboxylate (11)

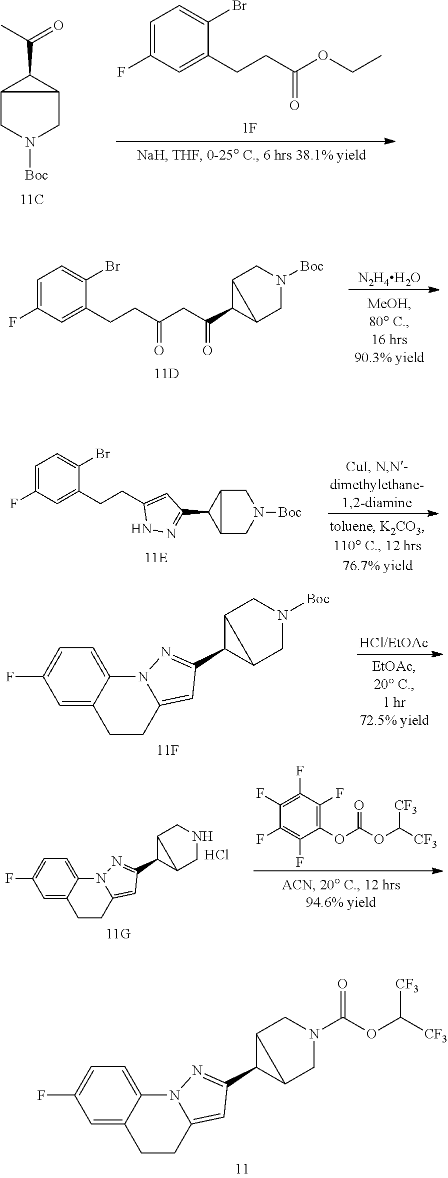

The compound was prepared according to the same synthesis step of example 1 starting from 11C to afford compound 11 (208 mg, 402 μmol, 94.6% yield, 98.1% purity) was obtained as a white solid.

LCMS: Rt=0.506 min, MS=464.3, M+H⁺

¹H NMR: (400 MHz, CDCl₃): δ 7.74-7.78 (m, 1H), 6.93-7.02 (m, 2H), 5.89 (s, 1H), 5.71-5.77 (m, 1H), 3.86-3.95 (m, 2H), 3.65-3.68 (m, 2H), 2.87-2.99 (m, 4H), 2.04-2.06 (m, 2H), 1.84 (t, q=3.2 Hz, 1H).

### Example 12: 1,1,1-trifluoro-3-hydroxypropan-2-yl 6-(7-fluoro-4,5-dihydropyrazolo[1,5-a]quinolin-2-yl)-3-azabicyclo[3.1.0]hexane-3-carboxylate

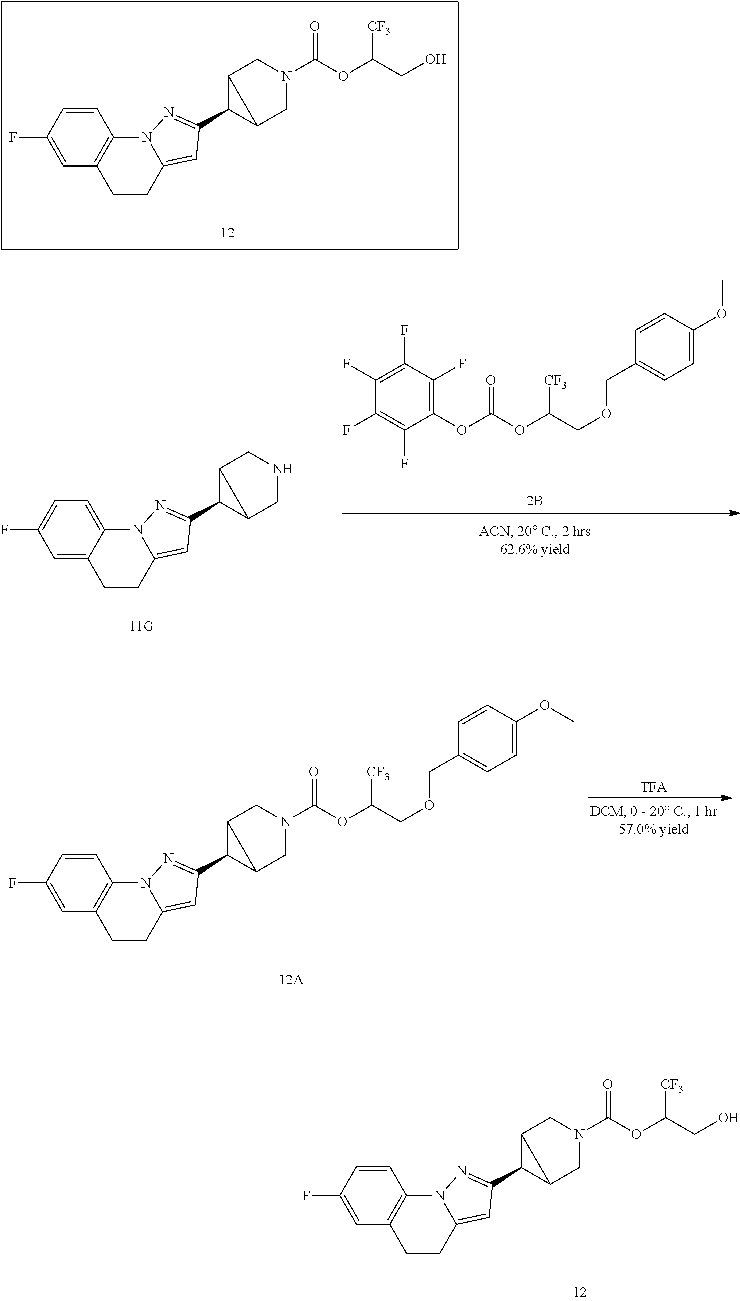

### Scheme-12: Synthesis of 1,1,1-trifluoro-3-hydroxypropan-2-yl 6-(7-fluoro-4,5-dihydropyrazolo[1,5-a]quinolin-2-yl)-3-azabicyclo[3.1.0]hexane-3-carboxylate (12)

### Procedure for preparation of 1,1,1-trifluoro-3-hydroxypropan-2-yl 6-(7-fluoro-4,5-dihydropyrazolo[1,5-a]quinolin-2-yl)-3-azabicyclo[3.1.0]hexane-3-carboxylate (12)

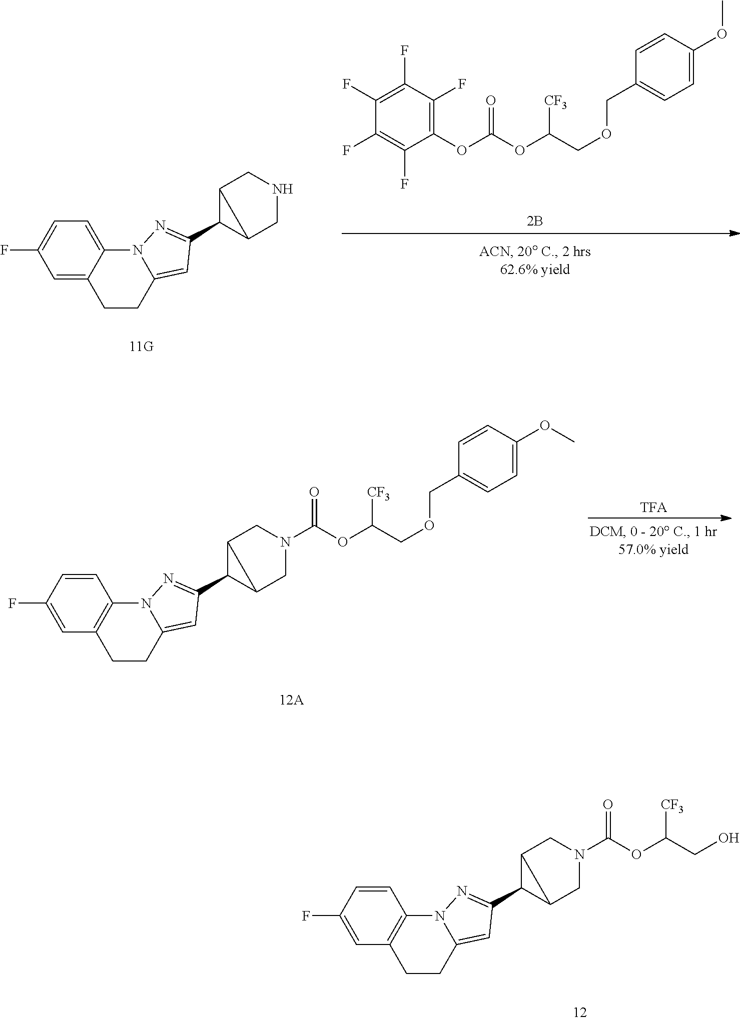

The compound was prepared according to the same synthesis step of example 2 starting from 11G to afford compound 12 (180 mg, 417 μmol, 57.0% yield, 98.7% purity) was obtained as a white solid.

LCMS: Rt=0.427 min, MS=426.3, M+H⁺

¹H NMR: (400 MHz, CDCl₃): δ 7.75-7.78 (m, 1H), 6.93-7.02 (m, 2H), 5.88 (d, q=2.8 Hz, 1H), 5.23-5.28 (m, 1H), 3.99-4.03 (m, 1H), 3.82-3.91 (m, 3H), 3.60-3.65 (m, 2H), 2.90-2.99 (m, 4H), 2.02 (s, 2H), 1.80-1.88 (m, 1H).

### Example 13: 1,1,1,3,3,3-hexafluoropropan-2-yl 4-(7-fluoro-4,5-dihydropyrazolo[1,5-a][1,8]naphthyridin-2-yl)piperidine-1-carboxylate

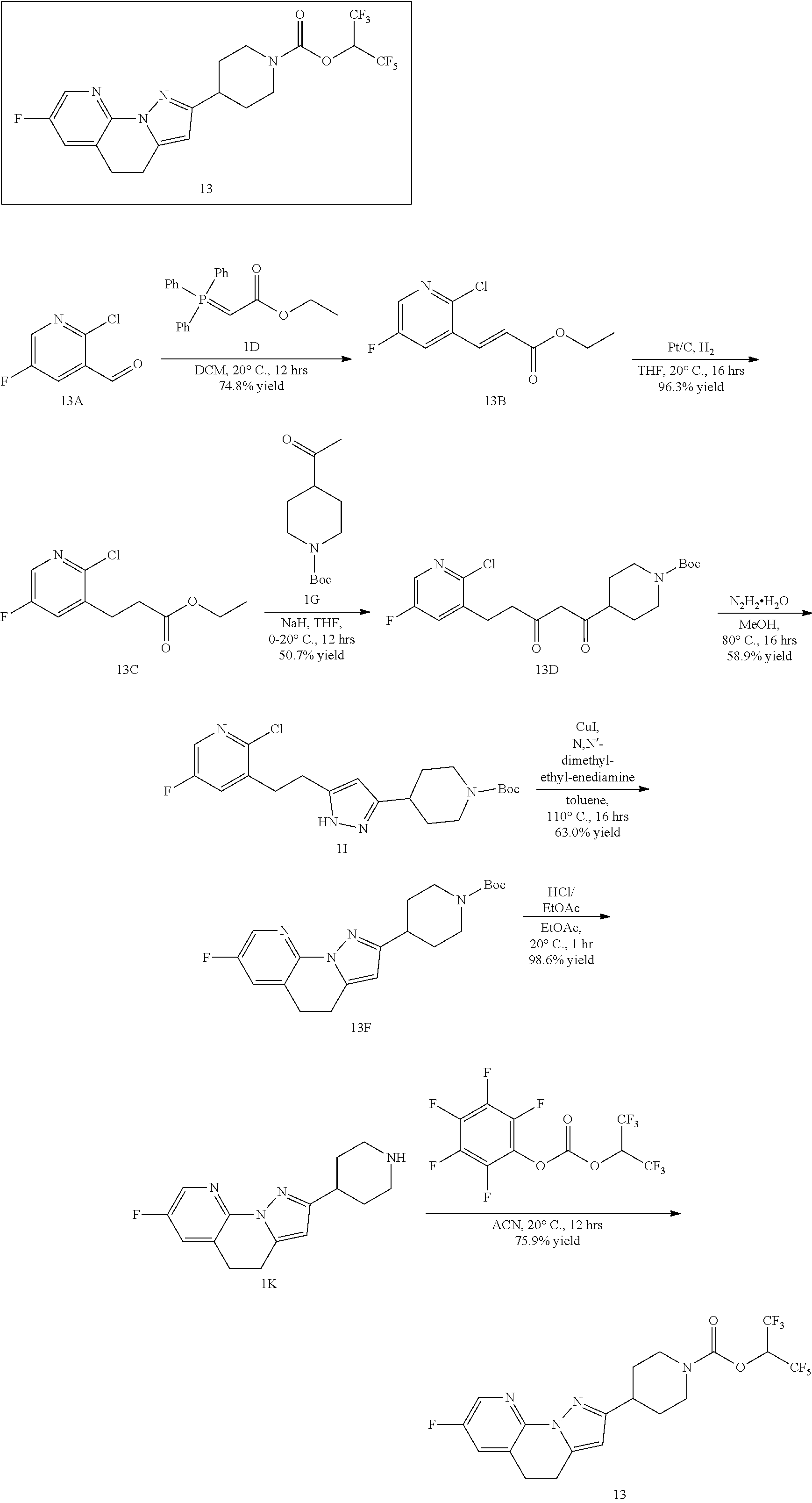

### Scheme-13: Synthesis of 1,1,1,3,3,3-hexafluoropropan-2-yl 4-(7-fluoro-4,5-dihydropyrazolo[1,5-a][1,8]naphthyridin-2-yl)piperidine-1-carboxylate (13)

### Procedure for preparation of 1,1,1,3,3,3-hexafluoropropan-2-yl 4-(7-fluoro-4,5-dihydropyrazolo[1,5-a][1,8]naphthyridin-2-yl)piperidine-1-carboxylate (13)

The compound was prepared according to the same synthesis step of example 1 starting from 13A to afford compound 13 (320 mg, 659 μmol, 75.9% yield, 96.1% purity) was obtained as a white solid.

LCMS: Rt=0.440 min, MS=467.2, M+H⁺

¹H NMR: (400 MHz, CDCl₃)

δ 8.26 (d, J=2.0 Hz, 1H), 7.36 (dd, J=2.4 Hz, J=8.0 Hz, 1H), 6.06 (s, 1H), 5.75-5.80 (m, 1H), 4.26 (t, J=13.6 Hz, 2H), 3.00-3.11 (m, 7H), 2.08 (d, J=12.4 Hz, 2H), 1.73-1.80 (m, 2H).

### Example 14: 1,1,1-trifluoro-3-hydroxypropan-2-yl 4-(7-fluoro-4,5-dihydropyrazolo[1,5-a][1,8]naphthyridin-2-yl)piperidine-1-carboxylate

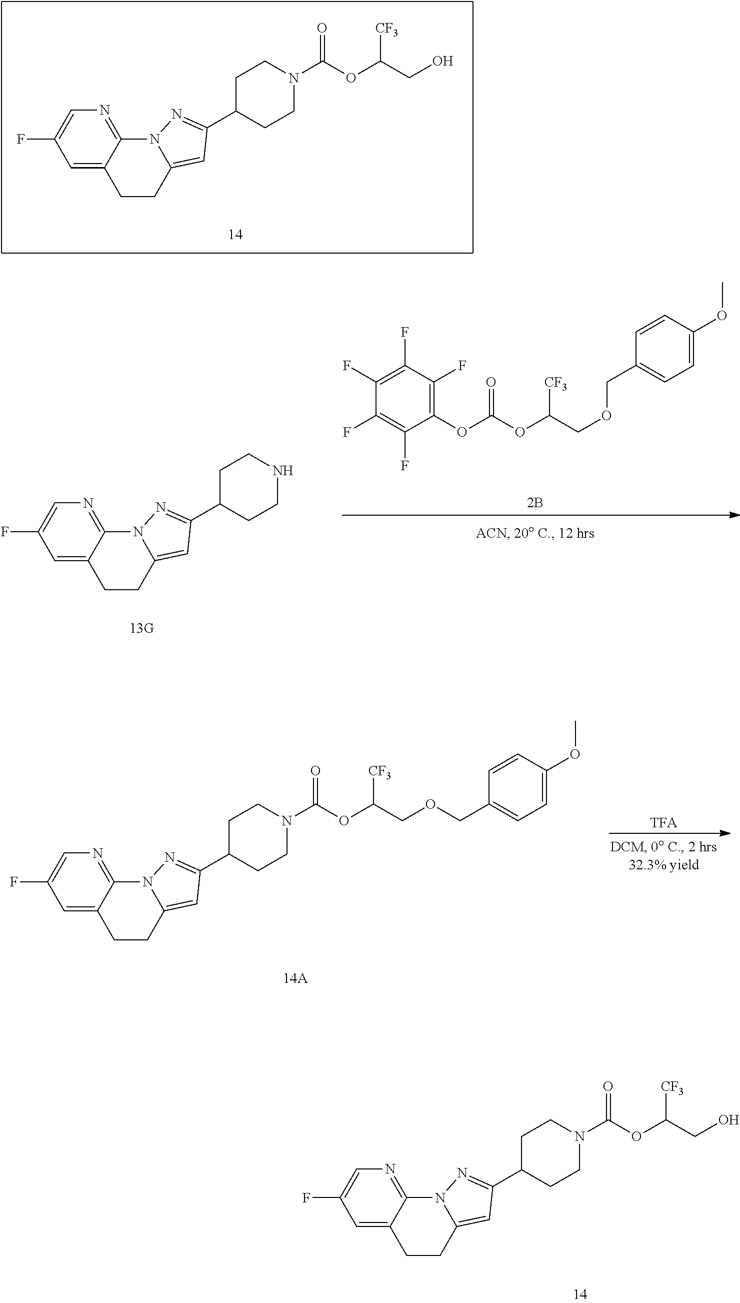 [SMILES: O=C(OC(CO)C(F)(F)F)N1CCC(c2cc3n(n2)-c2ncc(F)cc2CC3)CC1 ; Fc1cnc2c(c1)CCc1cc(C3CCNCC3)nn1-2 ; COc1ccc(COCC(OC(=O)Oc2c(F)c(F)c(F)c(F)c2F)C(F)(F)F)cc1 ; COc1ccc(COCC(OC(=O)N2CCC(c3cc4n(n3)-c3ncc(F)cc3CC4)CC2)C(F)(F)F)cc1 ; O=C(OC(CO)C(F)(F)F)N1CCC(c2cc3n(n2)-c2ncc(F)cc2CC3)CC1]

### Scheme-14: Synthesis of 1,1,1-trifluoro-3-hydroxypropan-2-yl 4-(7-fluoro-4,5-dihydropyrazolo[1,5-a][1,8]naphthyridin-2-yl)piperidine-1-carboxylate (14)

### Procedure for preparation of 1,1,1-trifluoro-3-hydroxypropan-2-yl 4-(7-fluoro-4,5-dihydropyrazolo[1,5-a][1,8]naphthyridin-2-yl)piperidine-1-carboxylate (14)

The compound was prepared according to the same synthesis step of example 2 starting from 13G to afford compound 14 (140 mg, 294 μmol, 32.3% yield, 90.1% purity) was obtained as a white solid.

LCMS: Rt=0.368 min, MS=429.2, M+H⁺

¹H NMR: (400 MHz, CDCl₃)

δ 8.25 (d, J=2.0 Hz, 1H), 7.36 (dd, J=2.4 Hz, J=8.0 Hz, 1H), 6.05 (s, 1H), 5.26-5.27 (m, 1H), 4.21-4.25 (m, 2H), 3.88-4.03 (m, 2H), 2.99-3.07 (m, 7H), 2.05 (d, J=12.4 Hz, 2H), 1.84-1.85 (m, 3H).

### Example 15: 1,1,1,3,3,3-hexafluoropropan-2-yl 4-(3-bromo-7-fluoro-4,5-dihydropyrazolo[1,5-a][1,8]naphthyridin-2-yl)piperidine-1-carboxylate

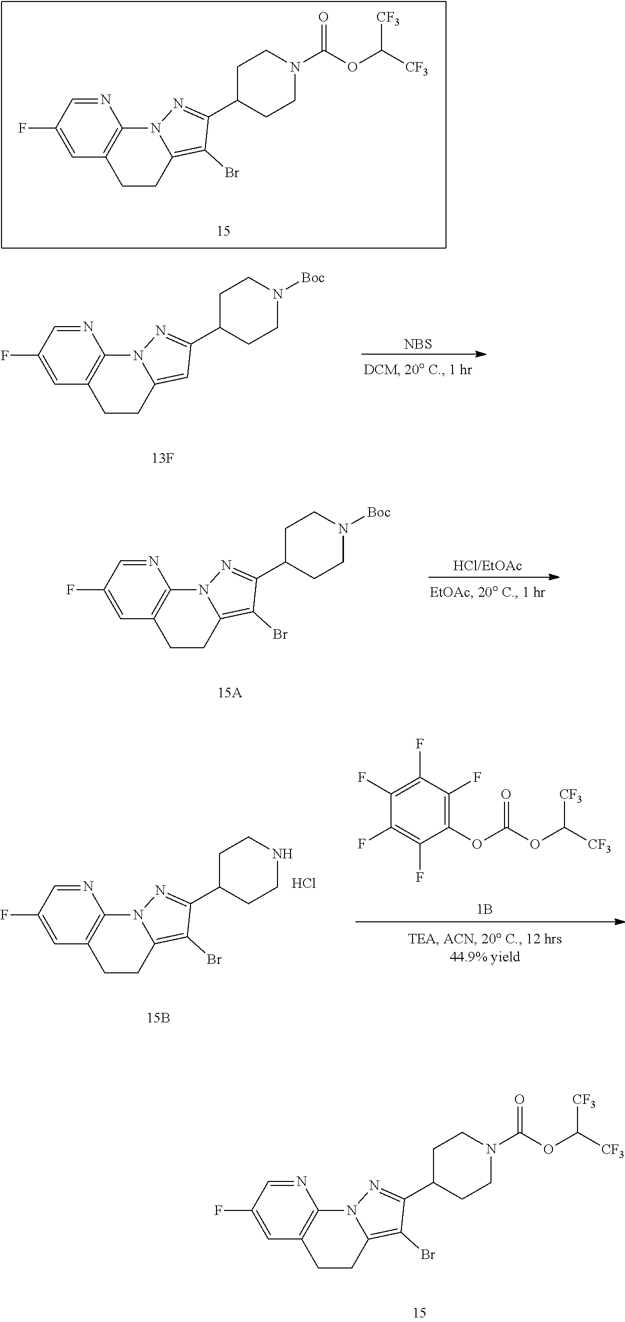

### Scheme-15: Synthesis of 1,1,1,3,3,3-hexafluoropropan-2-yl 4-(3-bromo-7-fluoro-4,5-dihydropyrazolo[1,5-a][1,8]naphthyridin-2-yl)piperidine-1-carboxylate (15)

### Procedure for preparation of 1,1,1-trifluoro-3-hydroxypropan-2-yl 4-(7-fluoro-4,5-dihydropyrazolo[1,5-a][1,8]naphthyridin-2-yl)piperidine-1-carboxylate (15A)

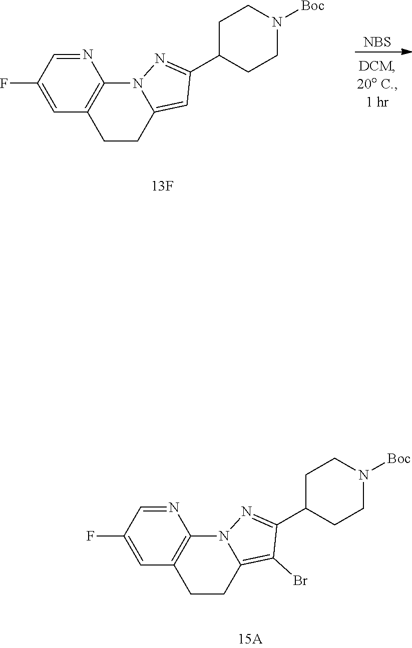

To a solution of compound 13F (500 mg, 1.34 mmol, 1.00 eq) in DCM (5.00 mL) was added NBS (358 mg, 2.01 mmol, 1.50 eq). The mixture was stirred at 20° C. for 1 hr. The residue was diluted with H₂O (3.00 mL) and extracted with EtOAc. The combined organic layers were washed brine, and dried over Na₂SO₄, filtered and concentrated under reduced pressure to give compound 15A (750 mg, crude) as a yellow solid.

LCMS: Rt=0.540 min, MS=453.0, M+2+H⁺

### Procedure for preparation of 1,1,1,3,3,3-hexafluoropropan-2-yl 4-(3-bromo-7-fluoro-4,5-dihydropyrazolo[1,5-a][1,8]naphthyridin-2-yl)piperidine-1-carboxylate (15)

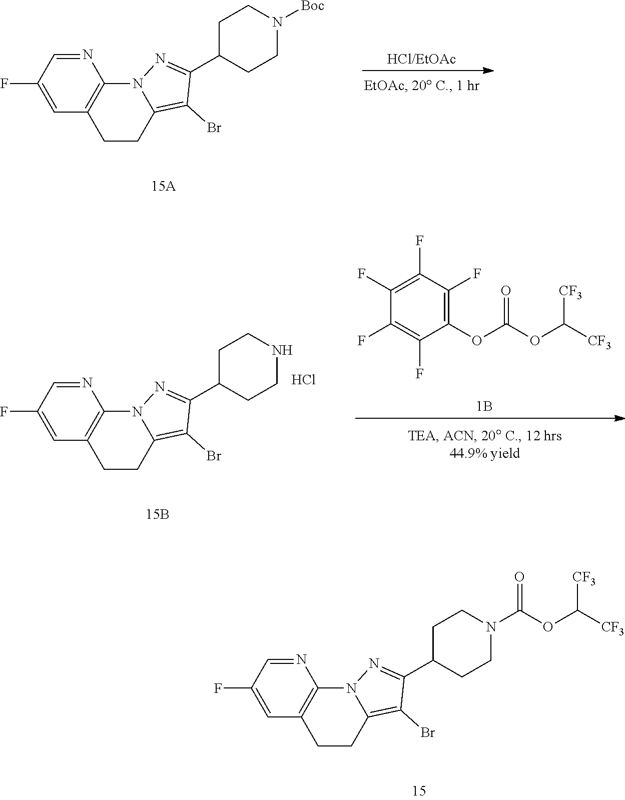 [SMILES: CC(C)(C)OC(=O)N1CCC(c2nn3c(c2Br)CCc2cc(F)cnc2-3)CC1 ; Fc1cnc2c(c1)CCc1c(Br)c(C3CCNCC3)nn1-2 ; Cl ; O=C(Oc1c(F)c(F)c(F)c(F)c1F)OC(C(F)(F)F)C(F)(F)F ; O=C(OC(C(F)(F)F)C(F)(F)F)N1CCC(c2nn3c(c2Br)CCc2cc(F)cnc2-3)CC1]

The compound was prepared according to the same synthesis step of example 1 starting from 15A to afford compound 15 (180 mg, 320 μmol, 44.9% yield, 97.0% purity) as a white solid.

LCMS: Rt=2.875 min, MS=544.2, M+H⁺

¹H NMR: (400 MHz, CDCl₃)

δ 8.28 (d, J=5.2 Hz, 1H), 7.36-7.39 (m, 1H), 5.76-5.79 (m, 1H), 4.30 (t, J=13.2 Hz, 2H), 2.98-3.08 (m, 7H), 2.01-2.09 (m, 4H).

### Example 16: 1,1,1-trifluoro-3-hydroxypropan-2-yl 4-(3-bromo-7-fluoro-4,5-dihydropyrazolo[1,5-a][1,8]naphthyridin-2-yl)piperidine-1-carboxylate

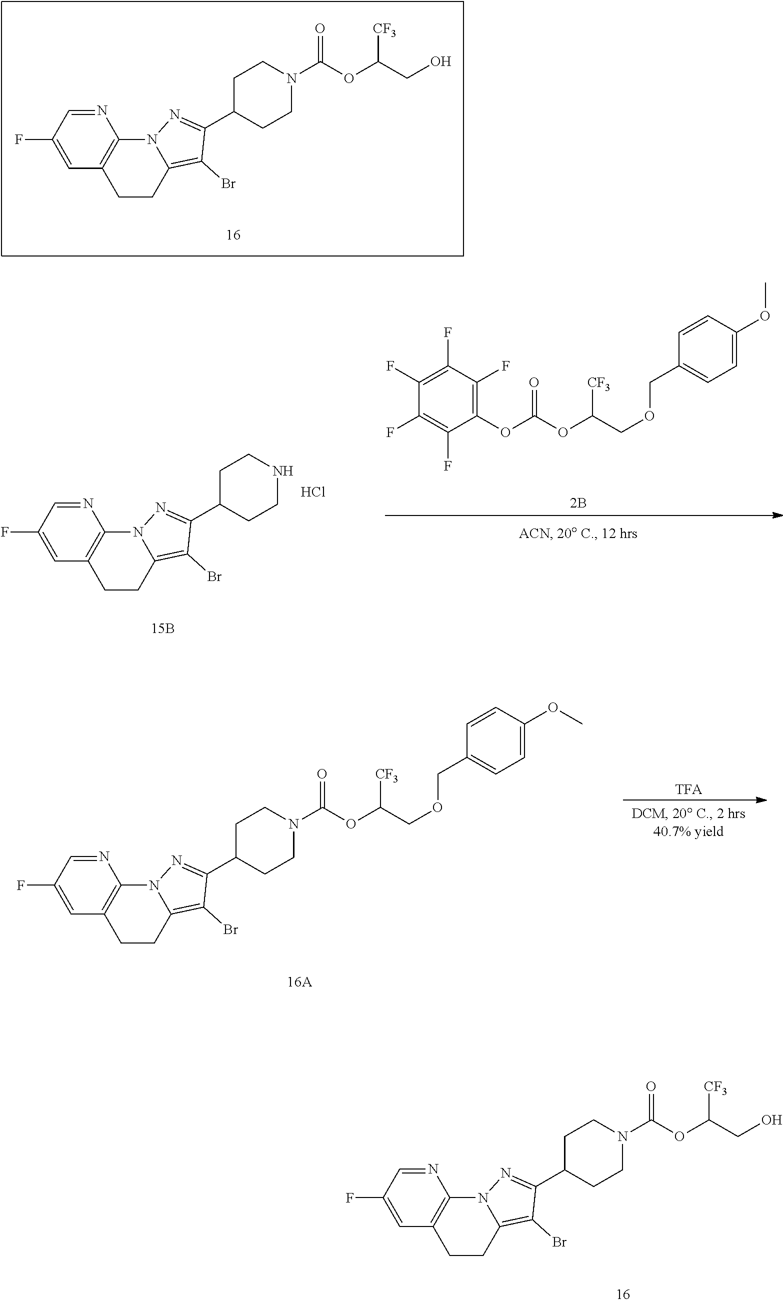 [SMILES: O=C(OC(CO)C(F)(F)F)N1CCC(c2nn3c(c2Br)CCc2cc(F)cnc2-3)CC1 ; Fc1cnc2c(c1)CCc1c(Br)c(C3CCNCC3)nn1-2 ; Cl ; COc1ccc(COCC(OC(=O)Oc2c(F)c(F)c(F)c(F)c2F)C(F)(F)F)cc1 ; COc1ccc(COCC(OC(=O)N2CCC(c3nn4c(c3Br)CCc3cc(F)cnc3-4)CC2)C(F)(F)F)cc1 ; O=C(OC(CO)C(F)(F)F)N1CCC(c2nn3c(c2Br)CCc2cc(F)cnc2-3)CC1]

### Scheme-16: Synthesis of 1,1,1-trifluoro-3-hydroxypropan-2-yl 4-(3-bromo-7-fluoro-4,5-dihydropyrazolo[1,5-a][1,8]naphthyridin-2-yl)piperidine-1-carboxylate (16)

### Procedure for preparation of 1,1,1-trifluoro-3-hydroxypropan-2-yl 4-(3-bromo-7-fluoro-4,5-dihydropyrazolo[1,5-a][1,8]naphthyridin-2-yl)piperidine-1-carboxylate (16)

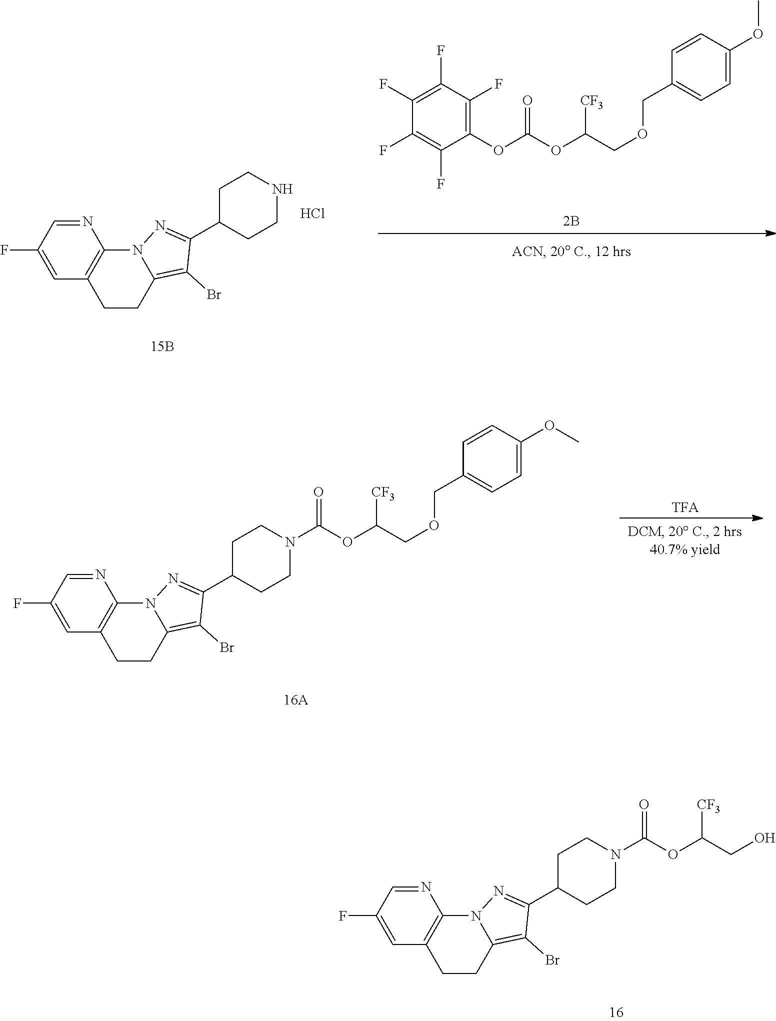 [SMILES: Fc1cnc2c(c1)CCc1c(Br)c(C3CCNCC3)nn1-2 ; Cl ; COc1ccc(COCC(OC(=O)Oc2c(F)c(F)c(F)c(F)c2F)C(F)(F)F)cc1 ; COc1ccc(COCC(OC(=O)N2CCC(c3nn4c(c3Br)CCc3cc(F)cnc3-4)CC2)C(F)(F)F)cc1 ; O=C(OC(CO)C(F)(F)F)N1CCC(c2nn3c(c2Br)CCc2cc(F)cnc2-3)CC1]

The compound was prepared according to the same synthesis step of example 2 starting from 15B to afford compound 16 (71.0 mg, 138 μmol, 40.7% yield, 98.7% purity) as a brown oil.

LCMS: Rt=0.441 min, MS=509.0, M+H⁺

¹HNMR: (400 MHz, MeOD)

δ 8.21 (d, J=2.4 Hz, 1H), 7.67 (dd, J₁=2.4 Hz, J₂=8.0 Hz, 1H), 5.25-5.36 (m, 1H), 4.20-4.33 (m, 2H), 3.86-3.92 (m, 1H), 3.76-3.85 (m, 1H), 2.99-3.13 (m, 7H), 1.84-2.02 (m, 4H).

### Example 17: 1,1,1,3,3,3-hexafluoropropan-2-yl 4-(7-fluoro-3-methyl-4,5-dihydropyrazolo[1,5-a][1,8]naphthyridin-2-yl)piperidine-1-carboxylate

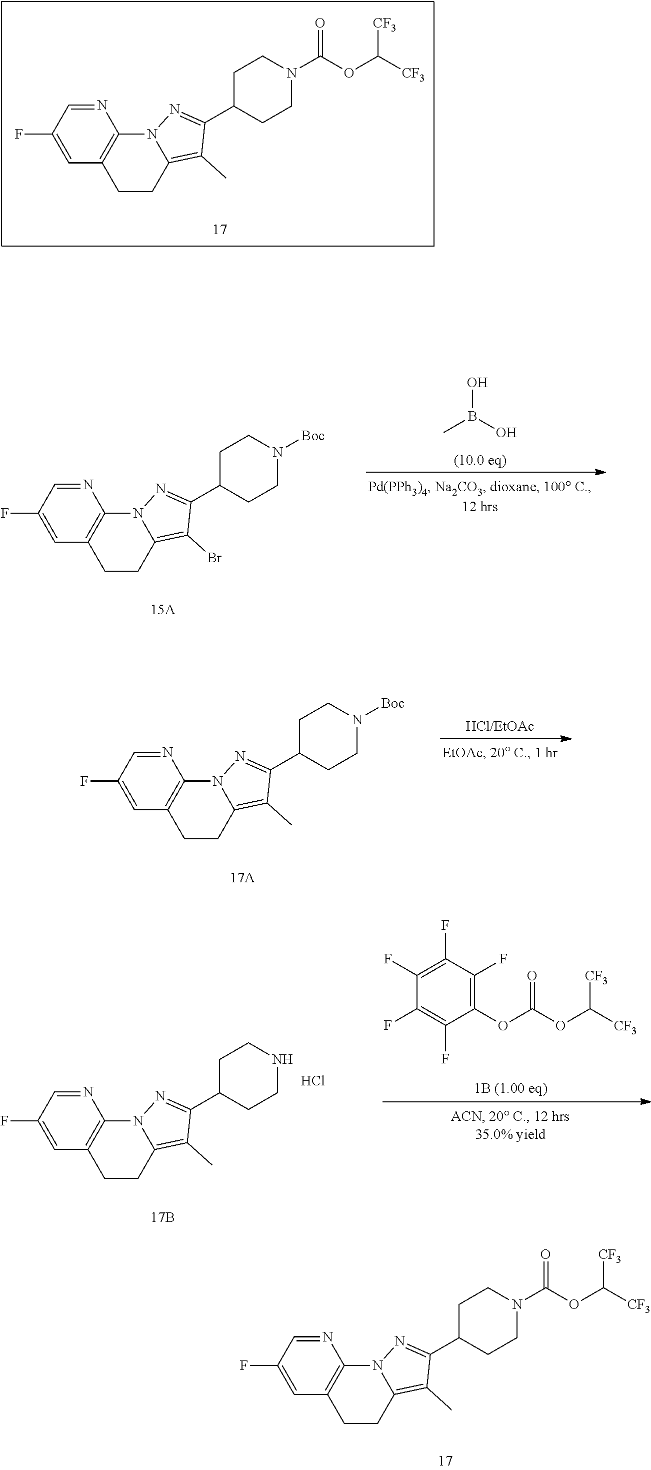

### Scheme-17: Synthesis of 1,1,1,3,3,3-hexafluoropropan-2-yl 4-(7-fluoro-3-methyl-4,5-dihydropyrazolo[1,5-a][1,8]naphthyridin-2-yl)piperidine-1-carboxylate (17)

### Procedure for preparation of tert-butyl 4-(7-fluoro-3-methyl-4,5-dihydropyrazolo[1,5-a][1,8]naphthyridin-2-yl)piperidine-1-carboxylate (17A)

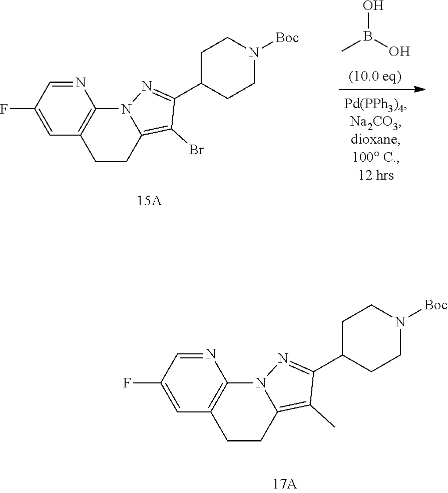

To a solution of compound 15A (500 mg, 1.11 mmol, 1.00 eq) in dioxane (5.00 mL) and H₂O (1.00 mL) and was added Pd(PPh₃)₄ (128 mg, 110 μmol, 0.10 eq) and Na₂CO₃ (352 mg, 3.32 mmol, 3.00 eq) and Methylboronic acid (663 mg, 11.0 mmol, 10.0 eq). The mixture was stirred at 100° C. for 12 hrs under N₂. The reaction mixture was quenched by addition H₂O 3.00 mL, and extracted with EtOAc. The combined organic layers were washed brine, and dried over Na₂SO₄, filtered and concentrated under reduced pressure to give the residue. The residue was purified by prep-HPLC (column: CD05-Phenomenex luna C18 150×40×10 um; mobile phase: [water (FA)-ACN]; gradient: 60%-90% B over 11 min) to give compound 17A (295 mg, crude) as a yellow solid.

LCMS: Rt=0.473 min, MS=387.2, M+H⁺

### Procedure for preparation of 1,1,1,3,3,3-hexafluoropropan-2-yl 4-(7-fluoro-3-methyl-4,5-dihydropyrazolo[1,5-a][1,8]naphthyridin-2-yl)piperidine-1-carboxylate (17)

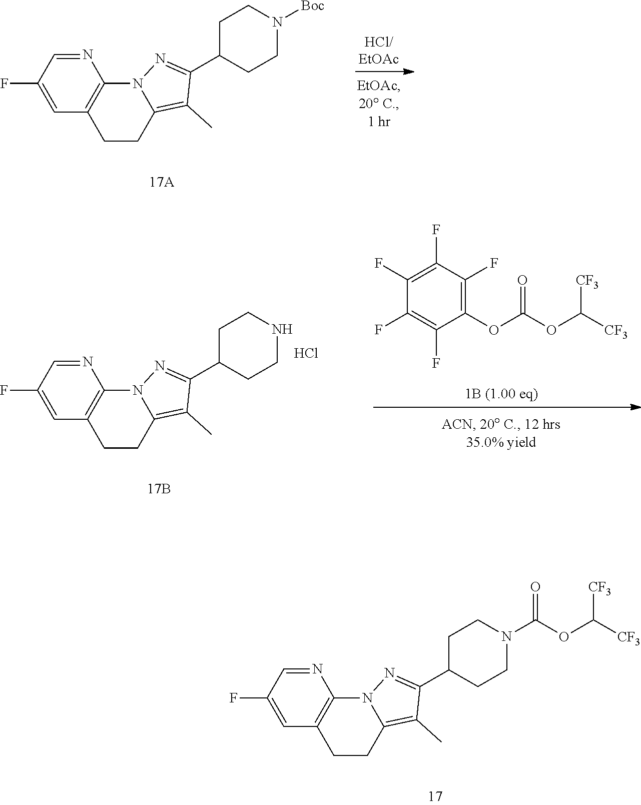 [SMILES: Cc1c(C2CCN(C(=O)OC(C)(C)C)CC2)nn2c1CCc1cc(F)cnc1-2 ; Cc1c(C2CCNCC2)nn2c1CCc1cc(F)cnc1-2 ; Cl ; O=C(Oc1c(F)c(F)c(F)c(F)c1F)OC(C(F)(F)F)C(F)(F)F ; Cc1c(C2CCN(C(=O)OC(C(F)(F)F)C(F)(F)F)CC2)nn2c1CCc1cc(F)cnc1-2]

The compound was prepared according to the same synthesis step of example 1 starting from 17A to afford compound 17 (105 mg, 244 μmol, 35.0% yield, 90.1% purity) as a white solid.

LCMS: Rt=2.307 min, MS=481.1, M+H⁺

¹H NMR: (400 MHz, CDCl₃)

δ 8.25 (s, 1H), 7.32-7.34 (m, 1H), 5.75-5.81 (m, 1H), 4.27-4.36 (t, J=16.0 Hz, 2H), 2.87-3.01 (m, 7H), 1.97-2.00 (m, 7H).

### Example 18: 1,1,1,3,3,3-hexafluoropropan-2-yl 4-(3-cyano-7-fluoro-4,5-dihydropyrazolo[1,5-a][1,8]naphthyridin-2-yl)piperidine-1-carboxylate

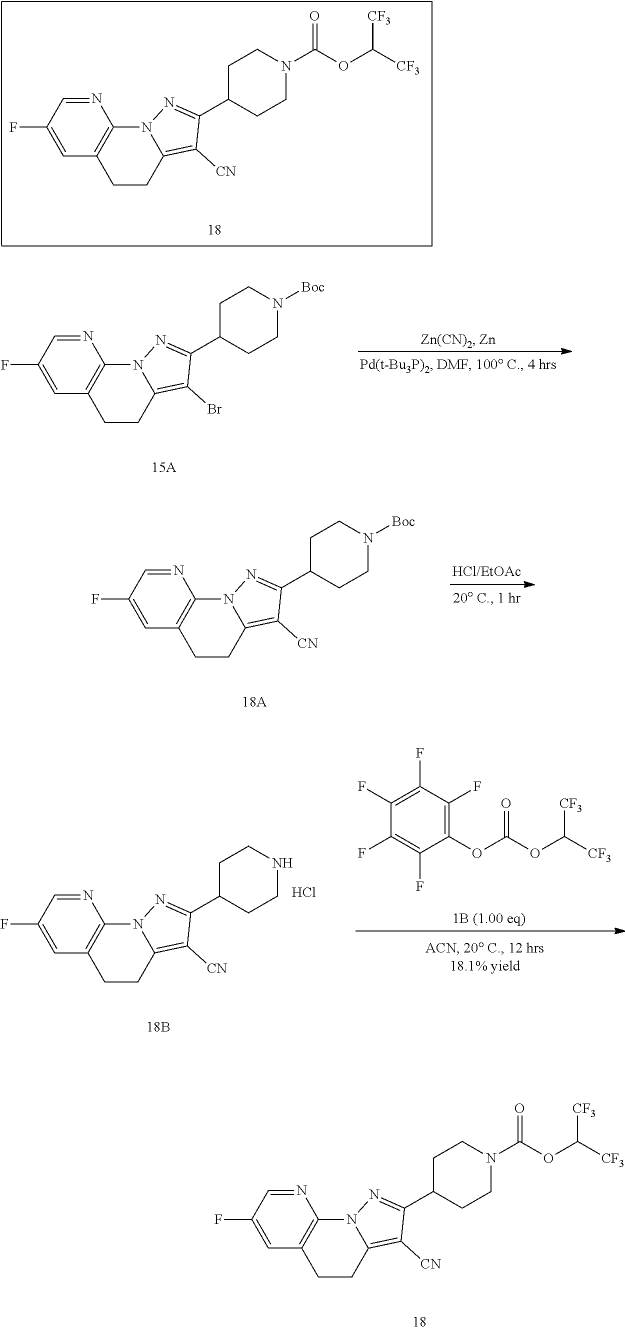

### Scheme-18: Scheme-18: 1,1,1,3,3,3-hexafluoropropan-2-yl 4-(3-cyano-7-fluoro-4,5-dihydropyrazolo[1,5-a][1,8]naphthyridin-2-yl)piperidine-1-carboxylate (18)

### Procedure for preparation of tert-butyl 4-(3-cyano-7-fluoro-4,5-dihydropyrazolo[1,5-a][1,8]naphthyridin-2-yl)piperidine-1-carboxylate (18A)

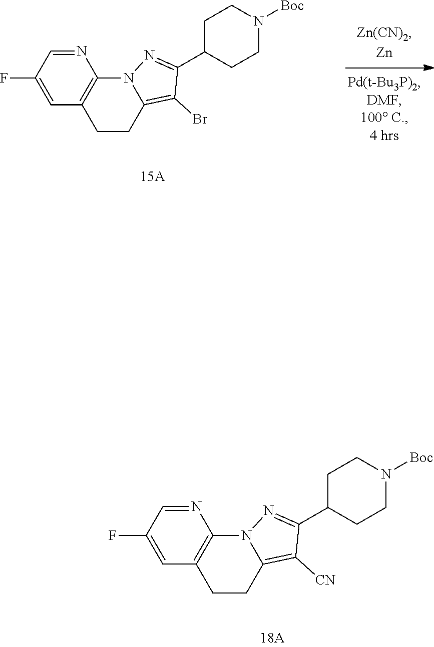 [SMILES: CC(C)(C)OC(=O)N1CCC(c2nn3c(c2Br)CCc2cc(F)cnc2-3)CC1 ; CC(C)(C)OC(=O)N1CCC(c2nn3c(c2C#N)CCc2cc(F)cnc2-3)CC1]

A mixture of compound 15A (300 mg, 602 μmol, 1.00 eq), Zn(CN)₂ (0.30 g, 2.55 mmol, 162.0 μL, 4.24 eq), Pd(t-Bu₃P)₂ (30.8 mg, 60.2 μmol, 0.10 eq), Zn (0.12 g, 1.84 mmol, 3.05 eq) in NMP (10.0 mL) was degassed and purged with N₂ for 3 times, and then the mixture was stirred at 100° C. for 4 hrs under N₂ atmosphere. The cooled mixture was diluted with EtOAc and washed sequentially with EtOAc and brine. The aqueous layers were extracted with EtOAc, and the combined organic layers were dried and concentrated. The crude product was used into the next step without further purification to give compound 18A (240 mg, crude) as a yellow solid.

LCMS: Rt=0.448 min, MS=298.0, M-99+H⁺

### Procedure for preparation of 1,1,1,3,3,3-hexafluoropropan-2-yl 4-(3-cyano-7-fluoro-4,5-dihydropyrazolo[1,5-a][1,8]naphthyridin-2-yl)piperidine-1-carboxylate (18)

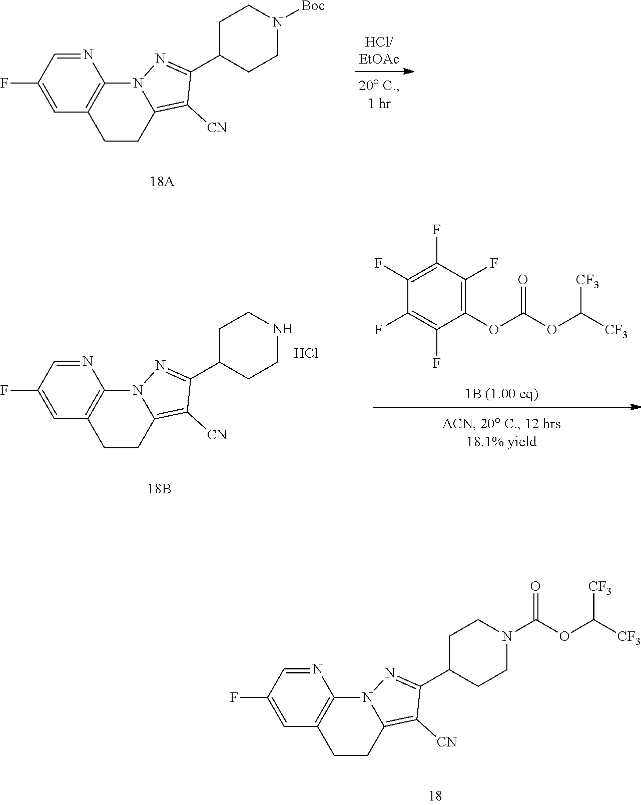 [SMILES: CC(C)(C)OC(=O)N1CCC(c2nn3c(c2C#N)CCc2cc(F)cnc2-3)CC1 ; N#Cc1c(C2CCNCC2)nn2c1CCc1cc(F)cnc1-2 ; Cl ; O=C(Oc1c(F)c(F)c(F)c(F)c1F)OC(C(F)(F)F)C(F)(F)F ; N#Cc1c(C2CCN(C(=O)OC(C(F)(F)F)C(F)(F)F)CC2)nn2c1CCc1cc(F)cnc1-2]

The compound was prepared according to the same synthesis step of example 1 starting from 18A to afford compound 18 (60.0 mg, 122 μmol, 18.1% yield, 99.9% purity) as a white solid.

LCMS: Rt=0.474 min, MS=492.0, M+H⁺

¹H NMR: (400 MHz, CDCl₃)

δ 8.32-8.33 (d, J=14.4 Hz, 1H), 7.27-7.45 (m, 1H), 5.75-5.81 (m, 1H), 4.31 (t, J=14.4 Hz, 2H), 3.16-3.23 (m, 2H), 3.04-3.16 (m, 5H), 2.00-2.09 (m, 4H)

### Example 19: 1,1,1,3,3,3-hexafluoropropan-2-yl 4-(3-cyclopropyl-7-fluoro-4,5-dihydropyrazolo[1,5-a][1,8]naphthyridin-2-yl)piperidine-1-carboxylate

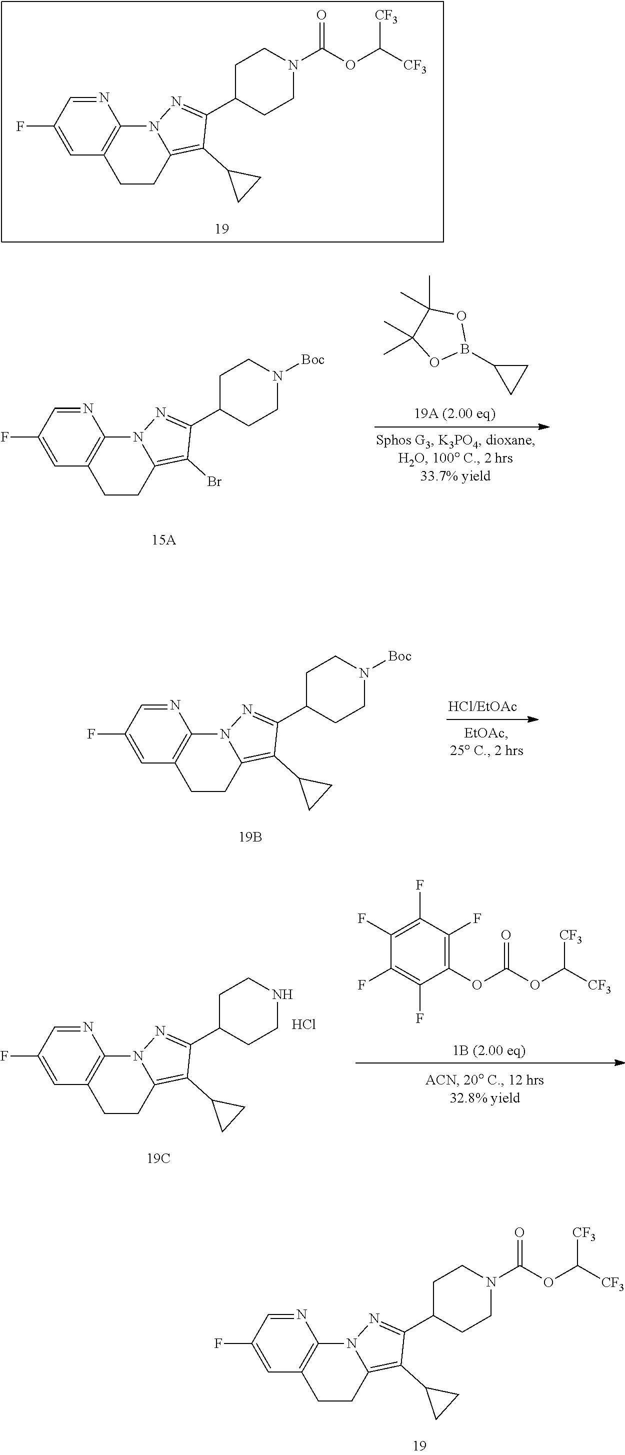

### Scheme-19: 1,1,1,3,3,3-hexafluoropropan-2-yl 4-(3-cyclopropyl-7-fluoro-4,5-dihydropyrazolo[1,5-a][1,8]naphthyridin-2-yl)piperidine-1-carboxylate (19)

### Procedure for preparation of tert-butyl 4-(3-cyclopropyl-7-fluoro-4,5-dihydropyrazolo[1,5-a][1,8]naphthyridin-2-yl)piperidine-1-carboxylate (19B)

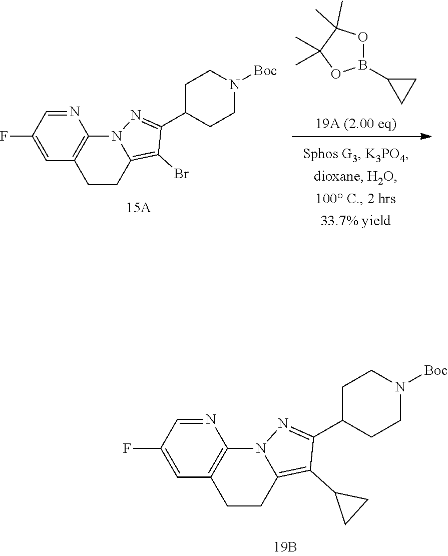

A mixture of compound 15A (200 mg, 444 μmol, 1.00 eq), compound 19A (149 mg, 888 μmol, 2.00 eq), K₃PO₄ (87.2 mg, 888 μmol, 2.00 eq), Sphos G3 (149 mg, 888 μmol, 2.00 eq) in dioxane (1.00 mL) and H₂O (0.25 mL) was degassed and purged with N₂ for 3 times, and then the mixture was stirred at 100° C. for 2 hrs under N₂ atmosphere. The reaction mixture was concentrated under reduced pressure to remove solvent. The residue was purified by column chromatography (SiO₂, Petroleum ether/Ethyl acetate=3/1, R_{f}=0.24) to give compound 19B (120 mg, 150 μmol, 33.7% yield, 51.5% purity) as a white solid.

### Procedure for preparation of 1,1,1,3,3,3-hexafluoropropan-2-yl 4-(3-cyclopropyl-7-fluoro-4,5-dihydropyrazolo[1,5-a][1,8]naphthyridin-2-yl)piperidine-1-carboxylate (19)

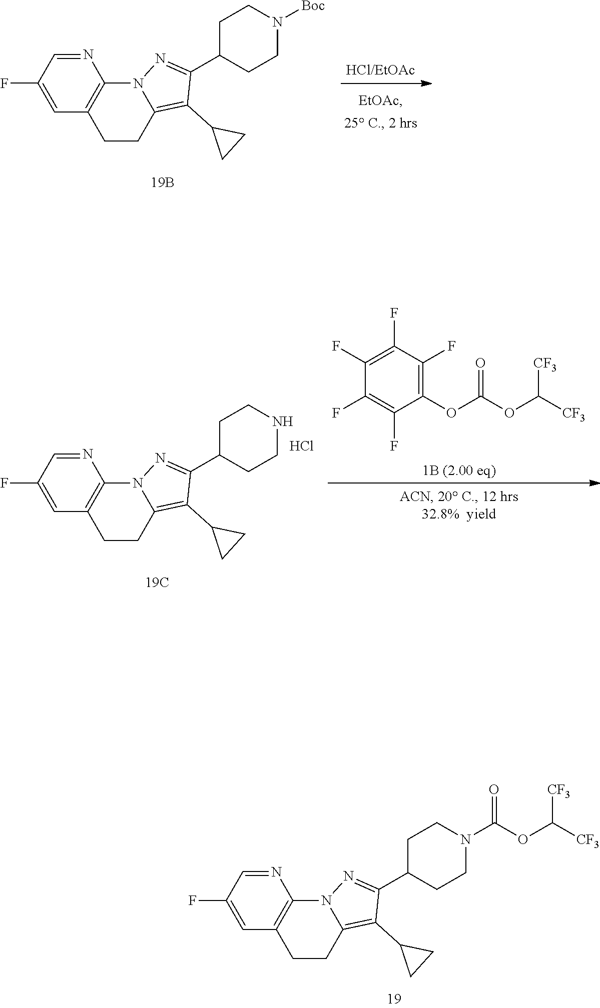 [SMILES: CC(C)(C)OC(=O)N1CCC(c2nn3c(c2C2CC2)CCc2cc(F)cnc2-3)CC1 ; Fc1cnc2c(c1)CCc1c(C3CC3)c(C3CCNCC3)nn1-2 ; Cl ; O=C(Oc1c(F)c(F)c(F)c(F)c1F)OC(C(F)(F)F)C(F)(F)F ; O=C(OC(C(F)(F)F)C(F)(F)F)N1CCC(c2nn3c(c2C2CC2)CCc2cc(F)cnc2-3)CC1]

The compound was prepared according to the same synthesis step of example 1 starting from 19B to afford compound 19 (30.0 mg, 53.2 μmol, 32.8% yield, 80.1% purity) as a white solid.

LCMS: Rt=0.517 min, MS=507.1, M+H⁺

¹H NMR: (400 MHz, CDCl₃) δ 8.22-8.22 (d, J=1.2 Hz, 1H), 7.68-7.15 (d, J=11.2 Hz, 1H), 6.37-6.40 (m, 1H), 4.06-4.09 (d, J=12 Hz, 2H), 3.11-3.24 (m, 3H), 2.97 (s, 4H), 1.95-1.96 (m, 2H), 1.72-1.75 (m, 2H), 1.58-1.72 (m, 1H), 0.84-0.85 (m, 2H), 0.53-0.54 (m, 2H)

### Example 20: 1,1,1,3,3,3-hexafluoropropan-2-yl 4-(7-fluoro-3-hydroxy-4,5-dihydropyrazolo[1,5-a][1,8]naphthyridin-2-yl)piperidine-1-carboxylate

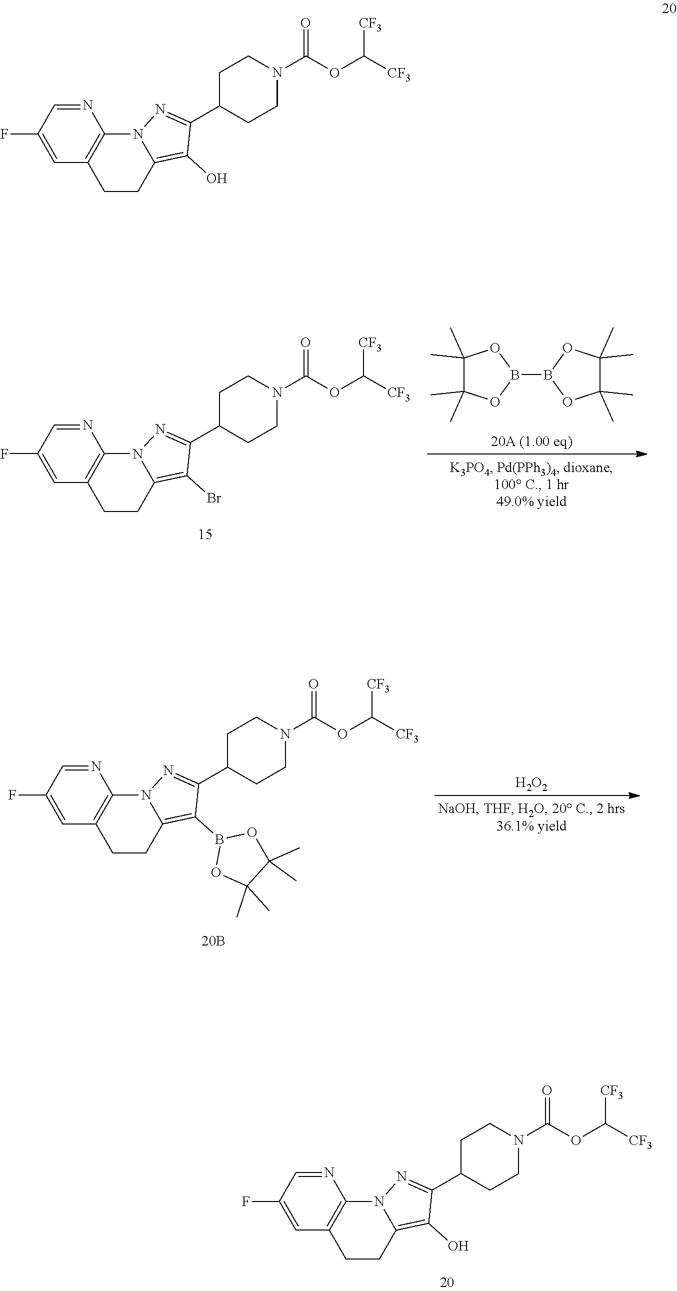

### Scheme-20: 1,1,1,3,3,3-hexafluoropropan-2-yl 4-(7-fluoro-3-hydroxy-4,5-dihydropyrazolo[1,5-a][1,8]naphthyridin-2-yl)piperidine-1-carboxylate (20)

### Procedure for preparation of 1,1,1,3,3,3-hexafluoropropan-2-yl 4-(7-fluoro-3-(4,4,5,5-tetramethyl-1,3,2-dioxaborolan-2-yl)-4,5-dihydropyrazolo[1,5-a][1,8]naphthyridin-2-yl)piperidine-1-carboxylate (20B)

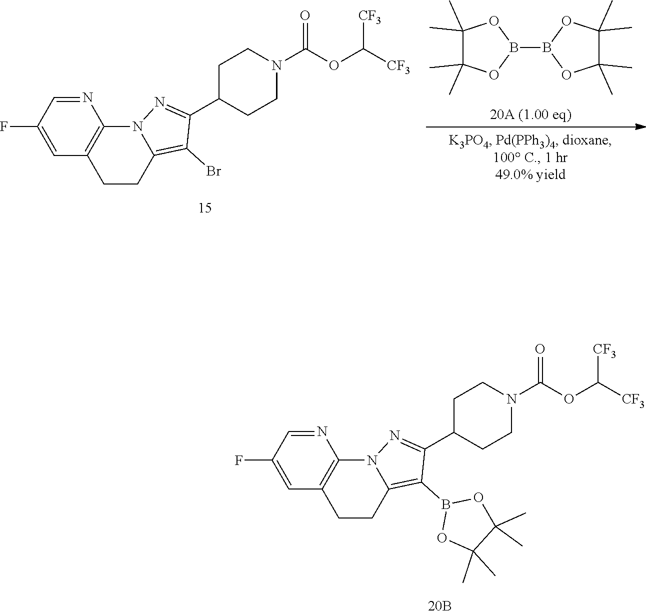

A mixture of compound 15 (310 mg, 569 μmol, 1.00 eq), compound 20A (144 mg, 569 μmol, 1.00 eq), K₃PO₄ (241 mg, 1.14 mmol, 2.00 eq), Pd(PPh₃)₄ (65.7 mg, 56.9 μmol, 0.10 eq) in dioxane (1.00 mL) was degassed and purged with N₂ for 3 times, and then the mixture was stirred at 100° C. for 1 hr under N₂ atmosphere. The cooled mixture was diluted with EtOAc and washed sequentially with EtOAc and brine. The aqueous layers were extracted with EtOAc, and the combined organic layers were dried and concentrated. The residue was purified by column chromatography (SiO₂, Petroleum ether/Ethyl acetate=10/1, R_{f}=0.40) to give compound 20B (165 mg, 279 μmol, 49.0% yield) as a yellow solid.

LCMS: Rt=0.557 min, MS=593.2, M+H⁺

### Procedure for preparation of 1,1,1,3,3,3-hexafluoropropan-2-yl 4-(7-fluoro-3-hydroxy-4,5-dihydropyrazolo[1,5-a][1,8]naphthyridin-2-yl)piperidine-1-carboxylate (20)

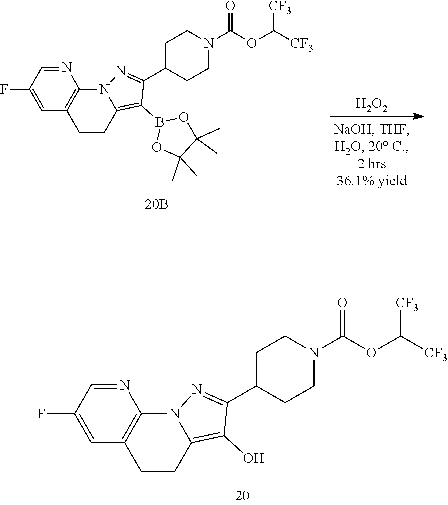 [SMILES: CC1(C)OB(c2c(C3CCN(C(=O)OC(C(F)(F)F)C(F)(F)F)CC3)nn3c2CCc2cc(F)cnc2-3)OC1(C)C ; "H2O2" ; O=C(OC(C(F)(F)F)C(F)(F)F)N1CCC(c2nn3c(c2O)CCc2cc(F)cnc2-3)CC1]

To a solution of compound 20B (165 mg, 279 μmol, 1.00 eq), H₂O₂ (1.11 g, 9.82 mmol, 944 μL, 30% purity, 35.3 eq) in THF (1.00 mL) was added dropwise NaOH (2.5 M, 223 μL, 2.00 eq) at 20° C. The resulting mixture was stirred at 20° C. for 2 hrs. The reaction mixture was quenched by addition Na₂SO₃ (1.00 mL) at 0° C., and then diluted with H₂O and extracted with EtOAc, the combined organic layers were washed with EtOAc, dried over Na₂SO₄, filtered and concentrated under reduced pressure to give a residue. The residue was purified by prep-HPLC (column: Phenomenex luna C18 150×25 mm×10 um; mobile phase: [water(HCl)-CAN]; gradient: 40%-70% B over 10 min) to give compound 20 (50.0 mg, 101 μmol, 36.1% yield, 97.0% purity) as a white solid.

LCMS: Rt=0.440 min, MS=483.1, M+H⁺

¹H NMR: (400 MHz, DMSO-d₆)

δ 8.21-8.21 (d, J=12 Hz, 2H), 7.73-7.76 (m, 1H), 6.57-6.60 (m, 1H), 4.03 (t, J=12 Hz, 2H), 2.93-2.97 (m, 2H), 2.89-2.97 (m, 5H), 1.93-1.94 (m, 2H), 1.23-1.66 (m, 2H)

### Example 21: 1,1,1,3,3,3-hexafluoropropan-2-yl 4-(7-fluoro-3-isopropyl-4,5-dihydropyrazolo[1,5-a][1,8]naphthyridin-2-yl)piperidine-1-carboxylate

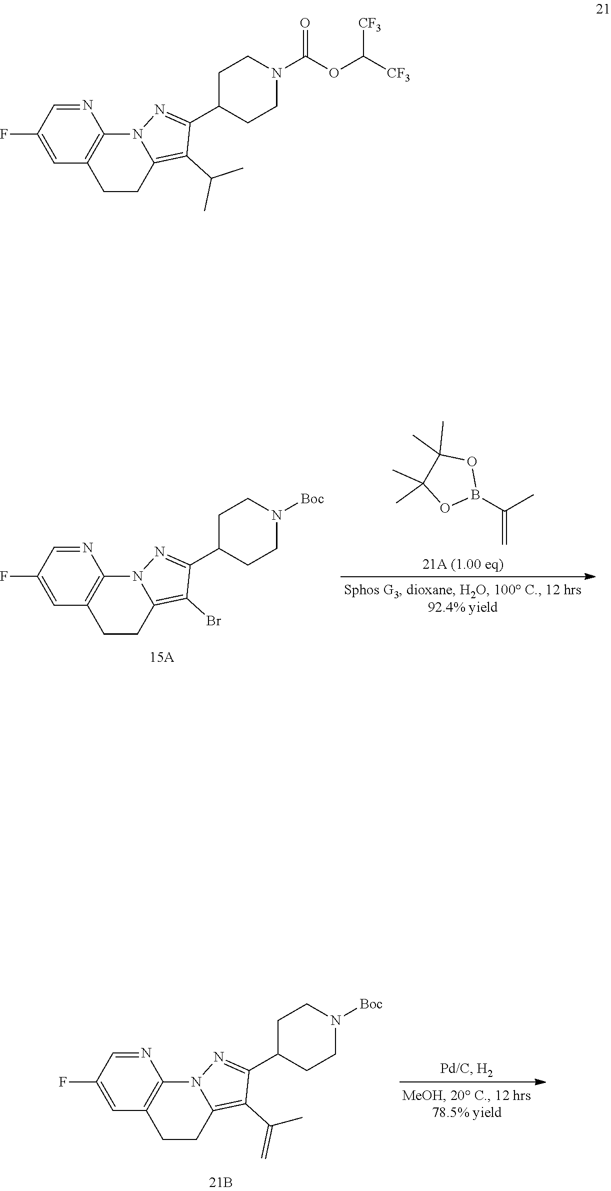 [SMILES: CC(C)c1c(C2CCN(C(=O)OC(C(F)(F)F)C(F)(F)F)CC2)nn2c1CCc1cc(F)cnc1-2 ; CC(C)(C)OC(=O)N1CCC(c2nn3c(c2Br)CCc2cc(F)cnc2-3)CC1 ; C=C(C)B1OC(C)(C)C(C)(C)O1 ; C=C(C)c1c(C2CCN(C(=O)OC(C)(C)C)CC2)nn2c1CCc1cc(F)cnc1-2]

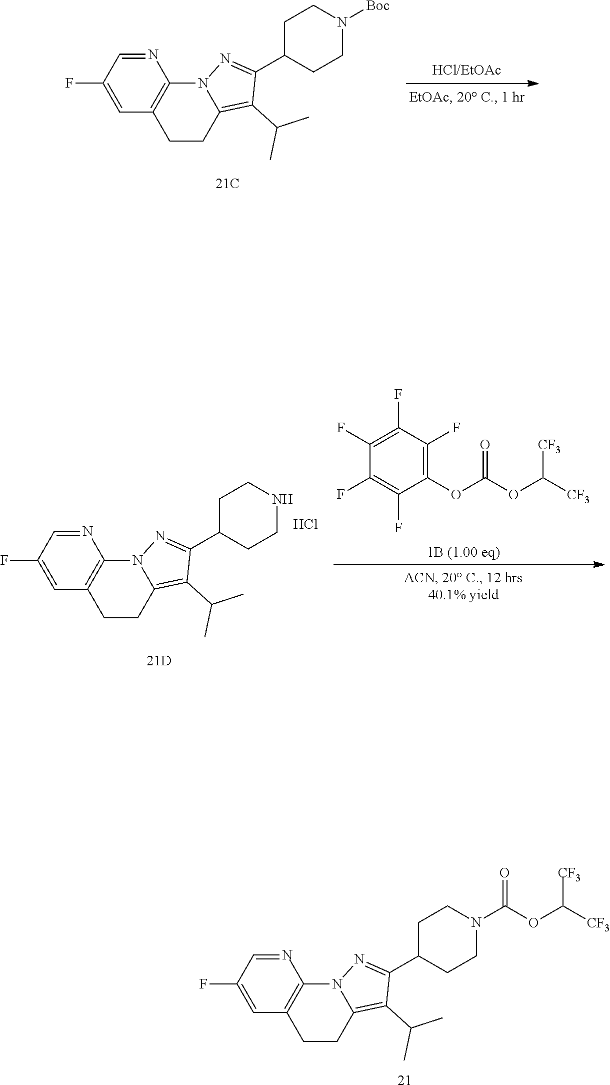

### Scheme-21: Scheme-21: 1,1,1,3,3,3-hexafluoropropan-2-yl 4-(7-fluoro-3-isopropyl-4,5-dihydropyrazolo[1,5-a][1,8]naphthyridin-2-yl)piperidine-1-carboxylate (21)

### Procedure for preparation of tert-butyl 4-(7-fluoro-3-(prop-1-en-2-yl)-4,5-dihydropyrazolo[1,5-a][1,8]naphthyridin-2-yl)piperidine-1-carboxylate (21B)

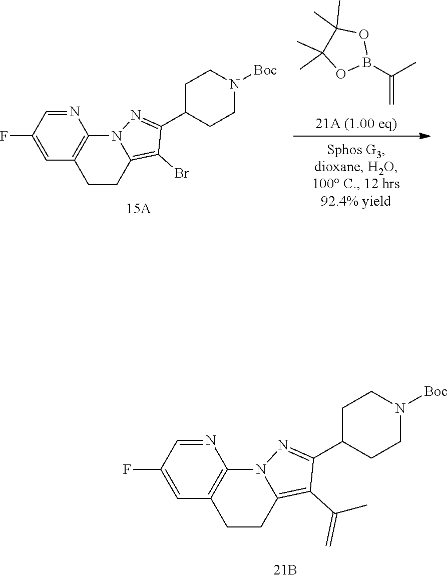 [SMILES: CC(C)(C)OC(=O)N1CCC(c2nn3c(c2Br)CCc2cc(F)cnc2-3)CC1 ; C=C(C)B1OC(C)(C)C(C)(C)O1 ; C=C(C)c1c(C2CCN(C(=O)OC(C)(C)C)CC2)nn2c1CCc1cc(F)cnc1-2]

A mixture of compound 15A (450 mg, 997 μmol, 1.00 eq), compound 21A (201 mg, 1.20 mmol, 1.00 eq), Sphos G3 (84.4 mg, 99.7 μmol, 0.10 eq), K₃PO₄ (635 mg, 2.99 mmol, 3.00 eq) in dioxane (5.00 mL) and H₂O (1.25 mL) was degassed and purged with N₂ for 3 times, and then the mixture was stirred at 100° C. for 12 hrs under N₂ atmosphere. The cooled mixture was diluted with EtOAc and extracted with EtOAc and washed with brine. The combined organic layers were dried over Na₂SO₄ and concentrated. The residue was purified by column chromatography (SiO₂, Petroleum ether/Ethyl acetate=5/1, R_{f}=0.40) to give compound 21B (380 mg, 921 μmol, 92.4% yield) as a white solid.

### Procedure for preparation of tert-butyl 4-(7-fluoro-3-isopropyl-4,5-dihydropyrazolo[1,5-a][1,8]naphthyridin-2-yl)piperidine-1-carboxylate (21C)

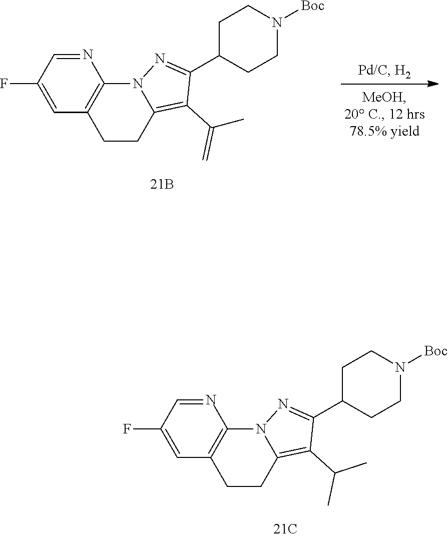 [SMILES: C=C(C)c1c(C2CCN(C(=O)OC(C)(C)C)CC2)nn2c1CCc1cc(F)cnc1-2 ; CC(C)c1c(C2CCN(C(=O)OC(C)(C)C)CC2)nn2c1CCc1cc(F)cnc1-2]

To a solution compound 21B (380 mg, 921 μmol, 1.00 eq) in MeOH (10.0 mL) was added Pd/C (98.0 mg, 92.1 μmol, 10.0% purity, 0.10 eq) under N₂ atmosphere. The suspension was degassed and purged with H₂ for 3 times. The mixture was stirred under H₂ (15 psi) at 20° C. for 2 hrs. The reaction mixture was concentrated under reduced pressure to give compound 21C (300 mg, 724 μmol, 78.5% yield) as a yellow solid. LCMS: Rt=0.499 min, MS=415.2, M+H⁺

### Procedure for preparation of 1,1,1,3,3,3-hexafluoropropan-2-yl 4-(7-fluoro-3-isopropyl-4,5-dihydropyrazolo[1,5-a][1,8]naphthyridin-2-yl)piperidine-1-carboxylate(21)

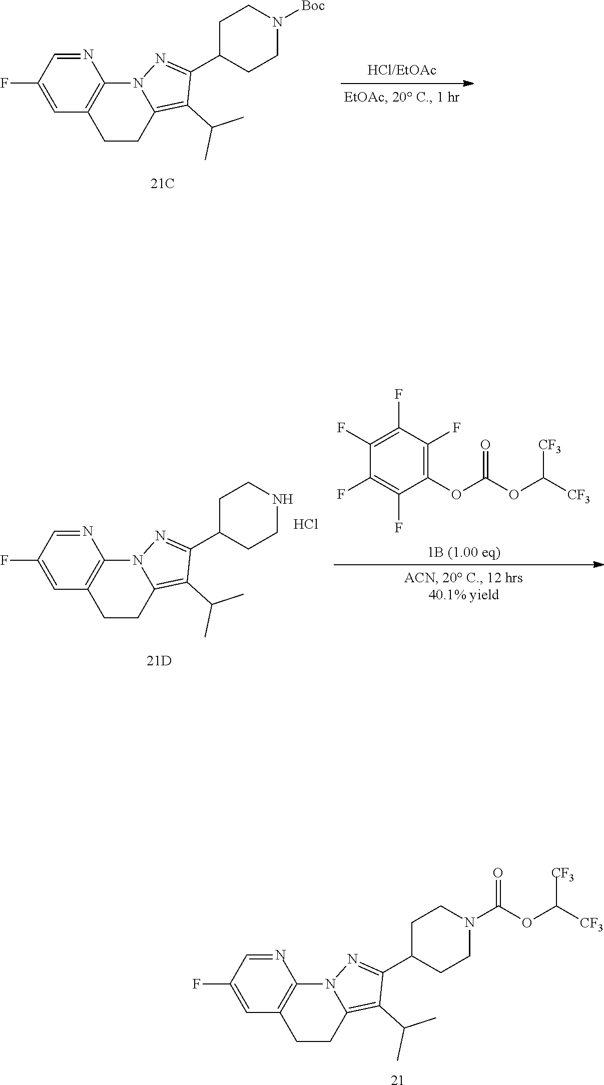

The compound was prepared according to the same synthesis step of example 1 starting from 21C to afford compound 21 (154 mg, 300 μmol, 40.1% yield, 99.2% purity) as a white solid.

LCMS: Rt=0.533 min, MS=509.2, M+H⁺,

¹H NMR: (400 MHz, CDCl₃)

δ 8.25 (s, 1H), 7.31-7.34 (d, J=12, 1H), 5.75-5.80 (m, 1H), 4.27-4.34 (m, 2H), 2.92-3.01 (m, 8H), 1.59-2.07 (m, 4H), 1.30-1.43 (m, 6H).

### Example 22:1,1,1,3,3,3-hexafluoropropan-2-yl 4-(7-fluoro-3-(pyridin-4-yl)-4,5-dihydropyrazolo[1,5-a][1,8]naphthyridin-2-yl)piperidine-1-carboxylate

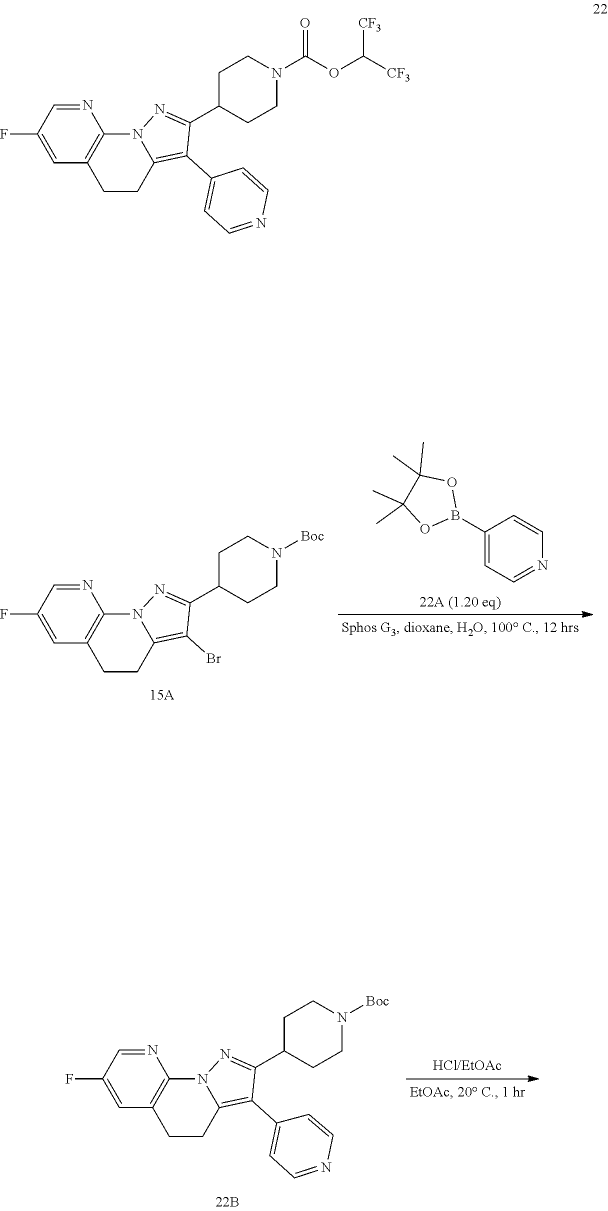 [SMILES: O=C(OC(C(F)(F)F)C(F)(F)F)N1CCC(c2nn3c(c2-c2ccncc2)CCc2cc(F)cnc2-3)CC1 ; CC(C)(C)OC(=O)N1CCC(c2nn3c(c2Br)CCc2cc(F)cnc2-3)CC1 ; CC1(C)OB(c2ccncc2)OC1(C)C ; CC(C)(C)OC(=O)N1CCC(c2nn3c(c2-c2ccncc2)CCc2cc(F)cnc2-3)CC1]

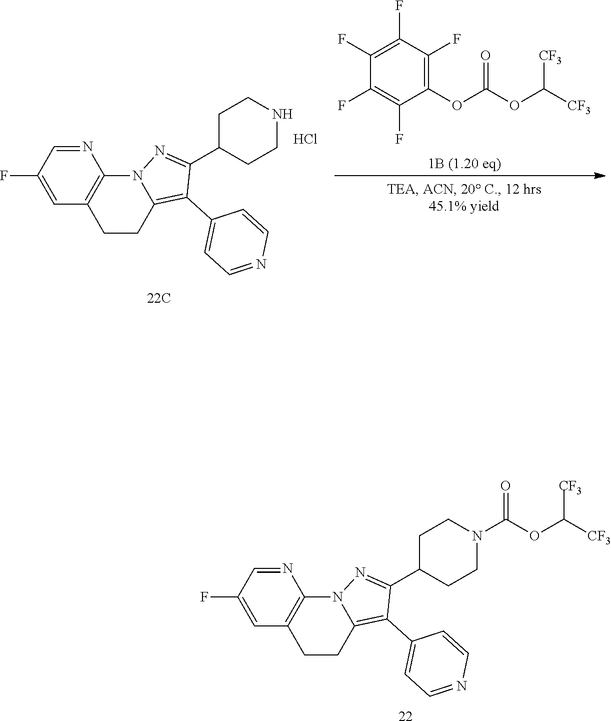

### Scheme-22: 1,1,1,3,3,3-hexafluoropropan-2-yl 4-(7-fluoro-3-(pyridin-4-yl)-4,5-dihydropyrazolo[1,5-a][1,8]naphthyridin-2-yl)piperidine-1-carboxylate(22)

### Procedure for preparation of tert-butyl 4-(7-fluoro-3-(pyridin-4-yl)-4,5-dihydropyrazolo[1,5-a][1,8]naphthyridin-2-yl)piperidine-1-carboxylate (22B)

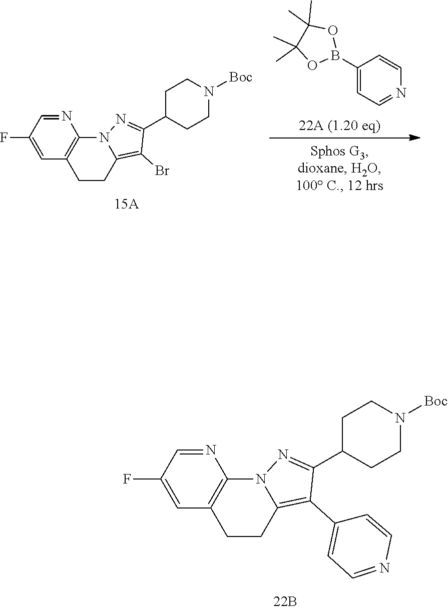 [SMILES: CC(C)(C)OC(=O)N1CCC(c2nn3c(c2Br)CCc2cc(F)cnc2-3)CC1 ; CC1(C)OB(c2ccncc2)OC1(C)C ; CC(C)(C)OC(=O)N1CCC(c2nn3c(c2-c2ccncc2)CCc2cc(F)cnc2-3)CC1]

To a solution of compound 15A (400 mg, 886 μmol, 1.00 eq) in dioxane (5.00 mL) and H₂O (1.00 mL) was added K₃PO₄ (282 mg, 1.33 mmol, 1.50 eq) and Sphos G3 (75.0 mg, 88.6 μmol, 0.10 eq) and compound 22A (218 mg, 1.06 mmol, 1.20 eq). The mixture was stirred at 100° C. for 12 hrs. The residue was diluted with H₂O (3.00 mL) and extracted with EtOAc. The combined organic layers were washed brine, and dried over Na₂SO₄, filtered, and concentrated under reduced pressure to give a residue. The residue was washed with MeOH (2.00 mL) and filtered and concentrated under reduced pressure to give compound 22B (300 mg, crude) as a yellow solid.

LCMS: Rt=0.366 min, MS=450.2, M+H⁺

### Procedure for preparation of 1,1,1,3,3,3-hexafluoropropan-2-yl 4-(7-fluoro-3-(pyridin-4-yl)-4,5-dihydropyrazolo[1,5-a][1,8]naphthyridin-2-yl)piperidine-1-carboxylate(22)

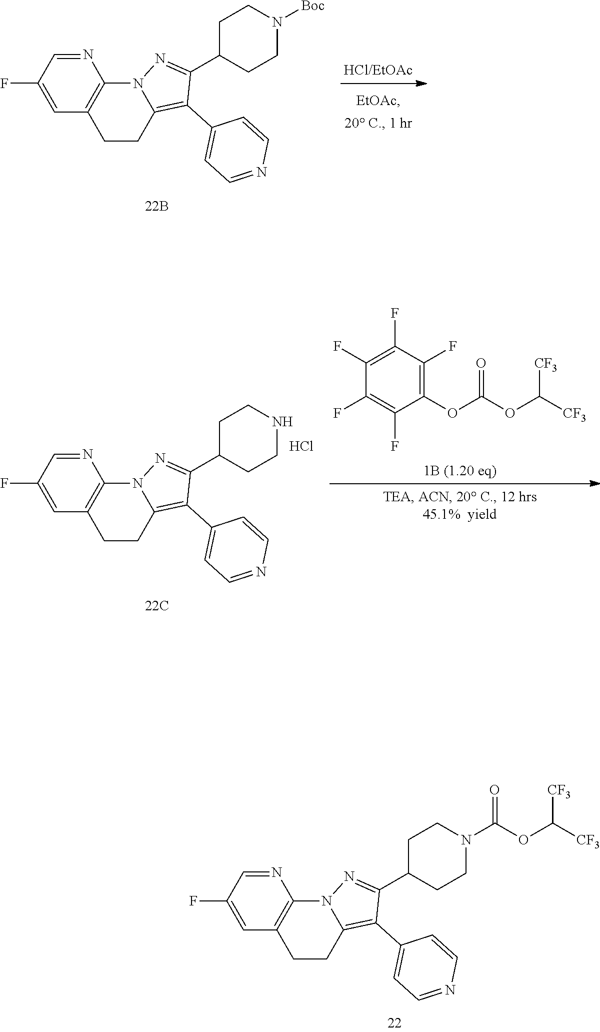 [SMILES: CC(C)(C)OC(=O)N1CCC(c2nn3c(c2-c2ccncc2)CCc2cc(F)cnc2-3)CC1 ; Fc1cnc2c(c1)CCc1c(-c3ccncc3)c(C3CCNCC3)nn1-2 ; Cl ; O=C(Oc1c(F)c(F)c(F)c(F)c1F)OC(C(F)(F)F)C(F)(F)F ; O=C(OC(C(F)(F)F)C(F)(F)F)N1CCC(c2nn3c(c2-c2ccncc2)CCc2cc(F)cnc2-3)CC1]

The compound was prepared according to the same synthesis step of example 1 starting from 22B to afford compound 22 (220 mg, 374 μmol, 45.1% yield, 92.5% purity) as a white solid.

LCMS: Rt=1.961 min, MS=544.2, M+H⁺

¹H NMR: (400 MHz, CDCl₃)

δ 8.71 (d, J=5.2 Hz, 2H), 8.33 (d, J=2.8 Hz, 1H), 7.38-7.41 (m, 1H), 7.21-7.23 (m, 2H), 5.73-5.78 (m, 1H), 4.26 (t, J=13.6 Hz, 2H), 2.98-3.06 (m, 6H), 2.89-2.95 (m, 1H), 2.04-2.13 (m, 2H), 1.91-1.94 (m, 2H).

### Example 23: 1,1,1,3,3,3-hexafluoropropan-2-yl 4-(3-amino-7-fluoro-4,5-dihydropyrazolo[1,5-a][1,8]naphthyridin-2-yl)piperidine-1-carboxylate

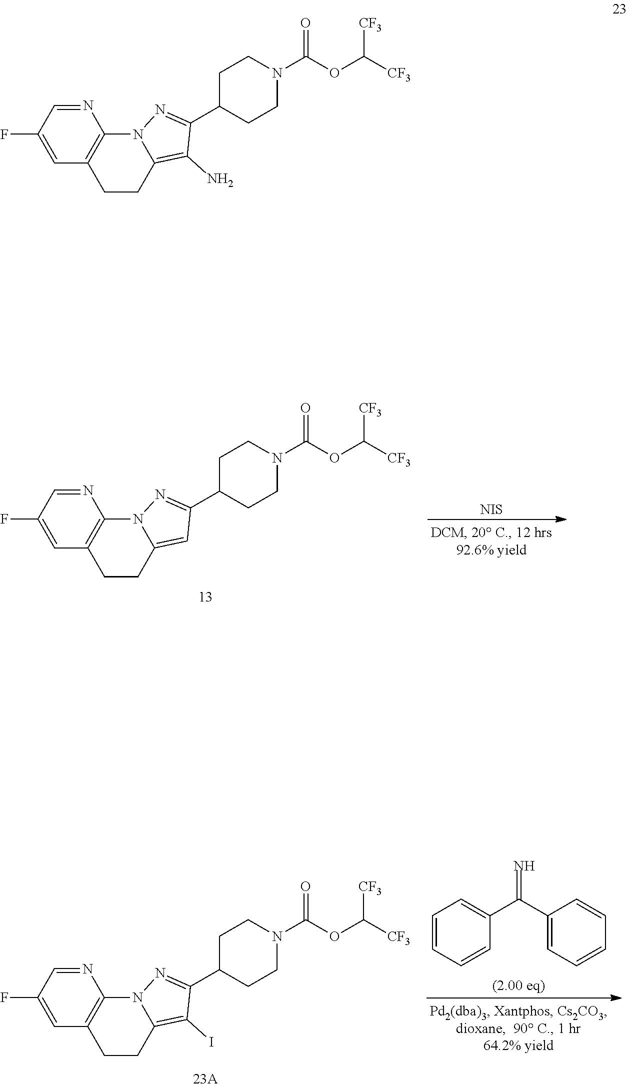

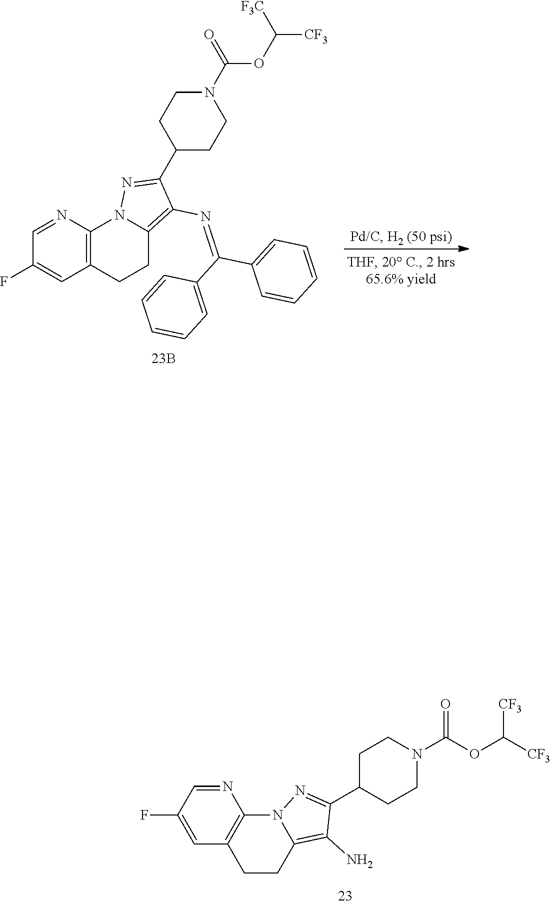 [SMILES: O=C(OC(C(F)(F)F)C(F)(F)F)N1CCC(c2nn3c(c2N=C(c2ccccc2)c2ccccc2)CCc2cc(F)cnc2-3)CC1 ; Nc1c(C2CCN(C(=O)OC(C(F)(F)F)C(F)(F)F)CC2)nn2c1CCc1cc(F)cnc1-2]

### Scheme-23: 1,1,1,3,3,3-hexafluoropropan-2-yl 4-(3-amino-7-fluoro-4,5-dihydropyrazolo[1,5-a][1,8]naphthyridin-2-yl)piperidine-1-carboxylate (23)

### Procedure for preparation of 1,1,1,3,3,3-hexafluoropropan-2-yl 4-(3-amino-7-fluoro-4,5-dihydropyrazolo[1,5-a][1,8]naphthyridin-2-yl)piperidine-1-carboxylate

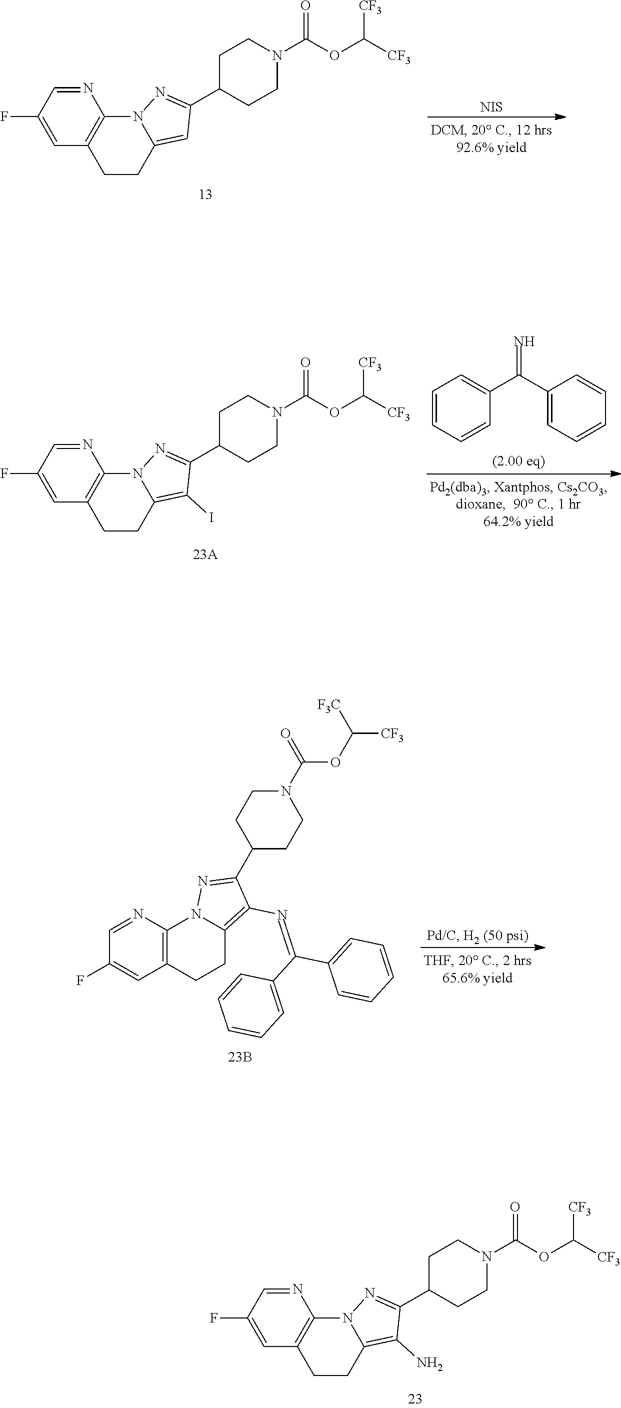

The compound was prepared according to the same synthesis step of example 9 starting from 13 to afford compound 23 (70.0 mg, 142 μmol, 65.6% yield, 97.8% purity) as a white solid.

LCMS: Rt=0.610 min, MS=482.2, M+H⁺

¹H NMR: (400 MHz, DMSO-d₆)

δ 8.19-8.19 (d, J=2.8 Hz, 1H), 7.73-7.71 (m, 1H), 6.57-6.64 (m, 1H), 4.03-4.09 (m, 2H), 3.97 (s, 2H), 2.91-2.95 (m, 2H), 2.84-2.93 (m, 5H), 1.61-1.63 (d, J=1.2 Hz, 2H), 1.57-1.61 (m, 2H)

### Example 24: 1,1,1,3,3,3-hexafluoropropan-2-yl 4-(7-fluoro-4,5-dihydro-[1,2,4]triazolo[1,5-a]quinolin-2-yl)piperidine-1-carboxylate

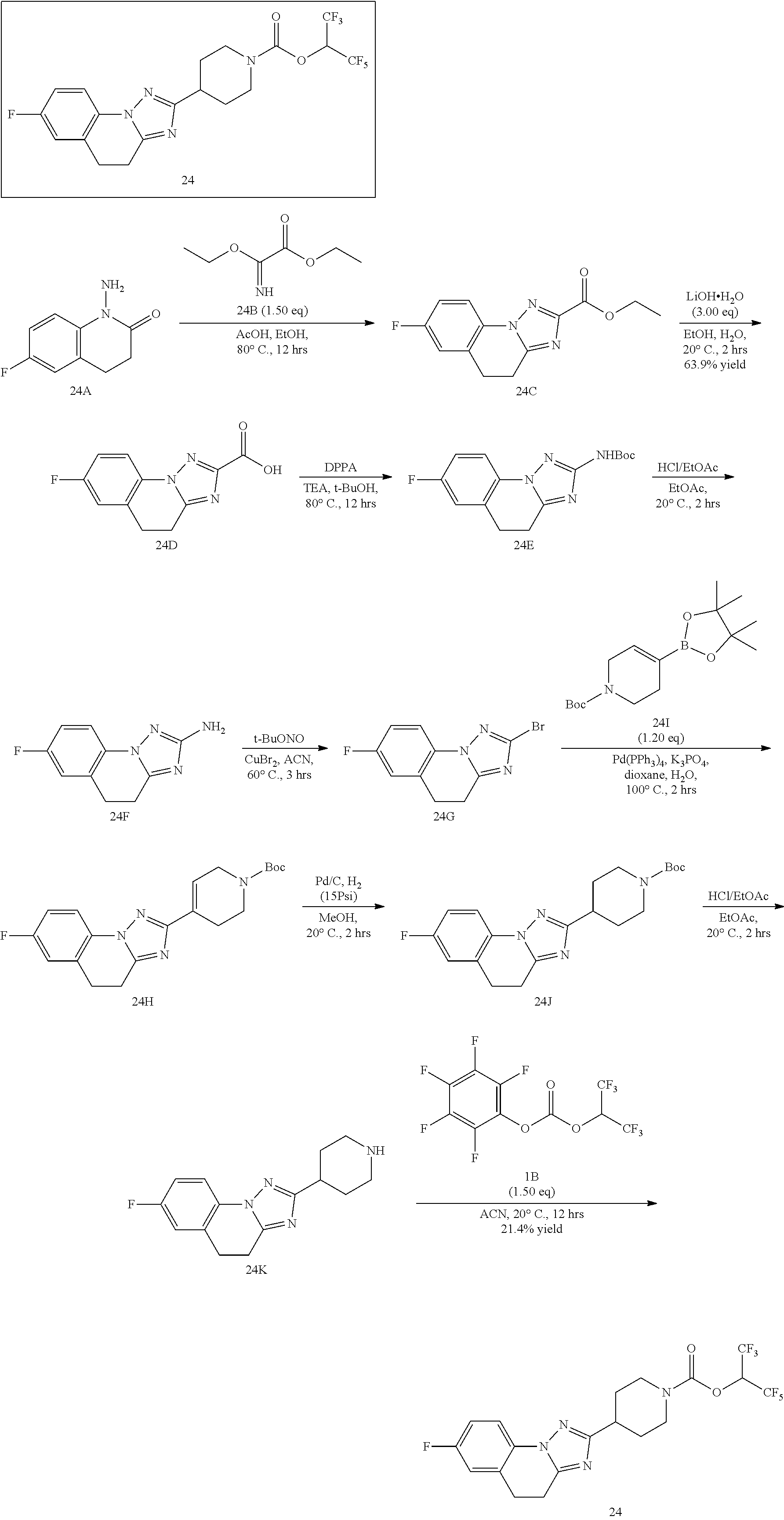

### Scheme-24: 1,1,1,3,3,3-hexafluoropropan-2-yl 4-(7-fluoro-4,5-dihydro-[1,2,4]triazolo[1,5-a]quinolin-2-yl)piperidine-1-carboxylate (23)

### Procedure for preparation of 1,1,1,3,3,3-hexafluoropropan-2-yl 4-(3-amino-7-fluoro-4,5-dihydropyrazolo[1,5-a][1,8]naphthyridin-2-yl)piperidine-1-carboxylate (24C)

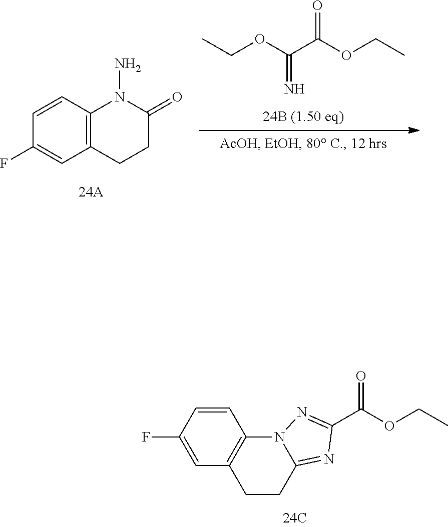

To a solution of compound 24A (150 mg, 832 μmol, 1.00 eq) in EtOH (1.00 mL) was added AcOH (149 mg, 2.50 mmol, 142 μL, 3.00 eq) and compound 24B (181 mg, 1.25 mmol, 1.50 eq). The mixture was stirred at 80° C. for 12 hrs. The reaction mixture was filtered and concentrated under reduced pressure to give a residue. The residue was purified by column chromatography (SiO₂, Petroleum ether/Ethyl acetate=0/1, R_{f}=0.50) to give compound 24C (250 mg, crude) as a yellow solid.

LCMS: Rt=0.382 min, MS=262.1, M+H⁺

### Procedure for preparation of 7-fluoro-4,5-dihydro-[1,2,4]triazolo[1,5-a]quinoline-2-carboxylic acid (24E)

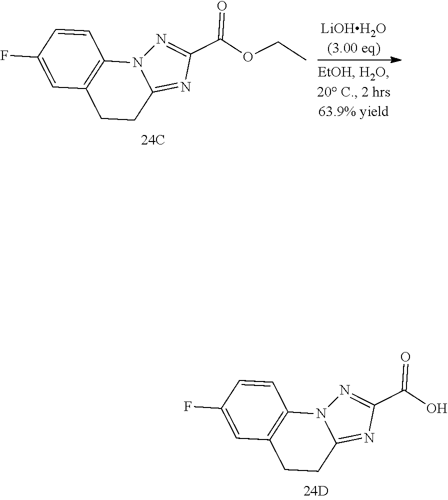 [SMILES: CCOC(=O)c1nc2n(n1)-c1ccc(F)cc1CC2 ; "LiOH·H2O(3.00 eq)" ; O=C(O)c1nc2n(n1)-c1ccc(F)cc1CC2]

To a solution of compound 24C (250 mg, 956 μmol, 1.00 eq) in THF (2.00 mL) and H₂O (0.40 mL) was added LiOH·H₂O (120 mg, 2.87 mmol, 3.00 eq). The mixture was stirred at 20° C. for 2 hrs. The reaction mixture was quenched by addition HCl 3.00 mL (1 M) at 0° C., and then diluted with H₂O 2.00 mL and extracted with EtOAc. The combined organic layers were washed with brine, dried over Na₂SO₄, filtered and concentrated under reduced pressure to give compound 24D (180 mg, 612 μmol, 63.9% yield, 79.3% purity) as a yellow solid. LCMS: Rt=0.297 min, MS=234.0, M+H⁺

### Procedure for preparation of tert-butyl (7-fluoro-4,5-dihydro-[1,2,4]triazolo[1,5-a]quinolin-2-yl)carbamate (24E)

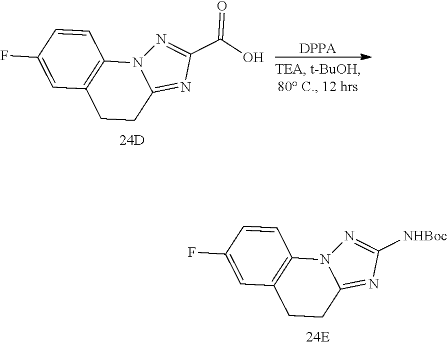

To a solution of compound 24D (150 mg, 643 μmol, 1.00 eq) in t-BuOH (2.00 mL) was added DPPA (194 mg, 707 μmol, 152 μL, 1.10 eq) and TEA (84.6 mg, 836 μmol, 116 μL, 1.30 eq). The mixture was stirred at 80° C. for 12 hrs. The residue was diluted with H₂O (2.00 mL) and extracted with EtOAc. The combined organic layers were washed brine, dried over Na₂SO₄, filtered and concentrated under reduced pressure to give compound 24E (150 mg, crude) as a yellow solid.

LCMS: Rt=0.400 min, MS=305.1, M+H⁺

### Procedure for preparation of 7-fluoro-4,5-dihydro-[1,2,4]triazolo[1,5-a]quinolin-2-amine (24F)

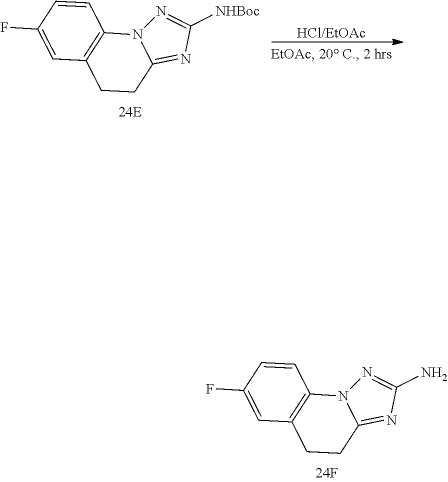 [SMILES: CC(C)(C)OC(=O)Nc1nc2n(n1)-c1ccc(F)cc1CC2 ; "HCl/EtOAc" ; Nc1nc2n(n1)-c1ccc(F)cc1CC2]

To a solution of compound 24E (150 mg, 492 μmol, 1.00 eq) in EtOAc (0.50 mL) was added HCl/EtOAc (2.0 M, 2.46 mL, 10.0 eq). The mixture was stirred at 20° C. for 2 hrs. The reaction mixture was concentrated under reduced pressure to give compound 24F (140 mg, crude) as a yellow solid.

LCMS: Rt=0.290 min, MS=205.1, M+H⁺.

### Procedure for preparation of 2-bromo-7-fluoro-4,5-dihydro-[1,2,4]triazolo[1,5-a]quinoline (24G)

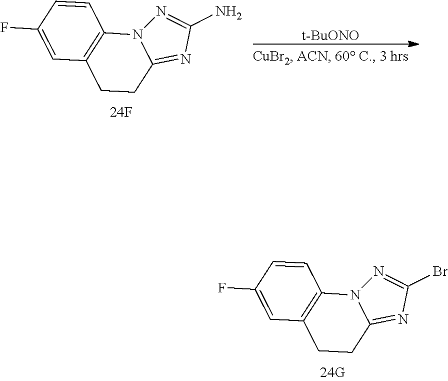 [SMILES: "t-BuONO" ; Nc1nc2n(n1)-c1ccc(F)cc1CC2 ; Fc1ccc2c(c1)CCc1nc(Br)nn1-2]

To a solution of compound 24F (100 mg, 489 μmol, 1.00 eq) in ACN (0.50 mL) was added CuBr₂ (164 mg, 734 μmol, 34.4 μL, 1.50 eq) and t-BuONO (75.7 mg, 734 μmol, 87.3 μL, 1.50 eq). The mixture was stirred at 60° C. for 3 hrs. The residue was diluted with H₂O (1.00 mL) and extracted with EtOAc (2.00 mL×3). The combined organic layers were washed brine (1.00 mL×3), dried over Na₂SO₄, filtered and concentrated under reduced pressure to give compound 24G (150 mg, crude) as a yellow solid.

LCMS: Rt=0.389 min, MS=269.9, M+2+H⁺,

### Procedure for preparation of tert-butyl 4-(7-fluoro-4,5-dihydro-[1,2,4]triazolo[1,5-a]quinolin-2-yl)-3,6-dihydropyridine-1(2H)-carboxylate (24H)

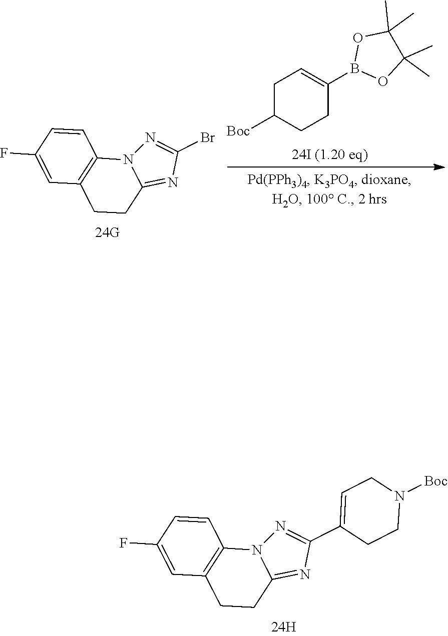

To a solution of compound 24G (85.0 mg, 317 μmol, 1.00 eq) in dioxane (1.00 mL) and H₂O (0.20 mL) was added K₃PO₄ (101 mg, 476 μmol, 1.50 eq) and Pd(PPh₃)₄ (26.8 mg, 31.7 μmol, 0.10 eq) and compound 2a (117 mg, 380 μmol, 1.20 eq). The mixture was stirred at 100° C. for 2 hrs. The residue was diluted with H₂O (2.00 mL) and extracted with EtOAc (3.00 mL×3). The combined organic layers were washed brine (2.00 mL×3), dried over Na₂SO₄, filtered and concentrated under reduced pressure. The residue was purified by prep-TLC (SiO₂, Petroleum ether/Ethyl acetate=2/1, R_{f}=0.50) to give compound 24H (100 mg, crude) as a yellow solid.

LCMS: Rt=0.477 min, MS=371.1, M+H⁺

### Procedure for preparation of tert-butyl 4-(7-fluoro-4,5-dihydro-[1,2,4]triazolo[1,5-a]quinolin-2-yl)piperidine-1-carboxylate (24J)

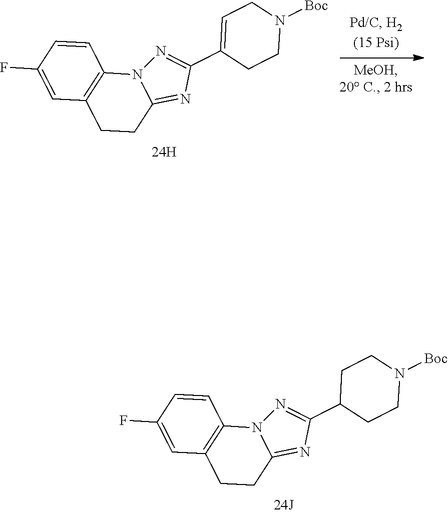 [SMILES: CC(C)(C)OC(=O)N1CC=C(c2nc3n(n2)-c2ccc(F)cc2CC3)CC1 ; CC(C)(C)OC(=O)N1CCC(c2nc3n(n2)-c2ccc(F)cc2CC3)CC1]

To a solution of compound 24H (100 mg, 269 μmol, 1.00 eq) in MeOH (1.00 mL) was added Pd/C (28.7 mg, 27.0 μmol, 10.0% purity, 0.10 eq) and H₂ (15 Psi). The mixture was stirred at 20° C. for 2 hrs. The reaction mixture was filtered and concentrated under reduced pressure to give compound 24J (80.0 mg, crude) as a yellow solid.

LCMS: Rt=0.454 min, MS=373.2, M+H⁺

### Procedure for preparation of tert-butyl 4-(7-fluoro-4,5-dihydro-[1,2,4]triazolo[1,5-a]quinolin-2-yl)piperidine-1-carboxylate (24)

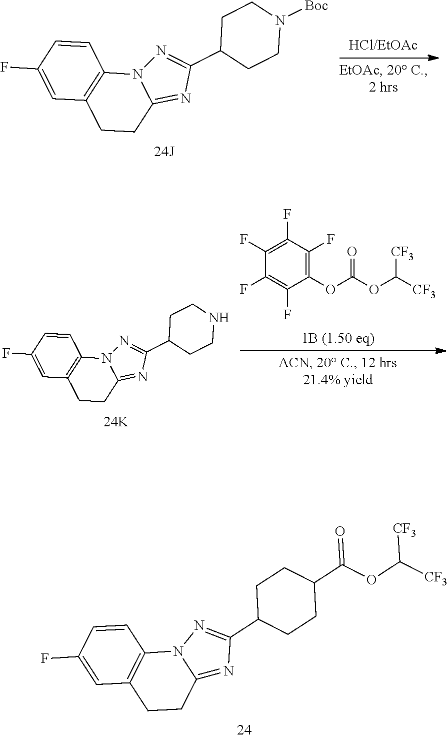

The compound was prepared according to the same synthesis step of example 1 starting from 24J to afford compound 24 (70.0 mg, 142 μmol, 65.6% yield, 97.8% purity) as a white solid.

LCMS: Rt=0.610 min, MS=482.2, M+H⁺

¹H NMR: (400 MHz, DMSO-d₆)

δ 8.19-8.19 (d, J=2.8 Hz, 1H), 7.73-7.71 (m, 1H), 6.57-6.64 (m, 1H), 4.03-4.09 (m, 2H), 3.97 (s, 2H), 2.91-2.95 (m, 2H), 2.84-2.93 (m, 5H), 1.61-1.63 (d, J=1.2 Hz, 2H), 1.57-1.61 (m, 2H).

### Example 25: 1,1,1-trifluoro-3-hydroxypropan-2-yl 4-(7-fluoro-4,5-dihydro-triazolo[1,5-a]quinolin-2-yl)piperidine-1-carboxylate

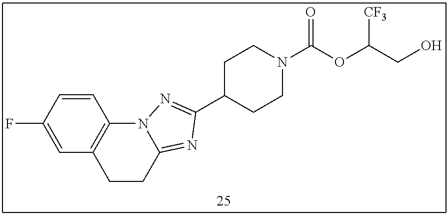 [SMILES: O=C(OC(CO)C(F)(F)F)N1CCC(c2nc3n(n2)-c2ccc(F)cc2CC3)CC1]

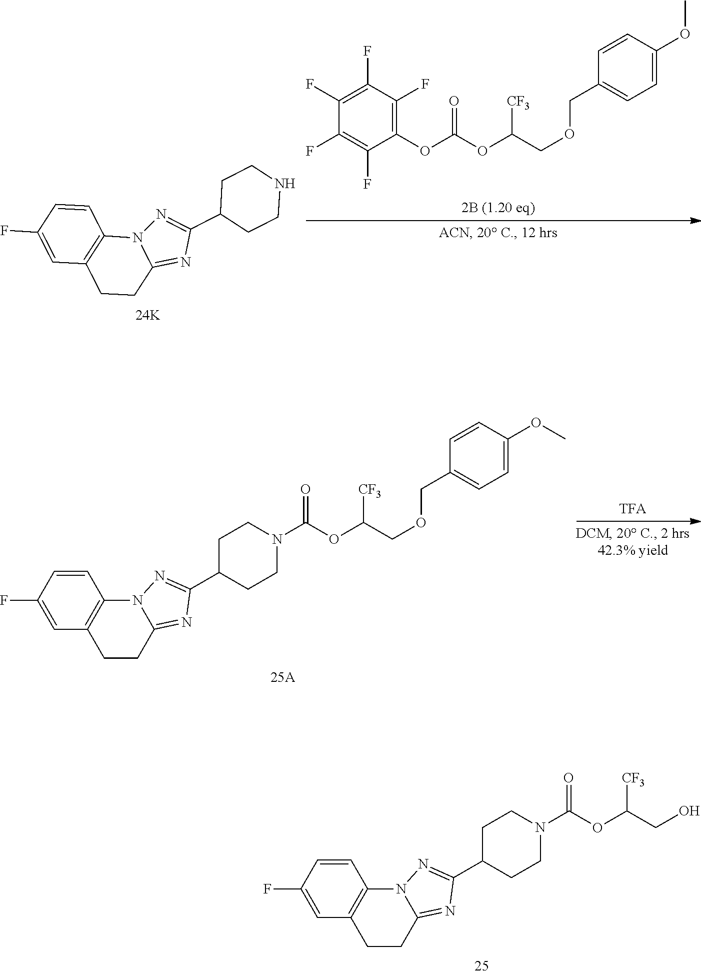

### Scheme-25: 1,1,1-trifluoro-3-hydroxypropan-2-yl 4-(7-fluoro-4,5-dihydro-[1,2,4]triazolo[1,5-a]quinolin-2-yl)piperidine-1-carboxylate (25)

### Procedure for preparation of 1,1,1-trifluoro-3-hydroxypropan-2-yl 4-(7-fluoro-4,5-dihydro-[1,2,4]triazolo[1,5-a]quinolin-2-yl)piperidine-1-carboxylate (25)

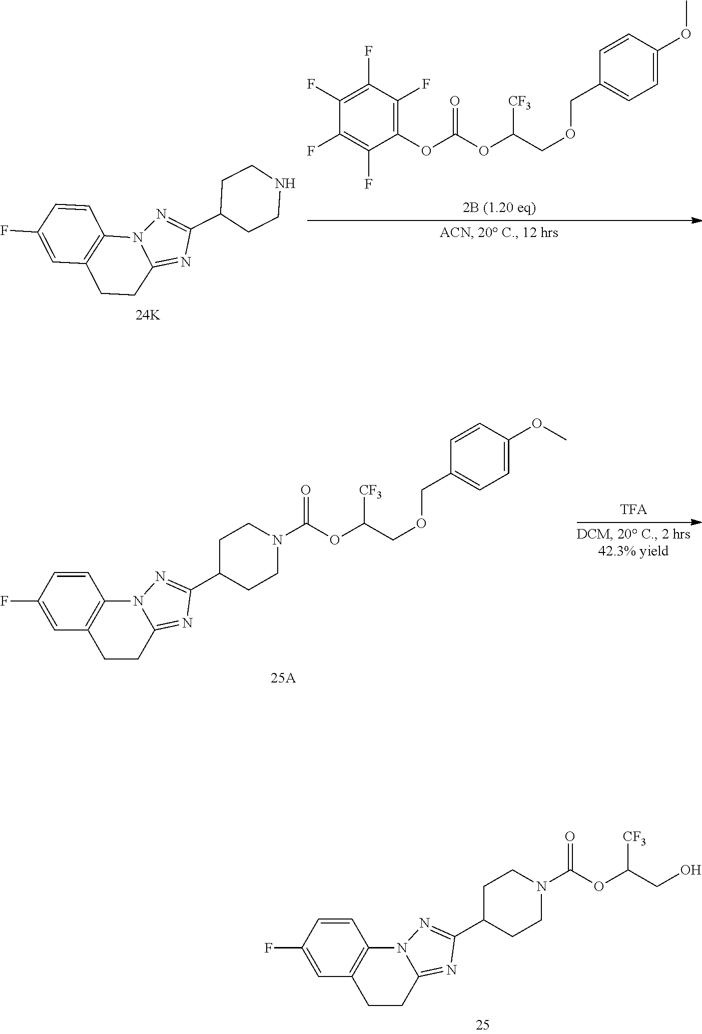

The compound was prepared according to the same synthesis step of example 2 starting from 24K to afford compound 25 (40.0 mg, 115 μmol, 42.3% yield, 99.2% purity) as a yellow solid.

LCMS: Rt=0.435 min, MS=429.2, M+H⁺

¹H NMR: (400 MHz, CDCl₃)

δ 7.72 (t, J=4.0 Hz, 1H), 7.00-7.05 (m, 2H), 4.40-4.46 (m, 3H), 4.17-4.23 (m, 3H), 3.08-3.17 (m, 7H), 2.07-2.11 (m, 2H), 1.87-1.93 (m, 2H).

### Example 26: 1,1,1-trifluoro-3-hydroxypropan-2-yl 4-(7-fluoro-4,5-dihydroimidazo[1,2-a]quinolin-2-yl)piperidine-1-carboxylate

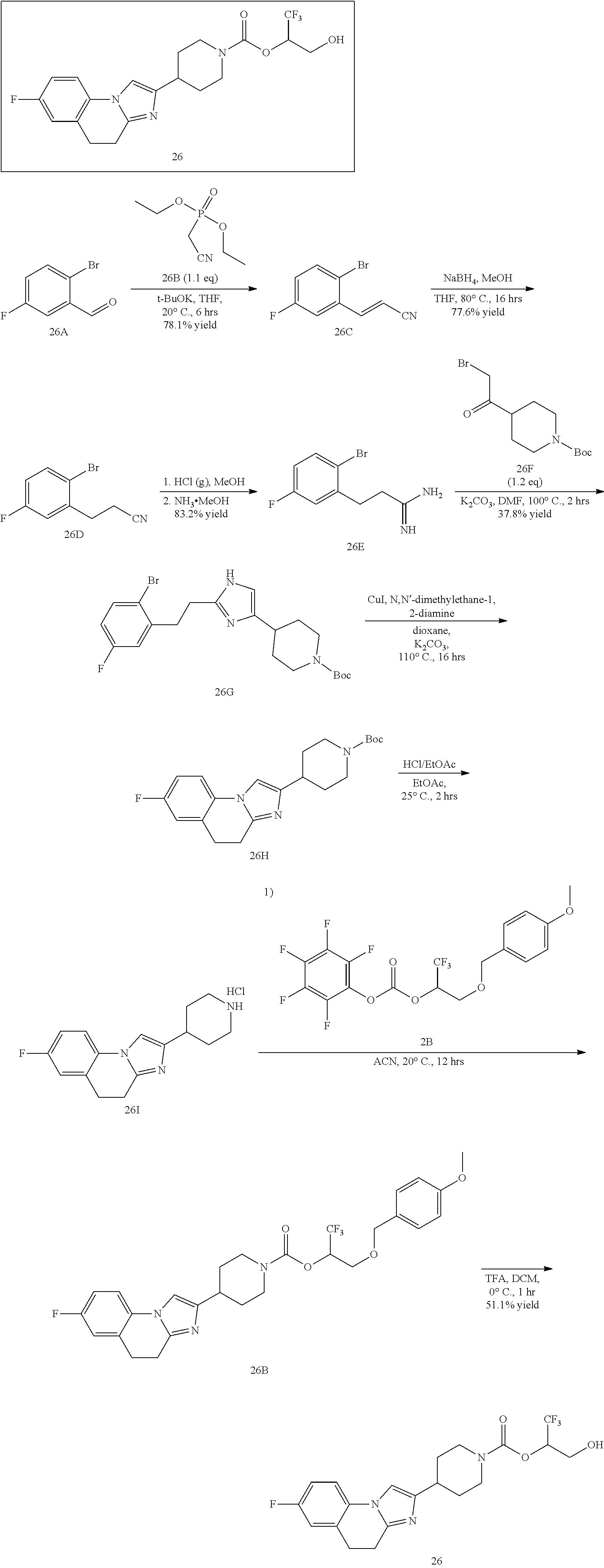

### Scheme-26: 1,1,1-trifluoro-3-hydroxypropan-2-yl 4-(7-fluoro-4,5-dihydroimidazo[1,2-a]quinolin-2-yl)piperidine-1-carboxylate (26)

### Procedure for preparation of (E)-3-(2-bromo-5-fluorophenyl)acrylonitrile (26C)

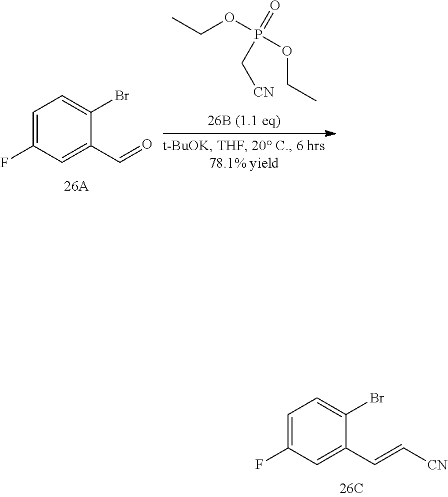

To a solution of compound 26A (10.0 g, 49.2 mmol, 1.00 eq) and compound 26B (9.60 g, 54.1 mmol, 8.77 mL, 1.10 eq) in THF (100 mL) was added t-BuOK (1 M, 59.1 m L, 1.20 eq) at 0° C. The mixture was stirred at 20° C. for 2 hrs. The reaction mixture was diluted with H₂O (200 mL) and extracted with EtOAc (200 mL). The organic layers were washed with brine (200 mL), dried over Na₂SO₄, filtered and concentrated under reduced pressure to give a residue. The residue was purified by column chromatography (SiO₂, Petroleum ether/Ethyl acetate=20/1, R_{f}=0.35) to give compound 26C (8.70 g, 38.4 mmol, 78.1% yield) as a yellow oil.

### Procedure for preparation of 3-(2-bromo-5-fluorophenyl)propanenitrile (26D)

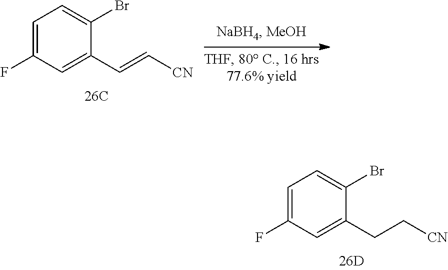 [SMILES: N#C/C=C/c1cc(F)ccc1Br ; N#CCCc1cc(F)ccc1Br]

To a solution of compound 26C (8.70 g, 38.4 mmol, 1.00 eq) in THF (90.0 mL) was added NaBH₄ (2.91 g, 76.9 mmol, 2.00 eq) at 0° C. The reaction mixture was stirred at 20° C. for 0.50 hr. Then was dropwise add MeOH (0.90 mL). The reaction mixture was stirred at 80° C. for 23.5 hrs. The reaction mixture was quenched by addition HCl 30.0 mL (1 M) at 0° C., and then diluted with H₂O (200 mL) and extracted with EtOAc. The combined organic layers were washed with brine, dried over Na₂SO₄, filtered and concentrated under reduced pressure to give a residue. The residue was purified by column chromatography (SiO₂, Petroleum ether/Ethyl acetate=5/1, R_{f}=0.54) to give compound 26D (7.00 g, 29.8 mmol, 77.6% yield, 97.3% purity) as a white solid.

LCMS: Rt=0.329 min, MS=228.0, M+H⁺

### Procedure for preparation of 3-(2-bromo-5-fluorophenyl)propanimidamide (26E)

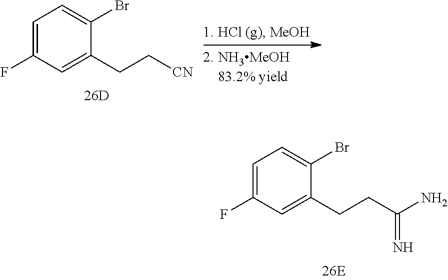 [SMILES: N#CCCc1cc(F)ccc1Br ; N=C(N)CCc1cc(F)ccc1Br]

To a solution of compound 26D (3.00 g, 12.8 mmol, 1.00 eq) in MeOH (30.0 mL) was added HCl (933 mg, 25.6 mmol, 915 μL, 2.00 eq). The mixture was stirred at 0° C. for 1 hr. The reaction mixture was concentrated under reduced pressure to give intermediate. Then To a solution of the intermediate in NH₃/MeOH (20.0 mL). The mixture was stirred at 40° C. for 12 hrs. The reaction mixture was concentrated under reduced pressure to give a residue. The residue was purified by prep-HPLC (column: Phenomenex luna C18 (250×70 mm, 10 um); mobile phase: [water (FA)-ACN]; gradient: 3%-33% B over 15 min) to give compound 26E (3.12 g, 10.5 mmol, 83.2% yield, 98.6% purity) as a white solid.

LCMS: Rt=0.979 min, MS=245.1, M+H⁺

### Procedure for preparation of tert-butyl 4-(2-(2-bromo-5-fluorophenethyl)-1H-imidazol-4-yl)piperidine-1-carboxylate (26G)

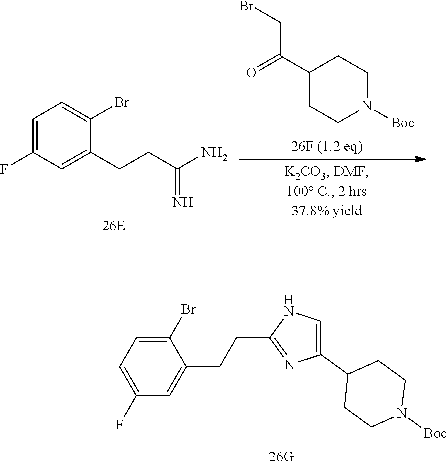 [SMILES: N=C(N)CCc1cc(F)ccc1Br ; CC(C)(C)OC(=O)N1CCC(C(=O)CBr)CC1 ; CC(C)(C)OC(=O)N1CCC(c2c[nH]c(CCc3cc(F)ccc3Br)n2)CC1]

To a solution of compound 26E (2.50 g, 8.59 mmol, 1.00 eq) in DMF (30.0 mL) was added K₂CO₃ (2.37 g, 17.1 mmol, 2.00 eq) and compound 2b (3.16 g, 10.3 mmol, 1.20 eq). The residue was diluted with H₂O (20.0 mL) and extracted with EtOAc. The combined organic layers were washed brine, dried over Na₂SO₄, filtered and concentrated under reduced pressure. The residue was purified by prep-HPLC (column: Daisogel C18 250×70 mm×10 um; mobile phase: [water (NH₃·H₂O)-ACN]; gradient: 30%-65% B over 25 mins) to give compound 26G (1.50 g, 3.25 mmol, 37.8% yield, 98.1% purity) as a yellow solid.

LCMS: Rt=2.583 min, MS=452.0, M+H⁺,

### Procedure for preparation of tert-butyl 4-(7-fluoro-4,5-dihydroimidazo[1,2-a]quinolin-2-yl)piperidine-1-carboxylate (26H)

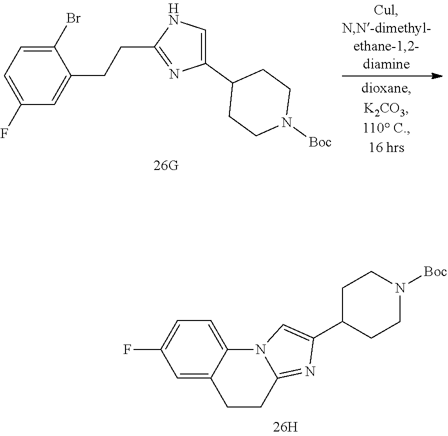 [SMILES: CC(C)(C)OC(=O)N1CCC(c2c[nH]c(CCc3cc(F)ccc3Br)n2)CC1 ; CC(C)(C)OC(=O)N1CCC(c2cn3c(n2)CCc2cc(F)ccc2-3)CC1]

To a solution of compound 26G (450 mg, 994 μmol, 1.00 eq) in toulene (5.00 mL) was added K₂C03 (412 mg, 2.98 mmol, 3.00 eq) and CuI (37.8 mg, 198 μmol, 0.20 eq) and N,N′-dimethylethane-1,2-diamine (17.5 mg, 198 μmol, 21.4 μL, 0.20 eq). The mixture was stirred at 110° C. for 16 hrs. The residue was diluted with H₂O (5.00 mL) and extracted with EtOAc. The combined organic layers were washed brine, dried over Na₂SO₄, filtered and concentrated under reduced pressure. The residue was purified by prep-HPLC (column: Daisogel C18 250×70 mm×10 um; mobile phase: [water (NH₃·H₂O)-ACN]; gradient: 30%-65% B over 25 min) to give compound 26H (300 mg, crude) as a yellow solid.

LCMS: Rt=0.358 min, MS=372.3, M+H⁺

### Procedure for preparation of 1,1,1-trifluoro-3-hydroxypropan-2-yl 4-(7-fluoro-4,5-dihydroimidazo[1,2-a]quinolin-2-yl)piperidine-1-carboxylate (26)

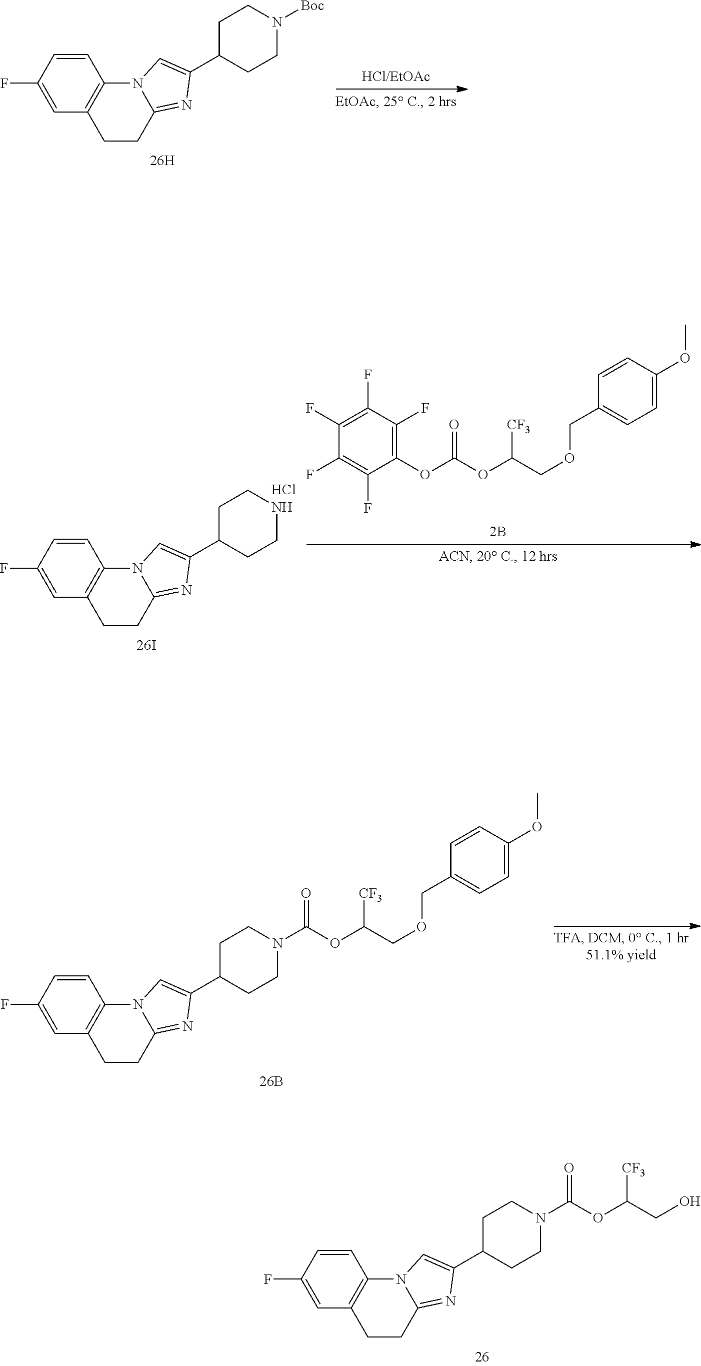

The compound was prepared according to the same synthesis step of example 2 starting from 26H to afford compound 26 (80.0 mg, 186 μmol, 51.1% yield, 99.8% purity) as a white solid.

LCMS: Rt=0.342 min, MS=428.1, M+H⁺

¹H NMR: (400 MHz, DMSO_d₆)

δ 7.54-7.57 (m, 2H), 7.23-7.26 (m, 1H), 7.14-7.18 (m, 1H), 5.21-5.30 (m, 2H), 4.01-4.04 (m, 2H), 3.66-3.73 (m, 2H), 3.23-3.27 (m, 3H), 2.95-2.98 (m, 2H), 2.66-2.74 (m, 1H), 2.31-2.33 (m, 1H), 1.94-1.97 (m, 2H), 1.45-1.51 (m, 2H),

### Example 27: 1,1,1-trifluoro-3-hydroxypropan-2-yl 4-(1-chloro-7-fluoro-4,5-dihydroimidazo[1,2-a]quinolin-2-yl)piperidine-1-carboxylate

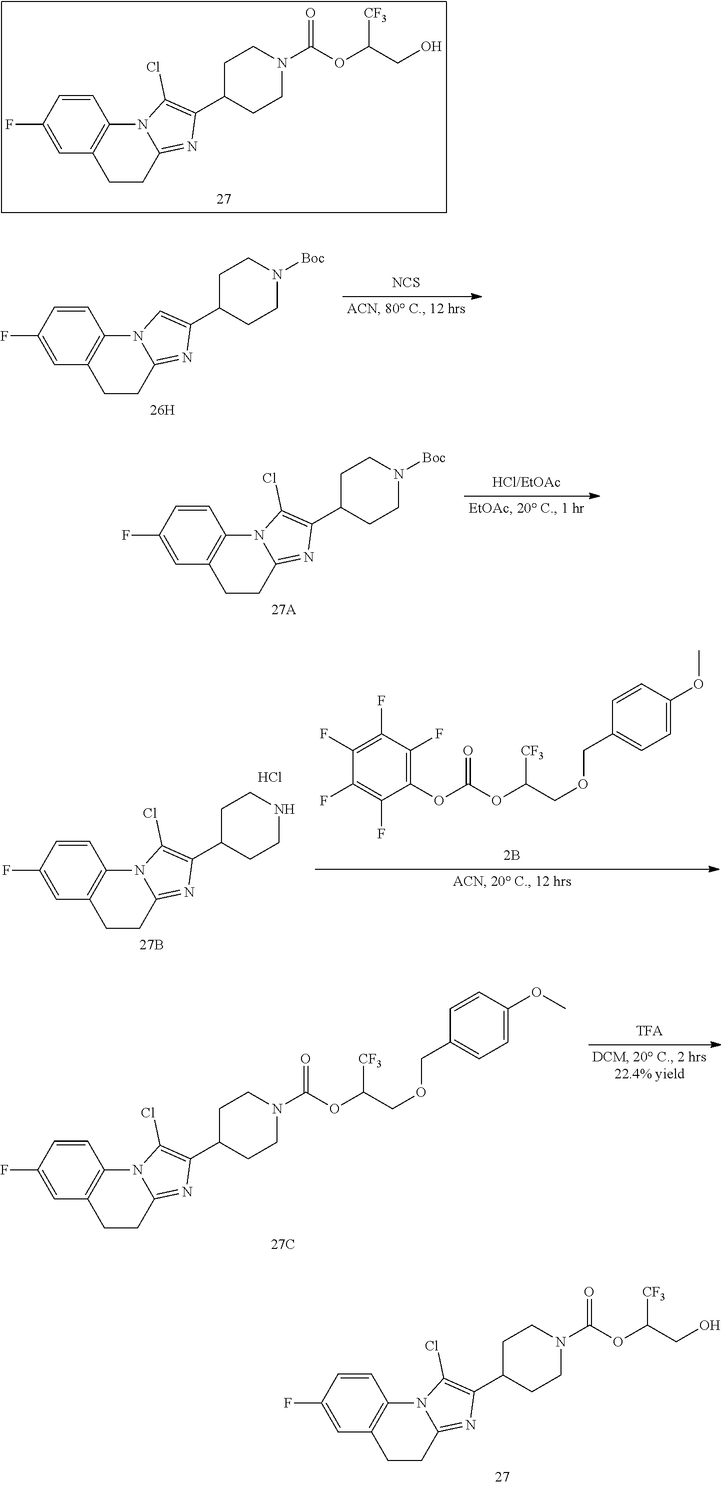

### Scheme-27: 1,1,1-trifluoro-3-hydroxypropan-2-yl 4-(1-chloro-7-fluoro-4,5-dihydroimidazo[1,2-a]quinolin-2-yl)piperidine-1-carboxylate(27)

### Procedure for preparation of tert-butyl 4-(1-chloro-7-fluoro-4,5-dihydroimidazo[1,2-a]quinolin-2-yl)piperidine-1-carboxylate (27A)

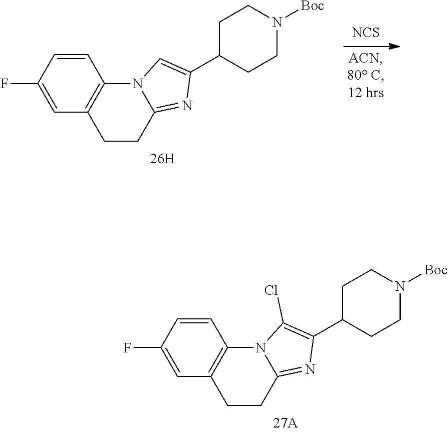 [SMILES: CC(C)(C)OC(=O)N1CCC(c2cn3c(n2)CCc2cc(F)ccc2-3)CC1 ; "ACN,80° C,12 hrs" ; "NCS" ; CC(C)(C)OC(=O)N1CCC(c2nc3n(c2Cl)-c2ccc(F)cc2CC3)CC1]

To a solution of compound 26H (170 mg, 457 μmol, 1.00 eq) in ACN (1.00 mL) was added NCS (91.7 mg, 686 μmol, 1.50 eq) at 0° C. The mixture was stirred at 25° C. for 12 hrs. The mixture was quenched by H₂O (10.0 mL) extracted with Ethyl acetate. Then the combined organic layers were washed with brine, dried over Na₂SO₄, filtered and concentrated under reduced pressure to give a residue to obtained compound 27A (198 mg, crude) as a red solid.

LCMS: Rt=0.460 min, MS=406.1, M+H⁺

### Procedure for preparation of 1,1,1-trifluoro-3-hydroxypropan-2-yl 4-(1-chloro-7-fluoro-4,5-dihydroimidazo[1,2-a]quinolin-2-yl)piperidine-1-carboxylate (27)

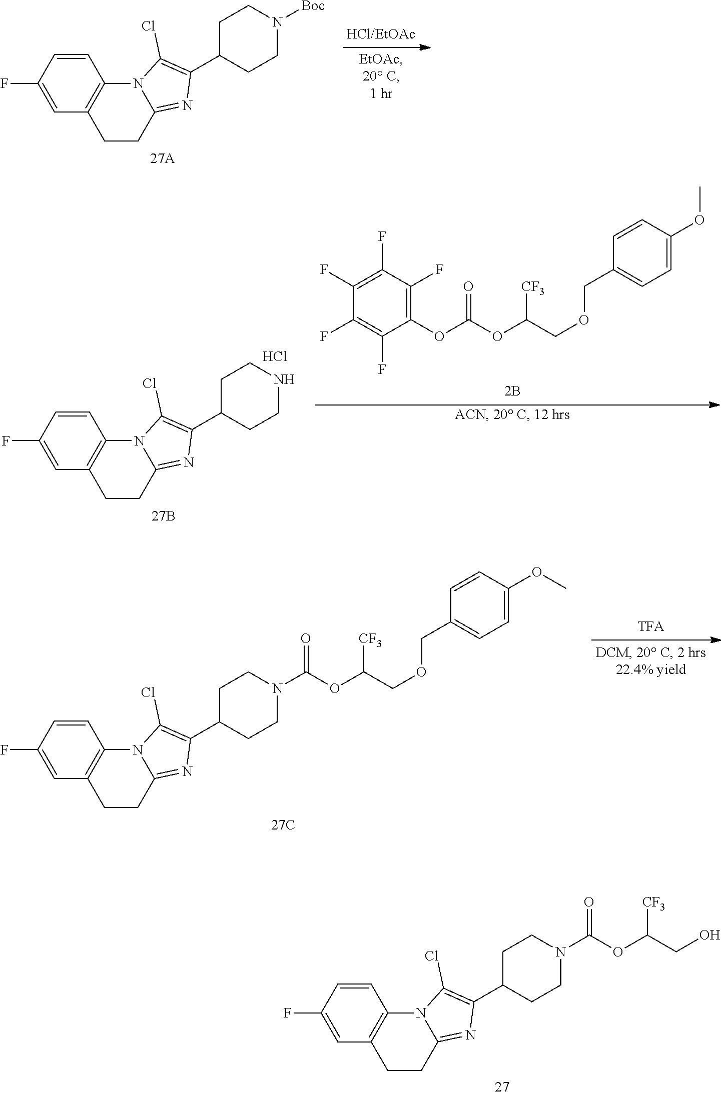

The compound was prepared according to the same synthesis step of example 2 starting from 27A to afford compound 27 (39.0 mg, 83.0 μmol, 22.4% yield, 98.3% purity) as an off-white solid.

LCMS: Rt=0.356 min, MS=462.0, M+H⁺

¹HNMR: (400 MHz, MeOD)

δ 8.02-8.08 (m, 1H), 7.10-7.25 (m, 2H), 5.26-5.38 (m, 1H), 4.25-4.35 (m, 2H), 3.77-3.95 (m, 2H), 2.93-3.06 (m, 2H), 1.75-1.95 (m, 4H).

### Example 28:1,1,1-trifluoro-3-hydroxypropan-2-yl 4-(1-bromo-7-fluoro-4,5-dihydroimidazo[1,2-a]quinolin-2-yl)piperidine-1-carboxylate

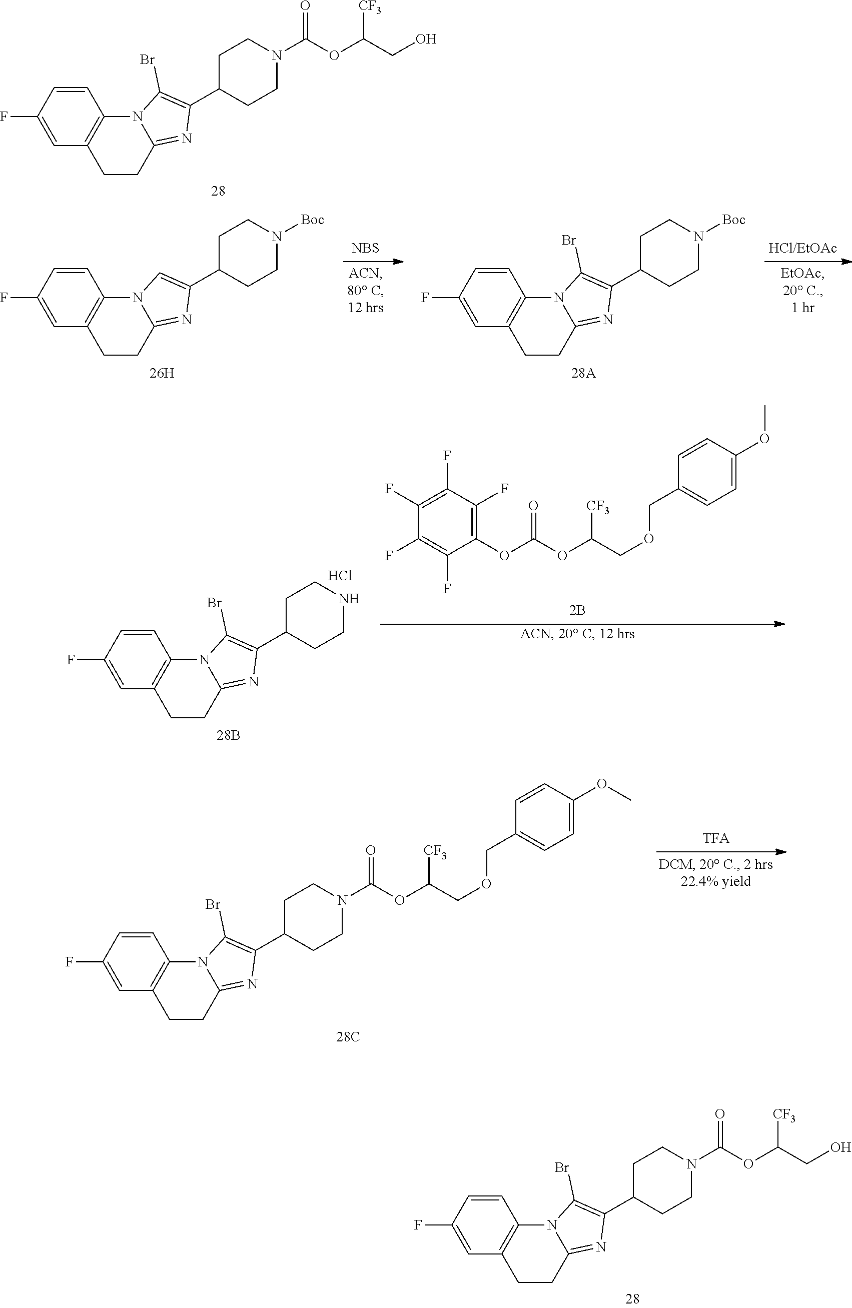

### Scheme-28: 1,1,1-trifluoro-3-hydroxypropan-2-yl 4-(1-bromo-7-fluoro-4,5-dihydroimidazo[1,2-a]quinolin-2-yl)piperidine-1-carboxylate (28)

### Procedure for preparation of tert-butyl 4-(1-chloro-7-fluoro-4,5-dihydroimidazo[1,2-a]quinolin-2-yl)piperidine-1-carboxylate (28A)

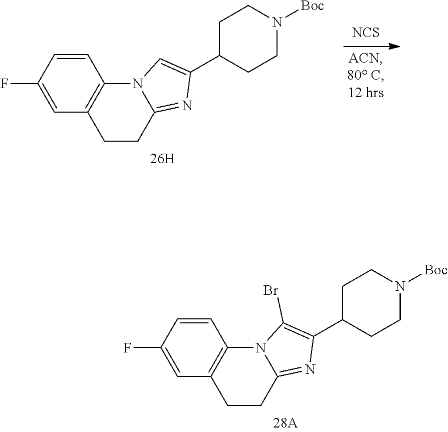 [SMILES: CC(C)(C)OC(=O)N1CCC(c2cn3c(n2)CCc2cc(F)ccc2-3)CC1 ; "ACN,80° C,12 hrs" ; "NCS" ; CC(C)(C)OC(=O)N1CCC(c2nc3n(c2Br)-c2ccc(F)cc2CC3)CC1]

To a solution of compound 26H (134 mg, 350 μmol, 1.00 eq) in DCM (2.00 mL) was added NBS (93.4 mg, 524 μmol, 1.50 eq). The mixture was stirred at 20° C. for 1 hr. The residue was diluted with H₂O (2.00 mL) and extracted with EtOAc. The combined organic layers were washed brine, dried over Na₂SO₄, filtered and concentrated under reduced pressure to give compound 28A (150 mg, crude) as a yellow solid.

LCMS: Rt=2.615 min, MS=450.2, M+H⁺

### Procedure for preparation of 1,1,1-trifluoro-3-hydroxypropan-2-yl 4-(1-bromo-7-fluoro-4,5-dihydroimidazo[1,2-a]quinolin-2-yl)piperidine-1-carboxylate (28)

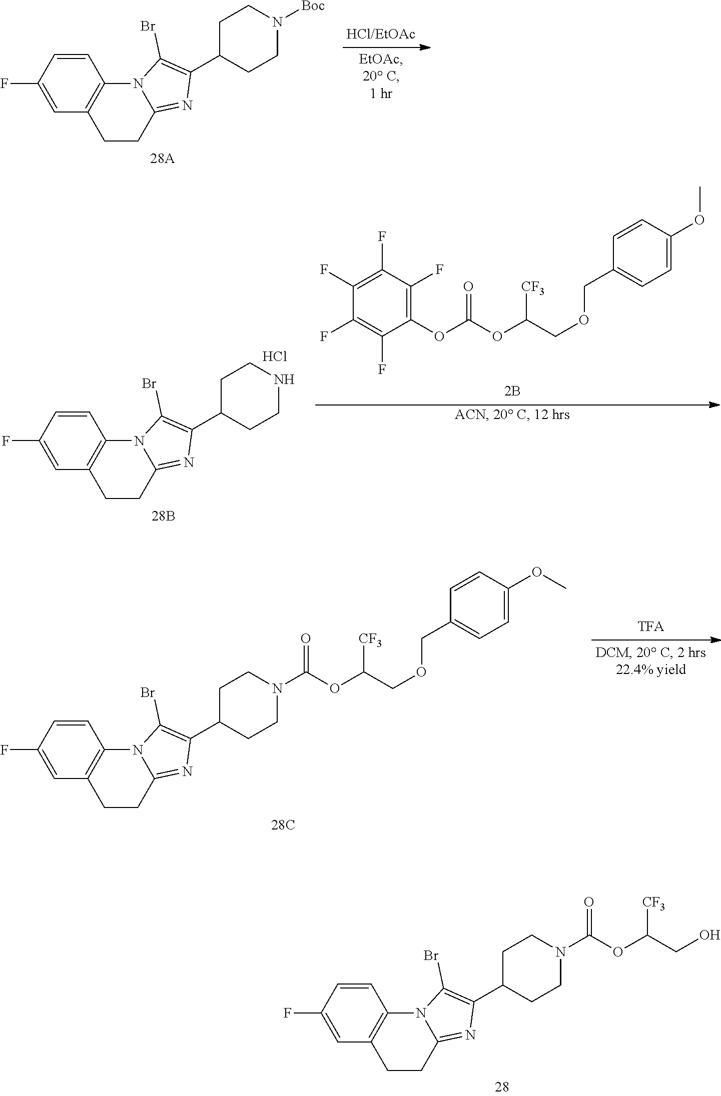

The compound was prepared according to the same synthesis step of example 2 starting from 28A to afford compound 28 (110 mg, 217 μmol, 45.3% yield, 100% purity) as a white solid.

LCMS: Rt=0.429 min, MS=507.9, M+2+H⁺

¹H NMR: (400 MHz, CDCl₃)

δ 8.03-8.07 (m, 1H), 7.01-7.05 (m, 2H), 5.23-5.36 (m, 1H), 4.25-4.33 (m, 2H), 4.02-4.04 (m, 1H), 3.88-3.99 (m, 1H), 2.89-2.03 (m, 7H), 1.82-1.93 (m, 4H).

### Example 29: 1,1,1-trifluoro-3-hydroxypropan-2-yl 4-(3,9-difluoro-5,6-dihydroimidazo[1,2-a][1,8]naphthyridin-8-yl)piperidine-1-carboxylate

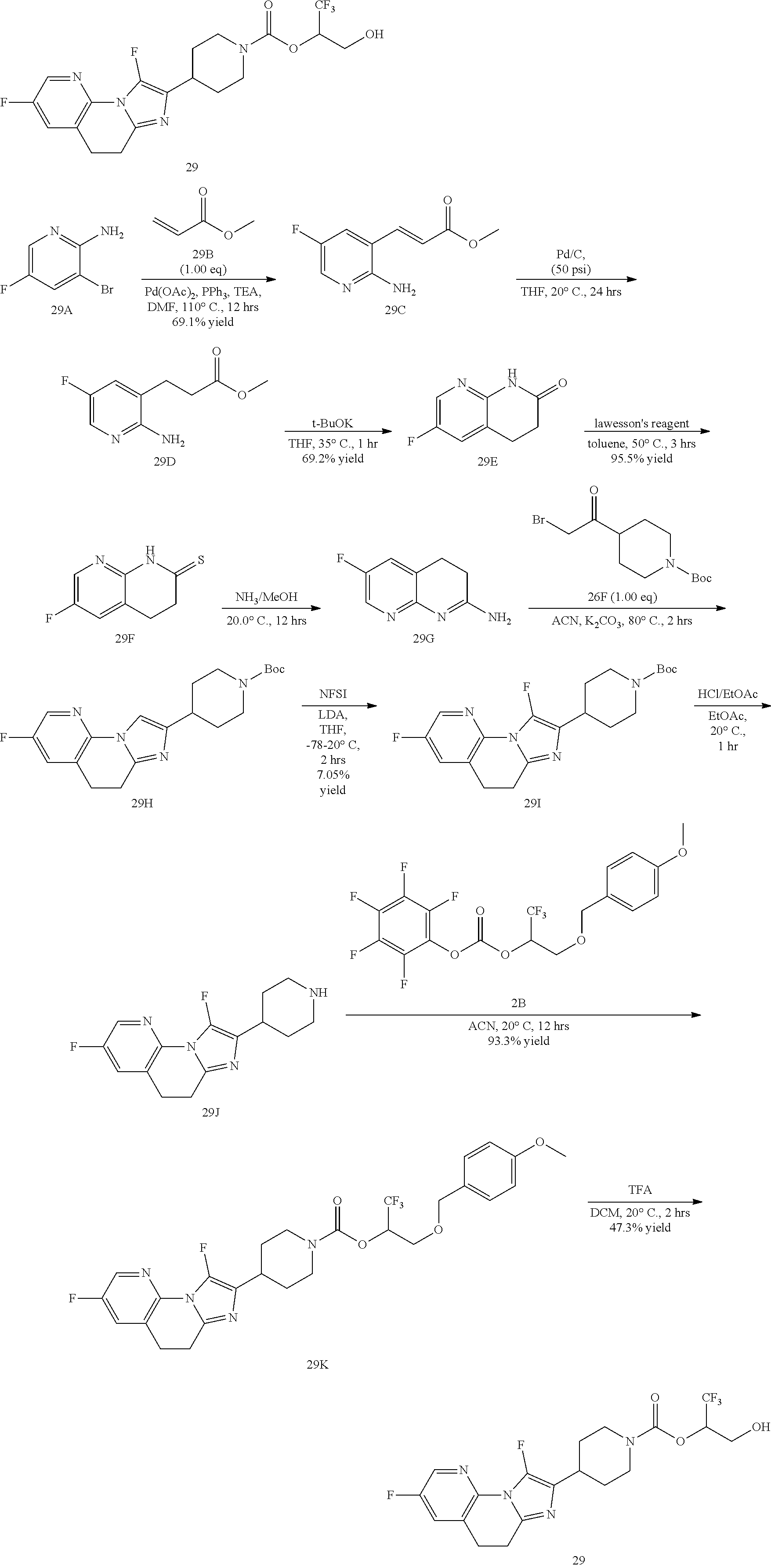

### Scheme-29: 1,1,1-trifluoro-3-hydroxypropan-2-yl 4-(3,9-difluoro-5,6-dihydroimidazo[1,2-a][1,8]naphthyridin-8-yl)piperidine-1-carboxyl ate (29)

### Procedure for preparation of methyl (E)-3-(2-amino-5-fluoropyridin-3-yl)acrylate (29C)

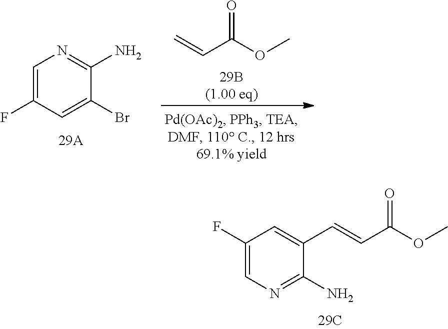

A mixture of compound 29A (25.0 g, 131 mmol, 1.00 eq), compound 29B (24.9 g, 289 mmol, 26.0 mL, 1.00 eq), PPh₃ (6.87 g, 26.2 mmol, 0.20 eq), Pd(OAc)₂ (2.94 g, 13.1 mmol, 0.10 eq) and TEA (14.6 g, 144 mmol, 20.04 mL, 1.10 eq) in DMF (200 mL) was degassed and purged with N₂ for 3 times, and then the mixture was stirred at 110° C. for 12 hrs under N₂ atmosphere. The reaction was quenched by addition of 100 mL of saturated ammonium chloride, then acidify the clear solution with diluted hydrochloride acid, extracted with EtOAc. The combined organic layers were washed with brine, dried over Na₂SO₄, filtered and concentrated under reduced pressure to give a residue. The residue was purified by column chromatography (SiO₂, Petroleum ether/Ethyl acetate=3/1, R_{f}=0.49) to give compound 29C (18.0 g, 90.4 mmol, 69.1% yield, 98.5% purity) as a yellow solid.

LCMS: Rt=0.254 min, MS=197.1, M+H⁺

### Procedure for preparation of methyl 3-(2-amino-5-fluoropyridin-3-yl)propanoate

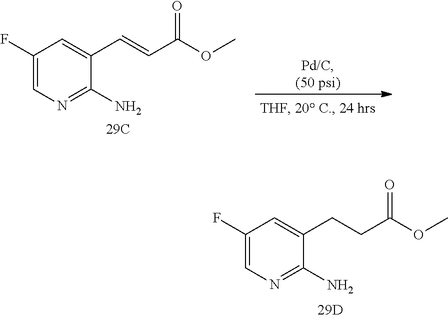

To a solution of compound 29C (8.00 g, 40.8 mmol, 1.00 eq) in THF (80.0 mL) was added Pd/C (4.34 g, 4.08 mmol, 10% purity, 0.10 eq) under N₂ atmosphere. The suspension was degassed and purged with H₂ for 3 times. The mixture was stirred under H₂ (50 Psi) at 25° C. for 24 hrs. The reaction mixture was concentrated under reduced pressure to remove solvent. The crude product was triturated with ethyl acetate (30.0 mL) at 25° C. for 30 min to give compound 29D (8.00 g, crude) as a white solid.

LCMS: Rt=0.075 min, MS=199.0, M+H⁺,

### Procedure for preparation of 6-fluoro-3,4-dihydro-1,8-naphthyridin-2(1H)-one (29E)

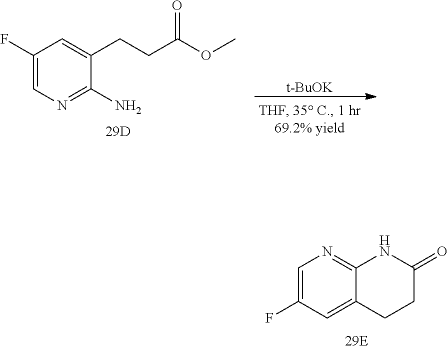

To a solution of compound 29D (4.50 g, 22.7 mmol, 1.00 eq) in EtOH (50.0 mL) was added KOtBu (1 M, 45.41 mL, 2.00 eq). The mixture was stirred at 35° C. for 1 hr. The reaction mixture was concentrated under reduced pressure to remove solvent. The crude product was triturated with H₂O at 25° C. for 30 min to give compound 29E (2.65 g, 15.7 mmol, 69.2% yield) as a white solid.

LCMS: Rt=0.161 min, MS=166.9, M+H⁺,

### Procedure for preparation of 6-fluoro-3,4-dihydro-1,8-naphthyridine-2(1H)-thione (29F)

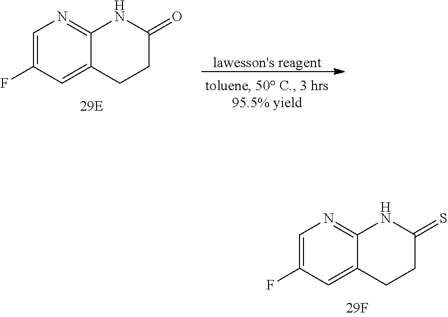 [SMILES: O=C1CCc2cc(F)cnc2N1 ; "lawesson's reagent" ; Fc1cnc2c(c1)CCC(=S)N2]

To a solution of compound 29E (1.30 g, 7.82 mmol, 1.00 eq) in toluene (13.0 mL) was added Iawesson's reagent (1.58 g, 3.91 mmol, 0.50 eq). The mixture was stirred at 50° C. for 3 hrs. The reaction mixture was concentrated under reduced pressure to remove solvent. The crude product was triturated with EtOAc (8.00 mL) at 20° C. for 30 min to give compound 29F (1.43 g, 7.47 mmol, 95.5% yield) as a yellow solid.

LCMS: Rt=0.223 min, MS=182.9, M+H⁺,

### Procedure for preparation of 6-fluoro-3,4-dihydro-1,8-naphthyridin-2-amine (29G)

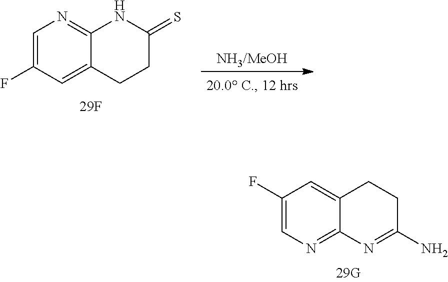 [SMILES: Fc1cnc2c(c1)CCC(=S)N2 ; NC1=Nc2ncc(F)cc2CC1]

To a solution of compound 29F (1.40 g, 7.68 mmol, 1.00 eq) in NH₃/MeOH (10.0 mL). The mixture was stirred at 20° C. for 12 hrs. The reaction mixture was concentrated under reduced pressure to give compound 29G (1.18 g, crude) as a yellow solid.

LCMS: Rt=0.336 min, MS=166.1, M+H⁺,

### Procedure for preparation of tert-butyl 4-(3-fluoro-5,6-dihydroimidazo[1,2-a][1,8]naphthyridin-8-yl)piperidine-1-carboxylate (29G)

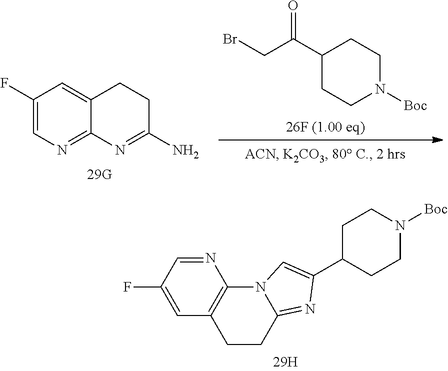 [SMILES: NC1=Nc2ncc(F)cc2CC1 ; CC(C)(C)OC(=O)N1CCC(C(=O)CBr)CC1 ; CC(C)(C)OC(=O)N1CCC(c2cn3c(n2)CCc2cc(F)cnc2-3)CC1]

To a solution of compound 29G (1.18 g, 7.14 mmol, 1.00 eq), compound 26F (2.19 g, 7.14 mmol, 1.00 eq) in ACN (12.0 mL) was added NaHCO₃ (1.80 g, 21.4 mmol, 834 μL, 3.00 eq). The mixture was stirred at 80° C. for 2 hrs. The mixture was added ice water (10.0 mL) at 0° C. and extracted with EtOAc. The combined organic layers were washed with brine (10.0 mL), dried over Na₂SO₄, filtered and concentrated under reduced pressure to give compound 29H (1.50 g, crude) as a yellow solid.

LCMS: Rt=0.304 min, MS=373.1, M+H⁺

### Procedure for preparation of tert-butyl 4-(3,9-difluoro-5,6-dihydroimidazo[1,2-a][1,8]naphthyridin-8-yl)piperidine-1-carboxylate (291)

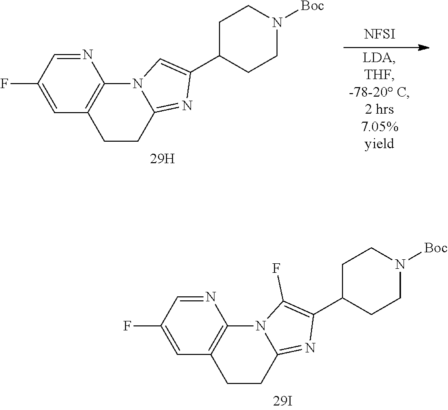

To a solution of LDA (1.5 M, 2.62 mL, 2.00 eq) in THF (2.00 mL) was added dropwise compound 29H (731 mg, 1.96 mmol, 1.00 eq) at −78° C. After addition, the mixture was stirred at this temperature for 1 hr, and then NFSI (1.24 g, 3.93 mmol, 2.00 eq) was added dropwise at 20° C., the mixture was stirred at 20° C. for 1 hr. The reaction was quenched by addition of (30.0 mL) of saturated ammonium chloride, then acidify the clear solution with diluted hydrochloride acid, extracted with Ethyl acetate. The combined organic layers were washed with brine, dried over Na₂SO₄, filtered and concentrated under reduced pressure to give a residue. The residue was purified by prep-HPLC (column: Phenomenex luna C18 150×25 mm×10 um; mobile phase: [water (FA)-ACN]; gradient: 34%-64% B over 10 min) to obtained compound 291 (60.0 mg, 138 μmol, 7.05% yield) as a white solid.

LCMS: Rt=0.359 min, MS=391.1, M+H⁺

### Procedure for preparation of 1,1,1-trifluoro-3-hydroxypropan-2-yl 4-(3,9-difluoro-5,6-dihydroimidazo[1,2-a][1,8]naphthyridin-8-yl)piperidine-1-carboxylate (29)

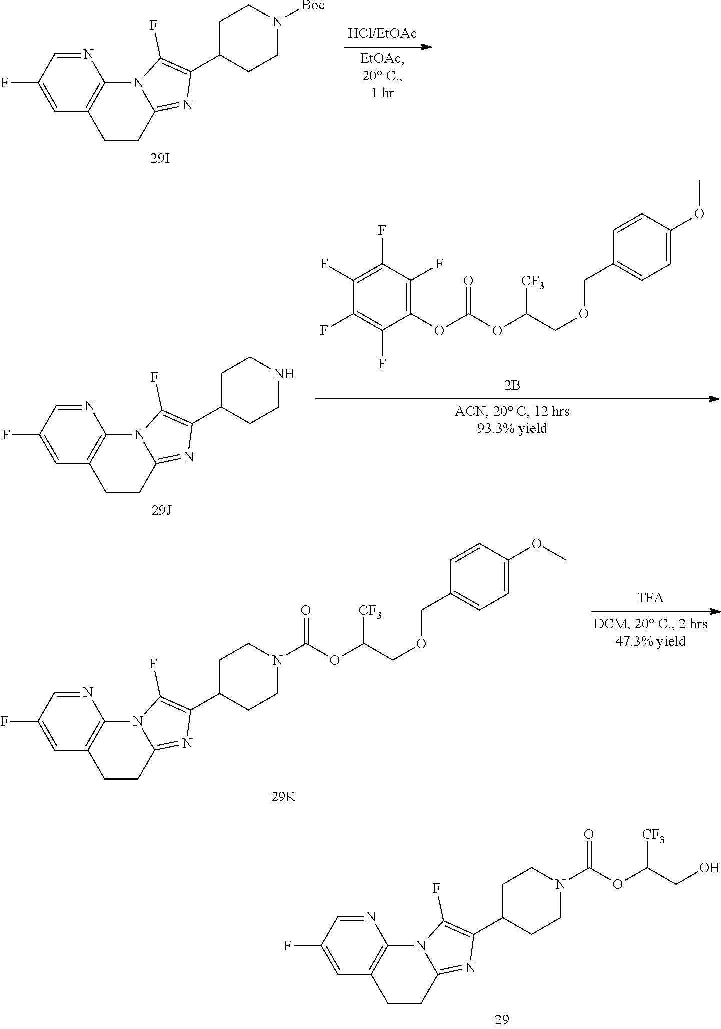

The compound was prepared according to the same synthesis step of example 2 starting from 291 to afford compound 29 (12.0 mg, 25.11 μmol, 47.3% yield, 93.4% purity) as a white solid.

LCMS: Rt=0.319 min, MS=447.1, M+H⁺

¹HNMR: (400 MHz, CDCl₃)

δ 8.23-8.24 (d, J=8, 1H), 7.40-7.42 (d, J=8, 1H), 5.24-5.28 (m, 1H), 4.39-4.43 (m, 1H), 4.19-4.25 (m, 2H), 3.99-4.02 (d, J=12, 1H), 3.90-3.91 (m, 1H), 3.00-3.09 (m, 7H), 2.86-2.97 (m, 1H), 1.88-1.98 (m, 2H), 1.81-1.84 (m, 2H)

### Example 30: 1,1,1-trifluoro-3-hydroxypropan-2-yl 4-(9-chloro-3-fluoro-5,6-dihydroimidazo[1,2-a][1,8]naphthyridin-8-yl)piperidine-1-carboxylate

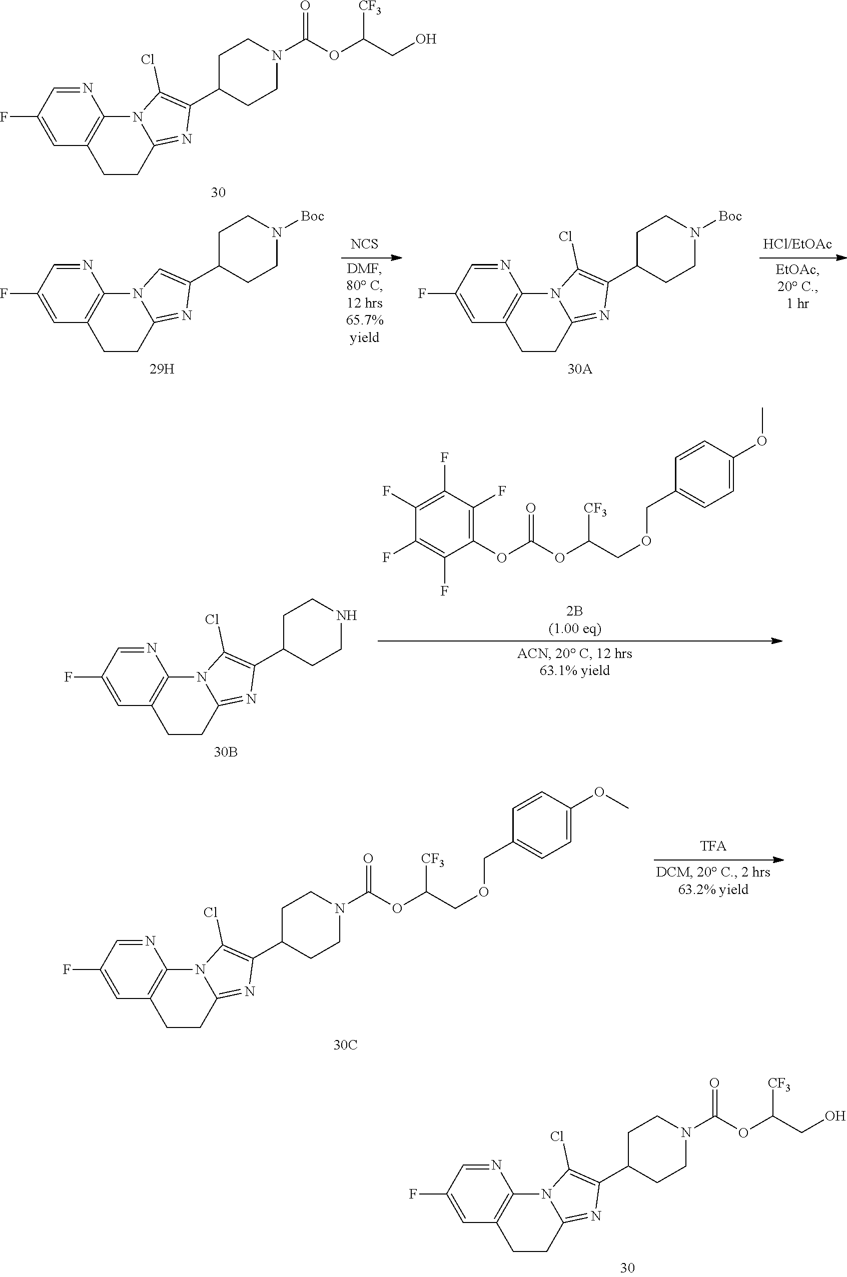

### Scheme-30: 1,1,1-trifluoro-3-hydroxypropan-2-yl 4-(9-chloro-3-fluoro-5,6-dihydroimidazo[1,2-a][1,8]naphthyridin-8-yl)piperidine-1-carboxylate(30)

### Procedure for preparation of tert-butyl 4-(1-chloro-7-fluoro-4,5-dihydroimidazo[1,2-a]quinolin-2-yl)piperidine-1-carboxylate (30A)

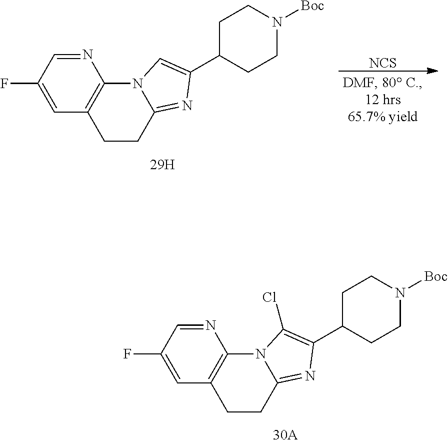 [SMILES: CC(C)(C)OC(=O)N1CCC(c2cn3c(n2)CCc2cc(F)cnc2-3)CC1 ; "NCS" ; CC(C)(C)OC(=O)N1CCC(c2nc3n(c2Cl)-c2ncc(F)cc2CC3)CC1]

To a solution of compound 29H (400 mg, 1.37 mmol, 1.00 eq) in DCM (5.00 mL) was added NCS (287 mg, 1.61 mmol, 1.10 eq). The mixture was stirred at 80° C. for 12 hrs. The reaction mixture was concentrated under reduced pressure to remove solvent. The residue was purified by flash silica gel chromatography (ISCO®; 12 g Sepa Flash® Silica Flash Column, Eluent of 0-68% Ethyl acetate/Petroleum ether gradient @100 mL/min) to give compound 30A (300 mg, 737 μmol, 65.7% yield) as a yellow solid.

LCMS: Rt=0.388 min, MS=407.1, M+H⁺

### Procedure for preparation of 1,1,1-trifluoro-3-hydroxypropan-2-yl 4-(9-chloro-3-fluoro-5,6-dihydroimidazo[1,2-a][1,8]naphthyridin-8-yl)piperidine-1-carboxylate (30)

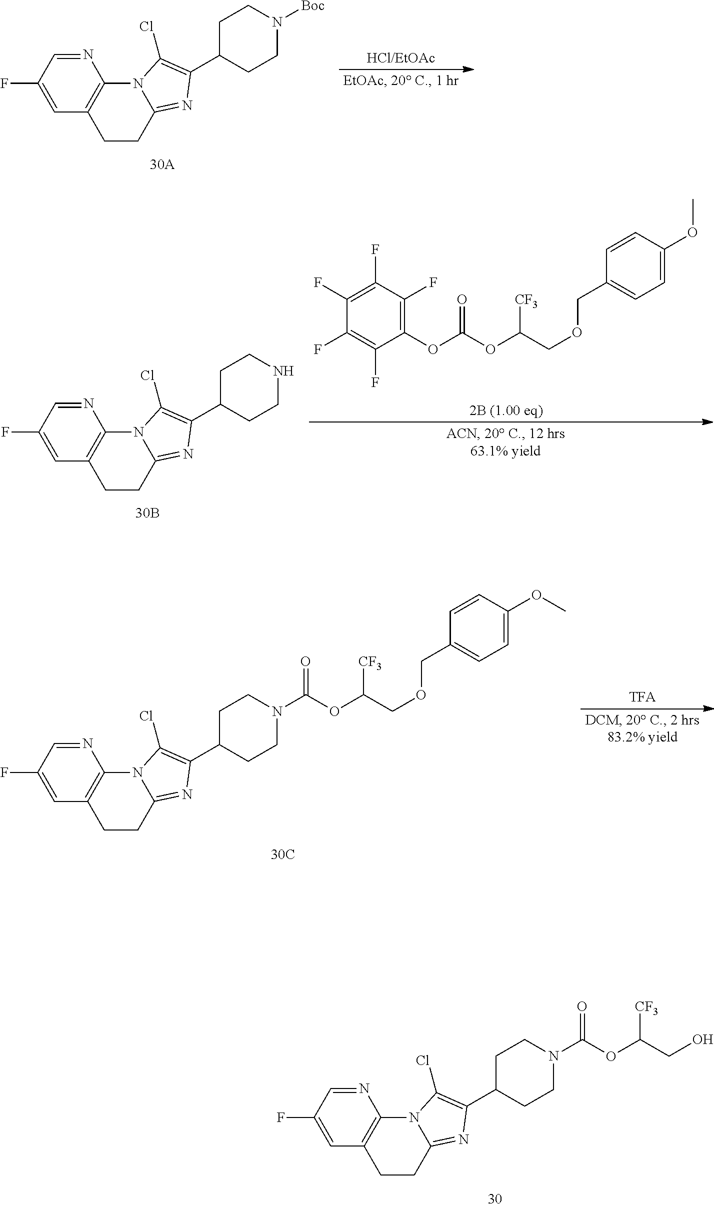

The compound was prepared according to the same synthesis step of example 2 starting from 30A to afford compound 30 (75.0 mg, 428 μmol, 83.2% yield, 99.1% purity) as a white solid.

LCMS: Rt=0.351 MS=463.0, M+H⁺

¹H NMR: (CDCl₃, 400 MHz)

δ 8.24-8.25 (d, J=4.0 Hz, 1H), 7.39-7.42 (d, J=12 Hz, 1H), 5.24-5.27 (m, 1H), 4.28-4.36 (m, 2H), 4.00-4.03 (d, J=12 Hz, 1H), 3.89-3.92 (m, 1H), 3.00-3.07 (m, 5H), 2.93-2.95 (m, 2H), 1.51-1.93 (m, 4H).

### Example 31: 1,1,1-trifluoro-3-hydroxypropan-2-yl 4-(9-bromo-3-fluoro-5,6-dihydroimidazo[1,2-a][1,8]naphthyridin-8-yl)piperidine-1-carboxylate

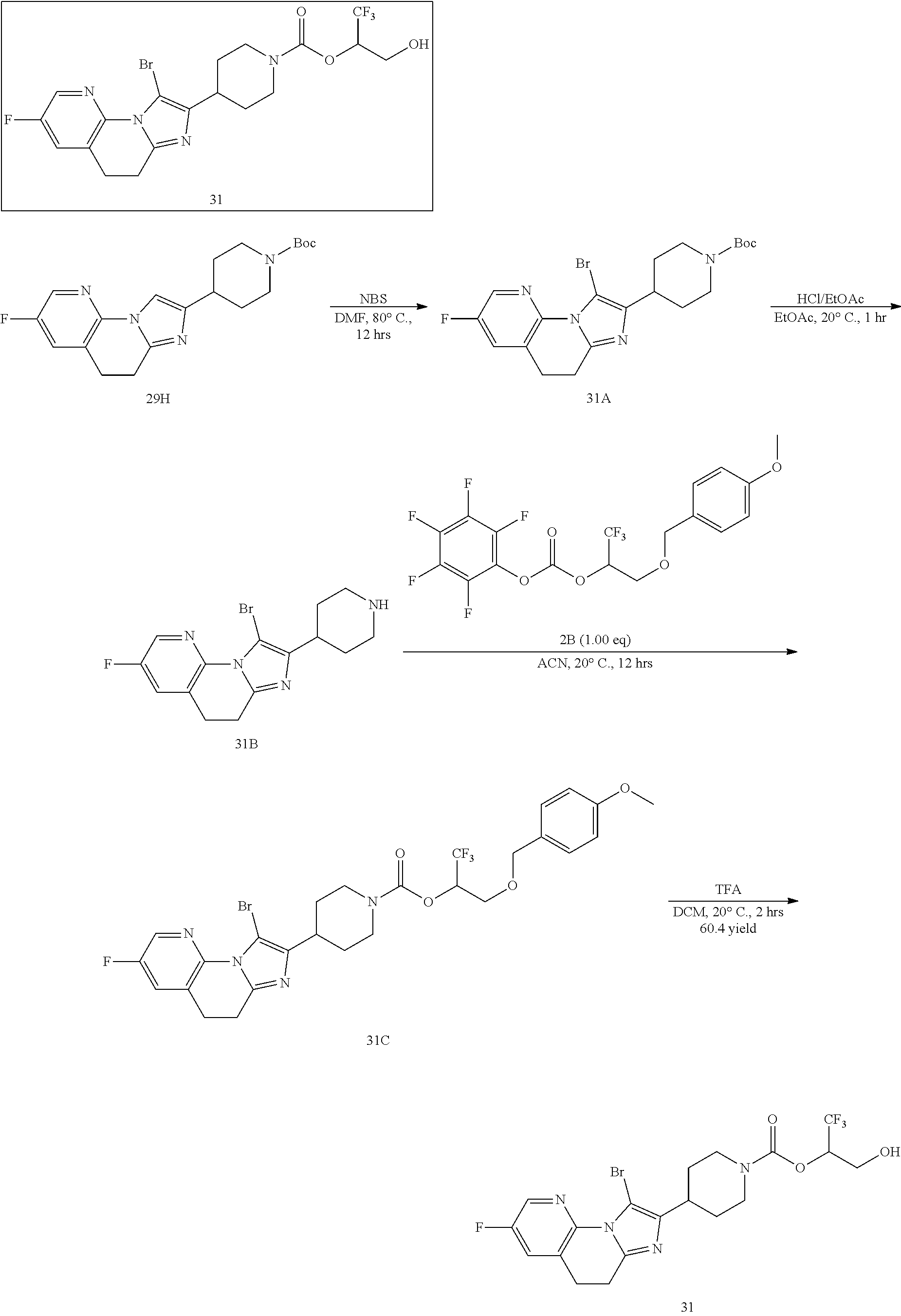

### Scheme-31: 1,1,1-trifluoro-3-hydroxypropan-2-yl 4-(9-bromo-3-fluoro-5,6-dihydroimidazo[1,2-a][1,8]naphthyridin-8-yl)piperidine-1-carboxylate(31)

### Procedure for preparation of tert-butyl 4-(9-bromo-3-fluoro-5,6-dihydroimidazo[1,2-a][1,8]naphthyridin-8-yl)piperidine-1-carboxylate (31A)

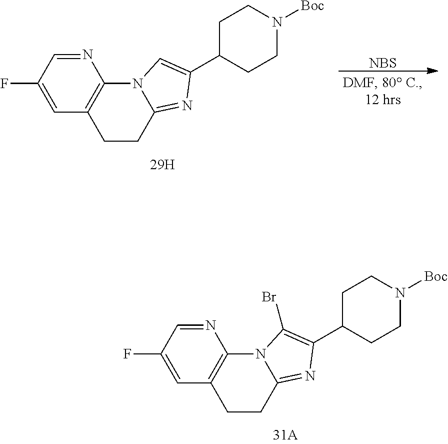 [SMILES: CC(C)(C)OC(=O)N1CCC(c2cn3c(n2)CCc2cc(F)cnc2-3)CC1 ; "NBS" ; CC(C)(C)OC(=O)N1CCC(c2nc3n(c2Br)-c2ncc(F)cc2CC3)CC1]

To a solution of compound 29H (500 mg, 1.34 mmol, 1.00 eq) in DCM (5.00 mL) was added NBS (287 mg, 1.61 mmol, 1.20 eq). The mixture was stirred at 20° C. for 12 hrs. The reaction mixture was concentrated under reduced pressure to remove solvent. The residue was purified by flash silica gel chromatography (ISCO®; 12 g Sepa Flash® Silica Flash Column, Eluent of 0-68% Ethyl acetate/Petroleum ether gradient @100 mL/min) to give compound 31A (397 mg, crude) as a yellow solid.

LCMS: Rt=0.387 min, MS=451.0, M+H⁺

### Procedure for preparation of 1,1,1-trifluoro-3-hydroxypropan-2-yl 4-(9-bromo-3-fluoro-5,6-dihydroimidazo[1,2-a][1,8]naphthyridin-8-yl)piperidine-1-carboxylate (31)

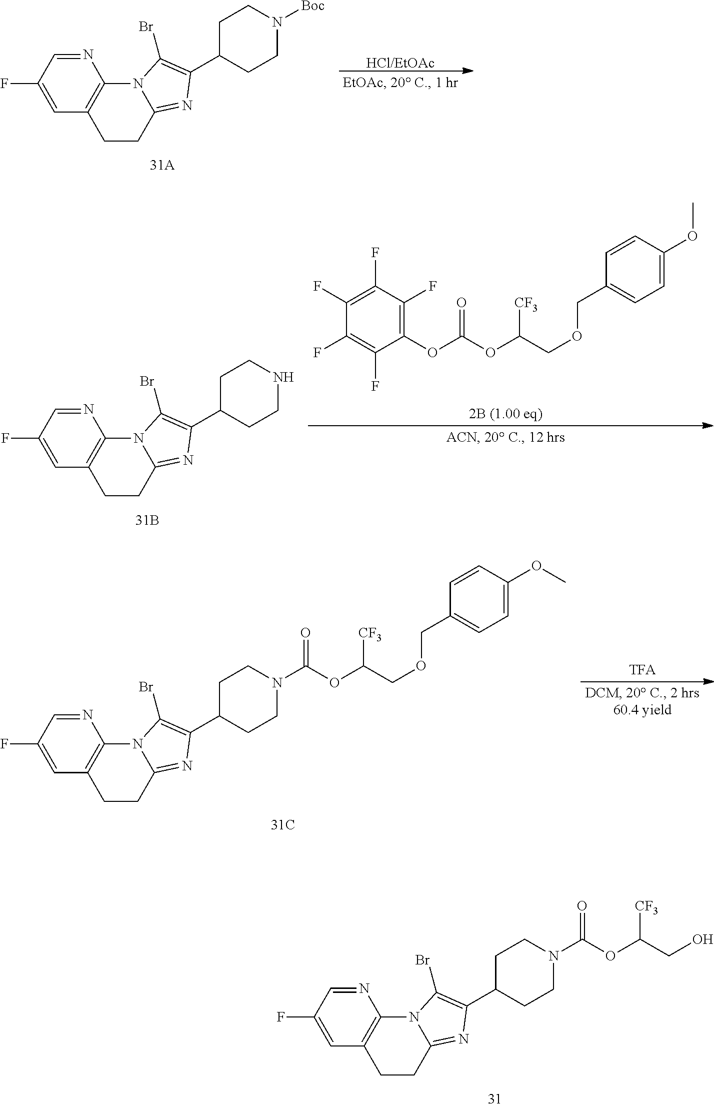

The compound was prepared according to the same synthesis step of example 2 starting from 31A to afford compound 31 (100 mg, 289 μmol, 60.4% yield, 97.7% purity) as a white solid.

LCMS: Rt=0.351, MS=509.0, M+H⁺

¹H NMR: (CDCl₃, 400 MHz)

δ 8.24-8.25 (d, J=4.0 Hz, 1H), 7.39-7.42 (d, J=12 Hz, 1H), 5.24-5.27 (m, 1H), 4.25-4.30 (m, 2H), 3.99-4.02 (d, J=12 Hz, 1H), 3.90-3.92 (m, 1H), 3.01-3.07 (m, 5H), 2.93-2.96 (m, 2H), 1.83-1.95 (m, 2H), 1.83-1.87 (m, 3H)

### Example 32: 1,1,1,3,3,3-hexafluoropropan-2-yl 4-(7-fluoro-4,5-dihydro-[1,2,4]triazolo[1,5-a][1,8]naphthyridin-2-yl)piperidine-1-carboxylate

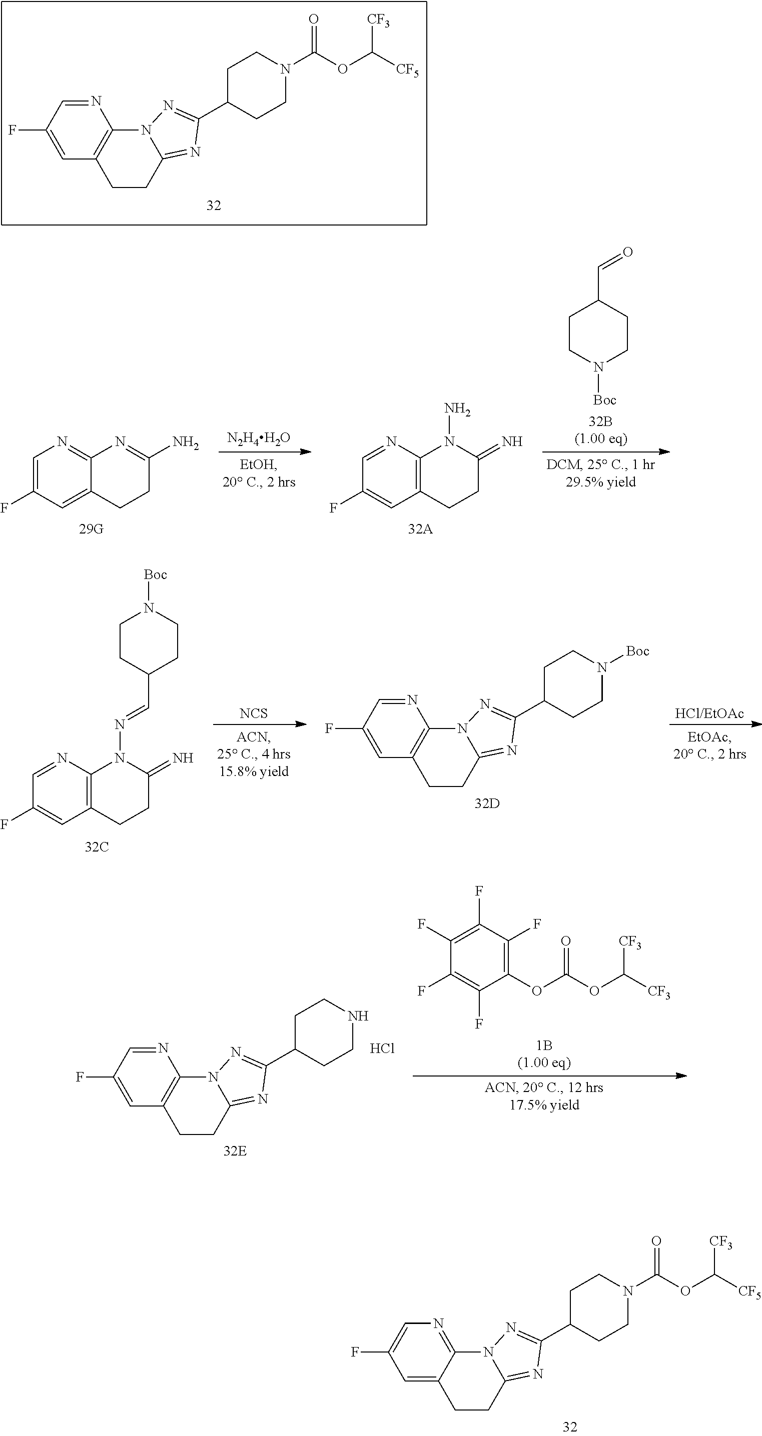

### Scheme-32: 1,1,1,3,3,3-hexafluoropropan-2-yl 4-(7-fluoro-4,5-dihydro-[1,2,4]triazolo[1,5-a][1,8]naphthyridin-2-yl)piperidine-1-carboxylate (32)

### Procedure for preparation of 6-fluoro-2-imino-3,4-dihydro-1,8-naphthyridin-1(2H)-amine (32A)

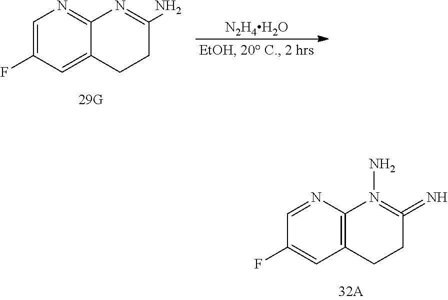

To a solution of compound 29G (620 mg, 3.75 mmol, 1.00 eq) in EtOH (10.0 mL) was added N₂H₄·H₂O (0.55 g, 10.8 mmol, 533 μL, 98.0% purity, 2.87 eq). The mixture was stirred at 20° C. for 2 hrs. The reaction mixture was concentrated under reduced pressure to remove solvent to give compound 32A (1.00 g, crude) as a white solid.

LCMS: Rt=0.457 min, MS=181.1, M+H⁺

### Procedure for preparation of tert-butyl (E)-4-(((6-fluoro-2-imino-3,4-dihydro-1,8-naphthyridin-1(2H)-yl)imino)methyl)piperidine-1-carboxylate (32C)

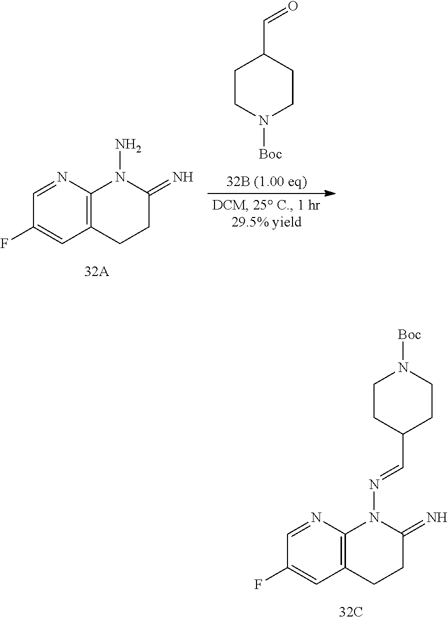 [SMILES: N=C1CCc2cc(F)cnc2N1N ; CC(C)(C)OC(=O)N1CCC(C=O)CC1 ; CC(C)(C)OC(=O)N1CCC(/C=N/N2C(=N)CCc3cc(F)cnc32)CC1]

A mixture of compound 32A (900 mg, 4.99 mmol, 1.00 eq), compound 32B (1.07 g, 4.99 mmol, 1.00 eq) in DCM (5.00 mL) was degassed and purged with N₂ for 3 times, and then the mixture was stirred at 25° C. for 1 hr under N₂ atmosphere. The reaction mixture was quenched by addition H₂O (10.0 mL) at 0° C., and then diluted with H₂O (10.0 mL) and extracted with EtOAc (10.0 mL×3). The combined organic layers were washed with brine (10.0 mL×2), dried over Na₂SO₄, filtered and concentrated under reduced pressure to give a residue. The residue was purified by column chromatography (SiO₂, Petroleum ether/Ethyl acetate=0/1, R_{f}=0.69) to give compound 32C (553 mg, 1.47 mmol, 29.5% yield) as a yellow solid.

LCMS: Rt=0.313 min, MS=376.3, M+H⁺

### Procedure for preparation of tert-butyl 4-(7-fluoro-4,5-dihydro-[1,2,4]triazolo[1,5-a][1,8]naphthyridin-2-yl)piperidine-1-carboxylate (32D)

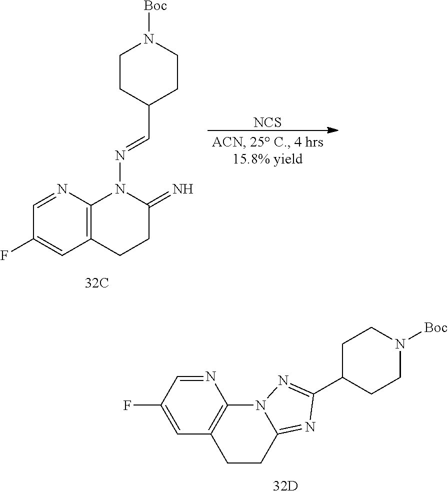 [SMILES: CC(C)(C)OC(=O)N1CCC(/C=N/N2C(=N)CCc3cc(F)cnc32)CC1 ; "NCS" ; CC(C)(C)OC(=O)N1CCC(c2nc3n(n2)-c2ncc(F)cc2CC3)CC1]

A mixture of compound 32C (510 mg, 1.36 mmol, 1.00 eq), NCS (272 mg, 2.04 mmol, 1.50 eq) in ACN (5.00 mL) was degassed and purged with N₂ for 3 times, and then the mixture was stirred at 25° C. for 4 hrs under N₂ atmosphere. The residue was purified by prep-HPLC (column: CD05-Phenomene X luna C18 150×40×10 um; mobile phase: [water (FA)-ACN]; gradient: 26%-56% B over 10 min) to give compound 32D (80.0 mg, 214 μmol, 15.8% yield) as a white solid.

LCMS: Rt=0.401 min, MS=374.2, M+H⁺

### Procedure for preparation of 1,1,1,3,3,3-hexafluoropropan-2-yl 4-(7-fluoro-4,5-dihydro-[1,2,4]triazolo[1,5-a][1,8]naphthyridin-2-yl)piperidine-1-carboxylate (32)

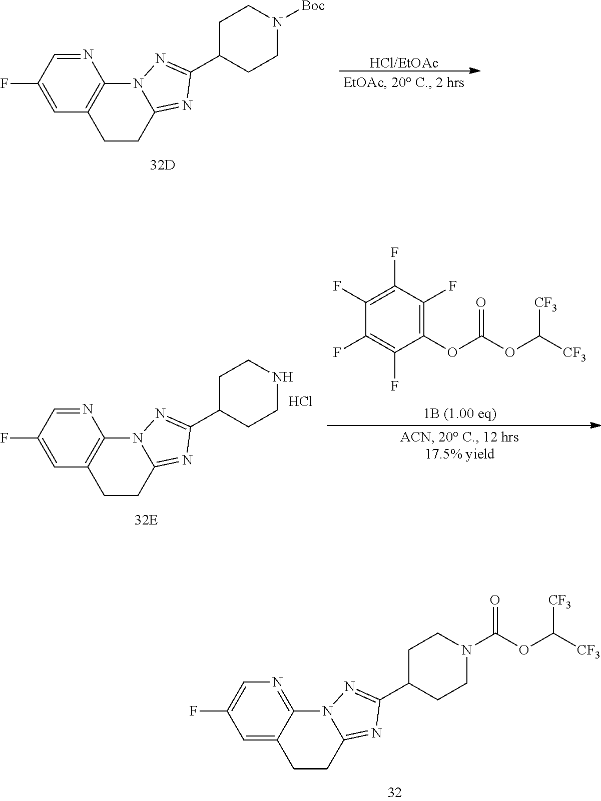 [SMILES: CC(C)(C)OC(=O)N1CCC(c2nc3n(n2)-c2ncc(F)cc2CC3)CC1 ; "HCl/EtOAc" ; Fc1cnc2c(c1)CCc1nc(C3CCNCC3)nn1-2 ; Cl ; O=C(Oc1c(F)c(F)c(F)c(F)c1F)OC(C(F)(F)F)C(F)(F)F ; O=C(OC(C(F)(F)F)C(F)(F)F)N1CCC(c2nc3n(n2)-c2ncc(F)cc2CC3)CC1]

The compound was prepared according to the same synthesis step of example 1 starting from 32D to afford compound 32 (6.00 mg, 12.8 μmol, 17.5% yield, 99.8% purity) as a white solid.

LCMS: Rt=1.778, MS=468.1, M+H⁺

¹H NMR: (400 MHz, CDCl₃)

δ 8.23 (s, 1H), 7.48-7.50 (d, J=6.0 Hz, 1H), 5.75-5.81 (m, 1H), 4.24-4.27 (m, 2H), 3.95 (t, J=9.6 Hz, 1H), 3.10-3.23 (m, 6H), 2.17 (d, J=3.2 Hz, 1H, 2H), 2.00-2.05 (m, 2H)

### Example 33: 1,1,1-trifluoro-3-hydroxypropan-2-yl 4-(7-fluoro-4,5-dihydro-[1,2,4]triazolo[1,5-a][1,8]naphthyridin-2-yl)piperidine-1-carboxylate

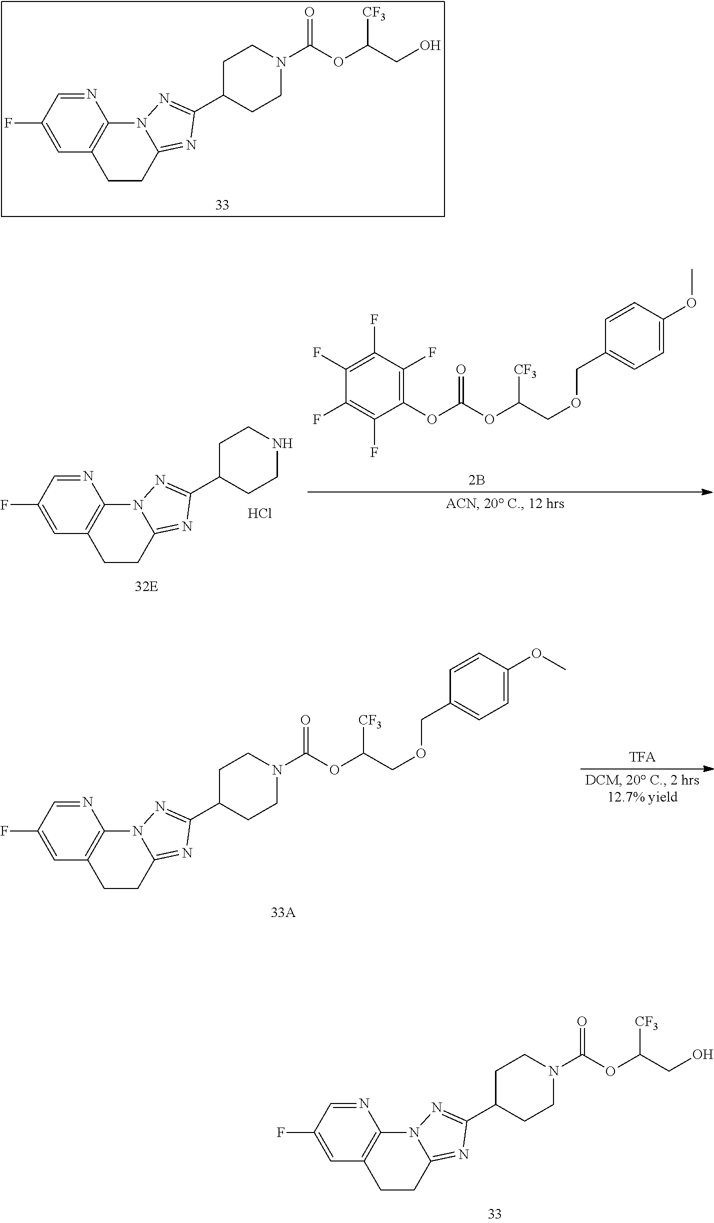

### Scheme-33: 1,1,1-trifluoro-3-hydroxypropan-2-yl 4-(7-fluoro-4,5-dihydro-[1,2,4]triazolo[1,5-a][1,8]naphthyridin-2-yl)piperidine-1-carboxylate (33)

### Procedure for preparation of 1,1,1-trifluoro-3-hydroxypropan-2-yl 4-(7-fluoro-4,5-dihydro-[1,2,4]triazolo[1,5-a][1,8]naphthyridin-2-yl)piperidine-1-carboxylate (33)

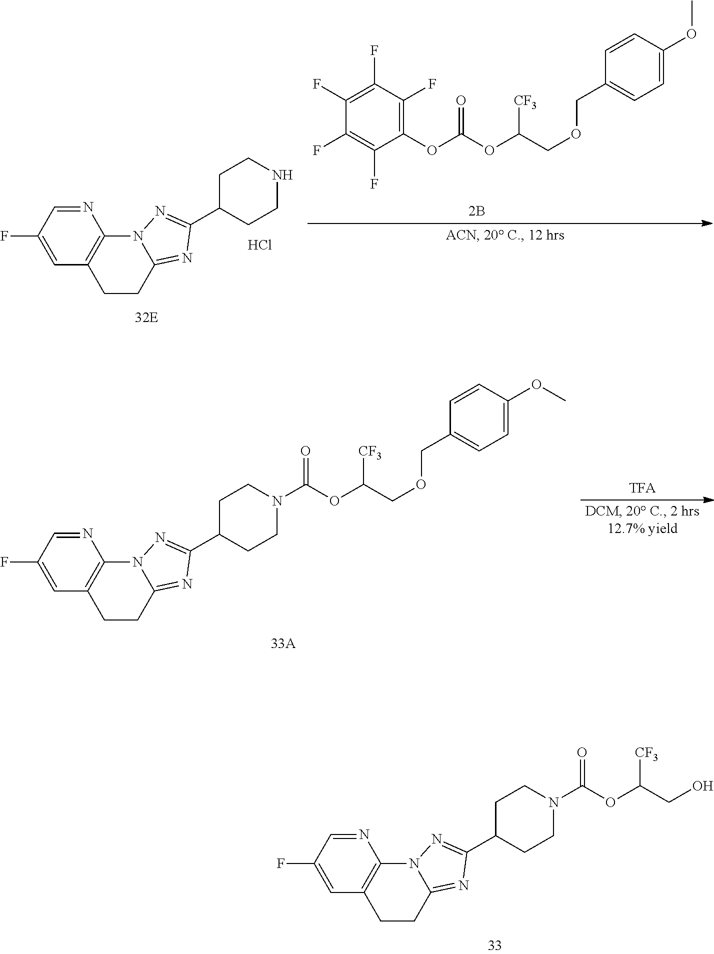

The compound was prepared according to the same synthesis step of example 2 starting from 32E to afford compound 33 (3.00 mg, 6.95 μmol, 12.7% yield, 99.4% purity) as a white solid.

LCMS: Rt=0.352 min, MS=430.1, M+H⁺

¹H NMR: (400 MHz, CDCl₃)

δ 8.25 (s, 1H), 7.48-7.51 (m, 1H), 5.27-5.29 (m, 1H), 4.24-4.28 (m, 2H), 4.00-4.03 (m, 1H), 3.90-3.93 (m, 2H), 3.22-3.23 (m, 2H), 3.17 (s, 4H), 2.04-2.18 (m, 3H), 1.66-1.99 (m, 2H)

### Example 34:1,1,1,3,3,3-hexafluoropropan-2-yl 4-(7-fluoro-3-isopropylpyrazolo[1,5-a][1,8]naphthyridin-2-yl)piperidine-1-carboxylate

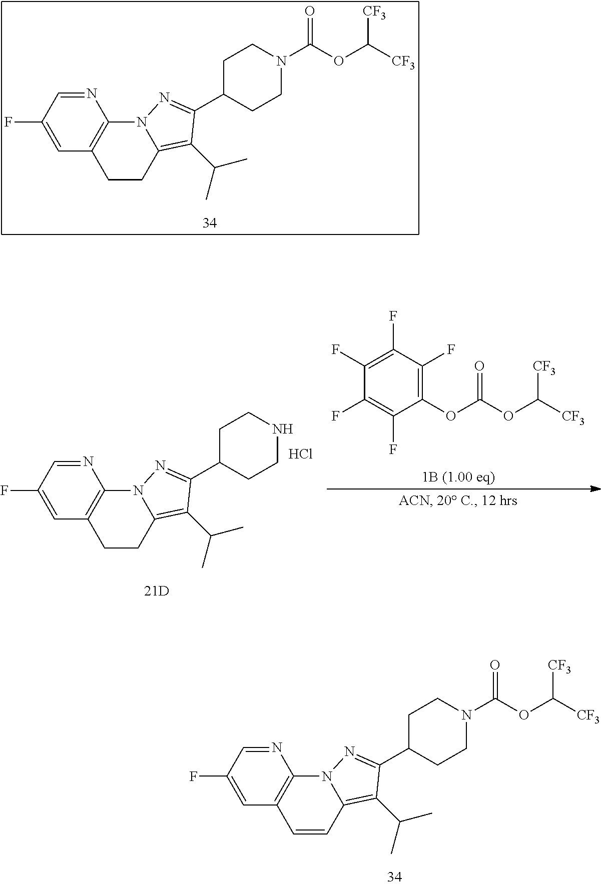 [SMILES: CC(C)c1c(C2CCN(C(=O)OC(C(F)(F)F)C(F)(F)F)CC2)nn2c1CCc1cc(F)cnc1-2 ; CC(C)c1c(C2CCNCC2)nn2c1CCc1cc(F)cnc1-2 ; Cl ; O=C(Oc1c(F)c(F)c(F)c(F)c1F)OC(C(F)(F)F)C(F)(F)F ; CC(C)c1c(C2CCN(C(=O)OC(C(F)(F)F)C(F)(F)F)CC2)nn2c1ccc1cc(F)cnc12]

### Scheme-34: 1,1,1,3,3,3-hexafluoropropan-2-yl 4-(7-fluoro-3-isopropylpyrazolo[1,5-a][1,8]naphthyridin-2-yl)piperidine-1-carboxylate (34)

### Procedure for preparation of 1,1,1,3,3,3-hexafluoropropan-2-yl 4-(7-fluoro-3-isopropylpyrazolo[1,5-a][1,8]naphthyridin-2-yl)piperidine-1-carboxylate (34)

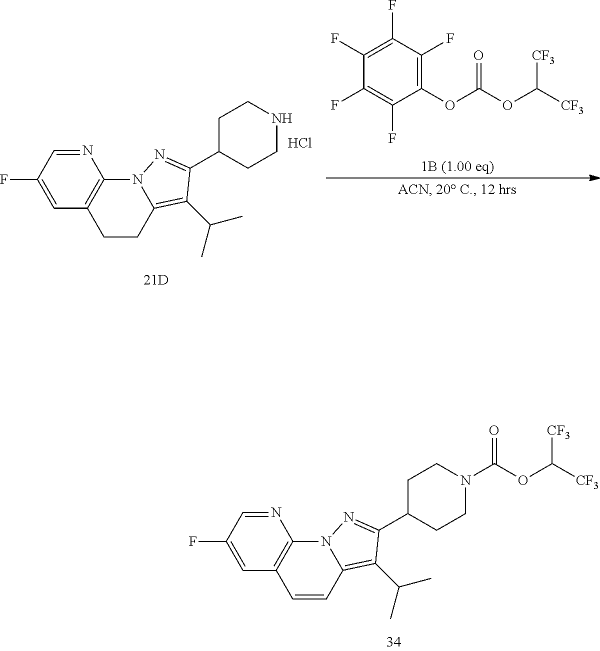 [SMILES: CC(C)c1c(C2CCNCC2)nn2c1CCc1cc(F)cnc1-2 ; Cl ; O=C(Oc1c(F)c(F)c(F)c(F)c1F)OC(C(F)(F)F)C(F)(F)F ; CC(C)c1c(C2CCN(C(=O)OC(C(F)(F)F)C(F)(F)F)CC2)nn2c1ccc1cc(F)cnc12]

A mixture of compound 21D (230 mg, 732 μmol, 1.00 eq), compound 1B (277 mg, 732 μmol, 1.00 eq) in ACN (1.00 mL) was degassed and purged with N₂ for 3 times, and then the mixture was stirred at 20° C. for 2 hrs under N₂ atmosphere. The reaction mixture was concentrated under reduced pressure to remove solvent. The residue was purified by prep-HPLC (column: Phenomenex luna C18 150×25 mm×10 um; mobile phase: [water (HCl)-ACN]; gradient: 56%-86% B over 10 min) to give compound 34 (16.0 mg, 31.1 μmol, 4.3% yield, 98.3% purity) as a white solid.

LCMS: Rt=0.540 min, m/z: 507.2, M+H⁺,

¹H NMR: (400 MHz, CDCl₃)

δ 8.65 (s, 1H), 7.60-7.77 (d, J=6.8, 1H), 7.58-7.60 (d, J=8.0, 1H), 7.21-7.23 (d, J=8.0, 1H), 5.76-5.83 (m, 1H), 4.32-4.40 (m, 2H), 3.00-3.26 (m, 4H), 2.22-2.26 (m, 2H), 1.98-2.01 (m, 2H), 1.30-1.43 (d, J=5.2, 6H).

### Example 35:1,1,1,3,3,3-hexafluoropropan-2-yl 4-(7-fluoro-4,5-dihydropyrazolo[1,5-a]quinolin-2-yl)piperazine-1-carboxylate

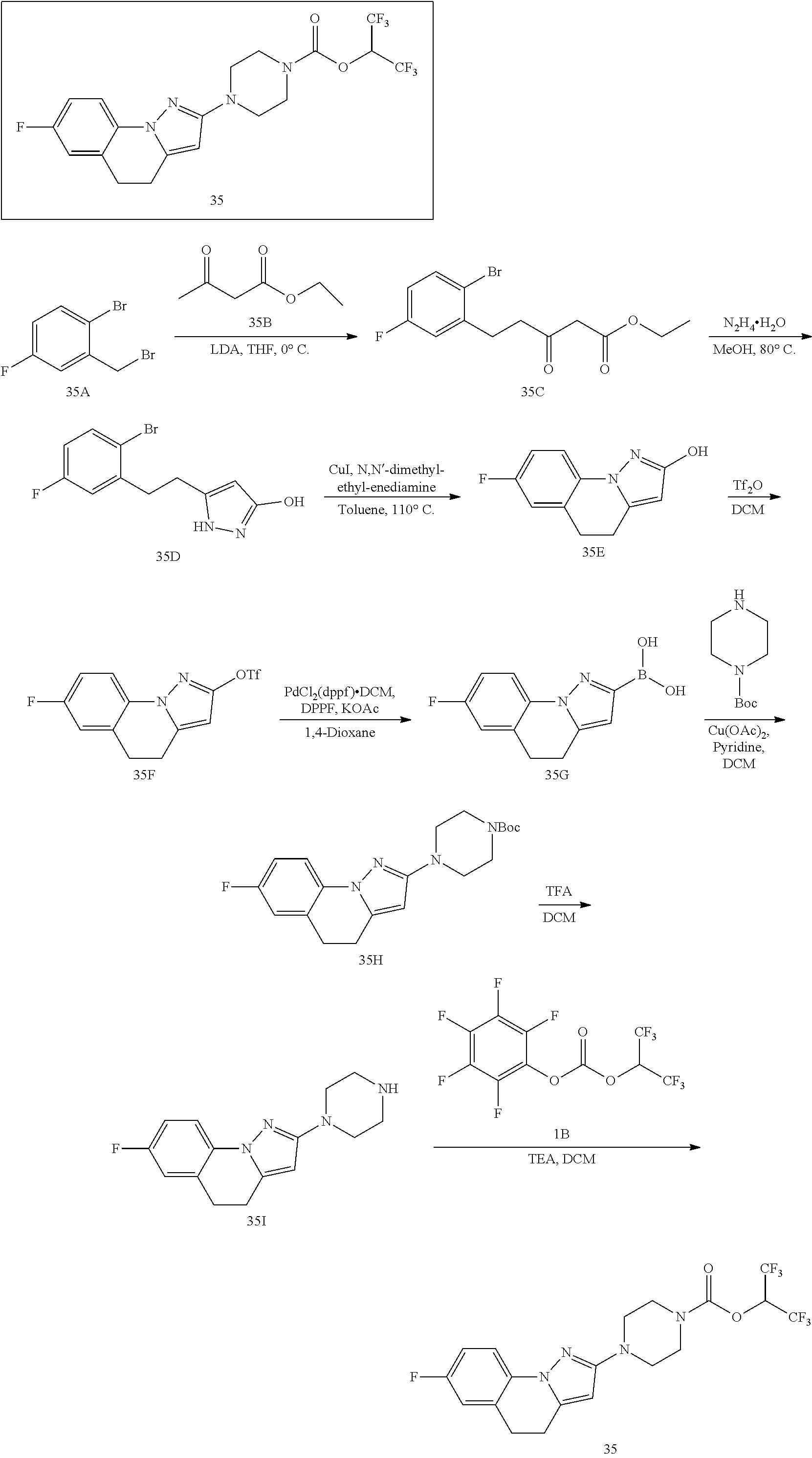

### Scheme-35: 1,1,1,3,3,3-hexafluoropropan-2-yl 4-(7-fluoro-4,5-dihydropyrazolo[1,5-a]quinolin-2-yl)piperazine-1-carboxylate (36)

### Procedure for preparation of ethyl 5-(2-bromo-5-fluorophenyl)-3-oxopentanoate (35C)

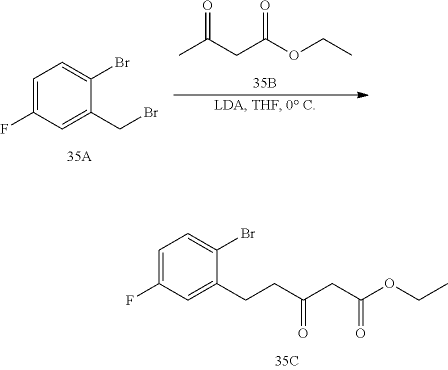

To a solution compound 35B (4.86 g, 37.34 mmol, 1.00 eq) in THF (100 mL) was added LDA (40 mL, 74.7 mmol, 2.00 eq) at 0° C. The suspension was degassed and purged with N₂ for 3 times. The mixture was stirred at 0° C. for 1 hr. Then a solution compound 35A (10.0 g, 37.34 mmol, 1.00 eq) in THF (20 mL) was added to the mixture. The mixture was stirred at 0° C. for another 1 hr. The reaction was quenched by addition of 100 mL of saturated ammonium chloride, then extracted with EtOAc. The combined organic layers were washed with brine, dried over Na₂SO₄, filtered and concentrated under reduced pressure to give a residue. The residue was purified by column chromatography (SiO₂, Petroleum ether/Ethyl acetate=20/1) to give compound 35C (8.5 g, 26.62 mmol, 71.3% yield, 98.0% purity) as a brown oil.

LCMS: Rt=0.385 min, MS=317.2, M+H⁺

### Procedure for preparation of 5-(2-bromo-5-fluorophenethyl)-1H-pyrazol-3-ol (35D)

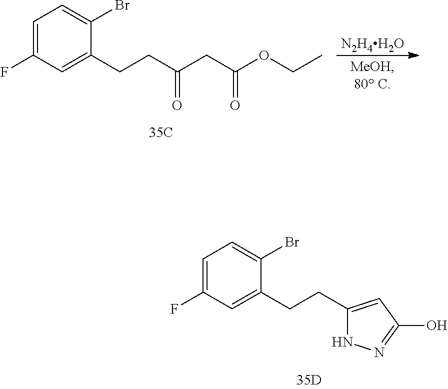

To a solution of 35C (8.50 g, 26.62 mmol, 1.00 eq) in MeOH (120.0 mL) was added NH₂NH₂·H₂O (3.4 g, 53.60 mmol, 2.00 eq). The mixture was stirred at 80° C. for 8 hrs. The reaction mixture was quenched by addition of H₂O at 20° C. Then the mixture was filtered and concentrated under reduced pressure to give a residue. The residue was dried under reduced pressure to obtained compound 35D (7.26 g, 25.46 mmol, 95.0% yield, 99.5% purity) as a light-yellow solid.

LCMS: Rt=0.265 min, MS=285.1, M+H⁺

### Procedure for preparation of 7-fluoro-4,5-dihydropyrazolo[1,5-a]quinolin-2-ol (35E)

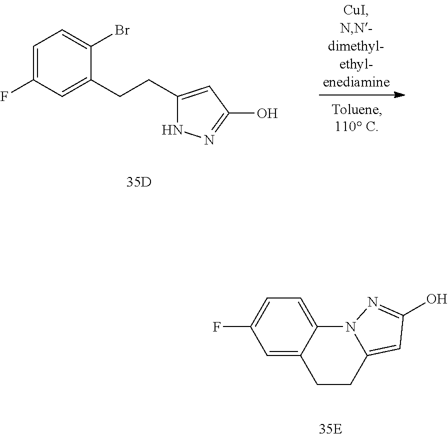 [SMILES: Oc1cc(CCc2cc(F)ccc2Br)[nH]n1 ; Oc1cc2n(n1)-c1ccc(F)cc1CC2]

To a solution of 35D (7.26 g, 25.46 mmol, 1.00 eq) in toluene (150.0 mL) was added CuI (243.8 mg, 1.27 mmol, 0.05 eq), K₂CO₃ (7.07 g, 50.92 mmol, 2.00 eq) and N,N′-dimethylethane-1,2-diamine (564.2 mg, 6.37 mmol, 0.25 eq). The mixture was stirred at 110° C. for 16 hrs. The reaction mixture was filtered and concentrated under reduced pressure to give a residue. The residue was purified by column chromatography (SiO₂, Petroleum ether/Ethyl acetate=5/1 to 1/1, R_{f}=0.42) to obtained compound 35E (2.65 g, 12.98 mmol, 50.8% yield, 100% purity) as a brown solid.

LCMS: Rt=0.362 min, MS=205.2, M+H⁺

### Procedure for preparation of 7-fluoro-4,5-dihydropyrazolo[1,5-a]quinolin-2-yl trifluoromethanesulfonate (35F)

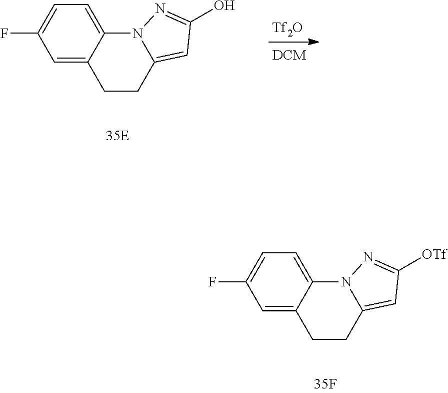 [SMILES: "Tf2O" ; Oc1cc2n(n1)-c1ccc(F)cc1CC2 ; O=S(=O)(Oc1cc2n(n1)-c1ccc(F)cc1CC2)C(F)(F)F]

To a solution compound 35E (2.65 g, 12.98 mmol, 1.00 eq) and TEA (1.97 g, 19.47 mmol, 1.50 eq) in DCM (100 mL) was added trifluoromethanesulfonic anhydride (4.39 g, 15.57 mmol, 1.20 eq) at 0° C. The mixture was stirred at 0° C. for another 2 hr. The reaction was quenched by addition of 100 mL of water, then extracted with DCM. The combined organic layers were washed with brine, dried over Na₂SO₄, filtered and concentrated under reduced pressure to give a compound 35F (4.0 g, 11.89 mmol, 91.8% yield, 97.8% purity) as a brown solid.

LCMS: Rt=0.438 min, MS=337.2, M+H⁺

### Procedure for preparation of (7-fluoro-4,5-dihydropyrazolo[1,5-a]quinolin-2-yl)boronic acid (35G)

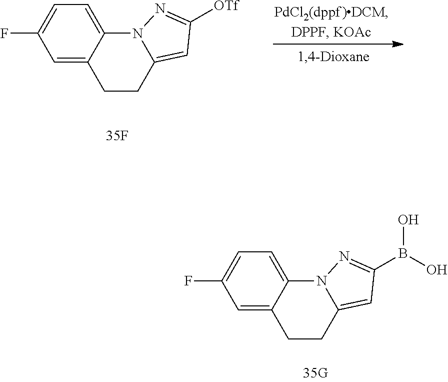 [SMILES: O=S(=O)(Oc1cc2n(n1)-c1ccc(F)cc1CC2)C(F)(F)F ; "PdCl2(dppf)·DCM,DPPF, KOAc" ; OB(O)c1cc2n(n1)-c1ccc(F)cc1CC2]

To a solution of compound 35F (4.0 g, 11.89 mmol, 1.00 eq), Bis (pinacolato)diboron (3.63 g, 14.27 mmol, 1.20 eq) KOAc (3.51 g, 35.69 mmol, 3.00 eq) in dioxane (120 mL) and was added PdCl₂(dppf)·DCM (485.7 mg, 0.59 mmol, 0.05 eq) and dppf (329.7 mg, 0.59 mmol, 0.05 eq). The suspension was degassed and purged with N₂ for 3 times. The mixture was stirred at 90° C. for 12 hrs. The reaction mixture was quenched by addition H₂O 3.00 mL and extracted with EtOAc. The combined organic layers were washed brine, and dried over Na₂SO₄, filtered and concentrated under reduced pressure to give the residue. The residue was purified by column chromatography (SiO₂, Petroleum ether/Ethyl acetate=10/1 to 5/1, R_{f}=0.38) to obtained compound 35G (2.44 g, 10.52 mmol, 88.2% yield, 96.3% purity) as a yellow solid.

LCMS: Rt=0.238 min, MS=233.2, M+H⁺

### Procedure for preparation of tert-butyl 4-(7-fluoro-4,5-dihydropyrazolo[1,5-a]quinolin-2-yl)piperazine-1-carboxylate (35H)

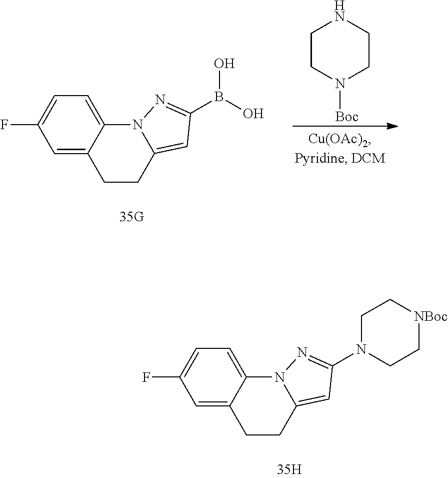 [SMILES: OB(O)c1cc2n(n1)-c1ccc(F)cc1CC2 ; CC(C)(C)OC(=O)N1CCNCC1 ; CC(C)(C)OC(=O)N1CCN(c2cc3n(n2)-c2ccc(F)cc2CC3)CC1]

To a solution of compound 35G (2.44 g, 10.52 mmol, 1.00 eq), 1-Boc-piperazine (2.35 g, 1.27 mmol, 1.20 eq), in DCM (100 mL) and was added Cu(OAc)₂ (3.82 g, 21.03 mmol, 2.00 eq) and pyridine (2.49 g, 31.55 mmol, 3.00 eq). The suspension was degassed and purged with N₂ for 3 times. The mixture was stirred at 25° C. for 16 hrs. The reaction mixture was quenched by addition H₂O and extracted with DCM. The combined organic layers were washed brine, and dried over Na₂SO₄, filtered and concentrated under reduced pressure to give the residue. The residue was purified by column chromatography (SiO₂, Petroleum ether/Ethyl acetate=8/1 to 5/1, R_{f}=0.20) to obtained compound 35H (1.20 g, 3.22 mmol, 30.8% yield, 96.3% purity) as a white solid.

LCMS: Rt=0.356 min, MS=373.39, M+H⁺

### Procedure for preparation of 1,1,1,3,3,3-hexafluoropropan-2-yl 4-(7-fluoro-4,5-dihydropyrazolo[1,5-a]quinolin-2-yl)piperazine-1-carboxylate (35)

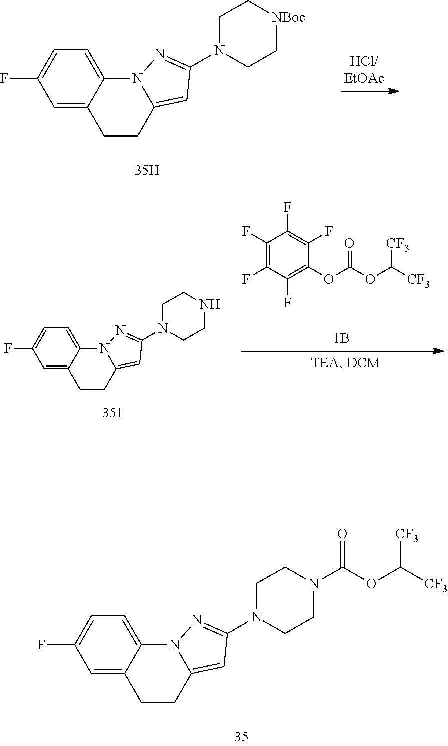 [SMILES: CC(C)(C)OC(=O)N1CCN(c2cc3n(n2)-c2ccc(F)cc2CC3)CC1 ; Fc1ccc2c(c1)CCc1cc(N3CCNCC3)nn1-2 ; O=C(Oc1c(F)c(F)c(F)c(F)c1F)OC(C(F)(F)F)C(F)(F)F ; O=C(OC(C(F)(F)F)C(F)(F)F)N1CCN(c2cc3n(n2)-c2ccc(F)cc2CC3)CC1]

The compound was prepared according to the same synthesis step of example 1 starting from 35H to afford compound 35 (36.0 mg, 0.08 mmol, 57.5% yield, 98.1% purity) as a light-yellow oil.

LCMS: Rt=0.396 min, MS=467.32, M+H⁺

### Example 36: 1,1,1-trifluoro-3-hydroxypropan-2-yl 4-(7-fluoro-4,5-dihydropyrazolo[1,5-a]quinolin-2-yl)piperazine-1-carboxylate

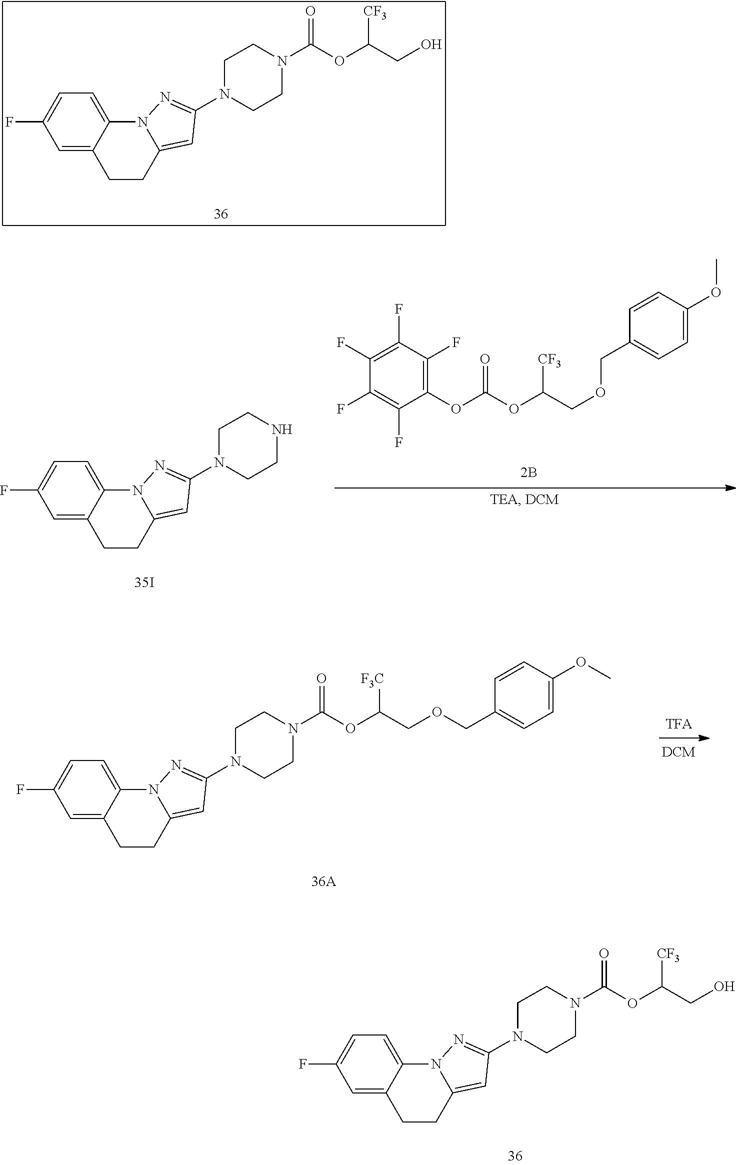 [SMILES: O=C(OC(CO)C(F)(F)F)N1CCN(c2cc3n(n2)-c2ccc(F)cc2CC3)CC1 ; Fc1ccc2c(c1)CCc1cc(N3CCNCC3)nn1-2 ; COc1ccc(COCC(OC(=O)Oc2c(F)c(F)c(F)c(F)c2F)C(F)(F)F)cc1 ; COc1ccc(COCC(OC(=O)N2CCN(c3cc4n(n3)-c3ccc(F)cc3CC4)CC2)C(F)(F)F)cc1 ; O=C(OC(CO)C(F)(F)F)N1CCN(c2cc3n(n2)-c2ccc(F)cc2CC3)CC1]

### Scheme-36: 1,1,1-trifluoro-3-hydroxypropan-2-yl 4-(7-fluoro-4,5-dihydropyrazolo[1,5-a]quinolin-2-yl)piperazine-1-carboxylate (36)

### Procedure for preparation of 1,1,1-trifluoro-3-hydroxypropan-2-yl 4-(7-fluoro-4,5-dihydropyrazolo[1,5-a]quinolin-2-yl)piperazine-1-carboxylate (36)

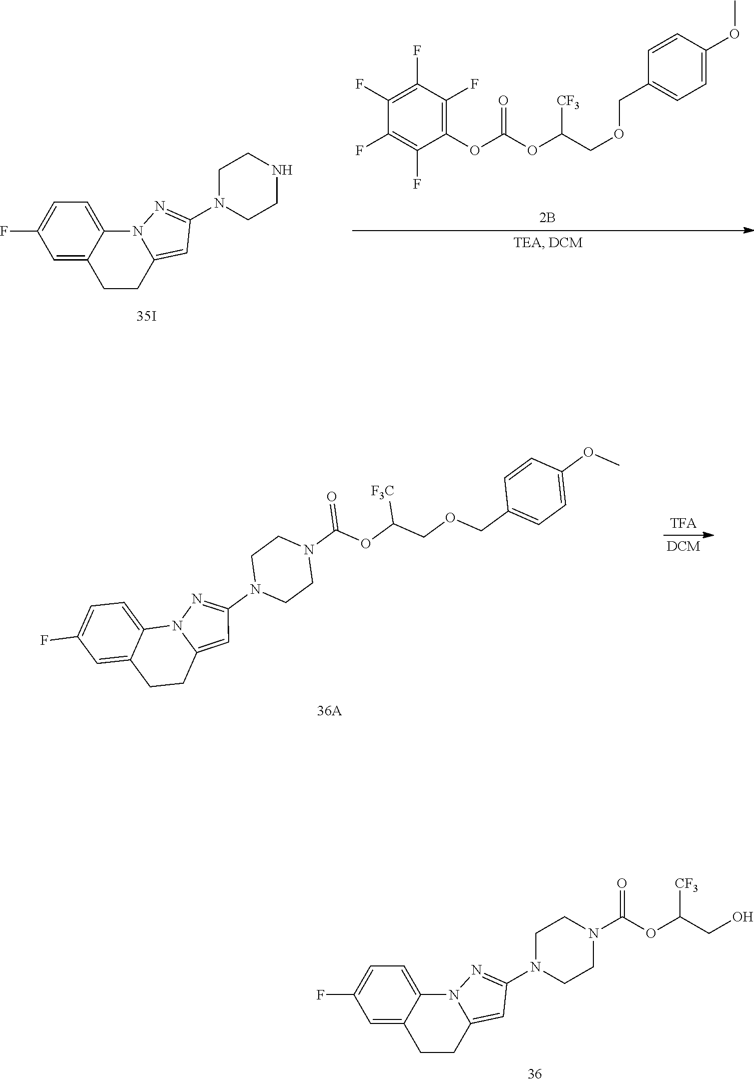 [SMILES: Fc1ccc2c(c1)CCc1cc(N3CCNCC3)nn1-2 ; COc1ccc(COCC(OC(=O)Oc2c(F)c(F)c(F)c(F)c2F)C(F)(F)F)cc1 ; COc1ccc(COCC(OC(=O)N2CCN(c3cc4n(n3)-c3ccc(F)cc3CC4)CC2)C(F)(F)F)cc1 ; O=C(OC(CO)C(F)(F)F)N1CCN(c2cc3n(n2)-c2ccc(F)cc2CC3)CC1]

The compound was prepared according to the same synthesis step of example 2 starting from 351 to afford compound 36 (15.2 mg, 57.5% yield, 97.6% purity) as a light-yellow oil.

LCMS: Rt=0.274 min, MS=429.45, M+H⁺

¹H NMR: (400 MHz, DMSO_d₆)

δ 7.52-7.58 (m, 1H), 7.19 (m, 1H), 7.12 (m, 1H), 5.88 (s, 1H), 5.23-5.29 (m, 1H), 3.74-3.79 (m, 1H), 3.66-3.71 (m, 1H), 3.57-3.62 (m, 4H), 3.16-3.24 (m, 4H), 2.84-2.94 (m, 4H).

### Example 37: 2,5-dioxopyrrolidin-1-yl 4-(7-fluoro-4,5-dihydropyrazolo[1,5-a]quinolin-2-yl)piperazine-1-carboxylate

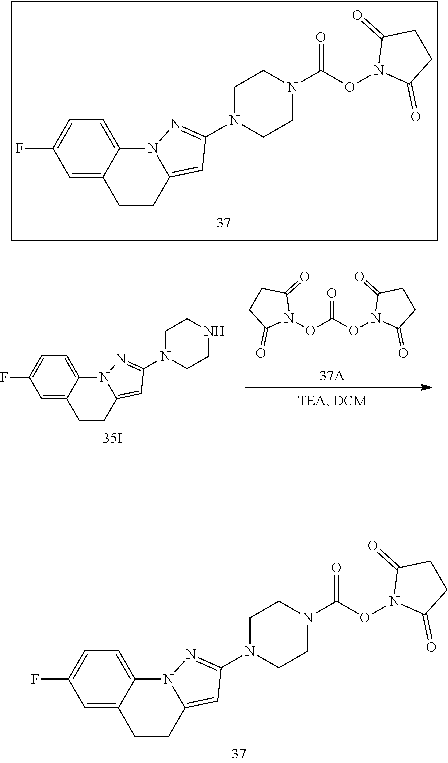

### Scheme-37: 2,5-dioxopyrrolidin-1-yl 4-(7-fluoro-4,5-dihydropyrazolo[1,5-a]quinolin-2-yl)piperazine-1-carboxylate (37)

### Procedure for preparation of 2,5-dioxopyrrolidin-1-yl 4-(7-fluoro-4,5-dihydropyrazolo[1,5-a]quinolin-2-yl)piperazine-1-carboxylate (37)

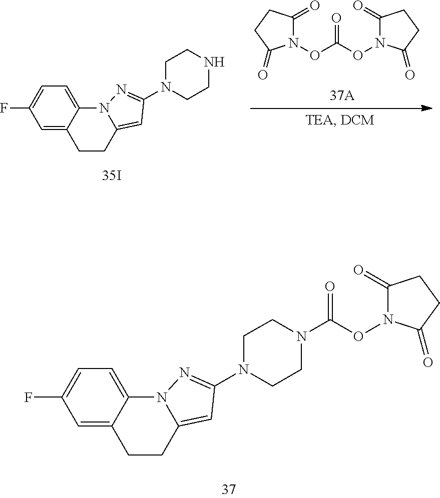 [SMILES: Fc1ccc2c(c1)CCc1cc(N3CCNCC3)nn1-2 ; O=C(ON1C(=O)CCC1=O)ON1C(=O)CCC1=O ; O=C(ON1C(=O)CCC1=O)N1CCN(c2cc3n(n2)-c2ccc(F)cc2CC3)CC1]

To a solution of 351 (30 mg, 0.11 mmol, 1.00 eq) and TEA (33.4 mg, 0.33 mmol, 3.00 eq) in DCM (2.30 mL) was added 37A (42.3 mg, 0.17 mmol, 1.50 eq). The mixture was stirred at 25° C. for 16 hrs. The reaction mixture was quenched by addition H₂O and extracted with DCM. The combined organic layers were washed brine, and dried over Na₂SO₄, filtered and concentrated under reduced pressure to give the residue. The residue was purified by column chromatography (SiO₂, Petroleum ether/Ethyl acetate=2/1 to 1/2) to obtained compound 37 (21.5 mg, 51.7% yield, 98.6% purity) as a white solid.

LCMS: Rt=0.196 min, MS=414.33, M+H⁺

¹H NMR: (400 MHz, DMSO_d₆)

δ 7.56 (dd, J=8.8, 5.1 Hz, 1H), 7.20 (dd, J=9.1, 2.6 Hz, 1H), 7.12 (td, J=8.7, 2.7 Hz, 1H), 5.90 (s, 1H), 3.69 (s, 2H), 3.55 (s, 2H), 3.28 (s, 4H), 2.95-2.86 (m, 4H), 2.80 (s, 4H).

### Example 38: 1H-1,2,4-triazol-1-yl 4-(7-fluoro-4,5-dihydropyrazolo[1,5-a]quinolin-2-yl)piperazine-1-carboxylate

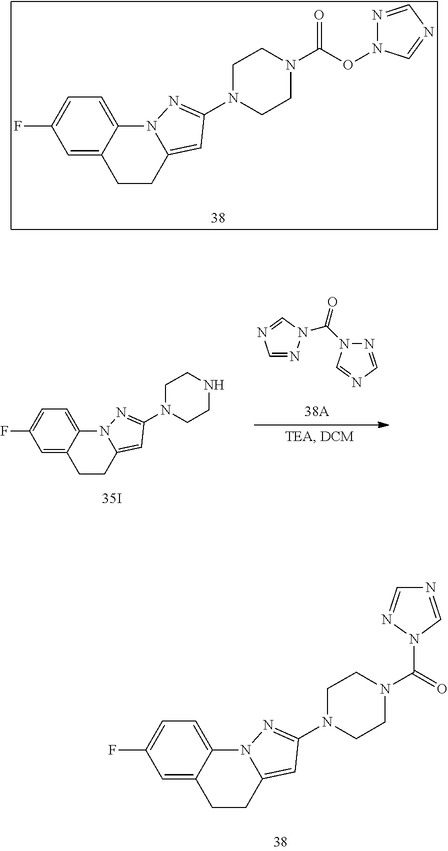 [SMILES: O=C(On1cncn1)N1CCN(c2cc3n(n2)-c2ccc(F)cc2CC3)CC1 ; Fc1ccc2c(c1)CCc1cc(N3CCNCC3)nn1-2 ; O=C(n1cncn1)n1cncn1 ; O=C(N1CCN(c2cc3n(n2)-c2ccc(F)cc2CC3)CC1)n1cncn1]

### Scheme-38: 1H-1,2,4-triazol-1-yl 4-(7-fluoro-4,5-dihydropyrazolo[1,5-a]quinolin-2-yl)piperazine-1-carboxylate (38)

### Procedure for preparation of 1H-1,2,4-triazol-1-yl 4-(7-fluoro-4,5-dihydropyrazolo[1,5-a]quinolin-2-yl)piperazine-1-carboxylate (38)

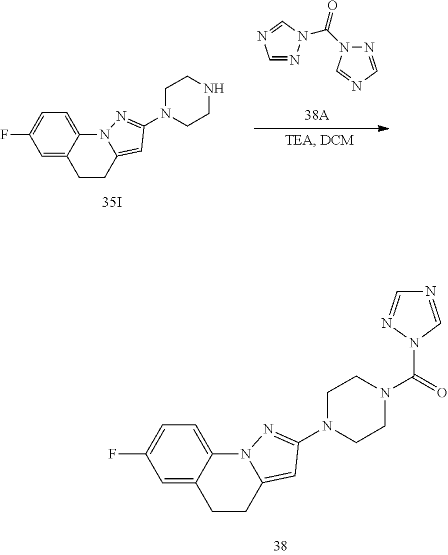 [SMILES: Fc1ccc2c(c1)CCc1cc(N3CCNCC3)nn1-2 ; O=C(n1cncn1)n1cncn1 ; O=C(N1CCN(c2cc3n(n2)-c2ccc(F)cc2CC3)CC1)n1cncn1]

To a solution of 351 (30 mg, 0.11 mmol, 1.00 eq) and TEA (33.4 mg, 0.33 mmol, 3.00 eq) in DCM (2.30 mL) was added 38A (27.1 mg, 0.17 mmol, 1.50 eq). The mixture was stirred at 25° C. for 16 hrs. The reaction mixture was quenched by addition H₂O and extracted with DCM. The combined organic layers were washed brine, and dried over Na₂SO₄, filtered and concentrated under reduced pressure to give the residue. The residue was purified by column chromatography (SiO₂, Petroleum ether/Ethyl acetate=2/1 to 1/2) to obtained compound 38 (24.7 mg, 60.8% yield, 97.5% purity) as a white solid.

LCMS: Rt=0.284 min, MS=368.32, M+H⁺

¹H NMR: (400 MHz, DMSO_d₆)

δ 9.10 (s, 1H), 8.29 (s, 1H), 7.55 (dd, J=8.7, 5.1 Hz, 1H), 7.19 (dd, J=9.0, 2.3 Hz, 1H), 7.12 (td, J=8.7, 2.5 Hz, 1H), 5.90 (s, 1H), 3.80 (s, 4H), 3.33 (s, 4H), 3.03-2.82 (m, 4H).

### Example 39: 1,1,1-trifluoro-3-hydroxypropan-2-yl 4-(3-cyano-7-fluoro-4,5-dihydropyrazolo[1,5-a]quinolin-2-yl)piperidine-1-carboxylate

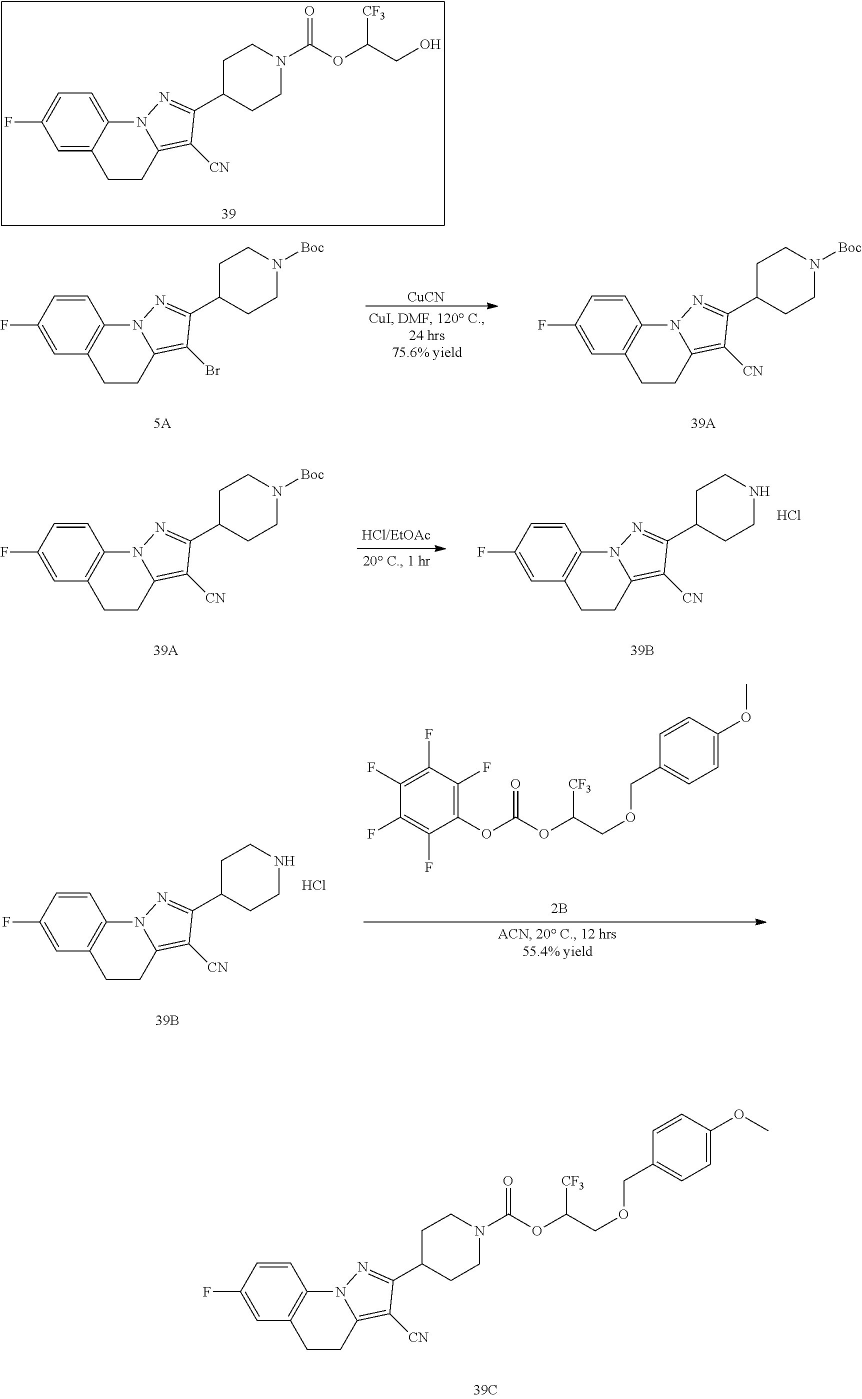

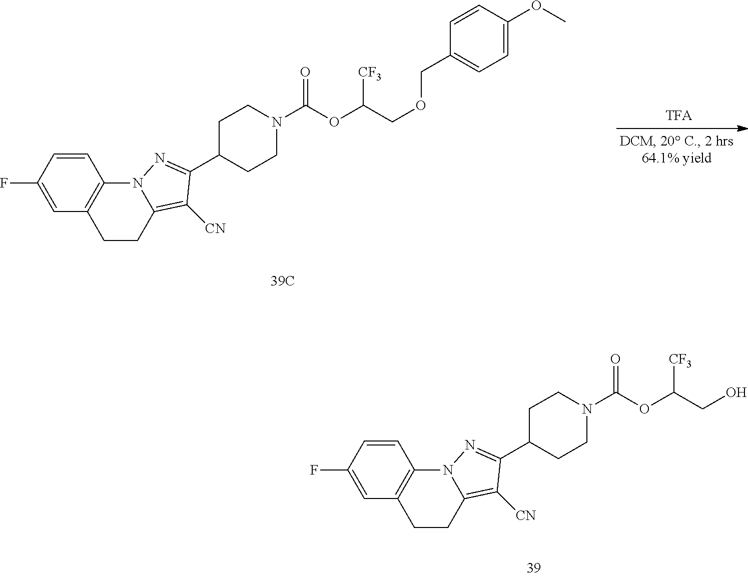 [SMILES: COc1ccc(COCC(OC(=O)N2CCC(c3nn4c(c3C#N)CCc3cc(F)ccc3-4)CC2)C(F)(F)F)cc1 ; N#Cc1c(C2CCN(C(=O)OC(CO)C(F)(F)F)CC2)nn2c1CCc1cc(F)ccc1-2]

### Scheme-39: 1,1,1-trifluoro-3-hydroxypropan-2-yl 4-(3-cyano-7-fluoro-4,5-dihydropyrazolo[1,5-a]quinolin-2-yl)piperidine-1-carboxylate (39)

### Procedure for preparation of tert-butyl 4-(3-cyano-7-fluoro-4,5-dihydropyrazolo[1,5-a]quinolin-2-yl)piperidine-1-carboxylate (39A)

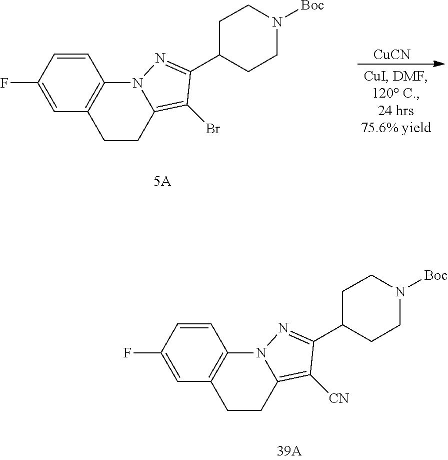 [SMILES: CC(C)(C)OC(=O)N1CCC(c2nn3c(c2Br)CCc2cc(F)ccc2-3)CC1 ; "CuCN" ; CC(C)(C)OC(=O)N1CCC(c2nn3c(c2C#N)CCc2cc(F)ccc2-3)CC1]

A mixture of compound 5A (200 mg, 444 μmol, 1.00 eq), CuI (8.46 mg, 44.4 μmol, 0.10 eq), CuCN (47.7 mg, 532 μmol, 116 μL, 1.20 eq) in DMF (10.0 mL) was degassed and purged with N₂ for 3 times, and then the mixture was stirred at 120° C. for 24 hrs under N₂ atmosphere. The reaction mixture was concentrated under reduced pressure to remove solvent. The residue was purified by flash silica gel chromatography (ISCO®; 4 g SepaFlash® Silica Flash Column, Eluent of 0-17% Ethyl acetate/Petroleum ether gradient @ 80 mL/min) to give compound 39A (135 mg, 335 μmol, 75.6% yield, 98.6% purity) as a white solid.

LCMS: Rt=0.469 min, MS=419.2, M+Na⁺.

### Procedure for preparation of 1,1,1-trifluoro-3-hydroxypropan-2-yl 4-(3-cyano-7-fluoro-4,5-dihydropyrazolo[1,5-a]quinolin-2-yl)piperidine-1-carboxylate (39)

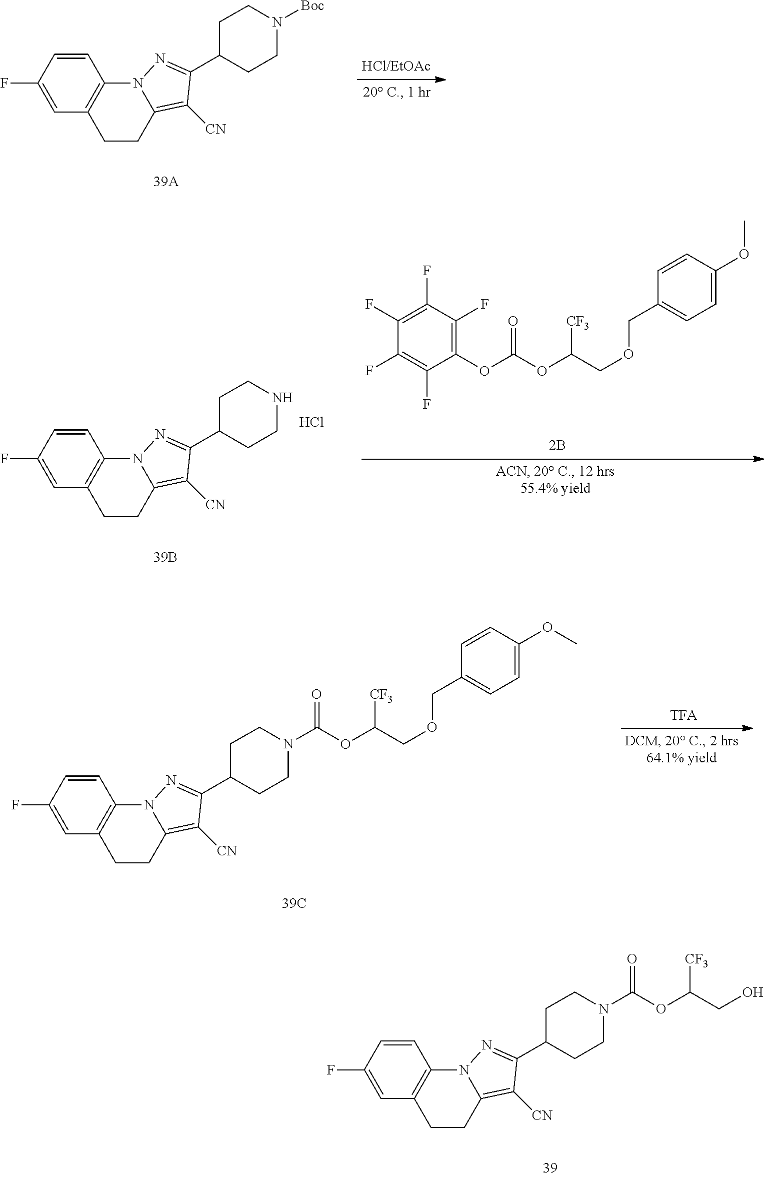 [SMILES: CC(C)(C)OC(=O)N1CCC(c2nn3c(c2C#N)CCc2cc(F)ccc2-3)CC1 ; N#Cc1c(C2CCNCC2)nn2c1CCc1cc(F)ccc1-2 ; Cl ; COc1ccc(COCC(OC(=O)Oc2c(F)c(F)c(F)c(F)c2F)C(F)(F)F)cc1 ; COc1ccc(COCC(OC(=O)N2CCC(c3nn4c(c3C#N)CCc3cc(F)ccc3-4)CC2)C(F)(F)F)cc1 ; N#Cc1c(C2CCN(C(=O)OC(CO)C(F)(F)F)CC2)nn2c1CCc1cc(F)ccc1-2]

The compound was prepared according to the same synthesis step of example 2 starting from 5A to afford compound 39 (50.0 mg, 109 μmol, 64.1% yield, 98.7% purity) as a white solid.

LCMS: Rt=0.461 min, MS=453.1 M+H⁺.

¹H NMR: (400 MHz, CDCl₃): δ 8.03-8.07 (m, 1H), 7.21-7.23 (m, 2H), 5.47-5.52 (m, 1H), 4.47-4.51 (m, 2H), 4.26-4.27 (m, 1H), 4.13-4.14 (m, 1H), 3.21-3.36 (m, 8H), 2.26-2.27 (m, 2H), 2.16-2.25 (m, 2H).

### Example 40: 1,1,1-trifluoro-3-hydroxypropan-2-yl 4-(3-cyclopropyl-7-fluoro-4,5-dihydropyrazolo[1,5-a]quinolin-2-yl)piperidine-1-carboxylate

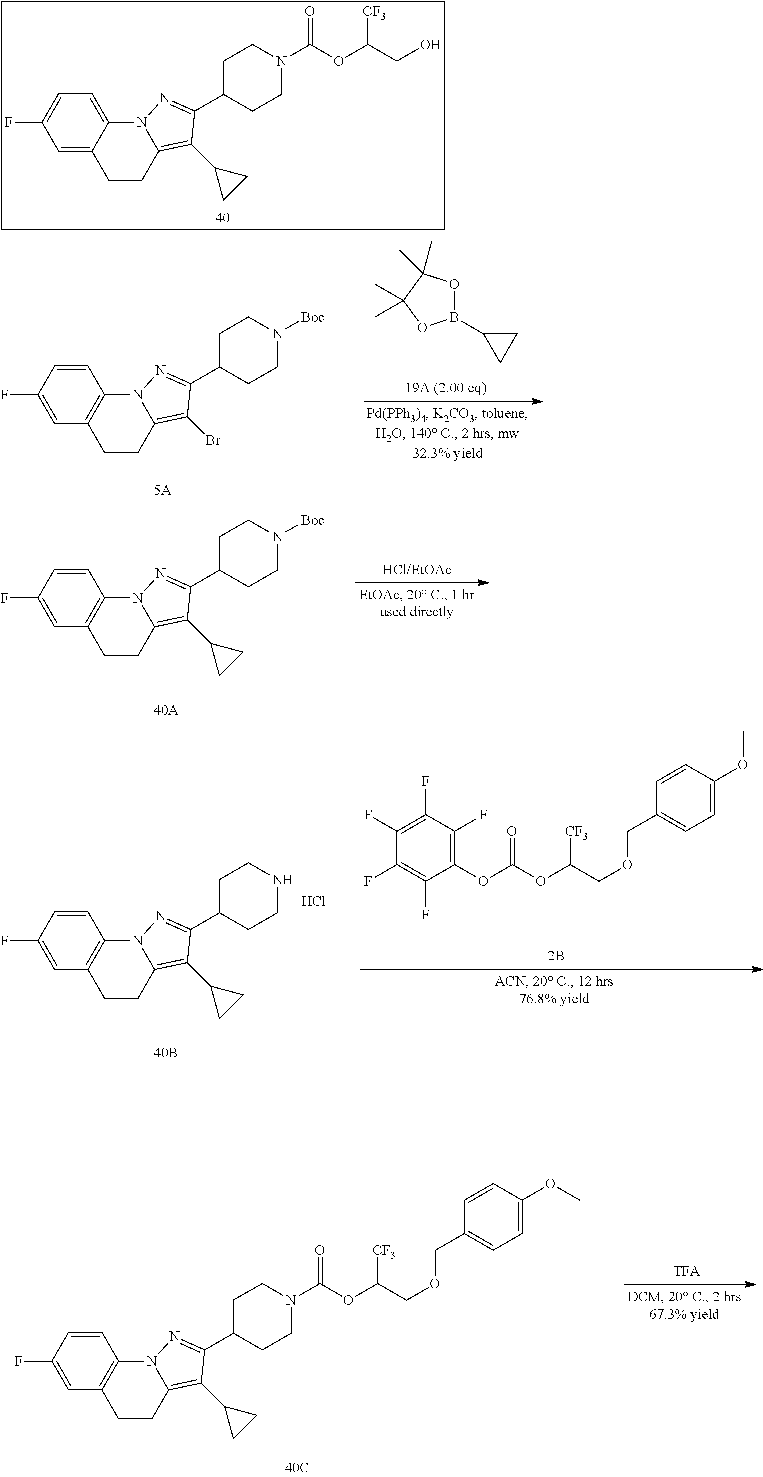

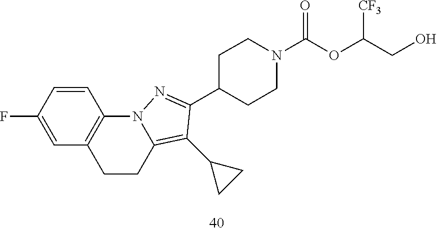 [SMILES: O=C(OC(CO)C(F)(F)F)N1CCC(c2nn3c(c2C2CC2)CCc2cc(F)ccc2-3)CC1]

### Scheme-40: 1,1,1-trifluoro-3-hydroxypropan-2-yl 4-(3-cyclopropyl-7-fluoro-4,5-dihydropyrazolo[1,5-a]quinolin-2-yl)piperidine-1-carboxylate (40)

### Procedure for preparation of tert-butyl 4-(3-cyclopropyl-7-fluoro-4,5-dihydropyrazolo[1,5-a]quinolin-2-yl)piperidine-1-carboxylate (40A)

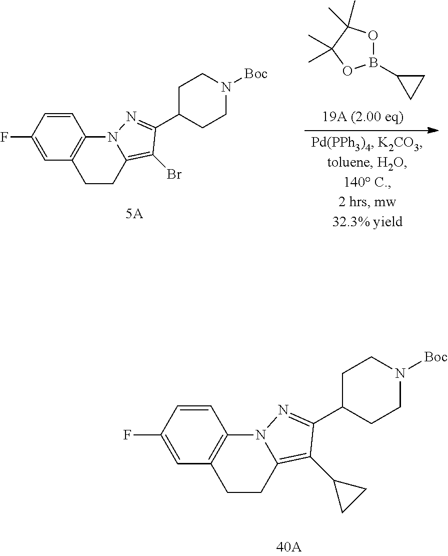 [SMILES: CC(C)(C)OC(=O)N1CCC(c2nn3c(c2Br)CCc2cc(F)ccc2-3)CC1 ; CC1(C)OB(C2CC2)OC1(C)C ; CC(C)(C)OC(=O)N1CCC(c2nn3c(c2C2CC2)CCc2cc(F)ccc2-3)CC1]

A mixture of compound 5A (300 mg, 666 umol, 1.00 eq), compound 19A (224 mg, 1.33 mmol, 2.00 eq), K₃CO₃ (424 mg, 2.00 mmol, 3.00 eq), Pd(PPh₃)₄ (56.4 mg, 66.6 umol, 0.10 eq) dioxane (4.00 mL) and H₂O (1.00 mL) was degassed and purged with N₂ for 3 times, and then the mixture was stirred at 140° C. for 2 hrs under N₂ atmosphere at microwave. The mixture was added ice water (10.0 m L) at 25° C. and extracted with EtOAc (10.0 mL×3). The combined organic layers were washed with brine (10.0 mL), dried over Na₂SO₄, filtered and concentrated under reduced pressure to give a residue. The residue was purified by flash silica gel chromatography (ISCO®; 4 g SepaFlash® Silica Flash Column, Eluent of 0-26% Ethyl acetate/Petroleum ether gradient @ 100 mL/min) to give compound 40A (150 mg, 216 umol, 32.3% yield, 59.2% purity) was obtained as a white solid.

LCMS: Rt=0.570 min, MS=412.2, M+H⁺.

### Procedure for preparation of 1,1,1-trifluoro-3-hydroxypropan-2-yl 4-(3-cyclopropyl-7-fluoro-4,5-dihydropyrazolo[1,5-a]quinolin-2-yl)piperidine-1-carboxylate (40)

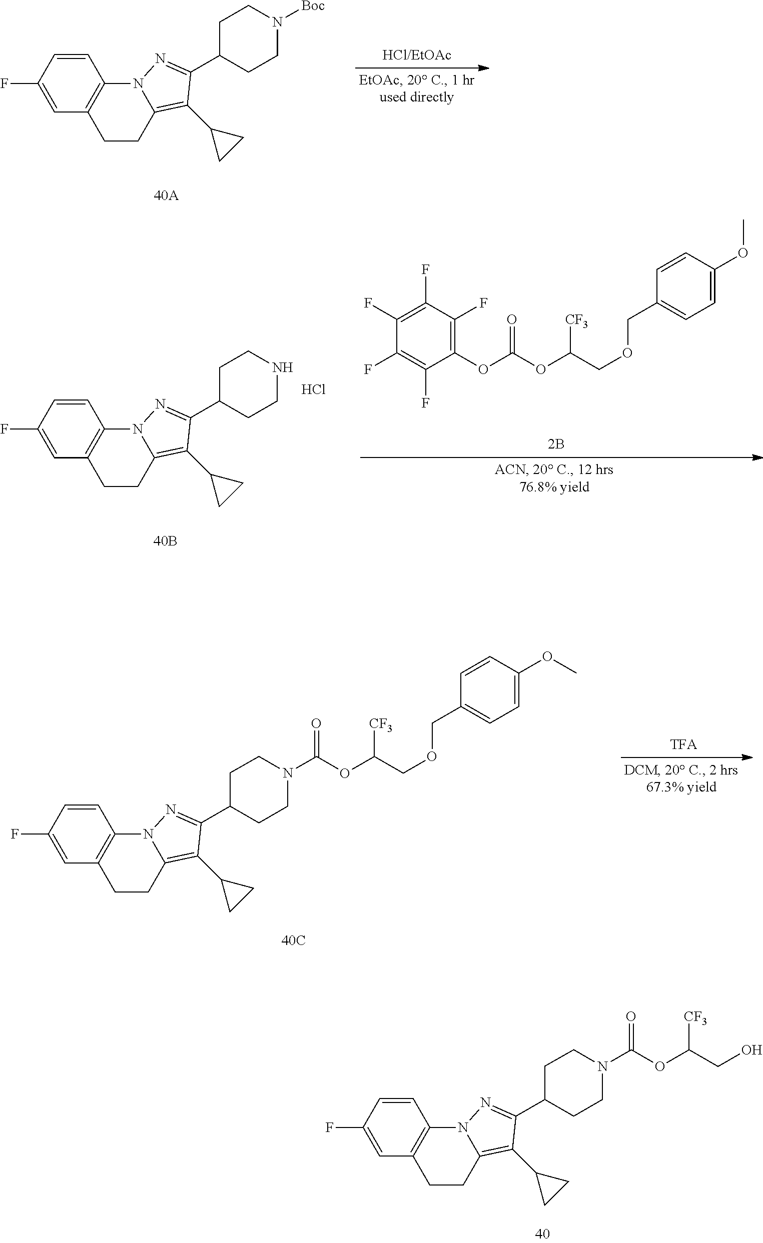 [SMILES: CC(C)(C)OC(=O)N1CCC(c2nn3c(c2C2CC2)CCc2cc(F)ccc2-3)CC1 ; Fc1ccc2c(c1)CCc1c(C3CC3)c(C3CCNCC3)nn1-2 ; Cl ; COc1ccc(COCC(OC(=O)Oc2c(F)c(F)c(F)c(F)c2F)C(F)(F)F)cc1 ; COc1ccc(COCC(OC(=O)N2CCC(c3nn4c(c3C3CC3)CCc3cc(F)ccc3-4)CC2)C(F)(F)F)cc1 ; O=C(OC(CO)C(F)(F)F)N1CCC(c2nn3c(c2C2CC2)CCc2cc(F)ccc2-3)CC1]

The compound was prepared according to the same synthesis step of example 2 starting from 5A to afford compound 40 (160 mg, 332 umol, 67.3% yield, 97.1% purity) was obtained as a white solid.

LCMS: Rt=0.514 min, MS=468.2, M+H⁺.

¹HNMR: (400 MHz, CDCl₃): δ 7.74-7.77 (m, 1H), 6.91-6.97 (m, 2H), 5.26-5.30 (m, 1H), 4.22-4.25 (m, 2H), 4.00-4.01 (m, 1H), 3.97-4.00 (m, 1H), 3.06-3.08 (m, 3H), 2.89-2.97 (m, 4H), 1.93-1.98 (m, 5H), 1.51-1.53 (m, 1H), 0.86-0.88 (m, 2H), 0.48-0.50 (m, 2H).

### Biological Study and Testing

### Example 41: In Vitro Enzyme Inhibition Assay

Monoacylglycerol Lipase Inhibitor Screening Assay provides a convenient method for screening human MAGL inhibitors. MAGL hydrolyzes 4-nitrophenylacetate resulting in a yellow product, 4-nitrophenol, with an absorbance of 405-412 nm. (Cayman Chemical, Item No. 705192)

### (1) Experimental Procedure

### Reagent Preparation

### 1. MAGL Assay Buffer (10×)

Dilute 3 ml of MAGL Assay Buffer (10×) concentrate with 27 ml of ultrapure water. This final 1× Assay Buffer (10 mM Tris-HCl, pH 7.2, containing 1 mM EDTA) should be used in the assay and for the dilution of MAGL enzyme, substrate, and MAGL inhibitor. The 1× Assay Buffer is stable for at least six months if stored at −20° C.

### 2. MAGL Substrate

The vial contains 0.5 ml of 17 mM ethanolic solution of 4-nitrophenylacetate. Mix 150 μL of this solution with 450 μl of 1× Assay Buffer prior to testing. This is enough substrate to assay 50 wells. Dilute additional substrate if using the entire plate.

### 3. MAGL (Human, Recombinant)

This vial contains 100 μl of a solution of human recombinant MAGL (monoacylglycerol lipase). The thawed enzyme should be stored on ice. Dilute 30 μl of enzyme with 570 μl of 1×MAGL Assay Buffer. This is enough enzyme to assay 50 wells. Dilute additional enzyme if using the entire plate. The diluted enzyme is stable for four hours on ice.

### 4. MAGL Inhibitor Assay Reagent

This vial contains 20 nmol of inhibitor. Resuspend in 125 μl of DMSO, then add 125 μl of 1× Assay Buffer and vortex to make an 80 μM stock. The addition of 10 μl to the assay yields a final concentration of 4.4 μM inhibitor in the well. ABX-1431 Inhibitor Assay Reagent can be used as a positive control in the assay.

### Performing the Assay

1). 100% Initial Activity Wells—add 150 μl of 1× Assay Buffer, 10 μl of MAGL enzyme, and 10 μl of solvent (same solvent used to dissolve the inhibitor) to three wells.

2). Background Wells—add 160 μl of 1× Assay Buffer and 10 μl of solvent (same solvent used to dissolve the inhibitor) to three wells.

3). Inhibitor/Positive Control Wells—add 150 μl of 1× Assay Buffer, 10 μl of MAGL enzyme, and 10 μl of inhibitor* or positive control inhibitor to the inhibitor wells.

4). Mix the contents of the wells by pipetting gently up and down and incubate for 15 minutes at room temperature. The incubation time for different inhibitors may vary and will need to be optimized by the user.

5). Initiate the reactions by adding 10 μl of MAGL Substrate to all the wells being used. Carefully shake the 96-well plate for 10 seconds to mix and cover with the plate cover. Incubate for ten minutes at room temperature.

6). Remove the plate cover and read the absorbance at 405-415 nm. (Tecan Spark 10M Multimode Plate Reader)

### Calculations

1). Determine the average absorbance of each sample.

2). Subtract the average absorbance of the background wells from the average absorbance of the 100% initial activity and the inhibitor wells.

3). Determine the percent inhibition or percent activity for each inhibitor using one of the following equations:

$$\% inhibition = [ \frac{\begin{matrix}( corrected 100 \% initial activity - \\ corrected inhibitor activity )\end{matrix}}{corrected 100 \% initial activity} ] \times 100 \\ \% activity = [ \frac{( corrected inhibitor activity )}{corrected 100 \% initial activity} ] \times 100$$

4). Graph the percent inhibition or percent initial activity as a function of the inhibitor concentration to determine the IC₅₀ value (concentration at which there is 50% inhibition).

### (2) Test Results

TABLE 1
In vitro MAGL enzyme inhibition activity of compounds

| Name | IC₅₀ (nM) |
|---|---|
| Compound 1 | <0.1 |
| Compound 2 | 16.05 |
| Compound 5 | 37.11 |
| Compound 6 | 16.71 |
| Compound 7 | 89.8 |
| Compound 13 | 9.1 |
| Compound 17 | 126.2 |
| Compound 18 | 78.35 |
| Compound 21 | 151.8 |
| Compound 26 | 83.4 |
| Compound 28 | 25.13 |
| Compound 31 | 32.67 |
| Compound 34 | 12.46 |
| Compound 35 | 13.08 |
| Compound 38 | 13.42 |
| Compound 40 | 18.79 |
| ABX-1431 | 71.08 |

### Example 42: Formalin-Induced Pain Model

A diluted formalin solution is injected subcutaneously into the hind paw of rats using a microsyringe, creating a continuous noxious stimulus that induces spontaneous pain behaviors in the animals. The experiment can record the number of lifts and licks of the rat's foot through an autonomous movement analyzer to assess the degree of pain. This experiment uses a 2% formalin model in SD rats and tests the analgesic activity of compounds by oral administration. After injecting 2% formalin, the number of movements (lifts and licks of the injected foot) from 0-60 min after injecting the formalin solution is recorded and analyzed.

The Maximum Possible Effect (MPE %) is calculated as a percentage:

MPE % = ((Vehicle-Sham) − (Treatment Group-Sham)) /
(Vehicle-Sham) × 100%

Note: \textcircled{1} MPE % for each group is calculated with sham group MPE=100% and vehicle group MPE=0% as standards. \textcircled{2} Movement counts of the sham group are subtracted to exclude the impact of background noise on the experiment. \textcircled{2} “Sham” represents the average value of the sham group; “Vehicle-Sham” represents the average value of the vehicle group minus the sham group.

Results are presented as mean values with standard error. Data from each group are analyzed and graphed using GraphPad Prism 8 software. Statistical analysis is conducted using one-way ANOVA to determine statistical differences between groups, with t-tests used for comparisons between two groups. A p-value <0.05 is considered statistically significant. The results are shown in the following table.

TABLE 2
Analgesic activity of compounds in the formalin model

| | Dosage | Administration | | Experimental |
|---|---|---|---|---|
| Compound Name | (mg/kg) | Method | MPE % | Results |
| Compound 1 | 3 | p.o. | 68 | effective |
| Compound 2 | 3 | p.o. | 45 | effective |
| Compound 3 | 3 | p.o. | 61 | effective |
| Compound 4 | 3 | p.o. | 51 | effective |
| Compound 5 | 3 | p.o. | 44 | effective |
| Compound 6 | 3 | p.o. | 44 | effective |
| Compound 7 | 3 | p.o. | 56 | effective |
| Compound 12 | 3 | p.o. | 64 | effective |
| Compound 13 | 3 | p.o. | 49 | effective |
| Compound 24 | 3 | p.o. | 50 | effective |
| Compound 40 | 3 | p.o. | 41 | effective |
| ABX-1431 | 3 | p.o. | 33 | effective |

What is claimed is:

1. A compound of formula (I), or its stereoisomer, pharmaceutically acceptable salt, solvate, deuterated derivative, metabolite, or prodrug; 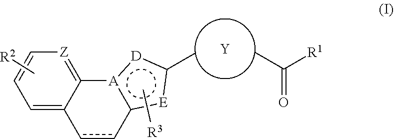
wherein,
R¹ is selected from the following: 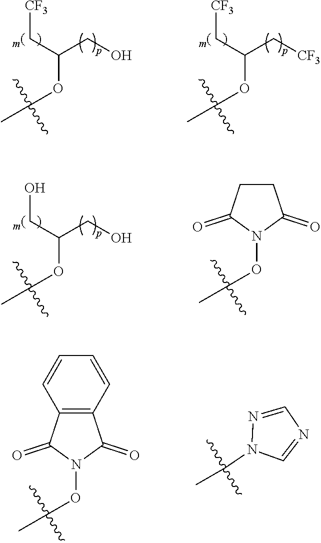
wherein, m=0, 1, 2; p=0, 1, 2;
Y ring is selected from the following: 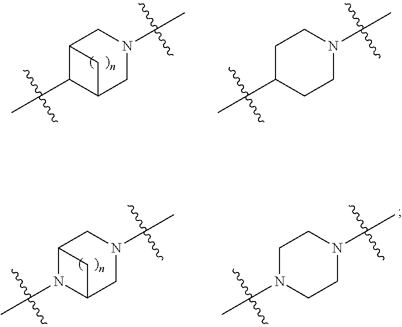
wherein, n=0, 1, 2;
A, D, and E are independently selected from C or N;
when both A and D are N, the two N atoms are connected by a single bond;
Z is C or N;
R² is H, deuterium, or halogen;
R³ is selected from the group consisting of H, deuterium, halogen, C1-C6 alkyl, substituted C1-C6 alkyl, C3-C6 cycloalkyl, substituted C3-C6 cycloalkyl, amino, nitrogen-containing alkyl, halogenated alkyl, hydroxyl, aryl, and substituted aryl;
wherein the substituted C1-C6 alkyl, substituted C3-C6 cycloalkyl, substituted aryl are substituted with 1-3 substituents independently selected from the following groups: halogen, C1-C3 alkyl, C1-C3 alkoxy, halogenated C1-C3 alkyl, heteroaryl, or heterocycle.

2. The compound of claim 1, wherein the compound is represented by formula (II), or its stereoisomers, pharmaceutically acceptable salts, solvates, deuterated derivatives, metabolites, or prodrugs; 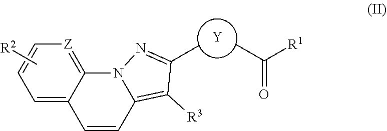
wherein,
R¹ is selected from the following: 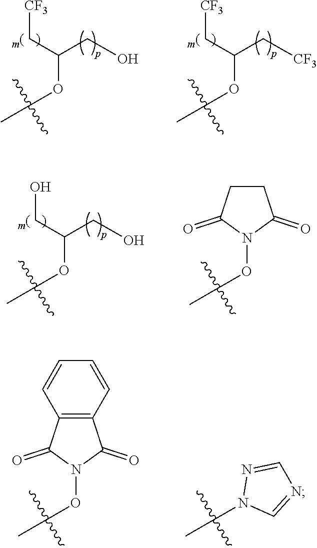
wherein, m=0, 1, 2; p=0, 1, 2;
Y ring is selected from the following: 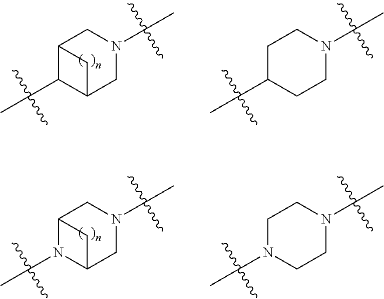
wherein, n=0, 1, 2;
Z is C or N;
R² is H, deuterium, or halogen;
R³ is selected from the group consisting of H, deuterium, halogen, C1-C6 alkyl, substituted C1-C6 alkyl, C3-C6 cycloalkyl, substituted C3-C6 cycloalkyl, amino, nitrogen-containing alkyl, halogenated alkyl, hydroxyl, aryl, and substituted aryl;
wherein the substituted C1-C6 alkyl, substituted C3-C6 cycloalkyl, and substituted aryl are substituted with one to three substituents independently selected from the group consisting of halogen, C1-C3 alkyl, C1-C3 alkoxy, halogenated C1-C3 alkyl, heteroaryl, and heterocycle.

3. The compound of claim 2, wherein the compound is represented by formulas (II-1), (II-2), or (II-3), or its stereoisomers, pharmaceutically acceptable salts, solvates, deuterated derivatives, metabolites, or prodrugs; 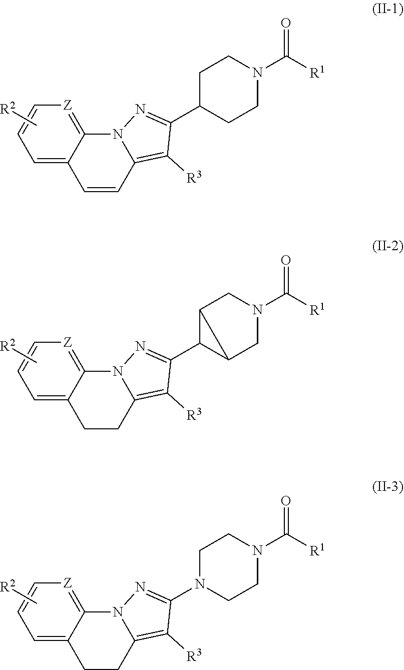
wherein,
R¹ is selected from the following: 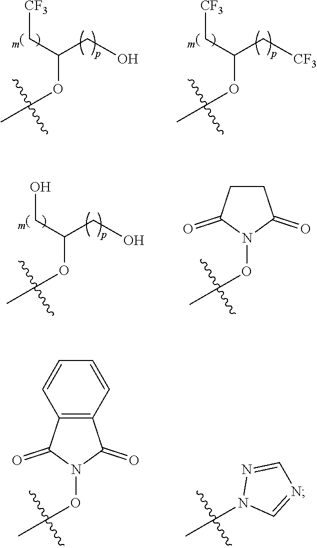
wherein, m=0, 1; p=0, 1;
Z is selected from C or N;
R² is selected from the group consisting of H, deuterium, fluorine, chlorine, bromine, and iodine;
R³ is selected from the group consisting of H, deuterium, fluorine, chlorine, bromine, iodine, methyl, ethyl, propyl, isopropyl, butyl, tert-butyl, cyclopropyl, cyclobutyl, cyclopentyl, cyclohexyl, amino, methylamino, ethylamino, dimethylamino, diethylamino, 1,4-oxazinyl, piperazinyl, piperidinyl, trifluoromethyl, hydroxyl, phenyl, and pyridinyl; wherein the substituted C1-C6 alkyl, substituted C3-C6 cycloalkyl, substituted aryl being substituted by 1-3 substituents independently selected from the group consisting of fluorine, chlorine, bromine, methyl, ethyl, propyl, methoxy, ethoxy, trifluoromethyl, pyridine, piperidine, and piperazine.

4. The compound of claim 1, wherein the compound is represented by formula (III), or its stereoisomers, pharmaceutically acceptable salts, solvates, deuterated derivatives, metabolites, or prodrugs; 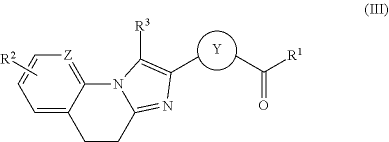
wherein,
R¹ is selected from the following: 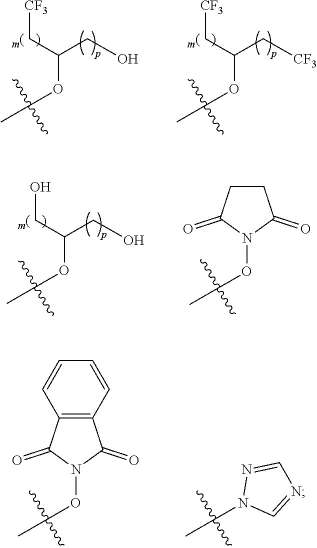
wherein, m=0, 1; p=0, 1;
Y ring is selected from the following: 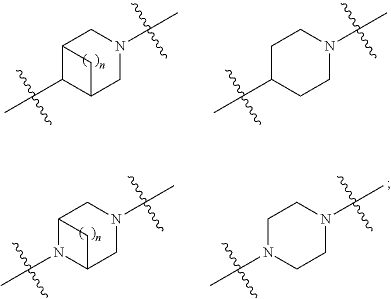
wherein, n=0;
Z is C or N;
R² is selected from the group consisting of H, deuterium, and halogens;
R³ is selected from the group consisting of H, deuterium, halogens, C1-C6 alkyl, substituted C1-C6 alkyl, C3-C6 cycloalkyl, substituted C3-C6 cycloalkyl, amino, nitrogen-containing alkyl, halogenated alkyl, hydroxyl, aryl, and substituted aryl; the substituted C1-C6 alkyl, substituted C3-C6 cycloalkyl, and substituted aryl are substituted by 1-3 substituents independently selected from the following groups: halogens, C1-C3 alkyl, C1-C3 alkoxy, halogenated C1-C3 alkyl, heteroaryl, heterocycle.

5. The compound of claim 4, wherein the compound is represented by formula (III-1) or (III-2), or its stereoisomers, pharmaceutically acceptable salts, solvates, deuterated derivatives, metabolites, or prodrugs; 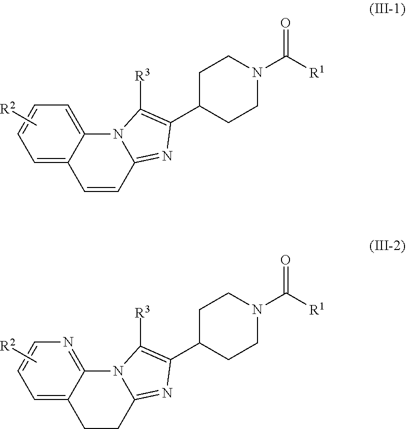
wherein,
R¹ is: 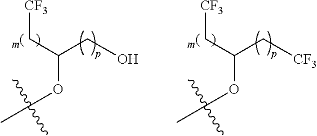
wherein, m=0, 1; p=0, 1;
R² is selected from the group consisting of H, deuterium, fluorine, chlorine, bromine, and iodine;
R³ is selected from the group consisting of H, deuterium, fluorine, chlorine, bromine, iodine, methyl, ethyl, propyl, isopropyl, butyl, tert-butyl, cyclopropyl, cyclobutyl, cyclopentyl, cyclohexyl, amino, methylamino, ethylamino, dimethylamino, diethylamino, 1,4-oxazinyl, piperazinyl, piperidinyl, trifluoromethyl, hydroxyl, phenyl, and pyridinyl; wherein the substituted C1-C6 alkyl, substituted C3-C6 cycloalkyl, substituted aryl are substituted by 1-3 substituents independently selected from the group consisting of fluorine, chlorine, bromine, methyl, ethyl, propyl, methoxy, ethoxy, trifluoromethyl, pyridine, piperidine, and piperazine.

6. The compound of claim 1, wherein the compound is represented by formula (IV), or its stereoisomers, pharmaceutically acceptable salts, solvates, deuterated derivatives, metabolites, or prodrugs; 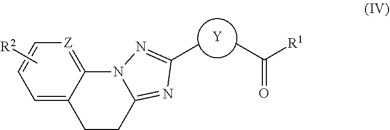
wherein,
R¹ is selected from the following: 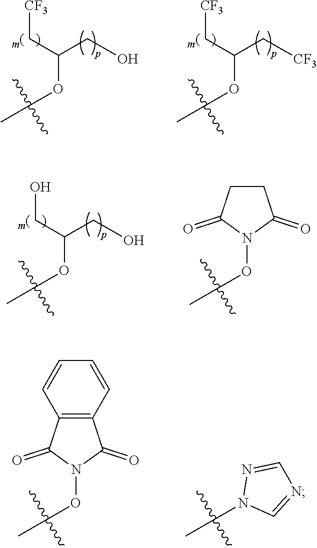
wherein, m=0, 1; p=0, 1;
Y ring is selected from the following: 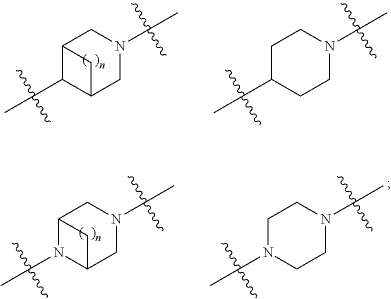
wherein, n=0, 1, 2;
R² is H, deuterium, or halogen.

7. The compound of claim 6, wherein the compound is represented by formula (IV-1) or (IV-2), or its stereoisomers, pharmaceutically acceptable salts, solvates, deuterated derivatives, metabolites, or prodrugs; 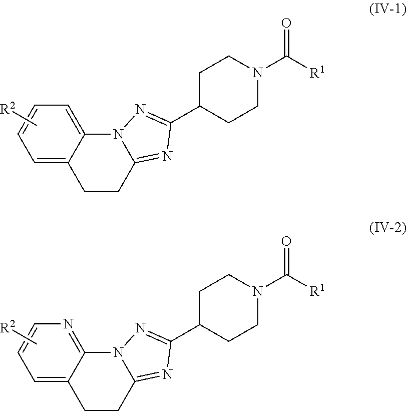
wherein,
R¹ is: 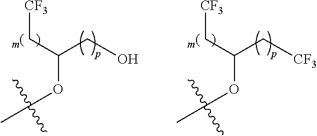
wherein, m=0, 1; p=0, 1;
R² is selected from the group consisting of H, deuterium, fluorine, chlorine, bromine, and iodine.

8. The compound of claim 1, wherein the compound of formula (I) is selected from the compounds or any pharmaceutically acceptable salt thereof consisting of:

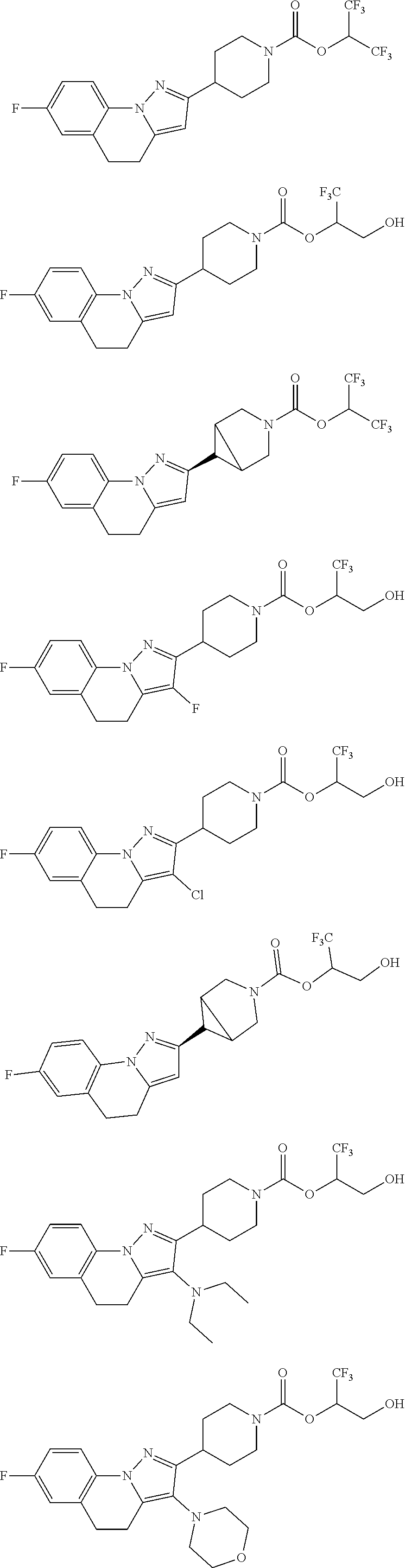

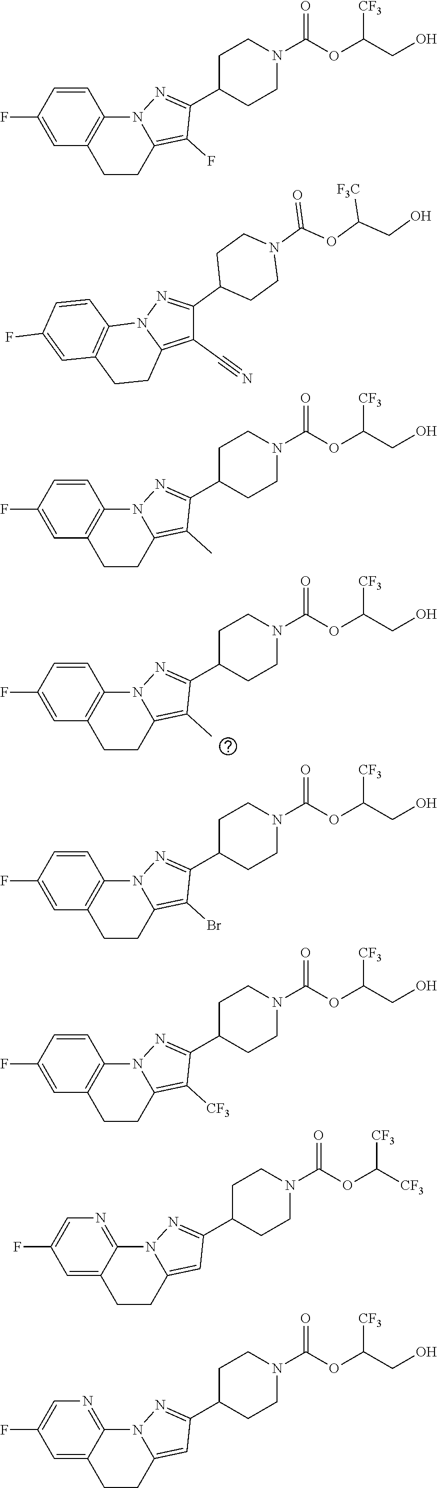

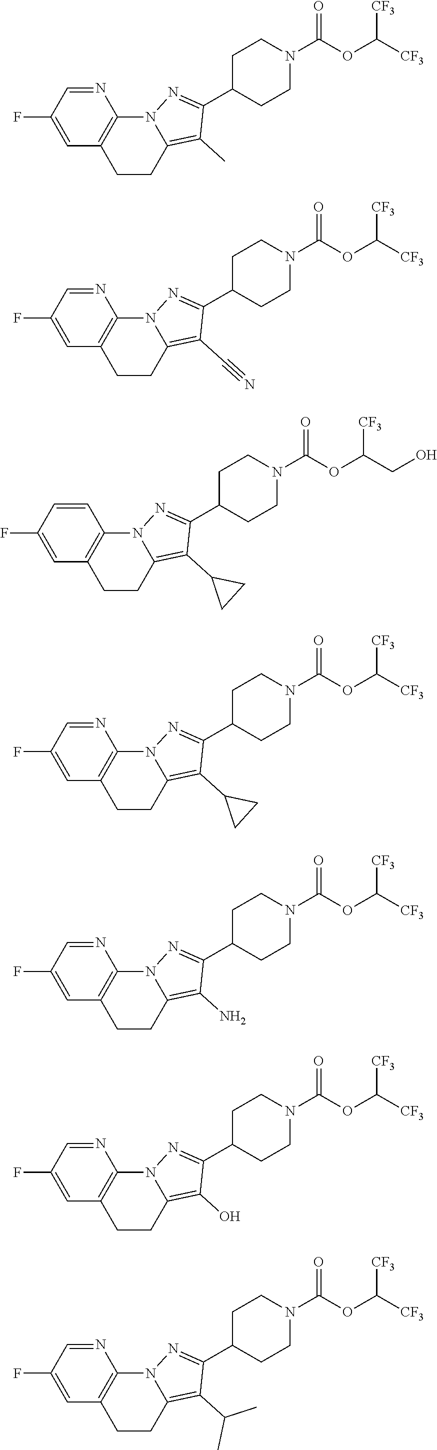

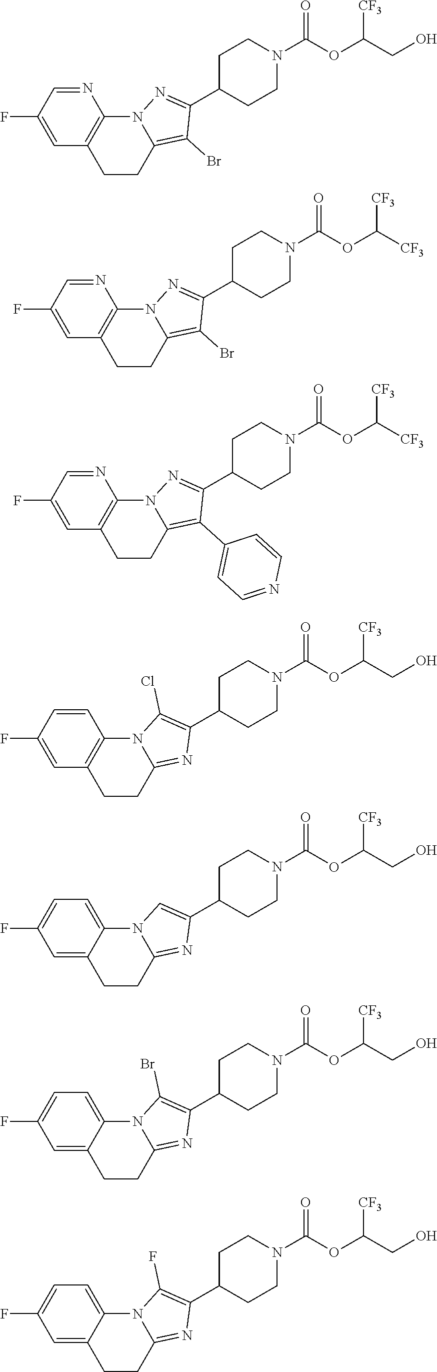

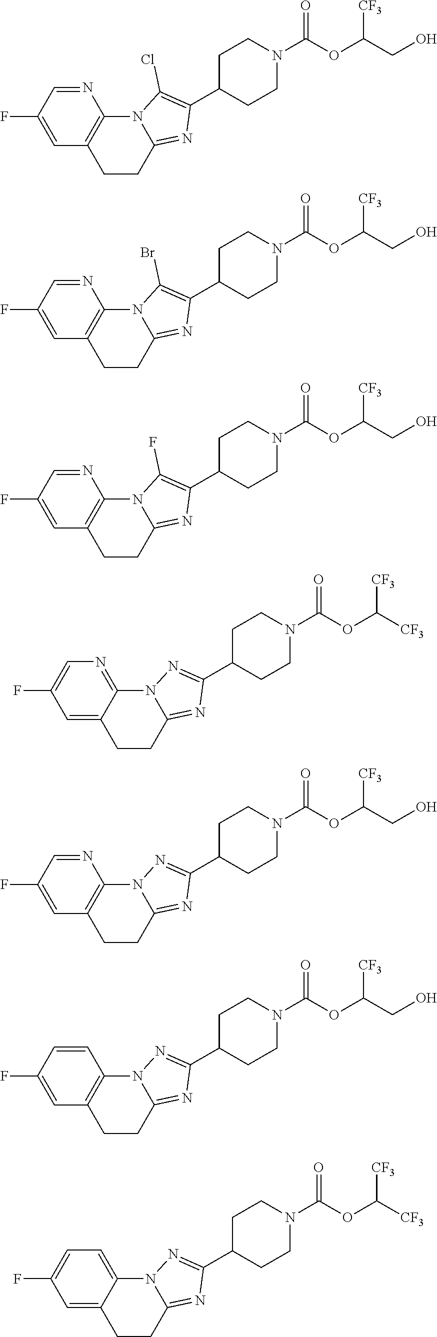

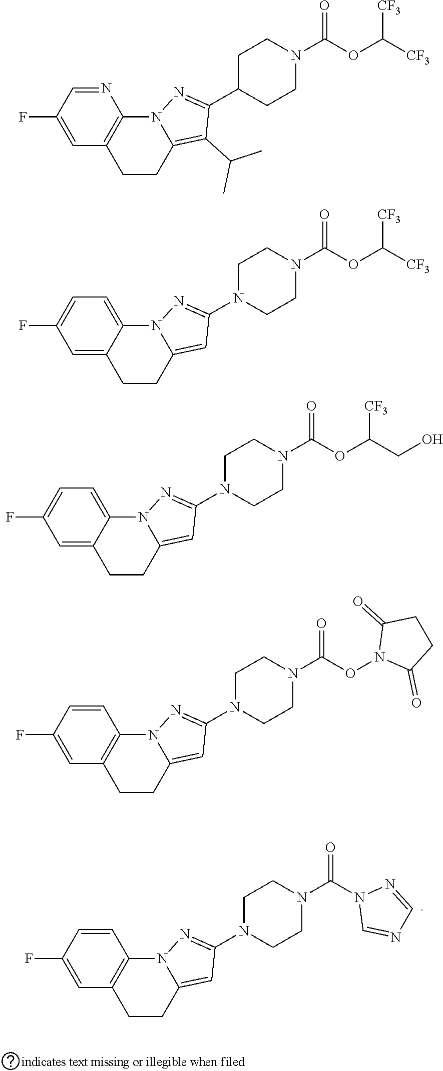 [SMILES: CC(C)c1c(C2CCN(C(=O)OC(C(F)(F)F)C(F)(F)F)CC2)nn2c1CCc1cc(F)cnc1-2 ; O=C(OC(C(F)(F)F)C(F)(F)F)N1CCN(c2cc3n(n2)-c2ccc(F)cc2CC3)CC1 ; O=C(OC(CO)C(F)(F)F)N1CCN(c2cc3n(n2)-c2ccc(F)cc2CC3)CC1 ; O=C(ON1C(=O)CCC1=O)N1CCN(c2cc3n(n2)-c2ccc(F)cc2CC3)CC1 ; O=C(N1CCN(c2cc3n(n2)-c2ccc(F)cc2CC3)CC1)n1cncn1]

9. A pharmaceutical composition comprising a compound or its stereoisomers, pharmaceutically acceptable salts, solvates, deuterated derivatives, metabolites, or prodrugs as described in claim 1, and a pharmaceutically acceptable carrier or excipient.

10. A method of inhibiting enzymatic activity against monoacylglycerol lipase (MAGL), the method comprising administering the compound or composition of claim 1.

11. A method of treating and/or preventing diseases or disorders related to monoacylglycerol lipase (MAGL), the method comprising administering the compound or composition of claim 1.

12. The method of claim 11, wherein the diseases or disorders related to MAGL comprise neuroinflammation, neurodegenerative diseases, pain, cancer, and/or psychiatric disorders.

13. The method of claim 11, wherein the diseases or disorders related to MAGL comprise multiple sclerosis, Alzheimer's disease, Parkinson's disease, amyotrophic lateral sclerosis, traumatic brain injury, neurotoxicity, stroke, epilepsy, anxiety, migraines, depression, hepatocellular carcinoma, colorectal cancer lesions, ovarian cancer, neuropathic pain, chemotherapy-induced neuropathy, acute pain, chronic pain, and/or spasticity conditions related to pain.
